(19) United States
(12) Patent Application Publication
(10) Pub. No.: US 2024/0382608 A1
(43) Pub. Date: Nov. 21, 2024

(54) NOVEL LIGAND-DRUG CONJUGATE OF CAMPTOTHECIN ANALOGS, INTERMEDIATES, PHARMACEUTICAL COMPOSITION AND APPLICATION THEREOF

(71) Applicant: SHANGHAI MICURX PHARMACEUTICAL CO., LTD., Shanghai (CN)

(72) Inventors: Hongyu YANG, Shanghai (CN); Xinghai WANG, Shanghai (CN)

(73) Assignee: SHANGHAI MICURX PHARMACEUTICAL CO., LTD., Shanghai (CN)

(21) Appl. No.: 18/661,518

(22) Filed: May 10, 2024

(30) Foreign Application Priority Data
May 10, 2023 (WO) PCT/CN2023/093278

(51) Int. Cl. A61K 47/68; A61P 35/00

(52) U.S. Cl. CPC A61K 47/68037; A61K 47/6851; A61K 47/6889; A61P 35/00

(57) ABSTRACT

The present disclosure relates to conjugates of novel camptothecin analogs with a cell binding molecule of formula (I). It also provides methods of making the conjugates of camptothecin analogs to a cell-binding agent, as well as methods of application the conjugates in targeted treating of cancer.

### FIELD

Provided are conjugates of camptothecin analogs with a cell-surface receptor-biding molecule for targeted therapy, as well as pharmaceutical compositions comprising such a conjugate. Also provided are camptothecin analogs, intermediates of conjugates of camptothecin analogs, preparation methods therefor. Also provided are uses of a camptothecin analog, a conjugate of a camptothecin analog, a pharmaceutical composition comprising a camptothecin analog, and a pharmaceutical composition comprising a conjugate of the camptothecin analog to a cell-binding molecule for targeted treatment of cancer.

### BACKGROUND

Cancer is a leading cause of death worldwide. Surgery, chemotherapy, radiotherapy and targeted therapy are the standard of care therapies. Althrough chemotherapy is widely applied, the use of most chemotherapies is limited by undesired side effects, mostly through action on cells beyond the tumor and its environment, resulting in systemic toxicity and a narrow therapeutic window. The discovery of the unique composition of cancer cell surfaces combined with the understanding of the strong and selective interaction between antibodies and cell-surface antigens opened the way to exploit antibodies as targeted delivery agents for chemotherapies, including highly toxic drugs (Drago, J. Z. et al., Nat. Rev. Clin. Oncol. 2021; Khongorzul, P. et al., Mol. Cancer Res. 2020, 18, 3-19; Joubert, N. et al.; The Last Decade. Pharmaceuticals 2020, 13, 245; Ravi V. J. Chari et al., Angew. Chem. Int. Ed. 2014, 53, 3796 3827). The resulting molecular entities, also known as antibody-drug conjugates (ADC) consist of three main parts: the antibody responsible for the selective recognition of the cancer cell surface antigen capable of internalizing the ADC, the drug payload responsible for killing the cancer cell once released inside it, and the linker connecting the antibody and payload parts.

Antibody-drug conjugates (ADCs), combining the selective targeting of tumor cells through antigen-directed recognition and potent cell-killing by cytotoxic payloads, have emerged in recent years as an efficient therapeutic approach for the treatment of various cancers (Nature review Drug Discovery, 2013, 12, 329-332). The first ADC (Mylotarg) was approved in 2000 (and following withdrawal in 2010, reapproved in 2017), and the second ADC (Adcetris) received accelerated approval in 2011 and full approval in 2015. The third (Kadcyla) and fourth (Besponsa) ADCs were approved in 2013 and 2017, respectively. Kadcyla is the first ADC approved for solid tumor treatment. Since 2019, more than ten ADCs have been approved, and there are more than 100 ADCs in clinical development.

It has been known that the payload-linker component in the ADC critically contribute to ADC homogeneity, circulation stability, pharmacokinetic profiles, tolerability and overall treatment efficacy (Acchionea, M. et al., mAbs. 2012, 4, 362; Zhao, R. Y. et al., J. Med. Chem. 2011, 54, 3606). Despite extensive study to improve these profiles, most payload used so far include DNA damaging agents (such as calicheamicins, PBD, and duocarmysins), microtubule disrupting agents (such as maytansins, like DM1 or DM4; auruistatins like MMAE or MMAF; tubulysins) and topoisomerase inhibitors (such as camptothecins like Dxd or SN-38). (Leung, D., et al., Antibodies (Basel).2020, 9, 2; Khongorzul, P., et al., Mol. Cancer. Res., 2020, 18, 3; Chau, C. H., et al., Lancet. 2019, 394, 793.)

Among these payloads, the camptothecins have proved a promising choice with a wider therapeutic index than many other payloads for ADC construction. Two of the approved ADCs, Enhertu and Trodelvy, which employ the camptothecin payloads Dxd and SN-38 respectively, have demonstrated significant clinical benefits (Progression-Free-Survival, PFS and Overall-Survival, O⁵) for solid tumors in many clinical trials (Pondé, N., et al., Curr Treat Options Oncol. 2019, 20, 37; Kaplon, H., et al., Mabs. 2020, 12, 1703531). By interacting with DNA enzyme topoisomerase I and then accumulating reversible enzyme-camptothecin-DNA ternary complexes, camptothecin can induce cell death.

Camptothecin is a potent antitumor antibiotic isolated in 1958 from extracts of Camptotheca acuminata, wherein the plant has been extensively used in traditional Chinese medicine for hundreds of years. Many camptothecin analogs have been disclosed, such as those shown below:

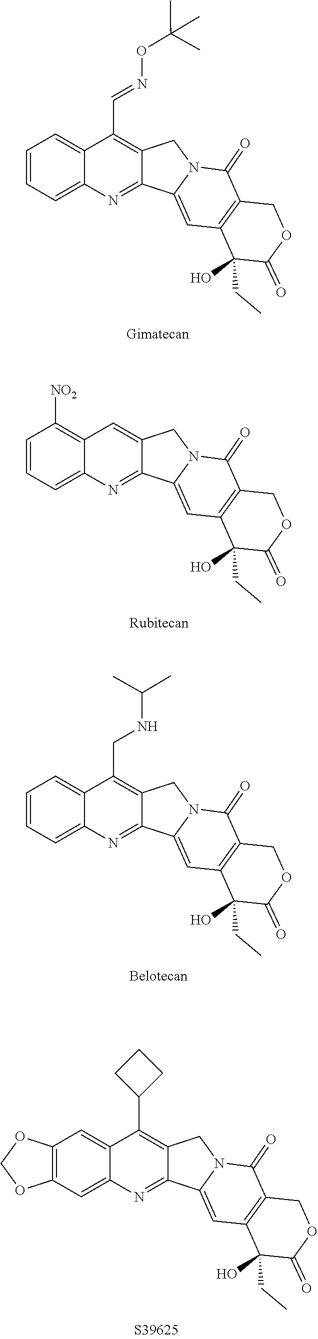 [SMILES: CC[C@@]1(O)C(=O)OCc2c1cc1n(c2=O)Cc2c-1nc1ccccc1c2/C=N/OC(C)(C)C ; CC[C@@]1(O)C(=O)OCc2c1cc1n(c2=O)Cc2cc3c([N+](=O)[O-])cccc3nc2-1 ; CC[C@@]1(O)C(=O)OCc2c1cc1n(c2=O)Cc2c-1nc1ccccc1c2CNC(C)C ; CC[C@@]1(O)C(=O)OCc2c1cc1n(c2=O)Cc2c-1nc1cc3c(cc1c2C1CCC1)OCO3]

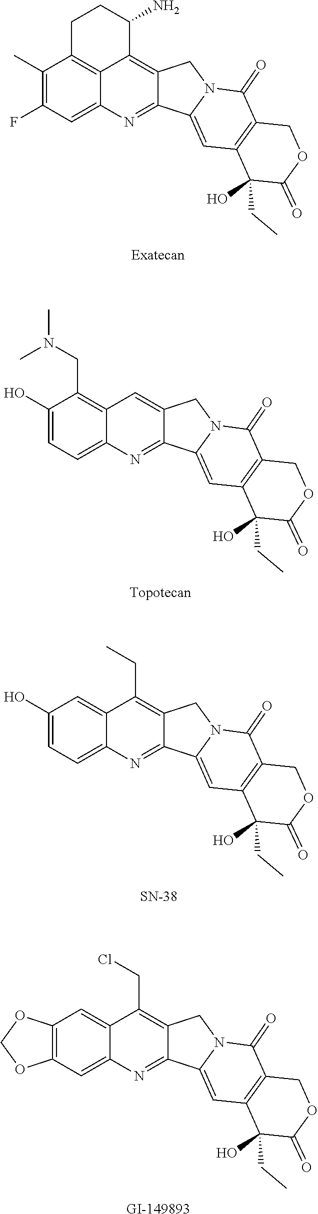 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2cc3c(CN(C)C)c(O)ccc3nc2-1 ; CCc1c2c(nc3ccc(O)cc13)-c1cc3c(c(=O)n1C2)COC(=O)[C@]3(O)CC ; "SN-38" ; CC[C@@]1(O)C(=O)OCc2c1cc1n(c2=O)Cc2c-1nc1cc3c(cc1c2CCl)OCO3 ; "GI-149893"]

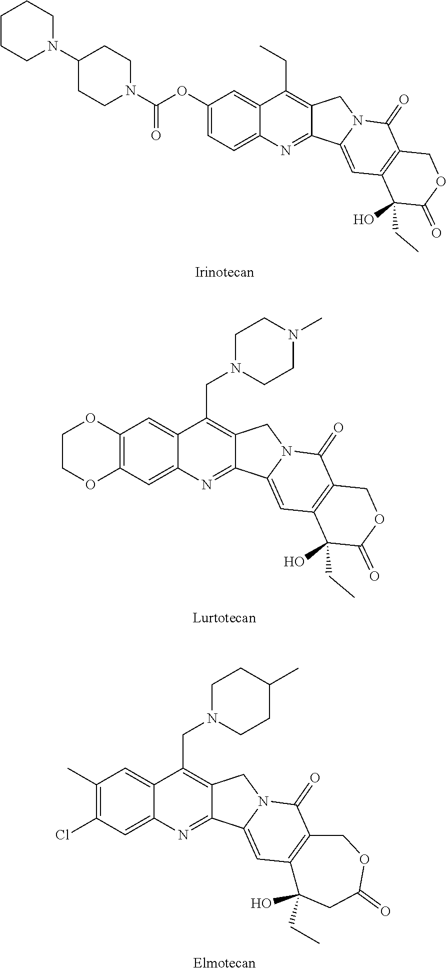 [SMILES: CCc1c2c(nc3ccc(OC(=O)N4CCC(N5CCCCC5)CC4)cc13)-c1cc3c(c(=O)n1C2)COC(=O)[C@]3(O)CC ; "Irinotecan" ; CC[C@@]1(O)C(=O)OCc2c1cc1n(c2=O)Cc2c-1nc1cc3c(cc1c2CN1CCN(C)CC1)OCCO3 ; "Lurtotecan" ; CC[C@@]1(O)CC(=O)OCc2c1cc1n(c2=O)Cc2c-1nc1cc(Cl)c(C)cc1c2CN1CCC(C)CC1 ; "Elmotecan"]

Camptothecin and most of its analogs are extremely insoluble in physiological buffer and have demonstrated high adverse drug reaction in the preliminary clinical trial since 1970s. The low solubility of camptothecin can cause their ADC conjugates to aggregate (Burke, P., et al. Bioconjugate Chem. 2009, 20, 6, 1242) which is problematic for scale-up manufacturing production and may cause systematic side-effects resulting from aggregation. So far the US FDA has only approved three water-soluble camptothecin analogs: topotecan, irinotecan and belotecan in cancer therapy (Palakurthi, S., Expert Opin Drug Deliv. 2015, 12(12), 1911). Most of the camptothecin payloads employed to date for ADC development suffer from low solubility, which further limits the Drug-to-Antibody Ratio and results in low potency.

Provided herein is a series of ligand-drug conjugates of camptothecin analogs.

### SUMMARY

Provided are conjugates of camptothecin analogs linked to a cell-binding molecule, camptothecin analog-linker compounds and camptothecin analogs, methods to prepare and to use them, and intermediates useful in the preparation thereof. The camptothecin analog conjugates of the present disclosure are stable in circulation, as well as providing high cytotoxicity once the free camptothecin analog or a metabolite of the camptothecin analog-linker compound is released from the conjugate in the vicinity of or within disordered cells.

In a general aspect: These compounds or pharmaceutically acceptable salt or solvate thereof have the general formula I:

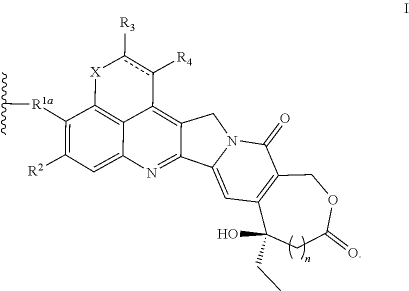

Aspect 1: The present disclosure provides a ligand-drug conjugate or a pharmaceutically acceptable salt or solvate thereof, wherein the ligand-drug conjugate comprises a drug unit D¹ of formula I:

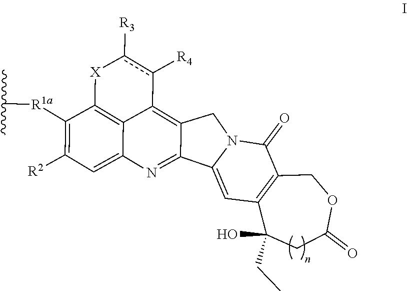

Wherein:
X is either absent, or selected from —O—, —S—, —S(O₂)—, or —S(O)—;
R^{1a} is —NH—R^{1b}—O—, —NH—, or —O—; optionally, R^{1a} is —NH— or —O—;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R² is selected from H, halo, hydroxy, cyano, nitro, C₃-C₈ cycloalkyl, C₁-C₈ alkyl, C₁-C₈ alkoxy, R⁵—(C₁-C₈ alkylene)-, amino, (C₁-C₈ alkyl)NHC(O)O—, (C₃-C₈ cycloalkyl)NHC(O)O—, (C₁-C₈ alkyl)NH—, azetidin-1-yl, pyrrolidin-1-yl, piperidin-1-yl, azepane-1-yl, (C₁-C₈ alkyl)C(O)O—, or (C₁-C₈ alkyl)C(O)NH—; wherein azetidin-1-yl, pyrrolidin-1-yl, piperidin-1-yl, azepane-1-yl, C₁-C₈ alkoxy, each “C₃-C₈ cycloalkyl”, and each “C₁-C₈ alkyl” are independently optionally substituted with one to four R⁶;
R³ and R⁴ are independently selected from H, halo, hydroxy, cyano, C₁-C₈ alkyl, C₁-C₈ alkoxy, C₃-C₈ cycloalkyl, aryl, heteroaryl, or amino; wherein C₁-C₈ alkyl, C₁-C₈ alkoxy, C₃-C₈ cycloalkyl, aryl, and heteroaryl are independently optionally substituted with one to four R⁶;
R⁵ is selected from C₁-C₈ alkoxy, C₃-C₈ cycloalkyl, aryl, or heteroaryl; wherein C₁-C₈ alkoxy, C₃-C₈ cycloalkyl, aryl, and heteroaryl are independently optionally substituted with one to four R⁶;
R⁶ at each occurrence is independently selected from halo, hydroxy, cyano, amino, C₁-C₈ alkyl, C₃-C₈ cycloalkyl, aryl, or heteroaryl;
and wherein a line with a dotted line represents either a single bond or a double bond;
n is an integer from 0 or 1.

In some embodiments of Aspect 1, including of the formula (I), wherein:
X is selected from —O—, —S—, —S(O₂)—, or —S(O)—;
R^{1a} is —NH—R^{1b}—O—, —NH—, or —O—; optionally, R^{1a} is —NH— or —O—;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R² is selected from H, halo, hydroxy, cyano, nitro, C₃-C₆ cycloalkyl, C₁-C₆ alkyl, C₁-C₆ alkoxy, R⁵—(C₁-C₃ alkylene)-, amino; wherein C₁-C₆ alkoxy, C₃-C₆ cycloalkyl, and C₁-C₆ alkyl are independently optionally substituted with one to four R⁶;
R³ and R⁴ are independently selected from H, halo, hydroxy, cyano, C₁-C₆ alkyl, C₁-C₆ alkoxy, or C₃-C₆ cycloalkyl; wherein C₁-C₆ alkyl, C₁-C₆ alkoxy, and C₃-C₆ cycloalkyl are independently optionally substituted with one to four R⁶;
R⁵ is C₁-C₆ alkoxy or C₃-C₆ cycloalkyl; wherein C₁-C₆ alkoxy and C₃-C₆ cycloalkyl are independently optionally substituted with one to four R⁶;
R⁶ at each occurrence is independently selected from halo, hydroxy, cyano, amino, C₁-C₃ alkyl, or C₃-C₆ cycloalkyl;
and wherein a line with a dotted line represents either a single bond or a double bond;
n is 0 or 1.

In some embodiments of Aspect 1, wherein D¹ is represented by Formula II:

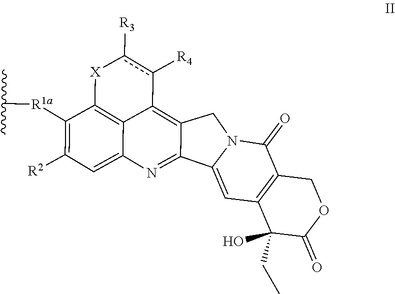

wherein: X is —O— or —S—; R^{1a} is —NH—R^{1b}—O—, —NH—, or —O—; optionally, R^{1a} is —NH— or —O—; R^{1b}, R², R³, R⁴, and a line with a dotted line are as defined as in formula (I) and any embodiments thereof.

In some embodiments of Aspect 1, including of any of the formula (I) and (II) and any embodiments thereof: wherein R², R³ and R⁴ are independently selected from H, halo, C₁-C₃ alkyl, or C₁-C₃ alkoxy and wherein C₁-C₃ alkoxy and C₁-C₃ alkyl are independently optionally substituted with one to four halo.

In some embodiments of Aspect 1, including of any of the formula (I) and (II) and any embodiments thereof: wherein a line with a dotted line represents a double bond.

In some embodiments of Aspect 1, including of any of the formula (I) and (II) and any embodiments thereof: wherein a line with a dotted line represents a single bond.

In some embodiments of Aspect 1, including of any of the formula (I) and (II) and any embodiments thereof: wherein R², R³ and R⁴ are independently selected from H, halo, C₁-C₃ alkyl, or C₁-C₃ alkoxy and wherein C₁-C₃ alkoxy and C₁-C₃ alkyl are independently optionally substituted with one to four halo; a line with a dotted line represents a double bond.

In some embodiments of Aspect 1, the D¹ includes, but is not limited to:

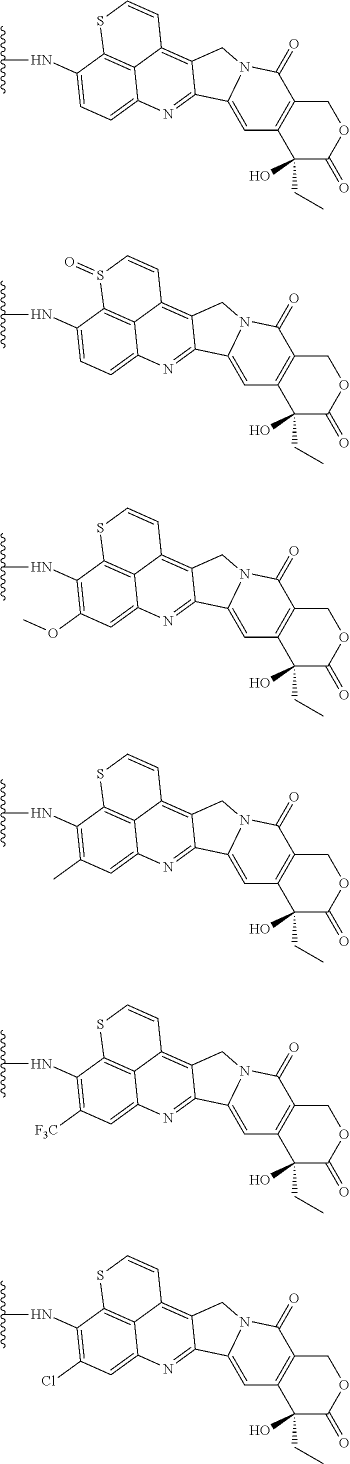 [SMILES: *Nc1ccc2nc3c(c4c2c1SC=C4)Cn1c-3cc2c(c1=O)COC(=O)[C@]2(O)CC ; *Nc1ccc2nc3c(c4c2c1S(=O)C=C4)Cn1c-3cc2c(c1=O)COC(=O)[C@]2(O)CC ; *Nc1c(OC)cc2nc3c(c4c2c1SC=C4)Cn1c-3cc2c(c1=O)COC(=O)[C@]2(O)CC ; *Nc1c(C)cc2nc3c(c4c2c1SC=C4)Cn1c-3cc2c(c1=O)COC(=O)[C@]2(O)CC ; *Nc1c(C(F)(F)F)cc2nc3c(c4c2c1SC=C4)Cn1c-3cc2c(c1=O)COC(=O)[C@]2(O)CC ; *Nc1c(Cl)cc2nc3c(c4c2c1SC=C4)Cn1c-3cc2c(c1=O)COC(=O)[C@]2(O)CC]

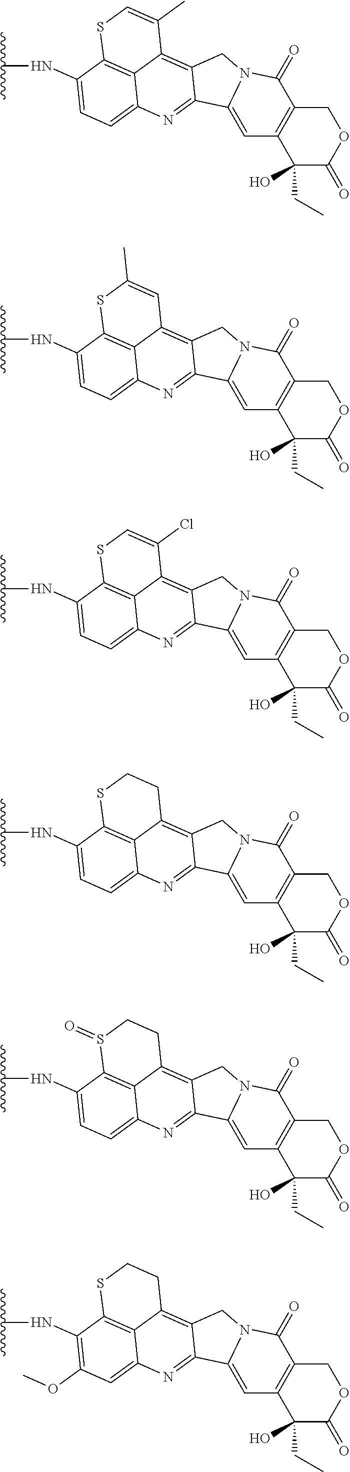 [SMILES: *Nc1ccc2nc3c(c4c2c1SC=C4C)Cn1c-3cc2c(c1=O)COC(=O)[C@]2(O)CC ; *Nc1ccc2nc3c(c4c2c1SC(C)=C4)Cn1c-3cc2c(c1=O)COC(=O)[C@]2(O)CC ; *Nc1ccc2nc3c(c4c2c1SC=C4Cl)Cn1c-3cc2c(c1=O)COC(=O)[C@]2(O)CC ; *Nc1ccc2nc3c(c4c2c1SCC4)Cn1c-3cc2c(c1=O)COC(=O)[C@]2(O)CC ; *Nc1ccc2nc3c(c4c2c1S(=O)CC4)Cn1c-3cc2c(c1=O)COC(=O)[C@]2(O)CC ; *Nc1c(OC)cc2nc3c(c4c2c1SCC4)Cn1c-3cc2c(c1=O)COC(=O)[C@]2(O)CC]

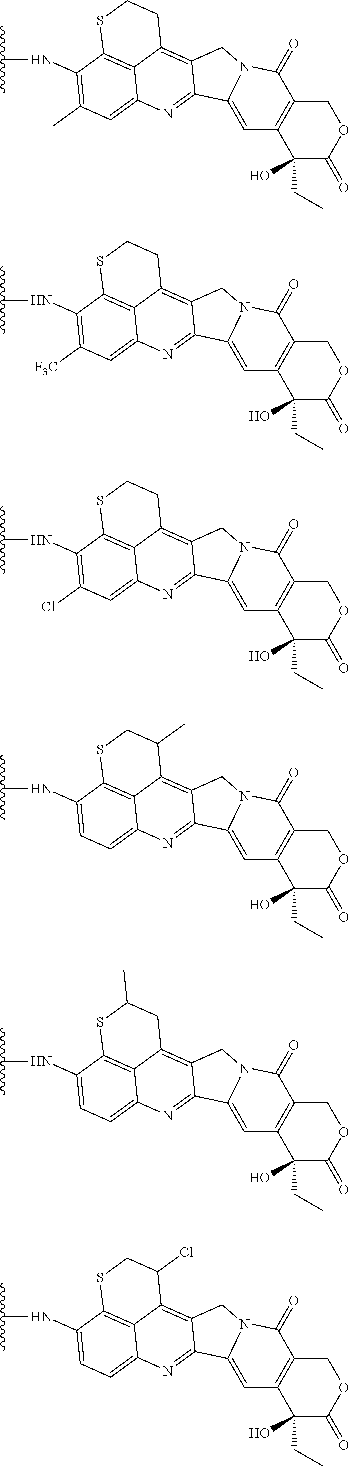 [SMILES: *Nc1c(C)cc2nc3c(c4c2c1SCC4)Cn1c-3cc2c(c1=O)COC(=O)[C@]2(O)CC ; *Nc1c(C(F)(F)F)cc2nc3c(c4c2c1SCC4)Cn1c-3cc2c(c1=O)COC(=O)[C@]2(O)CC ; *Nc1c(Cl)cc2nc3c(c4c2c1SCC4)Cn1c-3cc2c(c1=O)COC(=O)[C@]2(O)CC ; *Nc1ccc2nc3c(c4c2c1SCC4C)Cn1c-3cc2c(c1=O)COC(=O)[C@]2(O)CC ; *Nc1ccc2nc3c(c4c2c1SC(C)C4)Cn1c-3cc2c(c1=O)COC(=O)[C@]2(O)CC ; *Nc1ccc2nc3c(c4c2c1SCC4Cl)Cn1c-3cc2c(c1=O)COC(=O)[C@]2(O)CC]

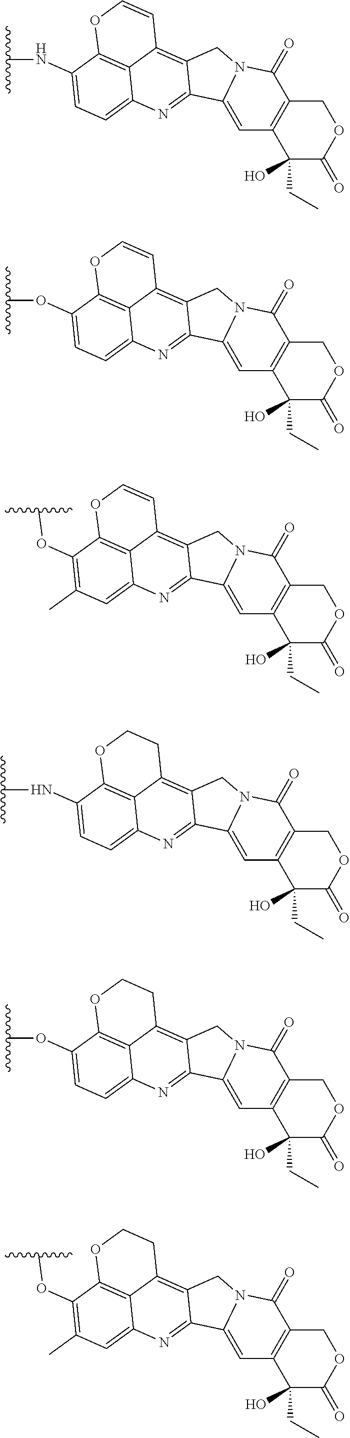 [SMILES: *Nc1ccc2nc3c(c4c2c1OC=C4)Cn1c-3cc2c(c1=O)COC(=O)[C@]2(O)CC ; *Oc1ccc2nc3c(c4c2c1OC=C4)Cn1c-3cc2c(c1=O)COC(=O)[C@]2(O)CC ; *Oc1c(C)cc2nc3c(c4c2c1OC=C4)Cn1c-3cc2c(c1=O)COC(=O)[C@]2(O)CC ; *Nc1ccc2nc3c(c4c2c1OCC4)Cn1c-3cc2c(c1=O)COC(=O)[C@]2(O)CC ; *Oc1ccc2nc3c(c4c2c1OCC4)Cn1c-3cc2c(c1=O)COC(=O)[C@]2(O)CC ; *Oc1c(C)cc2nc3c(c4c2c1OCC4)Cn1c-3cc2c(c1=O)COC(=O)[C@]2(O)CC]

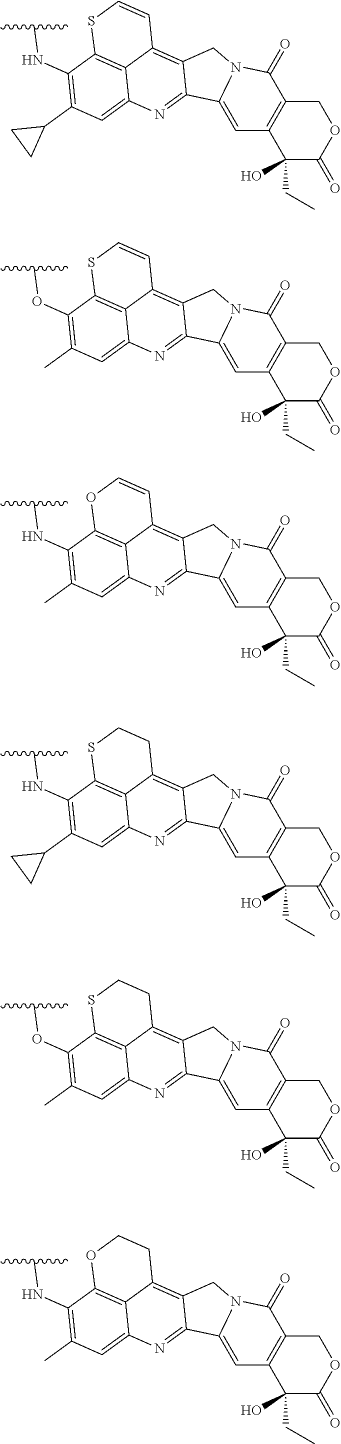 [SMILES: *Nc1c(C2CC2)cc2nc3c(c4c2c1SC=C4)Cn1c-3cc2c(c1=O)COC(=O)[C@]2(O)CC ; *Oc1c(C)cc2nc3c(c4c2c1SC=C4)Cn1c-3cc2c(c1=O)COC(=O)[C@]2(O)CC ; *Nc1c(C)cc2nc3c(c4c2c1OC=C4)Cn1c-3cc2c(c1=O)COC(=O)[C@]2(O)CC ; *Nc1c(C2CC2)cc2nc3c(c4c2c1SCC4)Cn1c-3cc2c(c1=O)COC(=O)[C@]2(O)CC ; *Oc1c(C)cc2nc3c(c4c2c1SCC4)Cn1c-3cc2c(c1=O)COC(=O)[C@]2(O)CC ; *Nc1c(C)cc2nc3c(c4c2c1OCC4)Cn1c-3cc2c(c1=O)COC(=O)[C@]2(O)CC]

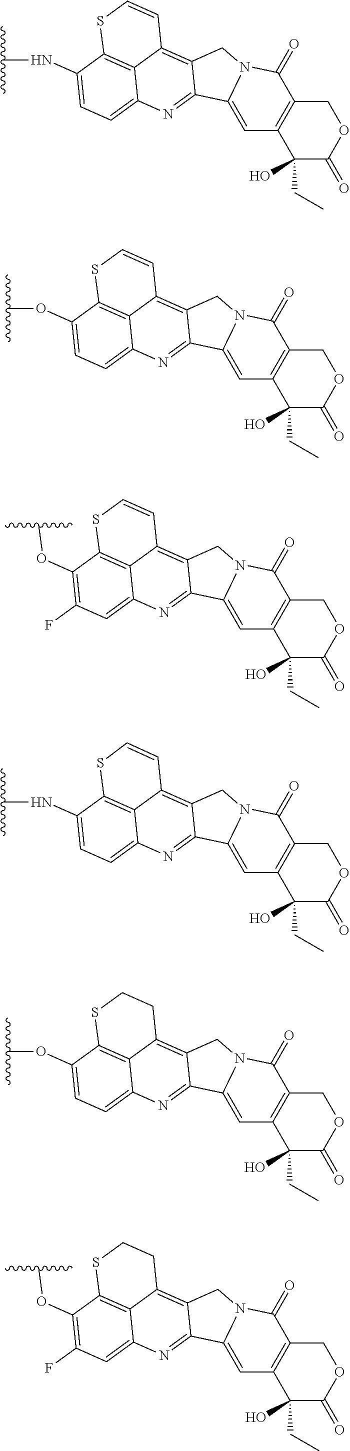 [SMILES: *Nc1ccc2nc3c(c4c2c1SC=C4)Cn1c-3cc2c(c1=O)COC(=O)[C@]2(O)CC ; *Oc1ccc2nc3c(c4c2c1SC=C4)Cn1c-3cc2c(c1=O)COC(=O)[C@]2(O)CC ; *Oc1c(F)cc2nc3c(c4c2c1SC=C4)Cn1c-3cc2c(c1=O)COC(=O)[C@]2(O)CC ; *Nc1ccc2nc3c(c4c2c1SC=C4)Cn1c-3cc2c(c1=O)COC(=O)[C@]2(O)CC ; *Oc1ccc2nc3c(c4c2c1SCC4)Cn1c-3cc2c(c1=O)COC(=O)[C@]2(O)CC ; *Oc1c(F)cc2nc3c(c4c2c1SCC4)Cn1c-3cc2c(c1=O)COC(=O)[C@]2(O)CC]

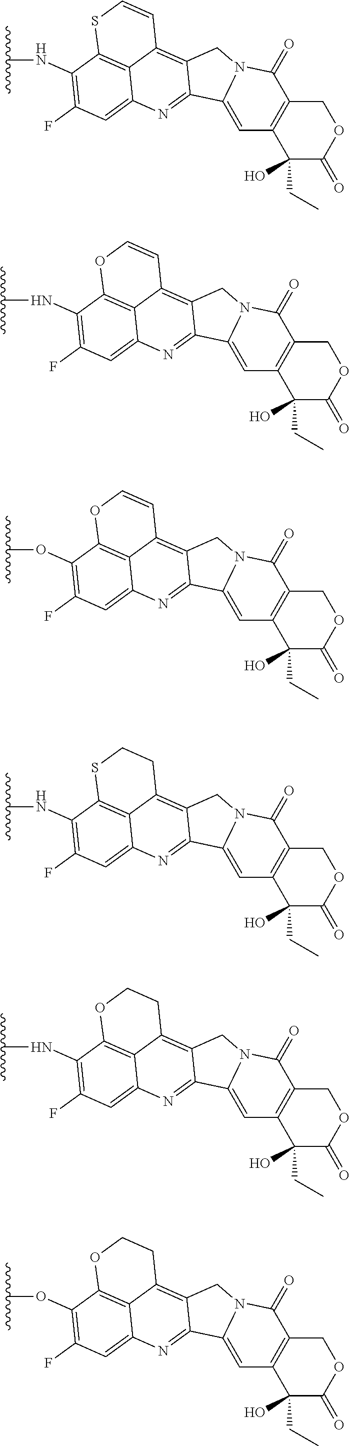 [SMILES: *Nc1c(F)cc2nc3c(c4c2c1SC=C4)Cn1c-3cc2c(c1=O)COC(=O)[C@]2(O)CC ; *Nc1c(F)cc2nc3c(c4c2c1OC=C4)Cn1c-3cc2c(c1=O)COC(=O)[C@]2(O)CC ; *Oc1c(F)cc2nc3c(c4c2c1OC=C4)Cn1c-3cc2c(c1=O)COC(=O)[C@]2(O)CC ; *Nc1c(F)cc2nc3c(c4c2c1SCC4)Cn1c-3cc2c(c1=O)COC(=O)[C@]2(O)CC ; *Nc1c(F)cc2nc3c(c4c2c1OCC4)Cn1c-3cc2c(c1=O)COC(=O)[C@]2(O)CC ; *Oc1c(F)cc2nc3c(c4c2c1OCC4)Cn1c-3cc2c(c1=O)COC(=O)[C@]2(O)CC]

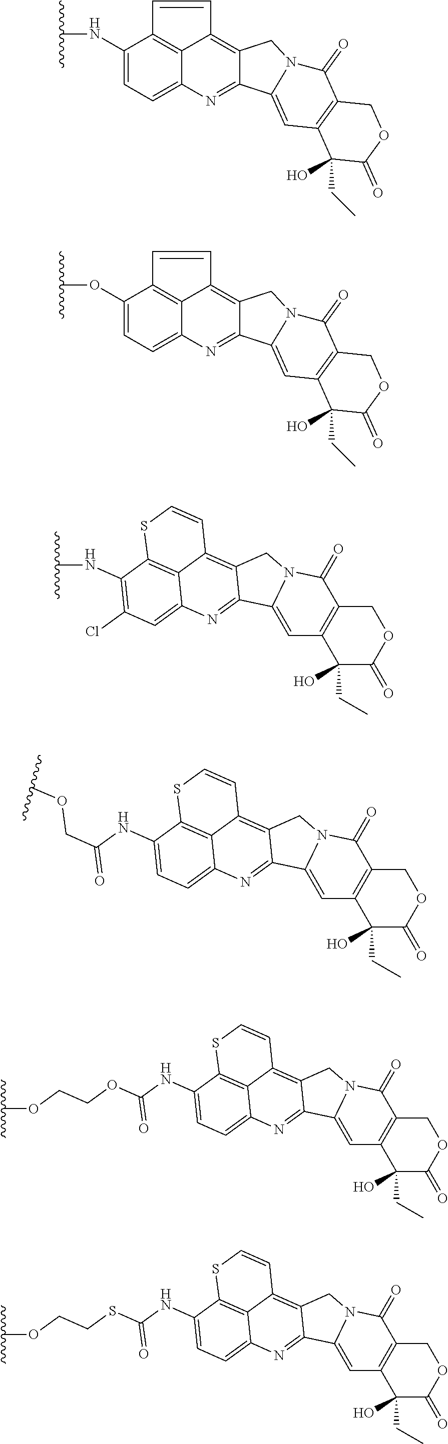 [SMILES: *Nc1ccc2nc3c(c4c2c1C=C4)Cn1c-3cc2c(c1=O)COC(=O)[C@]2(O)CC ; *Oc1ccc2nc3c(c4c2c1C=C4)Cn1c-3cc2c(c1=O)COC(=O)[C@]2(O)CC ; *Nc1c(Cl)cc2nc3c(c4c2c1SC=C4)Cn1c-3cc2c(c1=O)COC(=O)[C@]2(O)CC ; *OCC(=O)Nc1ccc2nc3c(c4c2c1SC=C4)Cn1c-3cc2c(c1=O)COC(=O)[C@]2(O)CC ; *OCCOC(=O)Nc1ccc2nc3c(c4c2c1SC=C4)Cn1c-3cc2c(c1=O)COC(=O)[C@]2(O)CC ; *OCCSC(=O)Nc1ccc2nc3c(c4c2c1SC=C4)Cn1c-3cc2c(c1=O)COC(=O)[C@]2(O)CC]

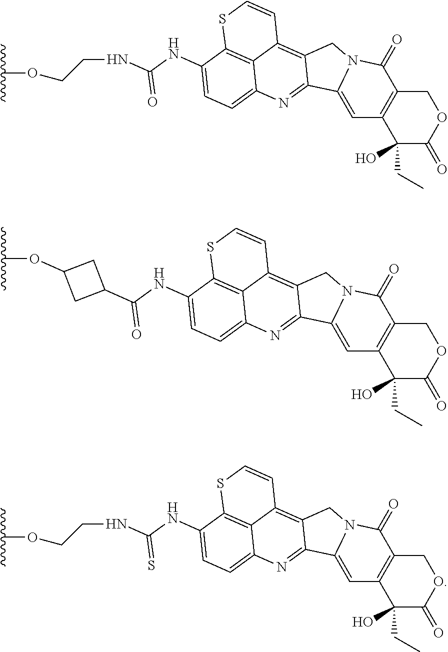 [SMILES: *OCCNC(=O)Nc1ccc2nc3c(c4c2c1SC=C4)Cn1c-3cc2c(c1=O)COC(=O)[C@]2(O)CC ; *OC1CC(C(=O)Nc2ccc3nc4c(c5c3c2SC=C5)Cn2c-4cc3c(c2=O)COC(=O)[C@]3(O)CC)C1 ; *OCCNC(=S)Nc1ccc2nc3c(c4c2c1SC=C4)Cn1c-3cc2c(c1=O)COC(=O)[C@]2(O)CC]

In some embodiments of Aspect 1, the provided ligand-drug conjugate or the pharmaceutically acceptable salt or solvate thereof is according to formula (III):

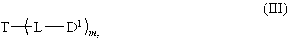

wherein:
T is a targeting or binding ligand;
L is a releasable linker;
D¹ is as defined in Aspect 1, including of any of the formula (I) and (II) and any embodiments thereof, and
m is an integer or a decimal from 1 to 10.

In some embodiments of Aspect 1, in the ligand-drug conjugate or the pharmaceutically acceptable salt or solvate thereof according to the present disclosure, including of any of the formula (I), (II), and (III) and any embodiments thereof, the linker unit L is -L¹-L²-L³-L⁴-, wherein:
L¹ is selected from the group consisting of

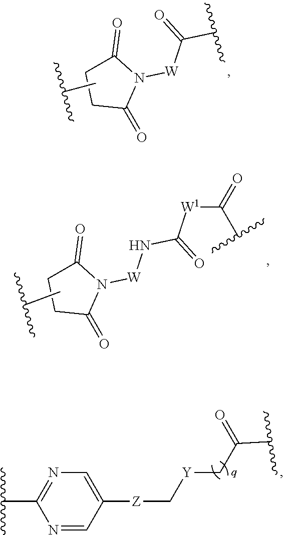

—CH₂—C(O)—NR⁷-W-C(O)—, —CH(COOH)—C(O)—NR⁷-W-C(O)—, and —C(O)—W-C(O)—; wherein W and W¹ are independently selected from the group consisting of C₁-C₈ alkylene, —(C₁-C₈ alkylene)-cycloalkylene-, arylene, -arylene-(C₁-C₈ alkylene)-, heteroarylene, and linear heteroalkylene, wherein the linear heteroalkylene comprise 1 to 8 carbon atom(s), and 1 to 6 heteroatom(s) selected from the group consisting of N, O and S, SO, SO₂, and wherein the —(C₁-C₈ alkylene)-cycloalkylene-, linear heteroalkylene, arylene, and heteroarylene are each independently optionally further substituted by one or more substituent(s) selected from the group consisting of halogen, hydroxy, cyano, amino, alkyl, chloroalkyl, deuterated alkyl, alkoxy and cycloalkyl; wherein q is an integer selected from 1 to 6; wherein Z is selected from the group consisting of C₁-C₆ alkylene, C₁-C₆ alkenylene, C₁-C₆ alkynylene, arylene, and heteroarylene; wherein Y is selected from the group consisting of —O—, —S—, —C(R⁷) (R⁸)—, and heteroarylene; wherein the left side of each of the L¹ groups provided above is attached to T;
L² is selected from the group consisting of —NR⁸ (CH₂CH₂O)ₚ¹CH₂CH₂C(O)—, —NR⁸(CH₂CH₂O)ₚ¹CH₂CH₂—, —NR⁸(CH₂CH₂O)ₚ¹CH₂C(O)—, —S(CH₂)ₚ¹C(O)—, —N R⁸—(CH₂)ₚ²-(1H-1,2,3-triazole-1,4-diyl)-(CH₂CH₂O)ₚ¹ CH₂C(O), —N R⁸—(CH₂)ₚ²-(1H-1,2,3-triazole-1,4-diyl)-(CH₂CH₂O)ₚ¹ CH₂CH₂NHC(O)CH₂OCH₂C(O), —N R⁸ (CH₂CH₂O)ₚ¹CH₂CH₂NHC(O)CH₂OCH₂C(O), and a chemical bond, wherein p¹ is an integer selected from 1 to 20; wherein p² is an integer selected from 1 to 3; wherein the left side of each of the L² groups provided above is attached to L¹;
L³ is a peptide residue composed of 2 to 7 amino acids, wherein the amino acids are optionally further substituted by one or more substituent(s) selected from the group consisting of halogen, hydroxy, cyano, amino, alkyl, chloroalkyl, deuterated alkyl, alkoxy and cycloalkyl; wherein the left side of each of the L³ groups provided above is attached to L²;
L⁴ is selected from the group consisting of —NR⁹(CR¹⁰R¹¹)ₜ—,

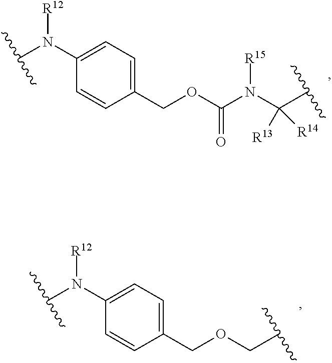

and a chemical bond, wherein t is an integer selected from 1 to 6; wherein in the presence of L⁴ groups, the left side of each of the L⁴ groups provided above is attached to the right side of L³ and the right side of each of the L⁴ groups is attached to D¹;
R⁷, R⁸, R⁹, and R¹² are each independently selected from the group consisting of H, alkyl, haloalkyl, deuterated alkyl and hydroxyalkyl;
R¹⁰, R¹¹, R¹³, and R¹⁴ are each independently selected from the group consisting of H, halogen, alkyl, haloalkyl, deuterated alkyl, and hydroxyalkyl; and
R¹⁵ is selected from —CH₂CH₂SO₂CH₃, and —CH₂CH₂N(CH₃)₂.

In some embodiments of Aspect 1, in the ligand-drug conjugate or the pharmaceutically acceptable salt or solvate thereof according to the present disclosure, including of any of the formula (I), (II), and (III) and any embodiments thereof, the linker unit L¹ is selected from the group consisting of

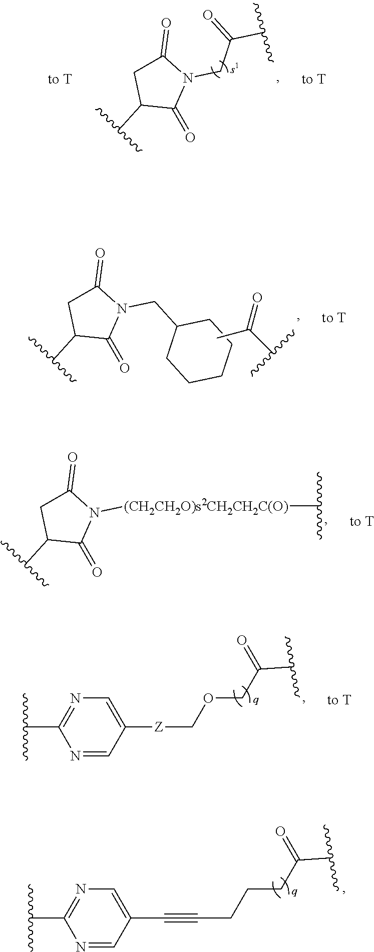

and —CH(COOH)—C(O)—NH—C₆H₄—(CH₂)s³-C(O)— (wherein the left hand side of this group is attached to T), wherein s¹ is an integer selected from 2 to 8; s² is an integer selected from 1 to 3; s³ is an integer selected from 1 to 8; q is an integer selected from 1 to 3; and Z is selected from the group consisting of C₁-C₆ alkylene, C₁-C₆ alkenylene, and C₁-C₆ alkynylene.

In some embodiments of Aspect 1, in the ligand-drug conjugate or the pharmaceutically acceptable salt or solvate thereof according to the present disclosure, including of any of the formula (I), (II), and (III) and any embodiments thereof, the linker unit L² is selected from the group consisting of —NR⁸(CH₂CH₂O)ₚ¹CH₂CH₂C(O)—, —NR⁸(CH₂CH₂O)ₚ¹CH₂C(O)—, —N R⁸—(CH₂)ₚ²-(1H-1,2,3-triazole-1,4-diyl)-(CH₂CH₂O)ₚ¹ CH₂C(O), and a chemical bond; wherein R⁸ is selected from the group consisting of H, alkyl, haloalkyl, deuterated alkyl and hydroxyalkyl; p¹ is an integer selected from 6 to 12; p² is an integer selected from 1 to 3; wherein the left side of each of the L² groups provided above is attached to L¹.

In some embodiments of Aspect 1, in the ligand-drug conjugate or the pharmaceutically acceptable salt or solvate thereof according to the present disclosure, including of any of the formula (I), (II), and (III) and any embodiments thereof, the linker unit L³ is a peptide residue composed of 2 to 7 amino acids, wherein the amino acids are selected from Phenylalanine (F), Glycine (G), Valine (V), Lysine (K), Citrulline, Serine (S), Alanine (A), Glutamic acid (E), and Aspartic acid (D). In certain embodiments, L³ is a peptide residue composed of 1, 2 or more Phenylalanine and Glycine. In certain embodiments, L³ is a peptide residue composed of 4 amino acids. In certain embodiments, L³ is a peptide residue GGFG or VA; wherein the left side of each of the L³ groups provided above is attached to L².

In some embodiments of Aspect 1, in the ligand-drug conjugate or the pharmaceutically acceptable salt or solvate thereof according to the present disclosure, including of any of the formula (I), (II), and (III) and any embodiments thereof, the linker unit L⁴ is —NR⁹(CR¹⁰R¹¹)ₜ—,

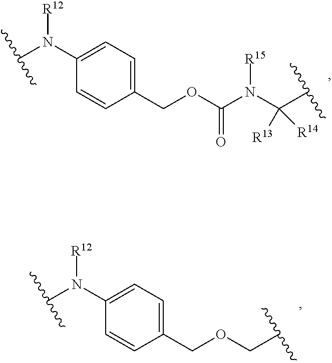

or a chemical bond; wherein R⁹, R¹⁰, R¹¹, R¹², R¹³, and R¹⁴ are each independently selected from H and alkyl; R¹¹ is selected from —CH₂CH₂SO₂CH₃, and —CH₂CH₂N(CH₃)₂; t is 1 or 2; wherein in the presence of L⁴ groups, the left side of each of the L⁴ groups provided above is attached to the right side of L³ and the right side of each of the L⁴ groups is attached to D¹. In certain embodiments, L⁴ is a chemical bond;

In some embodiments of Aspect 1, in the ligand-drug conjugate or the pharmaceutically acceptable salt or solvate thereof according to the present disclosure, including of any of the formula (I), (II), and (III) and any embodiments thereof, the linker unit L is -L¹-L²-L³-, wherein: L¹ is

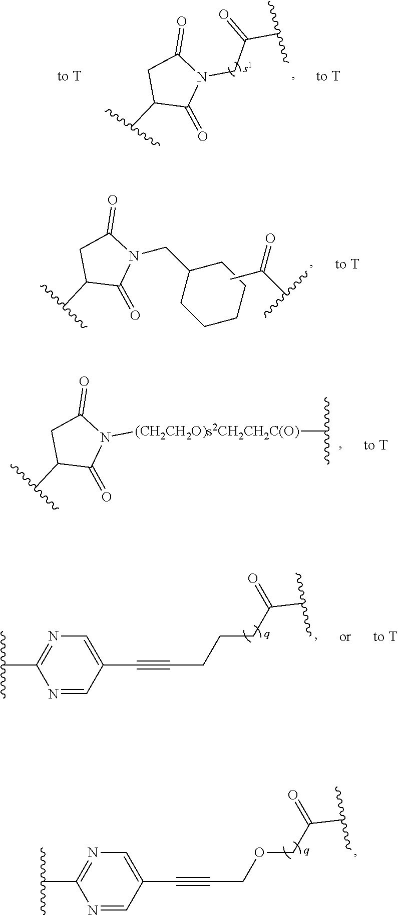

wherein s¹ is an integer selected from 2 to 8; s² is an integer selected from 1 to 3; and q is an integer selected from 1 to 3;
L² is selected from the group consisting of —NR⁸(CH₂CH₂O)ₚ¹CH₂CH₂C(O)—, —NR⁸(CH₂CH₂O)ₚ¹CH₂C(O)—, —NR⁸—(CH₂)ₚ²-(1H-1,2,3-triazole-1,4-diyl)-(CH₂CH₂O)ₚ¹ CH₂C(O), and a chemical bond, wherein R⁸ is selected from the group consisting of H, alkyl, haloalkyl, deuterated alkyl and hydroxyalkyl; p¹ is an integer selected from 6 to 12; p² is an integer selected from 1 to 3; wherein the left side of each of the L² groups provided above is attached to L¹;
L³ is a peptide residue GGFG or VA; wherein the left side of each of the L³ groups provided above is attached to L²;
and wherein the left side of each of the L¹ groups provided above is connected to the ligand, and the right side each of the L³ groups provided above is connected to D¹.

In some embodiments of Aspect 1, in the ligand-drug conjugate or the pharmaceutically acceptable salt or solvate thereof according to the present disclosure, including of any of the formula (I), (II), and (III) and any embodiments thereof, the linker unit L includes, but is not limited to:

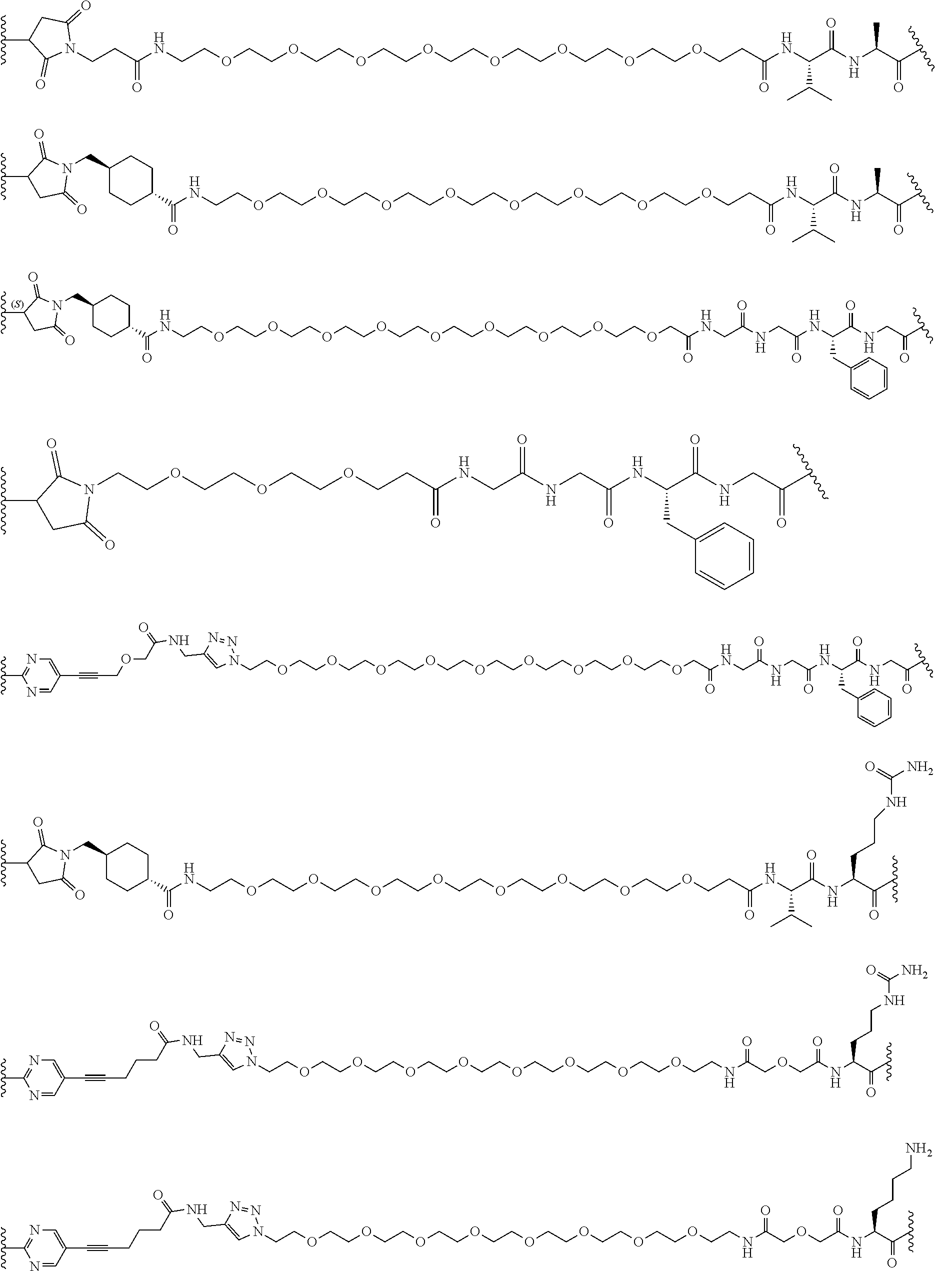

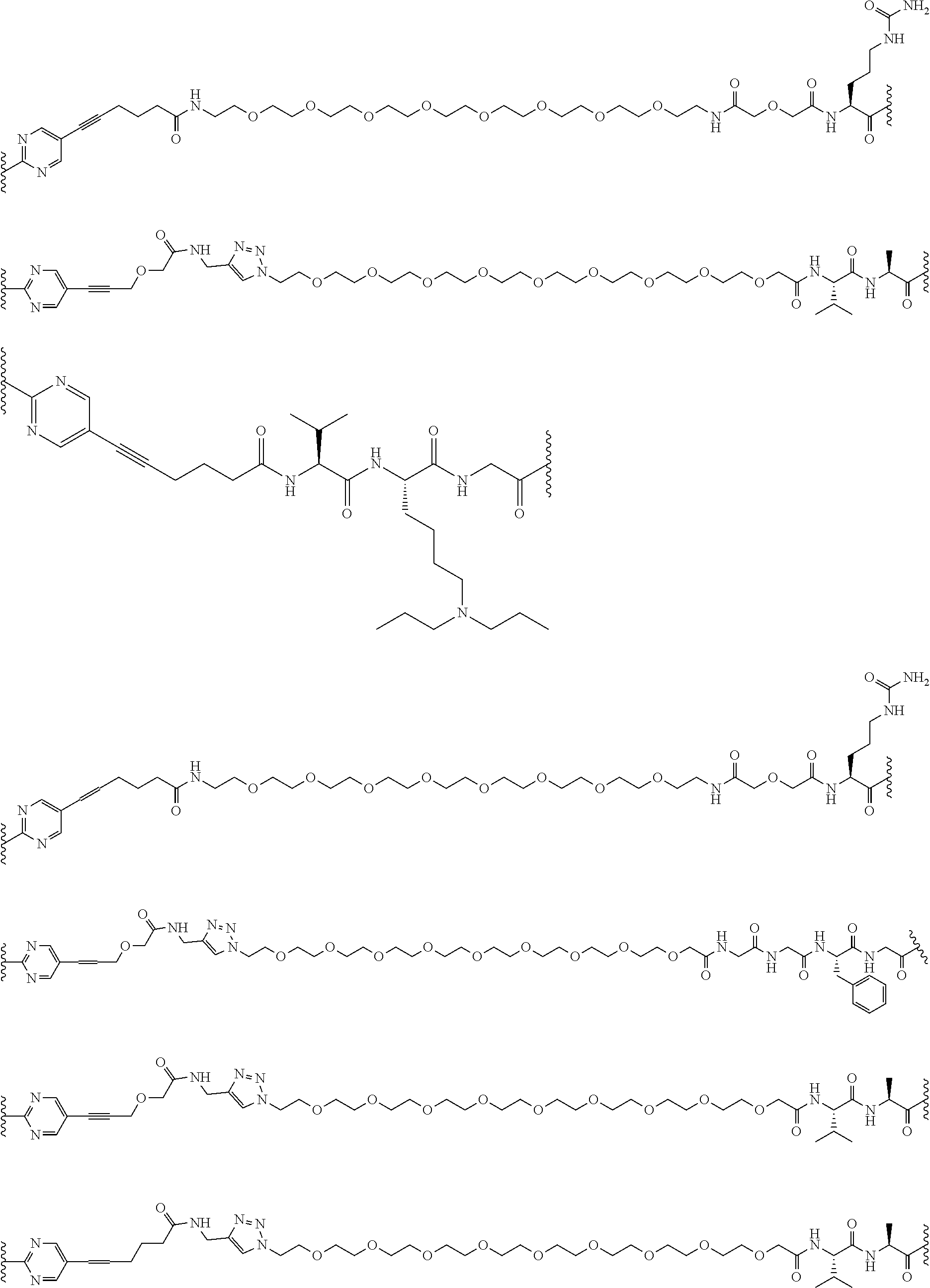

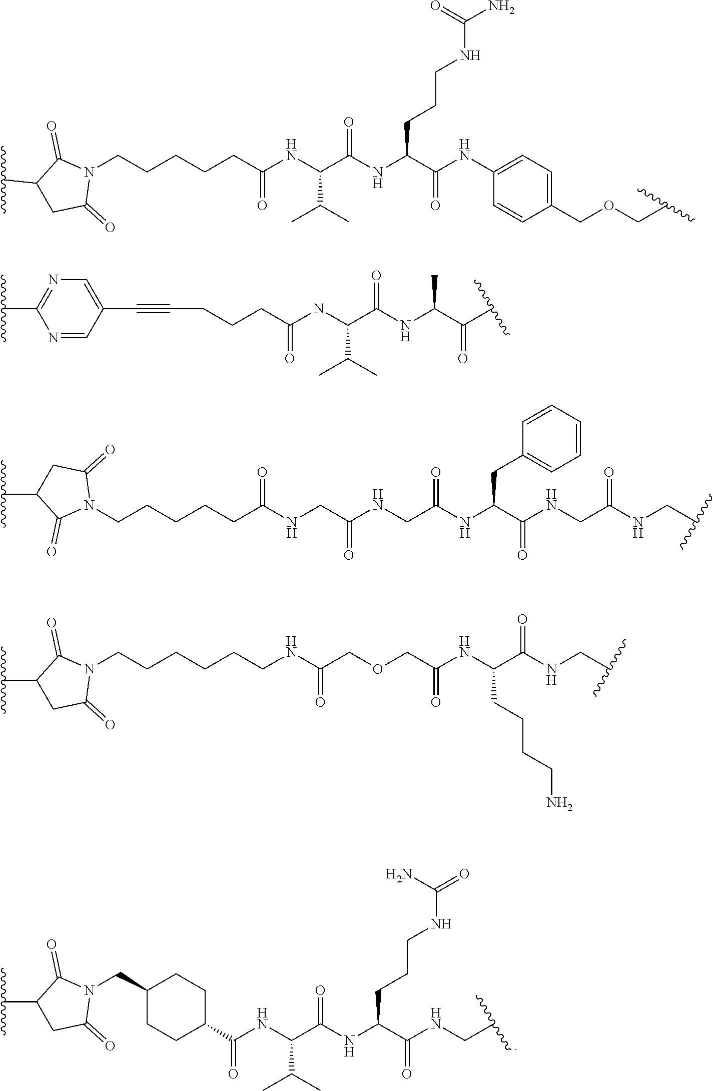

In some embodiments of Aspect 1, the ligand-drug conjugates include, but are not limited to:

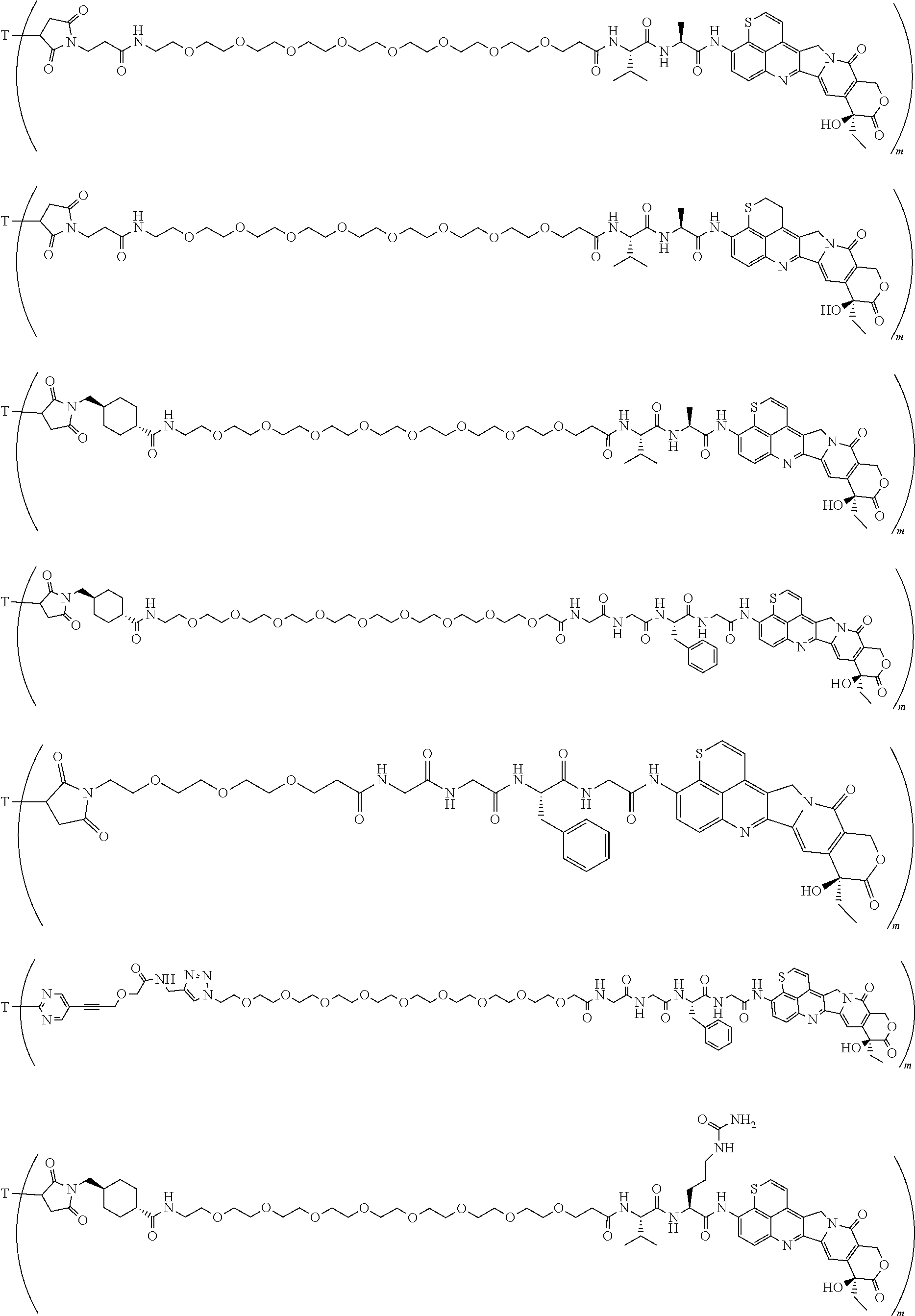

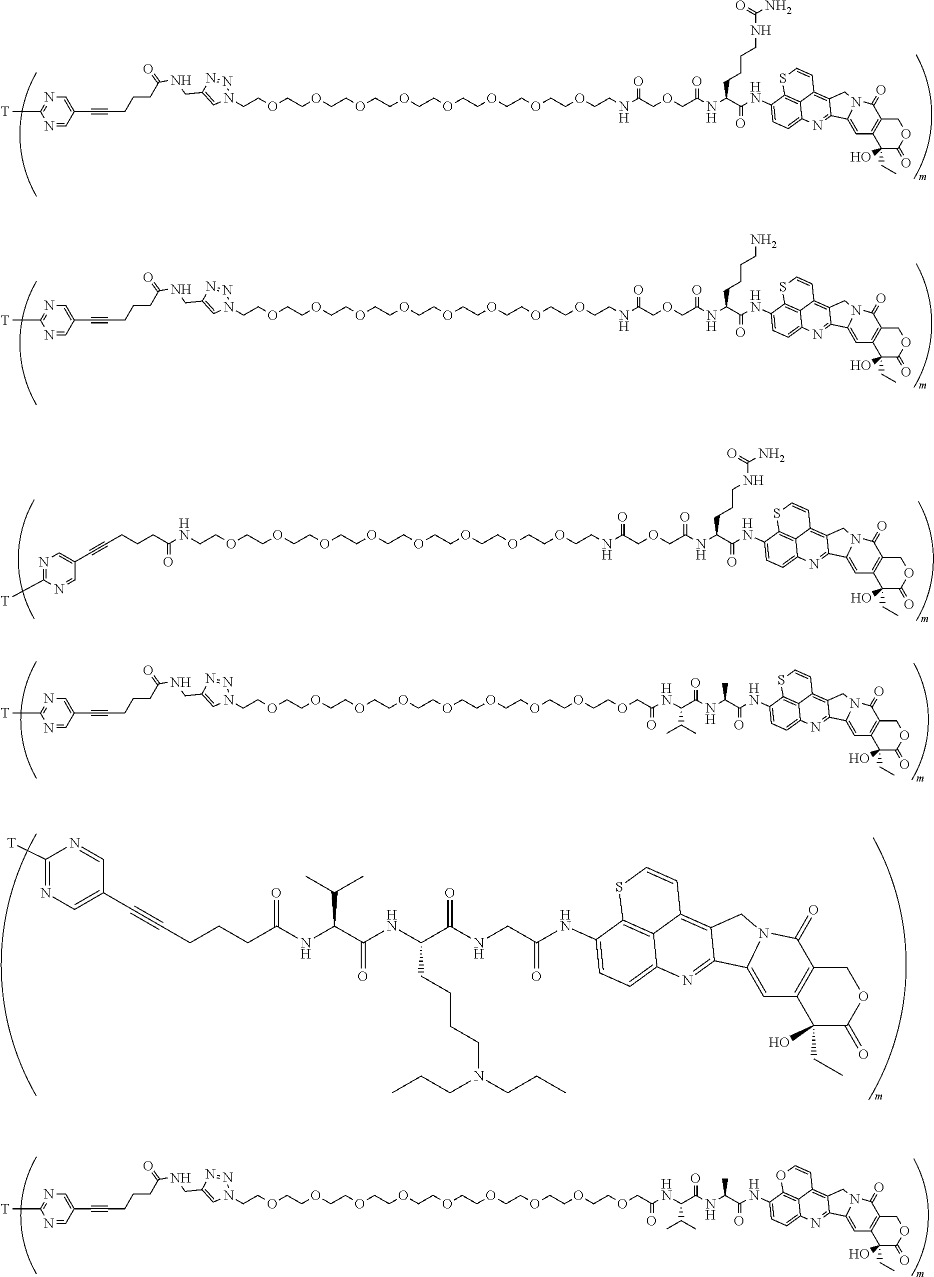

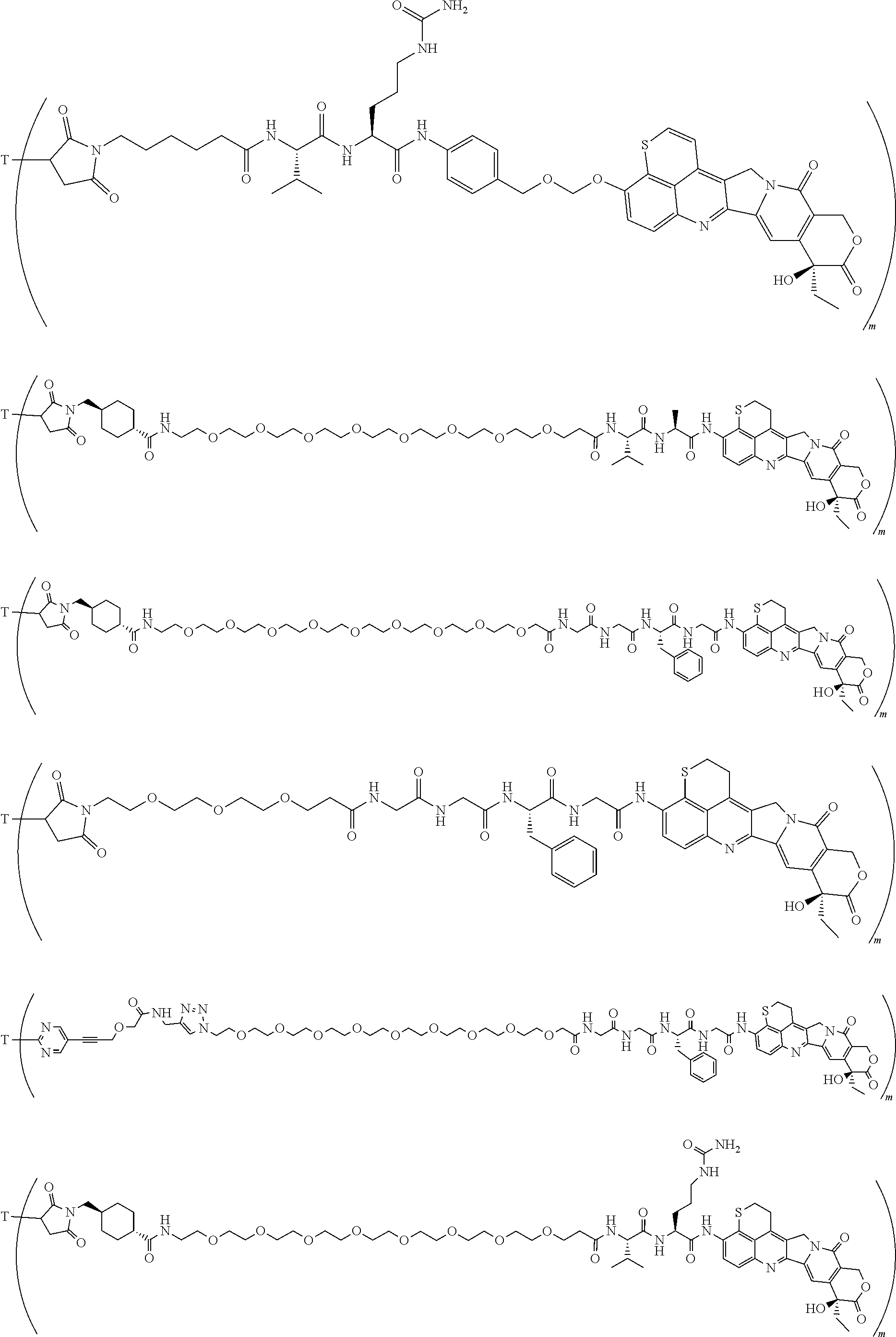

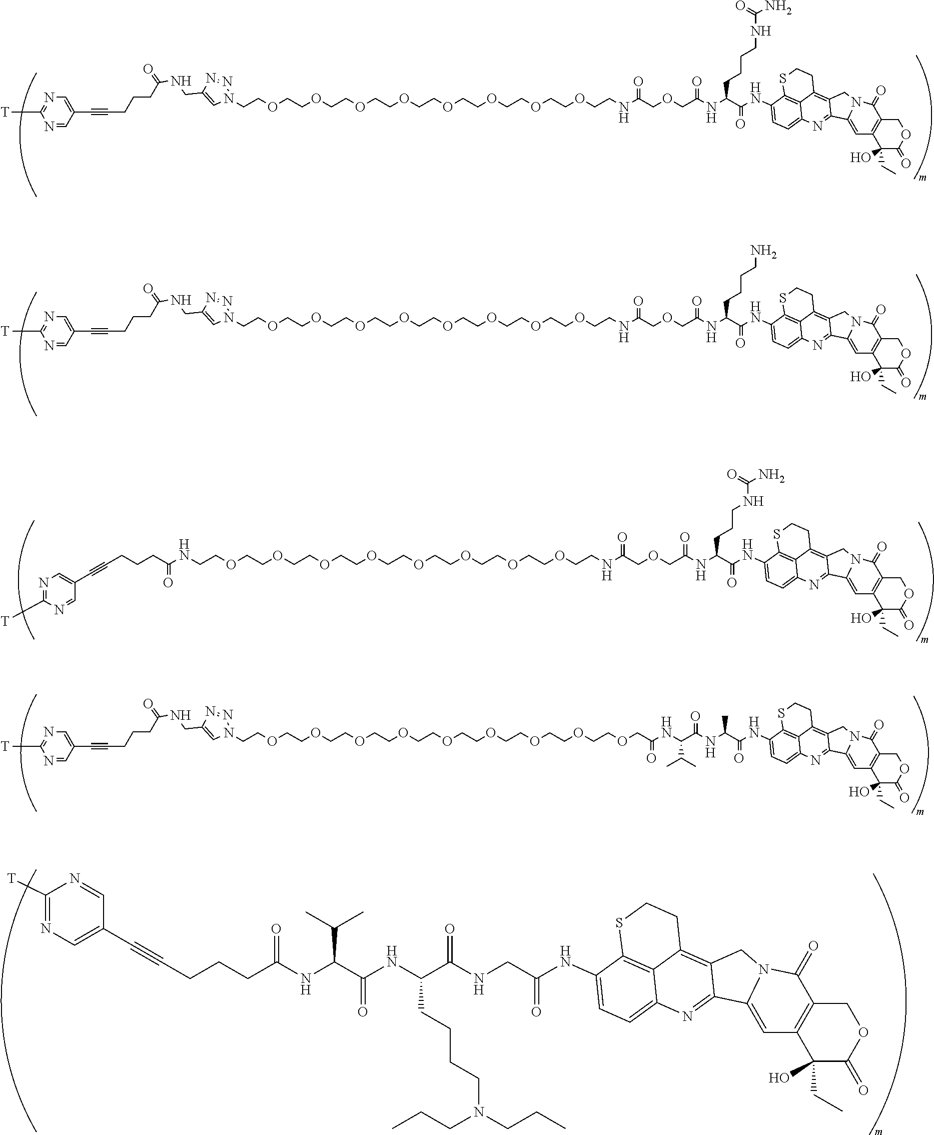

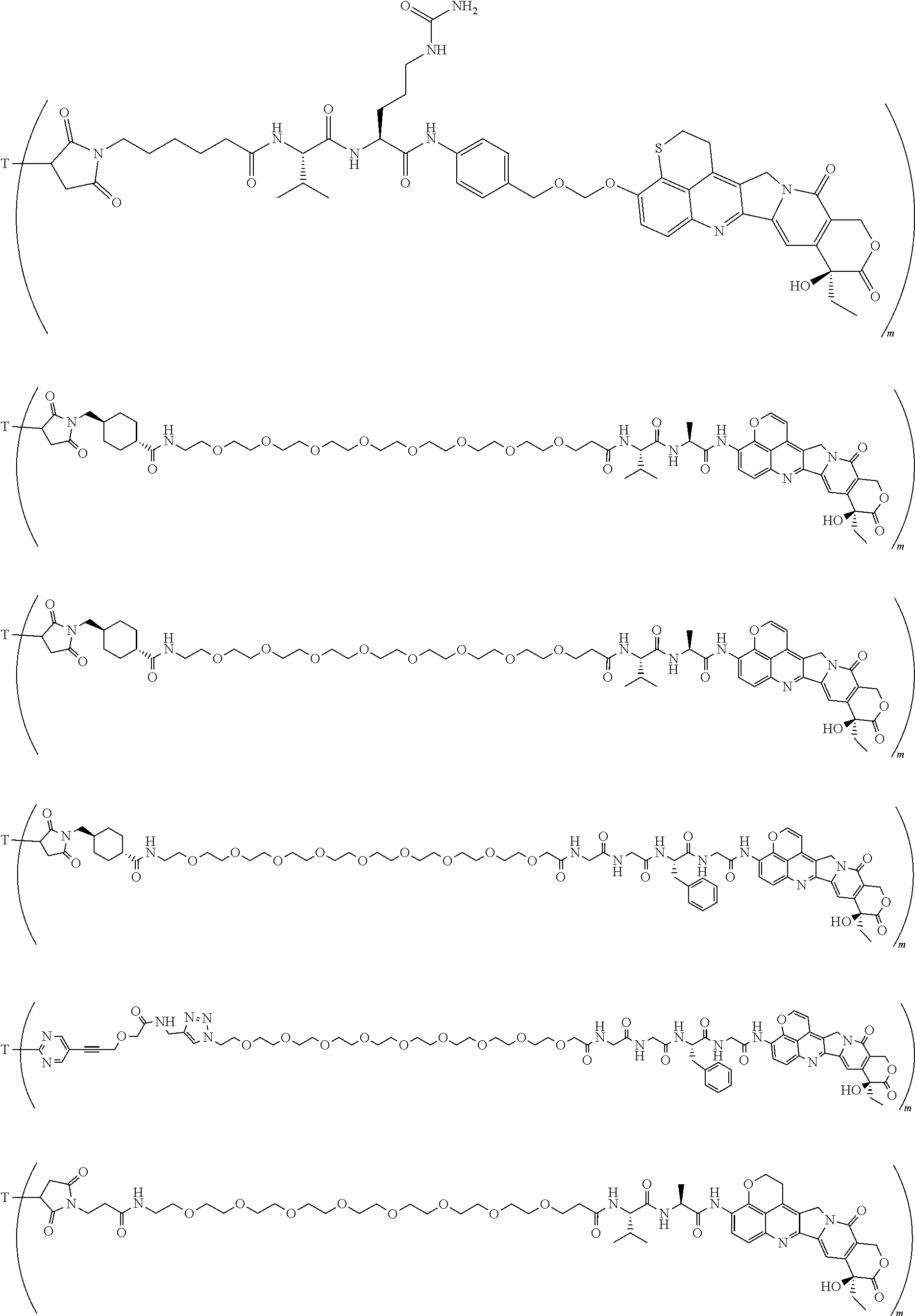

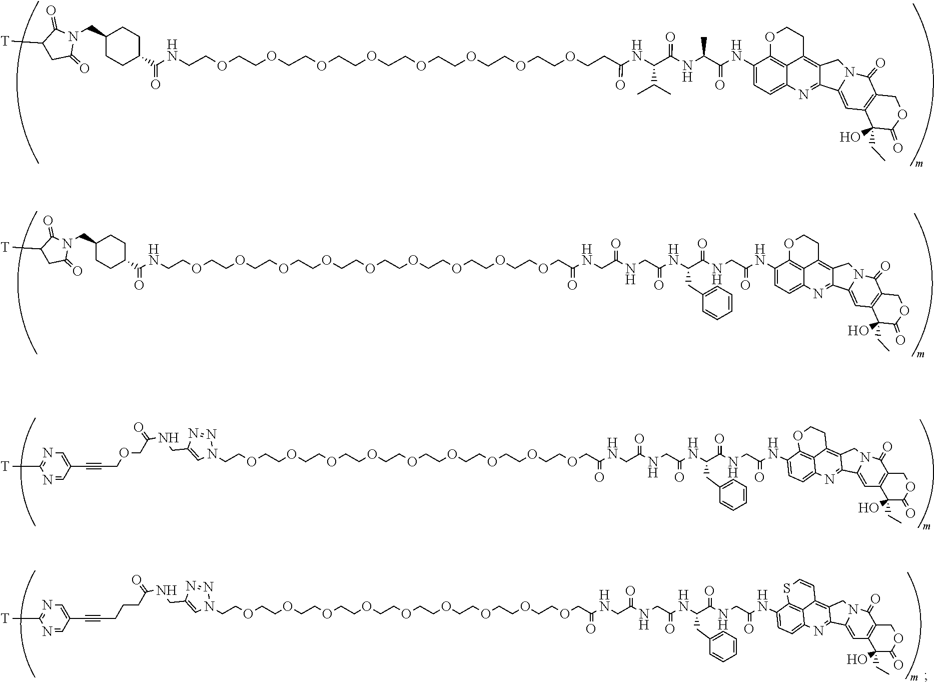

or a pharmaceutically acceptable salt or solvate thereof,
wherein T and m are defined as in formula (III) and any embodiments thereof, and wherein further embodiments of T are provided below.

In some embodiments of Aspect 1, provided is the ligand-drug conjugate or the pharmaceutically acceptable salt or solvate thereof, wherein:

T is a targeting antibody or ligand binding to antigen; wherein the antibody is selected from chimeric antibody, humanized antibody and human antibody; optionally, wherein T is a monoclonal antibody.

In some embodiments of Aspect 1, provided is the ligand-drug conjugate or the pharmaceutically acceptable salt or solvate thereof, wherein:

T is selected from anti-Her2(ErbB2) antibody, anti-EGFR antibody, anti-B₇H₃ antibody, anti-c-MET antibody, anti-Her3(ErbB3) antibody, anti-Her4(ErbB4) antibody, anti-CD20 antibody, anti-CD22 antibody, anti-CD30 antibody, anti-CD33 antibody, anti-CD44 antibody, anti-CD56 antibody, anti-CD70 antibody, anti-CD73 antibody, anti-CD105 antibody, anti-CEA antibody, anti-A33 antibody, anti-Cripto antibody, anti-EphA2 antibody, anti-G250 antibody, anti-MICI antibody, anti-Lewis Y antibody, anti-VEGFR antibody, anti-GPNMB antibody, anti-Integrin antibody, anti-PSMA antibody, anti-Tenascin-C antibody, anti-SLC44A4 antibody or anti-Mesothelin antibody, anti-ROR1 antibody or the fragment binding to the antigen.

In some embodiments of Aspect 1, provided is the ligand-drug conjugate or the pharmaceutically acceptable salt or solvate thereof, wherein: T is selected from Trastuzumab, Pertuzumab, Nimotuzumab, Enoblituzumab, Emibetuzumab, Inotuzumab, Pinatuzumab, Brentuximab, Gemtuzumab, Bivatuzumab, Lorvotuzumab, cBR96 and Glembatumumab or the fragment binding to the antigen.

In some embodiments of Aspect 1, provided is the ligand-drug conjugate or the pharmaceutically acceptable salt or solvate thereof, wherein: T is Trastuzumab, m is an integer or a decimal from 1 to 10; optionally m is an integer or a decimal from 3 to 8.

Aspect 2: In some other embodiments of the present disclosure, provided is a compound or a pharmaceutically acceptable salt, solvate, tautomer, mesomer, racemate, enantiomer, diastereomer, or combinations thereof, where the compound is of formula (IV) thereof:

L-D¹ (IV)
wherein: D¹ is represented by Formula I:

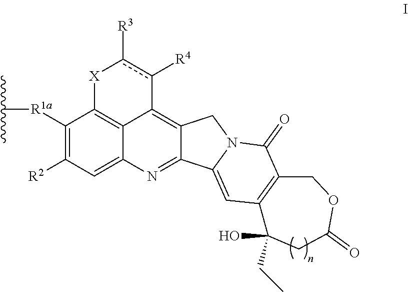

Wherein:
X is either absent, or selected from —O—, —S—, —S(O₂)—, or —S(O)—;
R^{1a} is —NH—R^{1b}—O—, —NH—, or —O—; optionally, R^{1a} is —NH— or —O—;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R² is selected from H, halo, hydroxy, cyano, nitro, C₃-C₈ cycloalkyl, C₁-C₈ alkyl, C₁-C₈ alkoxy, R⁵—(C₁-C₈ alkylene)-, amino, (C₁-C₈ alkyl)NHC(O)O—, (C₃-C₈ cycloalkyl)NHC(O)O—, (C₁-C₈ alkyl)NH—, azetidin-1-yl, pyrrolidin-1-yl, piperidin-1-yl, azepane-1-yl, (C₁-C₈ alkyl)C(O)O—, or (C₁-C₈ alkyl)C(O)NH—; wherein azetidin-1-yl, pyrrolidin-1-yl, piperidin-1-yl, azepane-1-yl, C₁-C₈ alkoxy, each “C₃-C₈ cycloalkyl”, and each “C₁-C₈ alkyl” are independently optionally substituted with one to four R⁶;
R³ and R⁴ are independently selected from H, halo, hydroxy, cyano, C₁-C₈ alkyl, C₁-C₈ alkoxy, C₃-C₈ cycloalkyl, aryl, heteroaryl, or amino; wherein C₁-C₈ alkyl, C₁-C₈ alkoxy, C₃-C₈ cycloalkyl, aryl, and heteroaryl are independently optionally substituted with one to four R⁶;
R⁵ is selected from C₁-C₈ alkoxy, C₃-C₈ cycloalkyl, aryl, or heteroaryl; wherein C₁-C₈ alkoxy, C₃-C₈ cycloalkyl, aryl, and heteroaryl are independently optionally substituted with one to four R⁶;
R⁶ at each occurrence is independently selected from halo, hydroxy, cyano, amino, C₁-C₈ alkyl, C₃-C₈ cycloalkyl, aryl, or heteroaryl;
and wherein a line with a dotted line represents either a single bond or a double bond;
n is an integer from 0 or 1;
and wherein L is L^{1a}-L²-L³-L⁴-,
L^{1a} is selected from the group consisting of

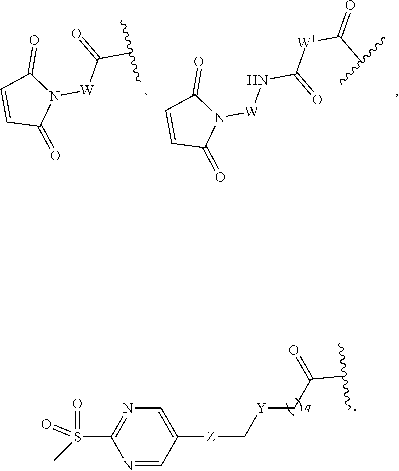

and COOH-W-C(O)—; wherein W and W¹ are independently selected from the group consisting of C₁-C₈ alkylene, —(C₁-C₈ alkylene)-cycloalkylene-, arylene, -arylene-(C₁-C₈ alkylene)-, heteroarylene, and linear heteroalkylene, wherein the linear heteroalkylene comprise 1 to 8 carbon atom(s), and 1 to 6 heteroatom(s) selected from the group consisting of N, O and S, SO, SO₂, and wherein the —(C₁-C₈ alkylene)-cycloalkylene-, linear heteroalkylene, arylene, and heteroarylene are each independently optionally further substituted by one or more substituent(s) selected from the group consisting of halogen, hydroxy, cyano, amino, alkyl, chloroalkyl, deuterated alkyl, alkoxy and cycloalkyl; wherein q is an integer selected from 1 to 6; wherein Z is selected from the group consisting of C₁-C₆ alkylene, C₁-C₆ alkenylene, C₁-C₆ alkynylene, arylene, and heteroarylene; wherein Y is selected from the group consisting of —O—, —S—, —C(R⁷) (R⁸)-, and heteroarylene;
L² is selected from the group consisting of —NR⁸(CH₂CH₂O)ₚ¹CH₂CH₂C(O)—, —NR⁸(CH₂CH₂O)ₚ¹CH₂CH₂—, —NR⁸(CH₂CH₂O)ₚ¹CH₂C(O)—, —S(CH₂)ₚ¹C(O)—, —N R⁸—(CH₂)ₚ²-(1H-1,2,3-triazole-1,4-diyl)-(CH₂CH₂O)ₚ¹ CH₂C(O), —N R⁸—(CH₂)ₚ²-(1H-1,2,3-triazole-1,4-diyl)-(CH₂CH₂O)ₚ¹ CH₂CH₂NHC(O)CH₂OCH₂C(O), —N R⁸ (CH₂CH₂O)ₚ¹ CH₂CH₂NHC(O)CH₂OCH₂C(O), and a chemical bond, wherein p¹ is an integer selected from 1 to 20; wherein p² is an integer selected from 1 to 3; wherein the left side of each of the L² groups provided above is attached to L¹;
L³ is a peptide residue composed of 2 to 7 amino acids, wherein the amino acids are optionally further substituted by one or more substituent(s) selected from the group consisting of halogen, hydroxy, cyano, amino, alkyl, chloroalkyl, deuterated alkyl, alkoxy and cycloalkyl; wherein the left side of each of the L³ groups provided above is attached to L²; L⁴ is selected from the group consisting of —NR⁹(CR¹⁰R¹¹)ₜ—,

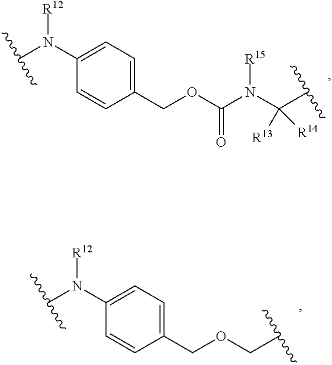

and a chemical bond, wherein t is an integer selected from 1 to 6; wherein the left side of each of the L⁴ groups provided above is attached to the right side of L³ and the right side of each of the L⁴ groups is attached to D¹;
R⁷, R⁸, R⁹, and R¹² are each independently selected from the group consisting of H, alkyl, haloalkyl, deuterated alkyl and hydroxyalkyl;
R¹⁰, R¹¹, R¹³, and R¹⁴ are each independently selected from the group consisting of H, halogen, alkyl, haloalkyl, deuterated alkyl, and hydroxyalkyl; and
R¹⁵ is selected from —CH₂CH₂SO₂CH₃, and —CH₂CH₂N(CH₃)₂.

In some embodiments of Aspect 2, wherein:
X is selected from —O—, —S—, —S(O₂)—, or —S(O)—;
R^{1a} is —NH—R^{1b}—O—, —NH—, or —O—; optionally, R^{1a} is —NH— or —O—;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R² is selected from H, halo, hydroxy, cyano, nitro, C₃-C₆ cycloalkyl, C₁-C₆ alkyl, C₁-C₆ alkoxy, R⁵—(C₁-C₃ alkylene)-, amino; wherein C₁-C₆ alkoxy, C₃-C₆ cycloalkyl, and C₁-C₆ alkyl are independently optionally substituted with one to four R⁶;
R³ and R⁴ are independently selected from H, halo, hydroxy, cyano, C₁-C₆ alkyl, C₁-C₆ alkoxy, or C₃-C₆ cycloalkyl; wherein C₁-C₆ alkyl, C₁-C₆ alkoxy, and C₃-C₆ cycloalkyl are independently optionally substituted with one to four R⁶;
R⁵ is C₁-C₆ alkoxy or C₃-C₆ cycloalkyl; wherein C₁-C₆ alkoxy and C₃-C₆ cycloalkyl are independently optionally substituted with one to four R⁶;
R⁶ at each occurrence is independently selected from halo, hydroxy, cyano, amino, C₁-C₃ alkyl, or C₃-C₆ cycloalkyl;
and wherein a line with a dotted line represents either a single bond or a double bond;
n is 1.

In some embodiments of Aspect 2, wherein D¹ is represented by Formula II:

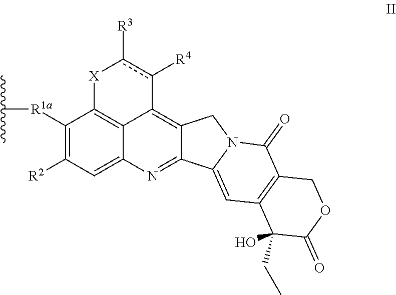

wherein: X is —O— or —S—; R^{1a} is —NH—R^{1b}—O—, —NH—, or —O—; optionally, R^{1a} is —NH— or —O—; R^{1b}, R², R³, R⁴, and a line with a dotted line are as defined as in formula (I) and (IV) and any embodiments thereof.

In some embodiments of Aspect 2, including of any of the formula (I), (II), and (IV) and any embodiments thereof: wherein R², R³ and R⁴ are independently selected from H, halo, C₁-C₃ alkyl, or C₁-C₃ alkoxy and wherein C₁-C₃ alkoxy and C₁-C₃ alkyl are independently optionally substituted with one to four halo.

In some embodiments of Aspect 2, including of any of the formula (I), (II), and (IV) and any embodiments thereof: wherein a line with a dotted line represents a double bond.

In some embodiments of Aspect 2, including of any of the formula (I), (II), and (IV) and any embodiments thereof: wherein R², R³ and R⁴ are independently selected from H, halo, C₁-C₃ alkyl, or C₁-C₃ alkoxy and wherein C₁-C₃ alkoxy and C₁-C₃ alkyl are independently optionally substituted with one to four halo; a line with a dotted line represents a double bond.

In some embodiments of Aspect 2, the D¹ includes, but is not limited to:

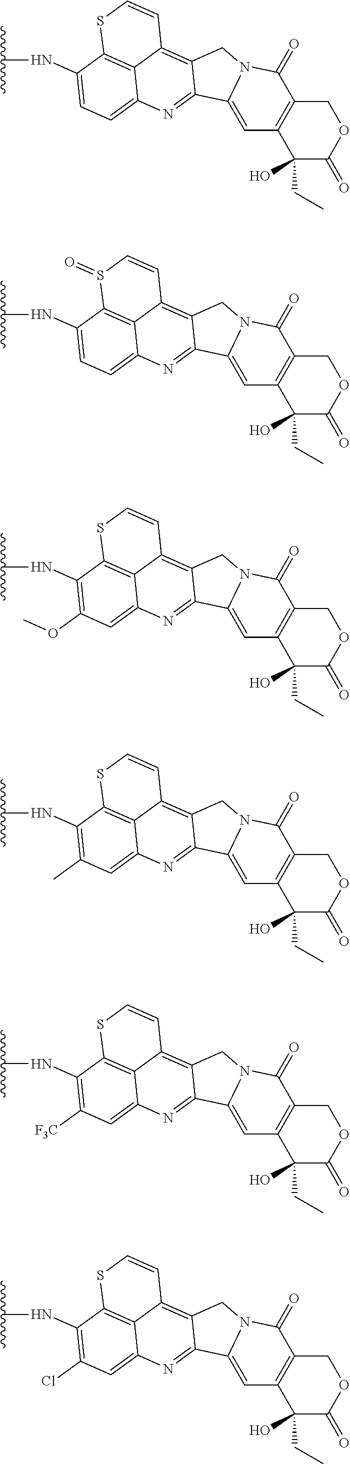 [SMILES: *Nc1ccc2nc3c(c4c2c1SC=C4)Cn1c-3cc2c(c1=O)COC(=O)[C@]2(O)CC ; *Nc1ccc2nc3c(c4c2c1S(=O)C=C4)Cn1c-3cc2c(c1=O)COC(=O)[C@]2(O)CC ; *Nc1c(OC)cc2nc3c(c4c2c1SC=C4)Cn1c-3cc2c(c1=O)COC(=O)[C@]2(O)CC ; *Nc1c(C)cc2nc3c(c4c2c1SC=C4)Cn1c-3cc2c(c1=O)COC(=O)[C@]2(O)CC ; *Nc1c(C(F)(F)F)cc2nc3c(c4c2c1SC=C4)Cn1c-3cc2c(c1=O)COC(=O)[C@]2(O)CC ; *Nc1c(Cl)cc2nc3c(c4c2c1SC=C4)Cn1c-3cc2c(c1=O)COC(=O)[C@]2(O)CC]

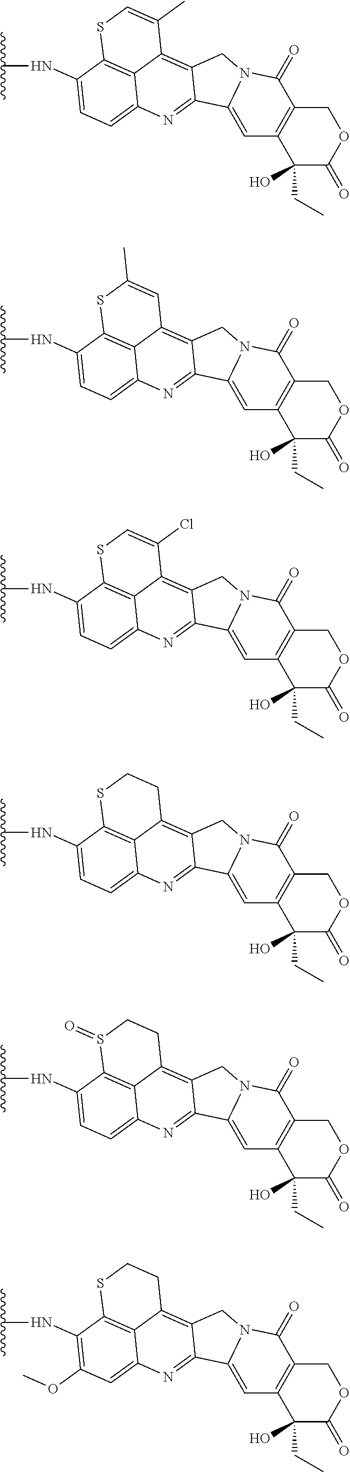 [SMILES: *Nc1ccc2nc3c(c4c2c1SC=C4C)Cn1c-3cc2c(c1=O)COC(=O)[C@]2(O)CC ; *Nc1ccc2nc3c(c4c2c1SC(C)=C4)Cn1c-3cc2c(c1=O)COC(=O)[C@]2(O)CC ; *Nc1ccc2nc3c(c4c2c1SC=C4Cl)Cn1c-3cc2c(c1=O)COC(=O)[C@]2(O)CC ; *Nc1ccc2nc3c(c4c2c1SCC4)Cn1c-3cc2c(c1=O)COC(=O)[C@]2(O)CC ; *Nc1ccc2nc3c(c4c2c1S(=O)CC4)Cn1c-3cc2c(c1=O)COC(=O)[C@]2(O)CC ; *Nc1c(OC)cc2nc3c(c4c2c1SCC4)Cn1c-3cc2c(c1=O)COC(=O)[C@]2(O)CC]

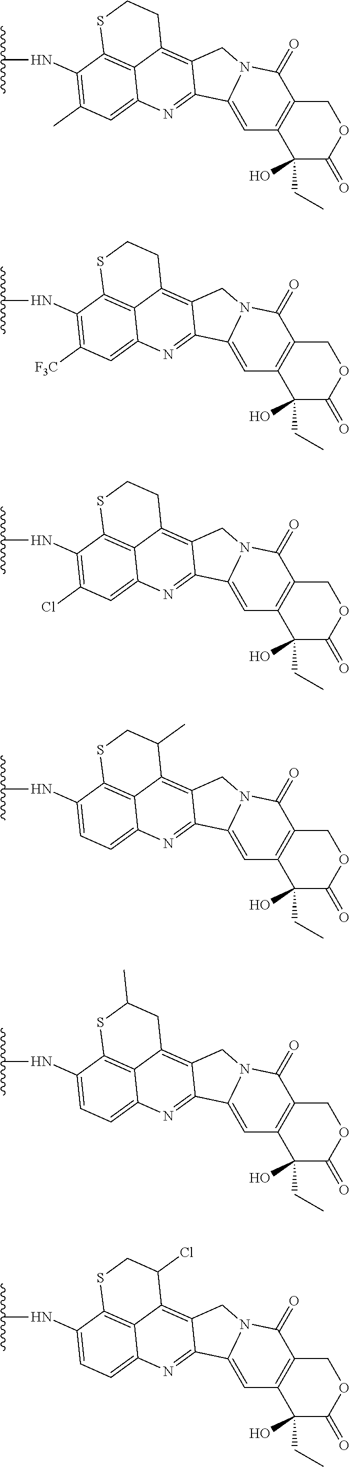 [SMILES: *Nc1c(C)cc2nc3c(c4c2c1SCC4)Cn1c-3cc2c(c1=O)COC(=O)[C@]2(O)CC ; *Nc1c(C(F)(F)F)cc2nc3c(c4c2c1SCC4)Cn1c-3cc2c(c1=O)COC(=O)[C@]2(O)CC ; *Nc1c(Cl)cc2nc3c(c4c2c1SCC4)Cn1c-3cc2c(c1=O)COC(=O)[C@]2(O)CC ; *Nc1ccc2nc3c(c4c2c1SCC4C)Cn1c-3cc2c(c1=O)COC(=O)[C@]2(O)CC ; *Nc1ccc2nc3c(c4c2c1SC(C)C4)Cn1c-3cc2c(c1=O)COC(=O)[C@]2(O)CC ; *Nc1ccc2nc3c(c4c2c1SCC4Cl)Cn1c-3cc2c(c1=O)COC(=O)[C@]2(O)CC]

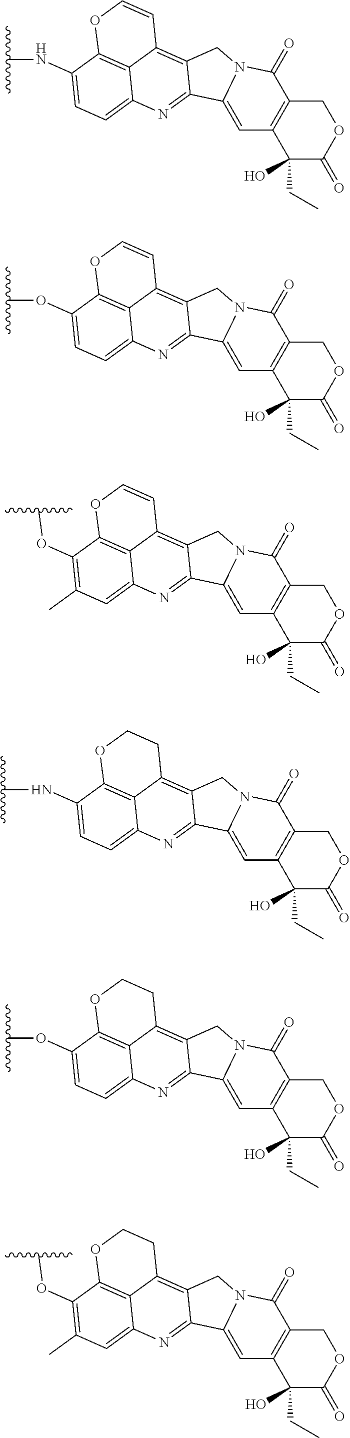 [SMILES: *Nc1ccc2nc3c(c4c2c1OC=C4)Cn1c-3cc2c(c1=O)COC(=O)[C@]2(O)CC ; *Oc1ccc2nc3c(c4c2c1OC=C4)Cn1c-3cc2c(c1=O)COC(=O)[C@]2(O)CC ; *Oc1c(C)cc2nc3c(c4c2c1OC=C4)Cn1c-3cc2c(c1=O)COC(=O)[C@]2(O)CC ; *Nc1ccc2nc3c(c4c2c1OCC4)Cn1c-3cc2c(c1=O)COC(=O)[C@]2(O)CC ; *Oc1ccc2nc3c(c4c2c1OCC4)Cn1c-3cc2c(c1=O)COC(=O)[C@]2(O)CC ; *Oc1c(C)cc2nc3c(c4c2c1OCC4)Cn1c-3cc2c(c1=O)COC(=O)[C@]2(O)CC]

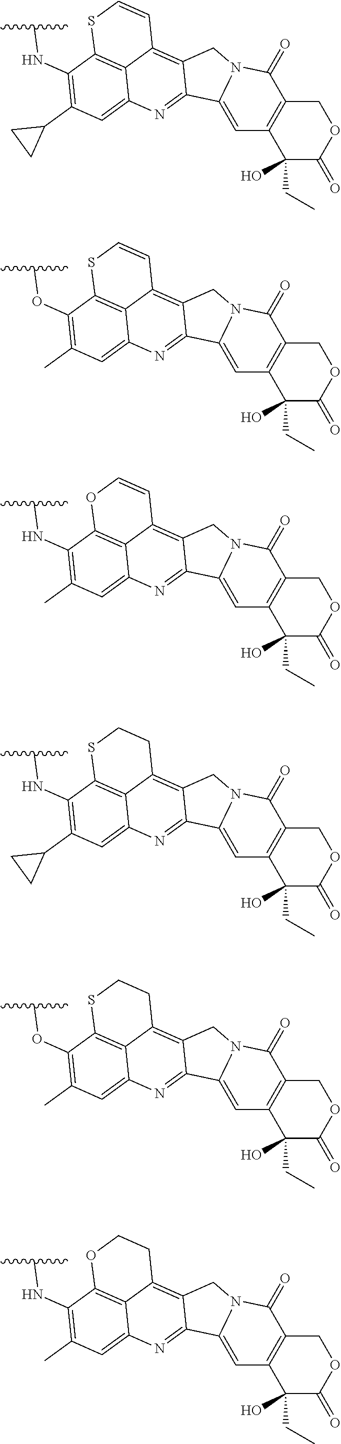 [SMILES: *Nc1c(C2CC2)cc2nc3c(c4c2c1SC=C4)Cn1c-3cc2c(c1=O)COC(=O)[C@]2(O)CC ; *Oc1c(C)cc2nc3c(c4c2c1SC=C4)Cn1c-3cc2c(c1=O)COC(=O)[C@]2(O)CC ; *Nc1c(C)cc2nc3c(c4c2c1OC=C4)Cn1c-3cc2c(c1=O)COC(=O)[C@]2(O)CC ; *Nc1c(C2CC2)cc2nc3c(c4c2c1SCC4)Cn1c-3cc2c(c1=O)COC(=O)[C@]2(O)CC ; *Oc1c(C)cc2nc3c(c4c2c1SCC4)Cn1c-3cc2c(c1=O)COC(=O)[C@]2(O)CC ; *Nc1c(C)cc2nc3c(c4c2c1OCC4)Cn1c-3cc2c(c1=O)COC(=O)[C@]2(O)CC]

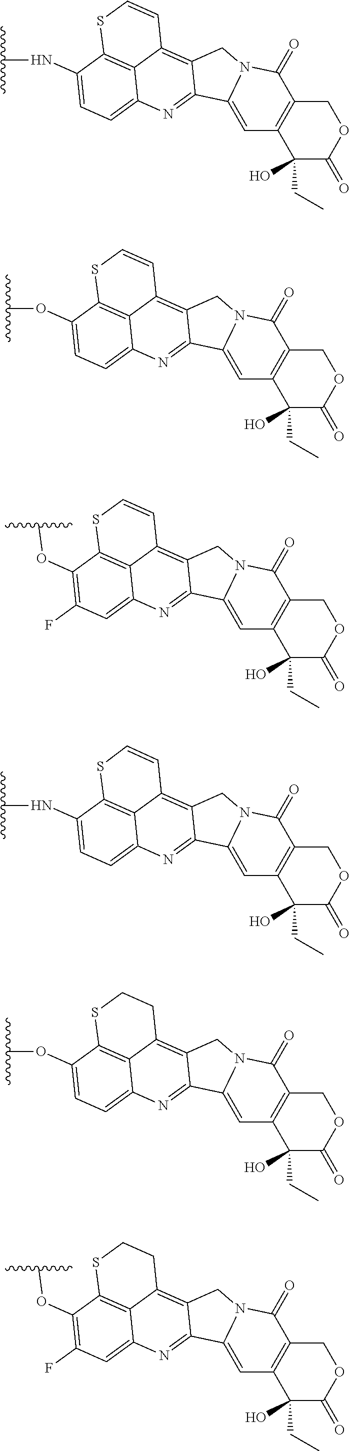 [SMILES: *Nc1ccc2nc3c(c4c2c1SC=C4)Cn1c-3cc2c(c1=O)COC(=O)[C@]2(O)CC ; *Oc1ccc2nc3c(c4c2c1SC=C4)Cn1c-3cc2c(c1=O)COC(=O)[C@]2(O)CC ; *Oc1c(F)cc2nc3c(c4c2c1SC=C4)Cn1c-3cc2c(c1=O)COC(=O)[C@]2(O)CC ; *Nc1ccc2nc3c(c4c2c1SC=C4)Cn1c-3cc2c(c1=O)COC(=O)[C@]2(O)CC ; *Oc1ccc2nc3c(c4c2c1SCC4)Cn1c-3cc2c(c1=O)COC(=O)[C@]2(O)CC ; *Oc1c(F)cc2nc3c(c4c2c1SCC4)Cn1c-3cc2c(c1=O)COC(=O)[C@]2(O)CC]

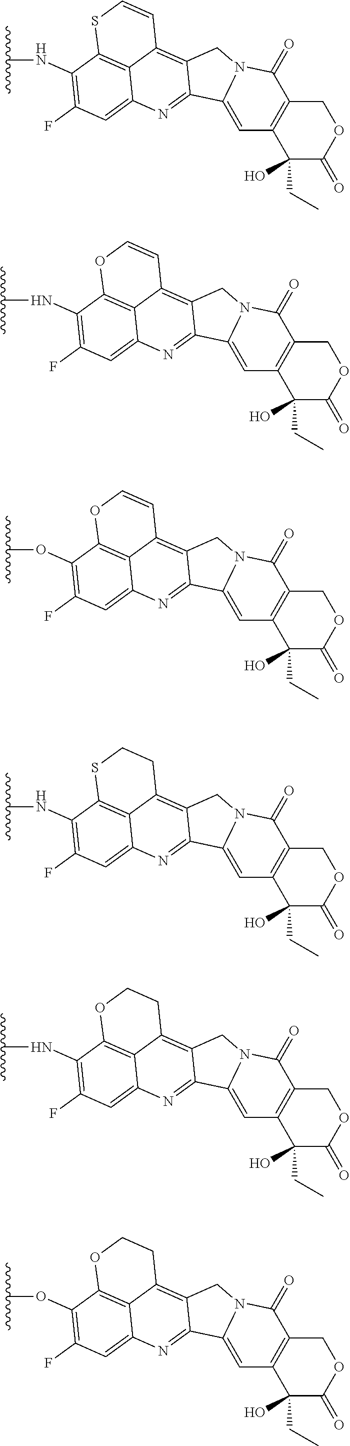 [SMILES: *Nc1c(F)cc2nc3c(c4c2c1SC=C4)Cn1c-3cc2c(c1=O)COC(=O)[C@]2(O)CC ; *Nc1c(F)cc2nc3c(c4c2c1OC=C4)Cn1c-3cc2c(c1=O)COC(=O)[C@]2(O)CC ; *Oc1c(F)cc2nc3c(c4c2c1OC=C4)Cn1c-3cc2c(c1=O)COC(=O)[C@]2(O)CC ; *Nc1c(F)cc2nc3c(c4c2c1SCC4)Cn1c-3cc2c(c1=O)COC(=O)[C@]2(O)CC ; *Nc1c(F)cc2nc3c(c4c2c1OCC4)Cn1c-3cc2c(c1=O)COC(=O)[C@]2(O)CC ; *Oc1c(F)cc2nc3c(c4c2c1OCC4)Cn1c-3cc2c(c1=O)COC(=O)[C@]2(O)CC]

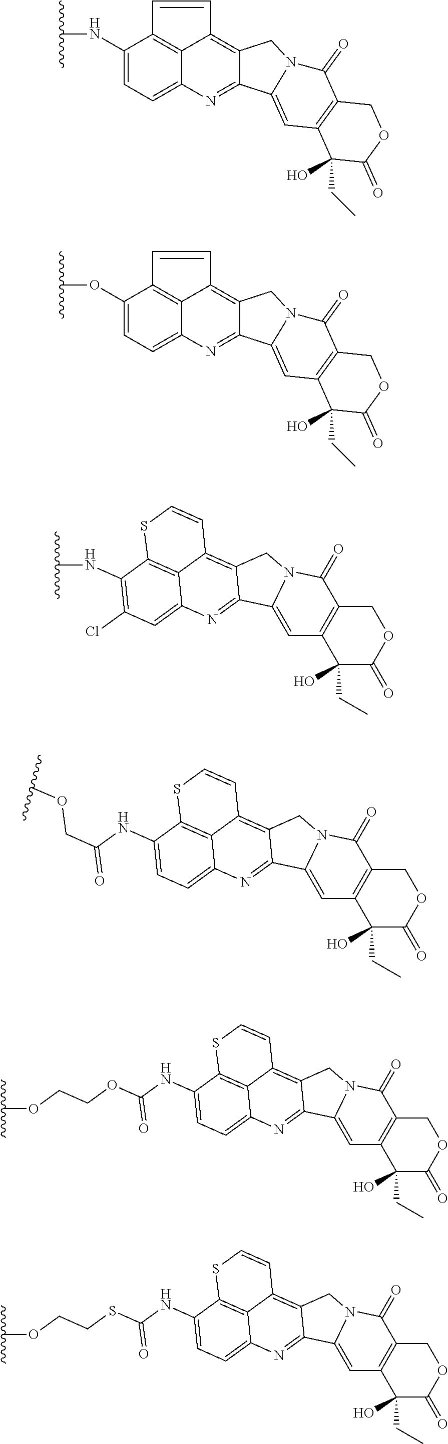 [SMILES: *Nc1ccc2nc3c(c4c2c1C=C4)Cn1c-3cc2c(c1=O)COC(=O)[C@]2(O)CC ; *Oc1ccc2nc3c(c4c2c1C=C4)Cn1c-3cc2c(c1=O)COC(=O)[C@]2(O)CC ; *Nc1c(Cl)cc2nc3c(c4c2c1SC=C4)Cn1c-3cc2c(c1=O)COC(=O)[C@]2(O)CC ; *OCC(=O)Nc1ccc2nc3c(c4c2c1SC=C4)Cn1c-3cc2c(c1=O)COC(=O)[C@]2(O)CC ; *OCCOC(=O)Nc1ccc2nc3c(c4c2c1SC=C4)Cn1c-3cc2c(c1=O)COC(=O)[C@]2(O)CC ; *OCCSC(=O)Nc1ccc2nc3c(c4c2c1SC=C4)Cn1c-3cc2c(c1=O)COC(=O)[C@]2(O)CC]

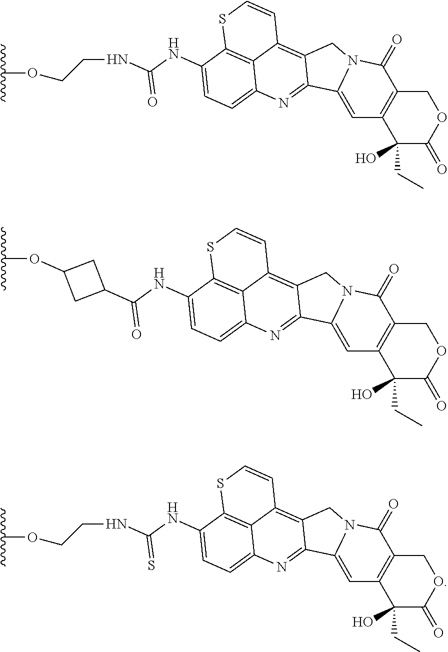 [SMILES: *OCCNC(=O)Nc1ccc2nc3c(c4c2c1SC=C4)Cn1c-3cc2c(c1=O)COC(=O)[C@]2(O)CC ; *OC1CC(C(=O)Nc2ccc3nc4c(c5c3c2SC=C5)Cn2c-4cc3c(c2=O)COC(=O)[C@]3(O)CC)C1 ; *OCCNC(=S)Nc1ccc2nc3c(c4c2c1SC=C4)Cn1c-3cc2c(c1=O)COC(=O)[C@]2(O)CC]

In some embodiments of Aspect 2, in the compound or a pharmaceutically acceptable salt, solvate, tautomer, mesomer, racemate, enantiomer, diastereomer, or combinations thereof according to the present disclosure, including of any of the formula (IV) and any embodiments thereof, the linker unit L^{1a} is selected from the group consisting of

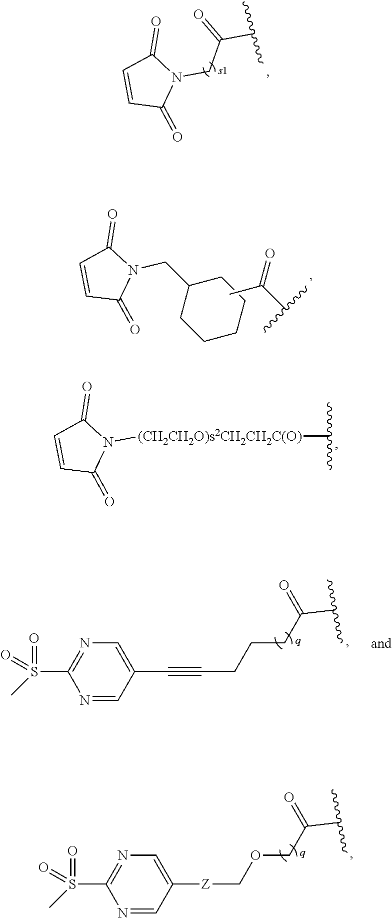

wherein s¹ is an integer selected from 2 to 8; s² is an integer selected from 1 to 3; q is an integer selected from 1 to 3; and Z is selected from the group consisting of C₁-C₆ alkylene, C₁-C₆ alkenylene, and C₁-C₆ alkynylene.

In some embodiments of Aspect 2, in the compound or a pharmaceutically acceptable salt, solvate, tautomer, mesomer, racemate, enantiomer, diastereomer, or combinations thereof according to the present disclosure, including of any of the formula (IV) and any embodiments thereof, the linker unit L² is selected from the group consisting of —NR⁸(CH₂CH₂O)ₚ¹CH₂CH₂C(O)—, —NR⁸(CH₂CH₂O)ₚ¹CH₂C(O)—, —N R⁸—(CH₂)ₚ²—(1H-1,2,3-triazole-1,4-diyl)-(CH₂CH₂O)ₚ¹ CH₂C(O), and a chemical bond; wherein R⁸ is selected from the group consisting of H, alkyl, haloalkyl, deuterated alkyl and hydroxyalkyl; p¹ is an integer selected from 6 to 12; p² is an integer selected from 1 to 3; wherein the left side of each of the L² groups provided above is attached to L¹.

In some embodiments of Aspect 2, in the compound or a pharmaceutically acceptable salt, solvate, tautomer, mesomer, racemate, enantiomer, diastereomer, or combinations thereof according to the present disclosure, including of any of the formula (IV) and any embodiments thereof, the linker unit L³ is a peptide residue composed of 2 to 7 amino acids, wherein the amino acids are selected from Phenylalanine (F), Glycine (G), Valine (V), Lysine (K), Citrulline, Serine (S), Alanine (A), Glutamic acid (E), and Aspartic acid (D). In certain embodiments, L³ is a peptide residue composed of 1, 2 or more Phenylalanine and Glycine. In certain embodiments, L³ is a peptide residue composed of 4 amino acids. In certain embodiments, L³ is a peptide residue GGFG or VA; wherein the left side of each of the L³ groups provided above is attached to L².

In some embodiments of Aspect 2, in the compound or a pharmaceutically acceptable salt, solvate, tautomer, mesomer, racemate, enantiomer, diastereomer, or combinations thereof according to the present disclosure, including of any of the formula (IV) and any embodiments thereof, the linker unit L⁴ is —NR⁹(CR¹⁰R¹¹)ₜ—,

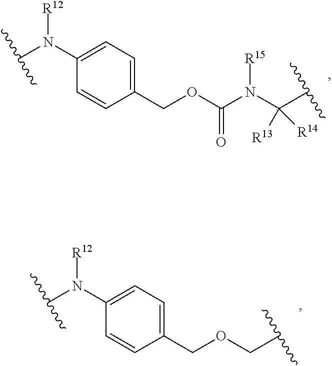

or a chemical bond; wherein R⁹, R¹⁰, R¹¹, R¹², R¹³, and R¹⁴ are each independently selected from H and alkyl; R¹⁵ is selected from —CH₂CH₂SO₂CH₃, and —CH₂CH₂N(CH₃)₂; t is 1 or 2; wherein in the presence of L⁴ groups, the left side of each of the L⁴ groups provided above is attached to the right side of L³ and the right side of each of the L⁴ groups is attached to D¹. In certain embodiments, L⁴ is a chemical bond;

In some embodiments of Aspect 2, in the compound or a pharmaceutically acceptable salt, solvate, tautomer, mesomer, racemate, enantiomer, diastereomer, or combinations thereof according to the present disclosure, including of any of the formula (IV) and any embodiments thereof, the linker unit L is L^{1a}-L²-L³-, wherein:
L^{1a} is

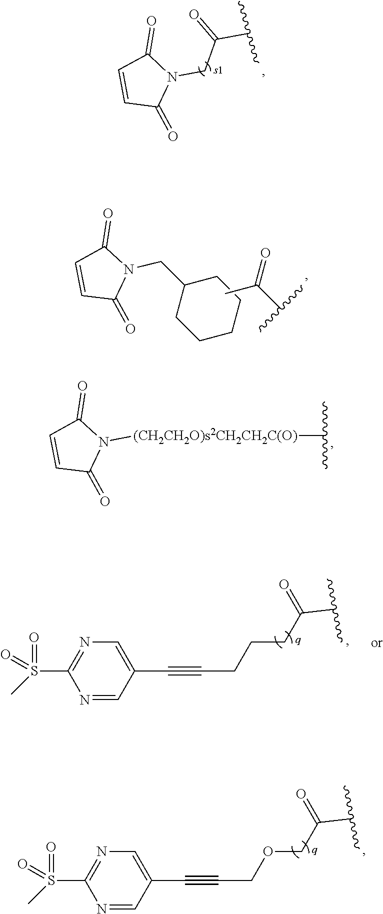

wherein s¹ is an integer selected from 2 to 8; s² is an integer selected from 1 to 3; and q is an integer selected from 1 to 3;
L² is selected from the group consisting of —NR⁸(CH₂CH₂O)ₚ¹CH₂CH₂C(O)—, —NR⁸(CH₂CH₂O)ₚ¹CH₂C(O)—, —N R⁸—(CH₂)ₚ²—(1H-1,2,3-triazole-1,4-diyl)-(CH₂CH₂O)ₚ¹ CH₂C(O), and a chemical bond, wherein R⁸ is selected from the group consisting of H, alkyl, haloalkyl, deuterated alkyl and hydroxyalkyl; p¹ is an integer selected from 6 to 12; p² is an integer selected from 1 to 3; wherein the left side of each of the L² groups provided above is attached to L¹;
L³ is a peptide residue GGFG or VA; wherein the left side of each of the L³ groups provided above is attached to L²;
and wherein the right side each of the L³ groups provided above is connected to D¹.

In some embodiments of Aspect 2, in the compound or a pharmaceutically acceptable salt, solvate, tautomer, mesomer, racemate, enantiomer, diastereomer, or combinations thereof according to the present disclosure, including of any of the formula (IV) and any embodiments thereof, the linker unit L includes, but is not limited to:

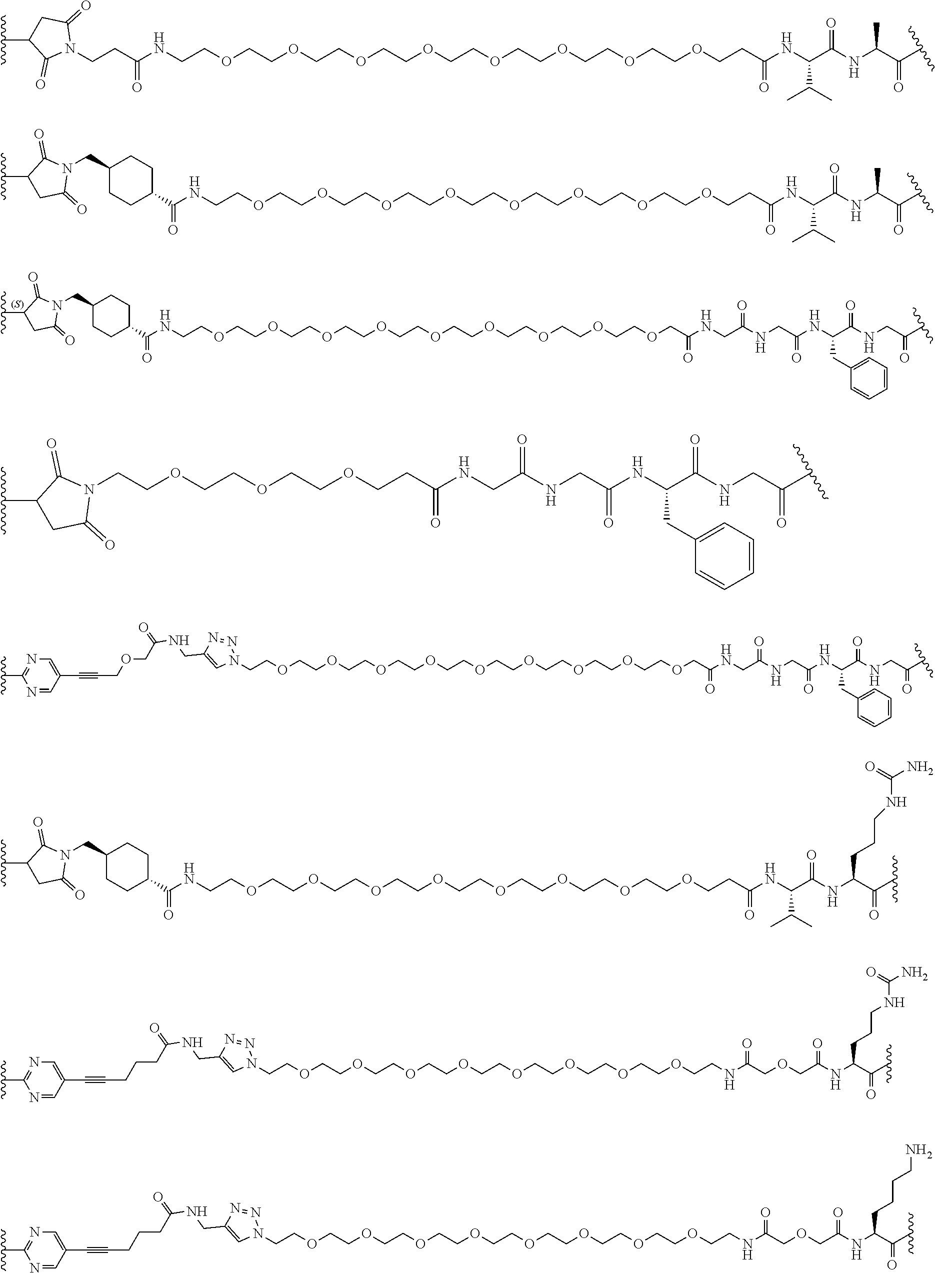

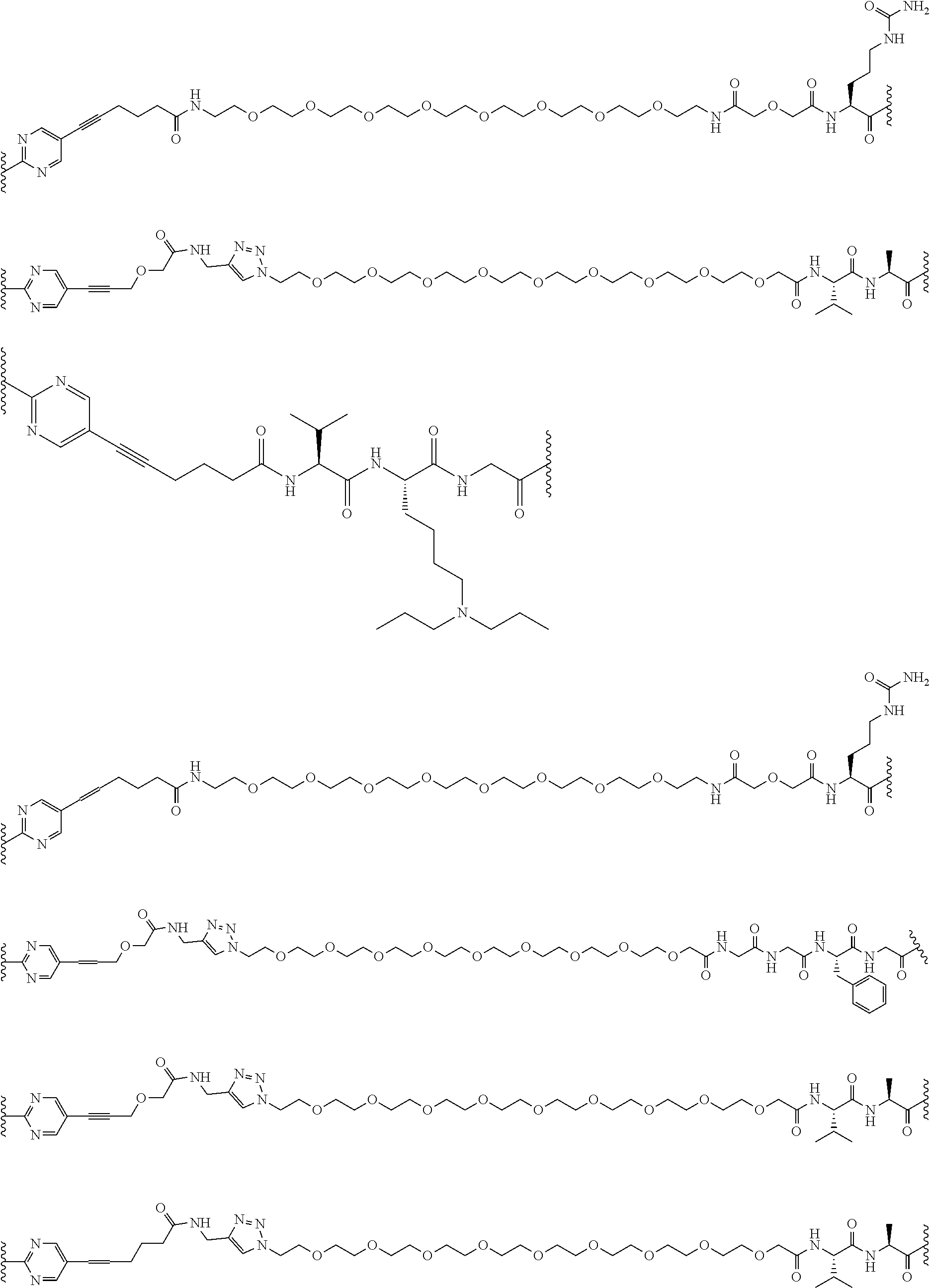

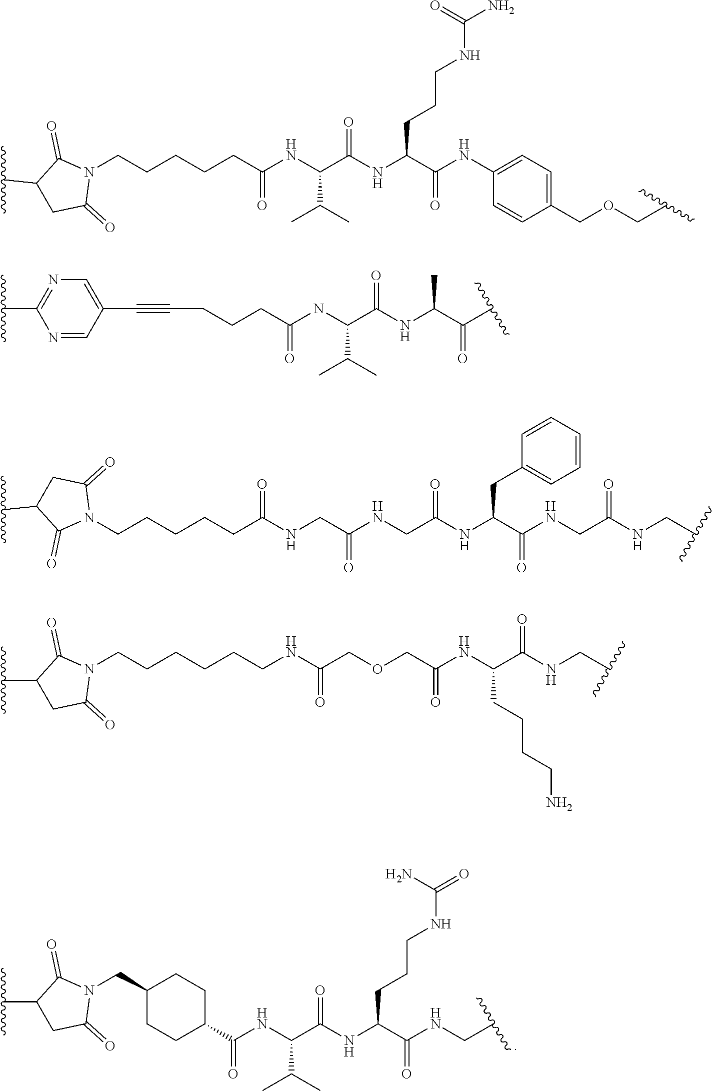

In some embodiments of Aspect 2, the compounds include, but are not limited to:

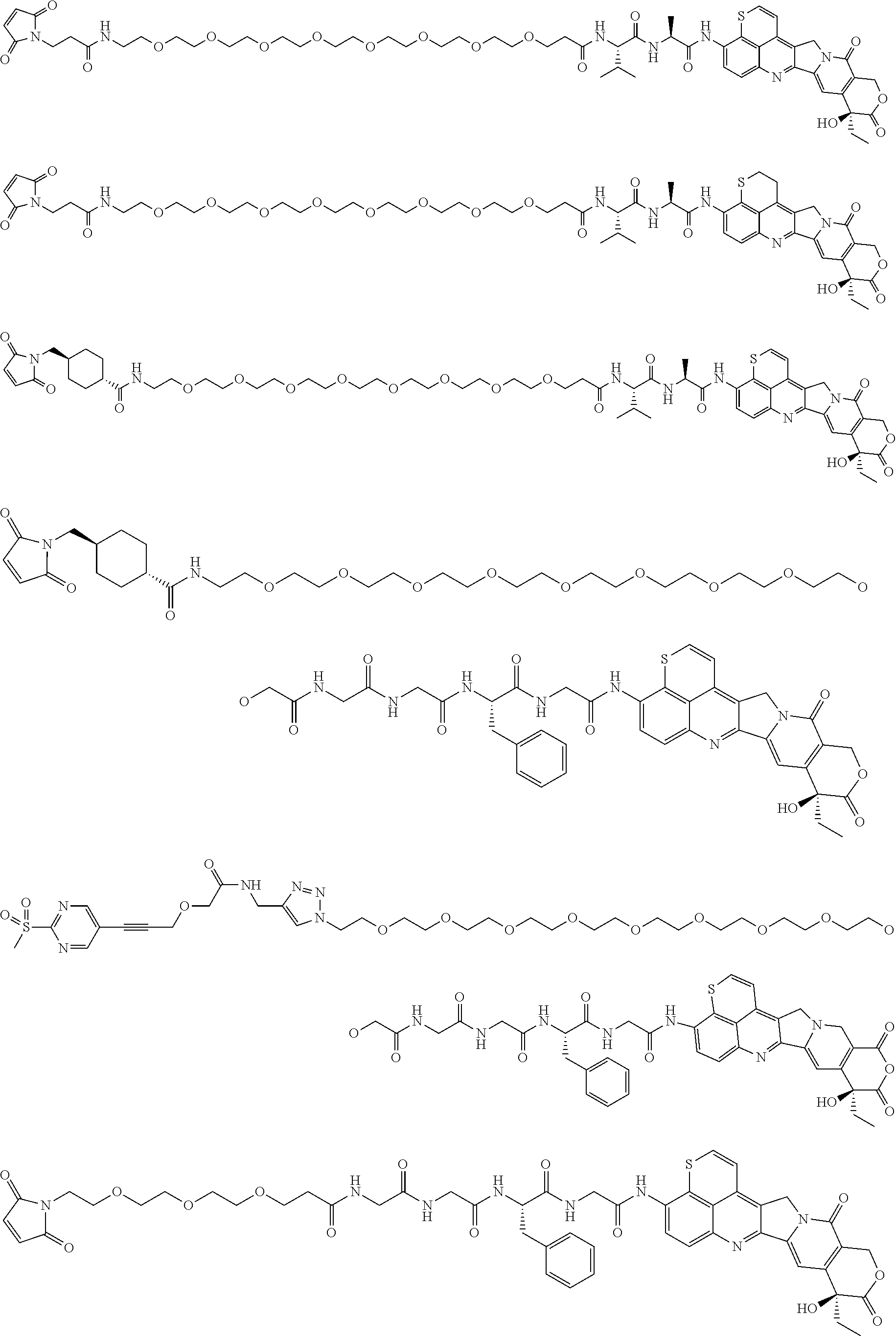

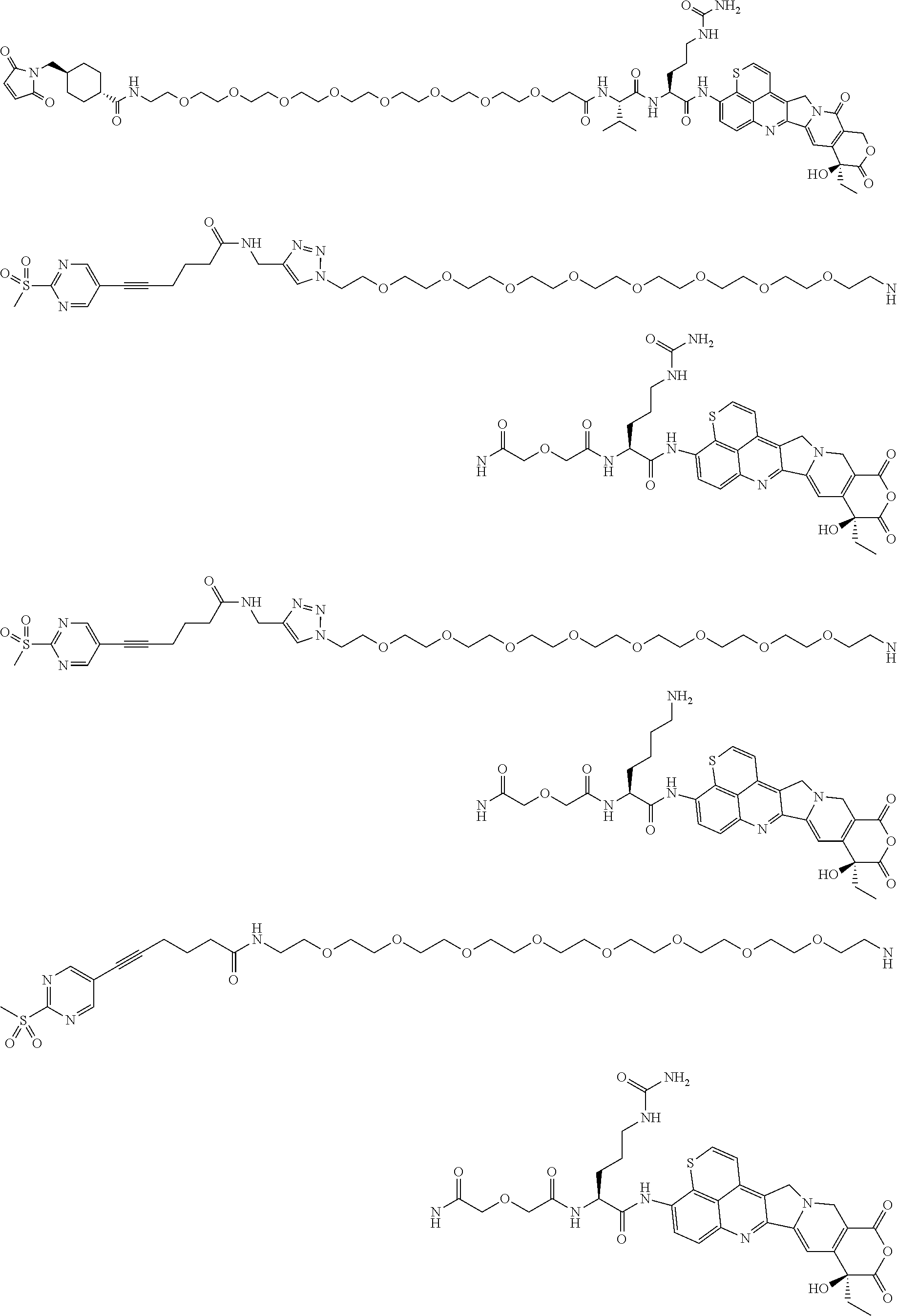

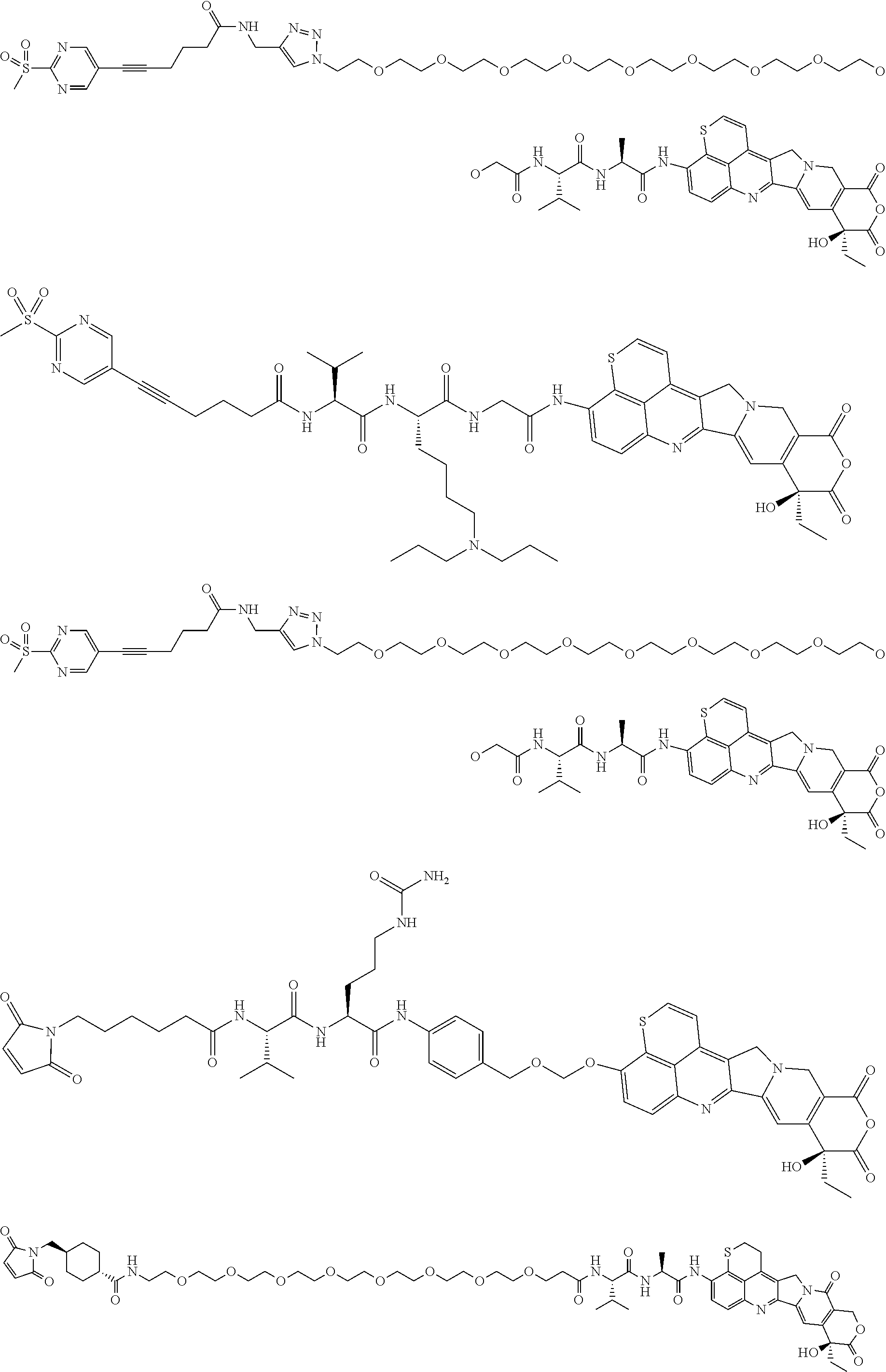

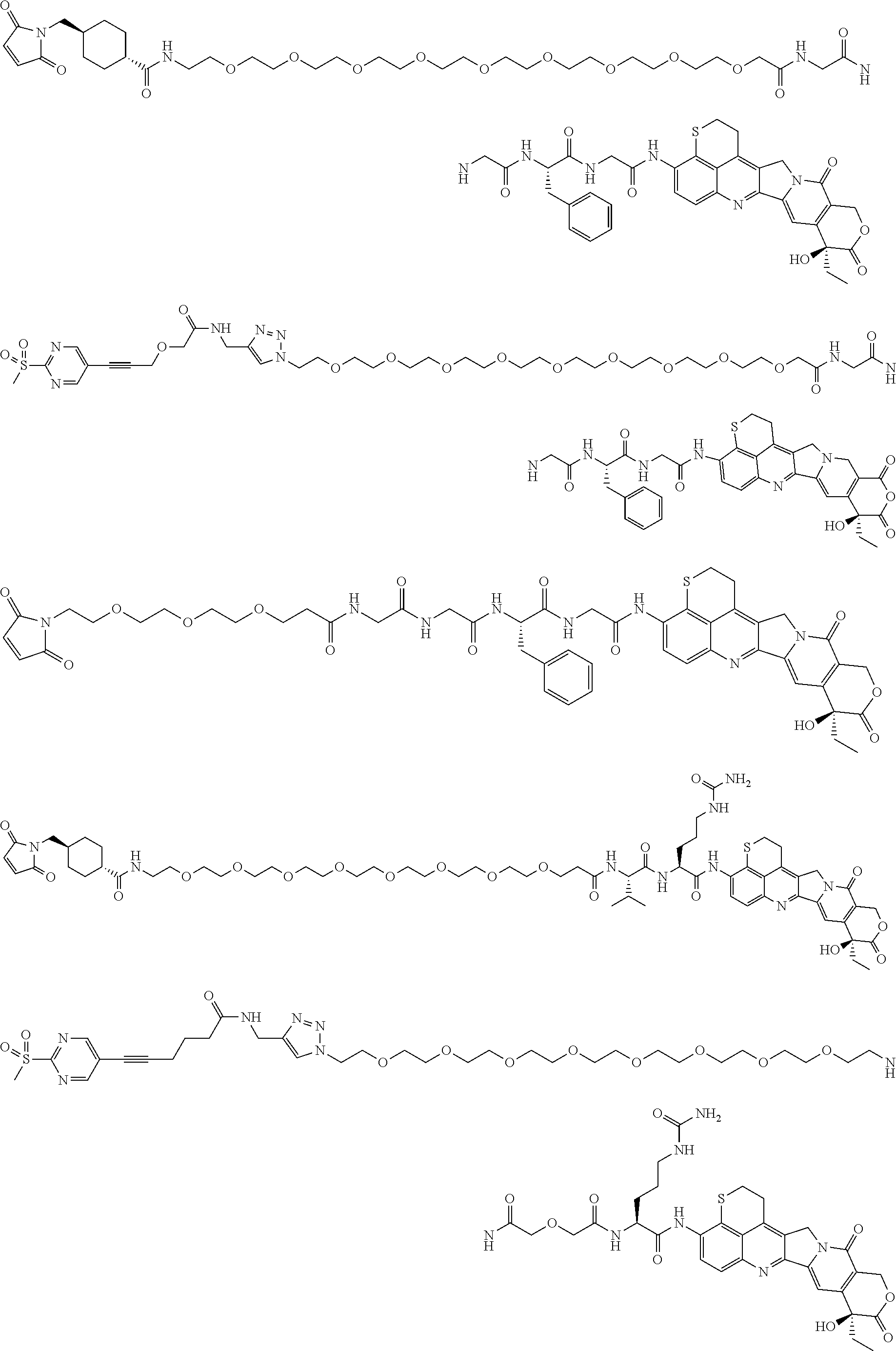

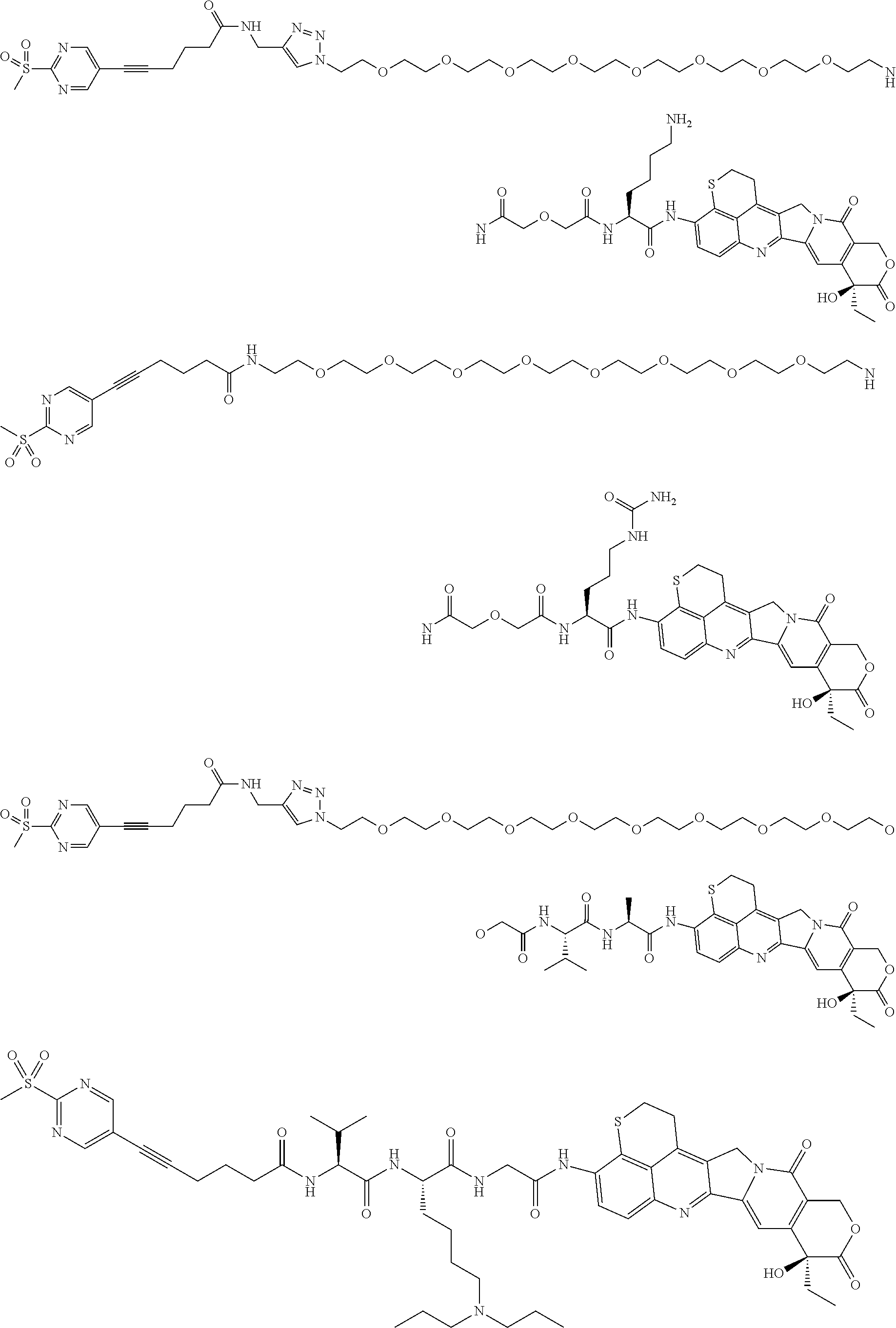

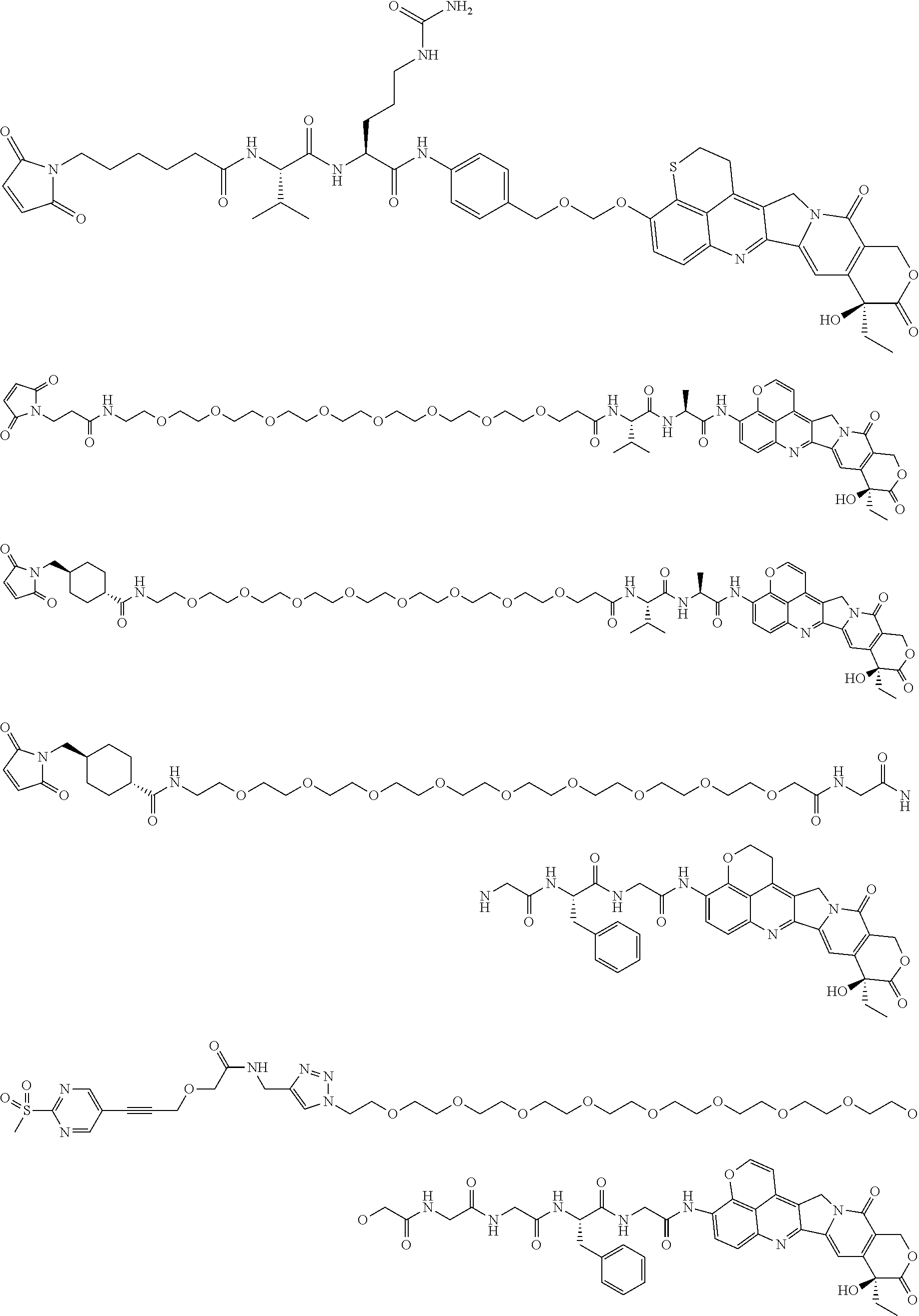

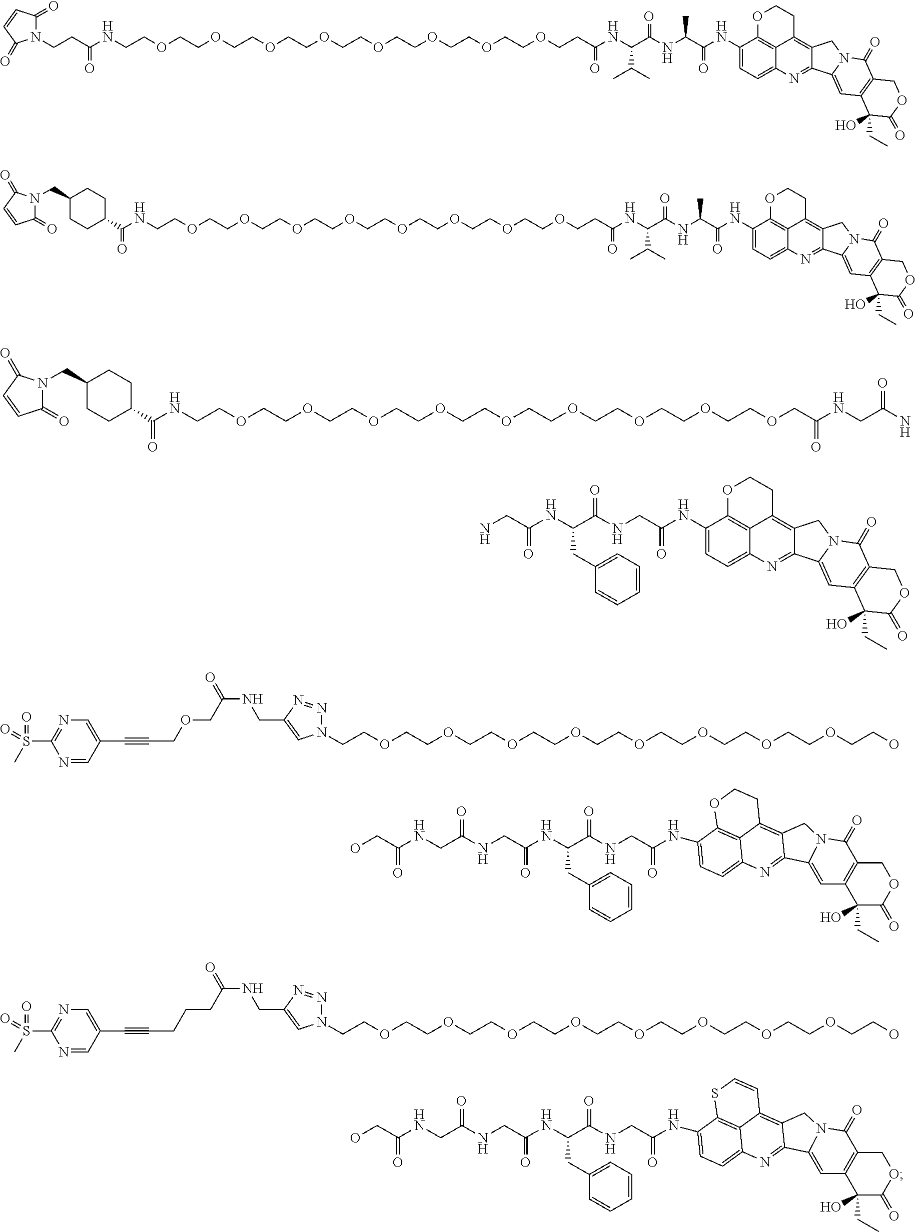

or a pharmaceutically acceptable salt, solvate, tautomer, mesomer, racemate, enantiomer, diastereomer, or combinations thereof.

Aspect 3: In some embodiments of the present disclosure, provided is a compound or a pharmaceutically acceptable salt, solvate, tautomer, mesomer, racemate, enantiomer, diastereomer, or combinations thereof, where the compound of formula (V):

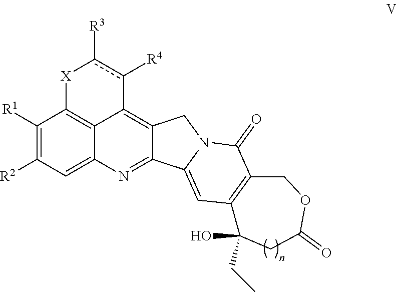

Wherein:
X is either absent, or selected from —O—, —S—, —S(O₂)—, or —S(O)—;
R¹ is —NH—R^{1b}—OH, hydroxy or amino; optionally, R¹ is hydroxy or amino;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶
R² is selected from H, halo, hydroxy, cyano, nitro, C₃-C₈ cycloalkyl, C₁-C₈ alkyl, C₁-C₈ alkoxy, R⁵—(C₁-C₈ alkylene)-, amino, (C₁-C₈ alkyl)NHC(O)—, (C₃-C₈ cycloalkyl)NHC(O)O—, (C₁-C₈ alkyl)NH—, azetidin-1-yl, pyrrolidin-1-yl, piperidin-1-yl, azepane-1-yl, (C₁-C₈ alkyl)C(O)O—, or (C₁-C₈ alkyl)C(O)NH—; wherein azetidin-1-yl, pyrrolidin-1-yl, piperidin-1-yl, azepane-1-yl, C₁-C₈ alkoxy, each “C₃-C₈ cycloalkyl”, and each “C₁-C₈ alkyl” are independently optionally substituted with one to four R⁶;
R³ and R⁴ are independently selected from H, halo, hydroxy, cyano, C₁-C₈ alkyl, C₁-C₈ alkoxy, C₃-C₈ cycloalkyl, aryl, heteroaryl, or amino; wherein C₁-C₈ alkyl, C₁-C₈ alkoxy, C₃-C₈ cycloalkyl, aryl, and heteroaryl are independently optionally substituted with one to four R⁶;
R⁵ is selected from C₁-C₈ alkoxy, C₃-C₈ cycloalkyl, aryl, or heteroaryl; wherein C₁-C₈ alkoxy, C₃-C₈ cycloalkyl, aryl, and heteroaryl are independently optionally substituted with one to four R⁶;
R⁶ at each occurrence is independently selected from halo, hydroxy, cyano, amino, C₁-C₈ alkyl, C₃-C₈ cycloalkyl, aryl, or heteroaryl;
and wherein a line with a dotted line represents either a single bond or a double bond;
n is an integer from 0 or 1;
provided that when X is —O— or —S—, and a line with a dotted line represents a single bond, then R², R³, and R⁴ are not all H and are not amino.

In some embodiments of Aspect 3, provided is a compound or a pharmaceutically acceptable salt, solvate, tautomer, mesomer, racemate, enantiomer, diastereomer, or combinations thereof, where the compound of formula (VI):

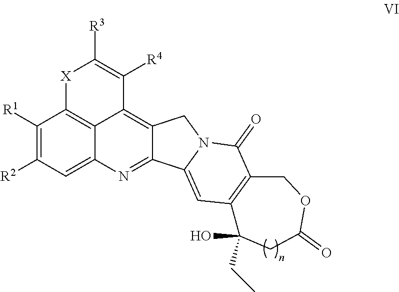

wherein: R¹, R², R³, R⁴, X, and n are as defined as in formula (V) and any embodiments thereof.

In some embodiments of Aspect 3, provided is a compound or a pharmaceutically acceptable salt, solvate, tautomer, mesomer, racemate, enantiomer, diastereomer, or combinations thereof, where the compound of formula (VII):

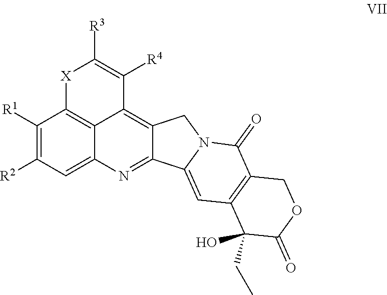

Wherein:
X is selected from —O—, —S—, —S(O₂)—, or —S(O)—;
R¹ is —NH—R^{1b}—OH, hydroxy or amino; optionally, R¹ is hydroxy or amino;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R² is selected from H, halo, hydroxy, cyano, nitro, C₃-C₆ cycloalkyl, C₁-C₆ alkyl, C₁-C₆ alkoxy, R⁵—(C₁-C₃ alkylene)-, amino; wherein C₁-C₆ alkoxy, C₃-C₆ cycloalkyl, and C₁-C₆ alkyl are independently optionally substituted with one to four R⁶;
R³ and R⁴ are independently selected from H, halo, hydroxy, cyano, C₁-C₆ alkyl, C₁-C₆ alkoxy, or C₃-C₆ cycloalkyl; wherein C₁-C₆ alkyl, C₁-C₆ alkoxy, and C₃-C₆ cycloalkyl are independently optionally substituted with one to four R⁶;
R⁵ is C₁-C₆ alkoxy or C₃-C₆ cycloalkyl; wherein C₁-C₆ alkoxy and C₃-C₆ cycloalkyl are independently optionally substituted with one to four R⁶;
R⁶ at each occurrence is independently selected from halo, hydroxy, cyano, amino, C₁-C₃ alkyl, or C₃-C₆ cycloalkyl.

In some embodiments of Aspect 3, in compound or a pharmaceutically acceptable salt, solvate, tautomer, mesomer, racemate, enantiomer, diastereomer, or combinations thereof according to the present disclosure, including of any of the formula (V), (VI), and (VII) and any embodiments thereof, wherein X is —O— or —S—.

In some embodiments of Aspect 3, in compound or a pharmaceutically acceptable salt, solvate, tautomer, mesomer, racemate, enantiomer, diastereomer, or combinations thereof according to the present disclosure, including of any of the formula (V), (VI), and (VII) and any embodiments thereof, wherein R², R³ and R⁴ are independently selected from H, halo, C₁-C₃ alkyl, or C₁-C₃ alkoxy and wherein C₁-C₃ alkoxy and C₁-C₃ alkyl are independently optionally substituted with one to four halo.

In some embodiments of Aspect 3, in compound or a pharmaceutically acceptable salt, solvate, tautomer, mesomer, racemate, enantiomer, diastereomer, or combinations thereof according to the present disclosure, including of any of the formula (V), (VI), and (VII) and any embodiments thereof, wherein:
X is —S—;
R¹ is —NH—R^{1b}—OH or amino; optionally, R¹ is amino;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R², R³ and R⁴ are independently selected from H, halo, C₁-C₃ alkyl, or C₁-C₃ alkoxy and wherein C₁-C₃ alkoxy and C₁-C₃ alkyl are independently optionally substituted with one to four halo.

In some embodiments of Aspect 3, the compounds include, but are not limited to:

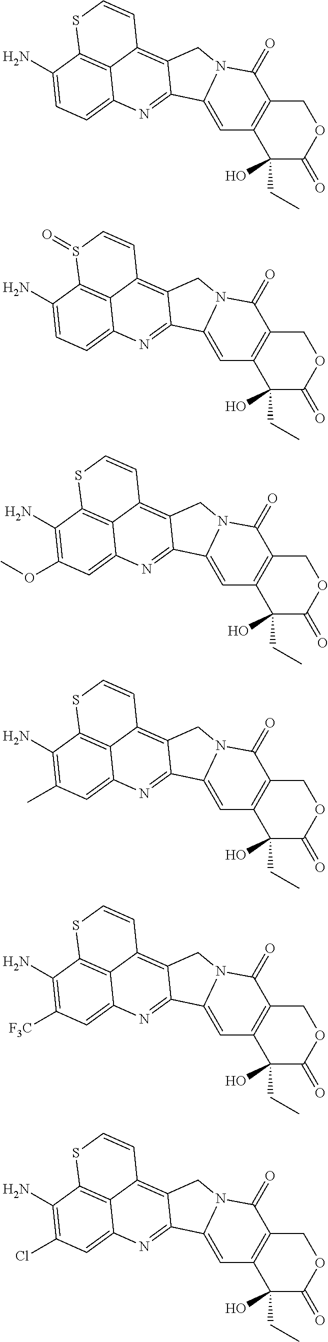 [SMILES: CC[C@@]1(O)C(=O)OCc2c1cc1n(c2=O)Cc2c-1nc1ccc(N)c3c1c2C=CS3 ; CC[C@@]1(O)C(=O)OCc2c1cc1n(c2=O)Cc2c-1nc1ccc(N)c3c1c2C=CS3=O ; CC[C@@]1(O)C(=O)OCc2c1cc1n(c2=O)Cc2c-1nc1cc(OC)c(N)c3c1c2C=CS3 ; CC[C@@]1(O)C(=O)OCc2c1cc1n(c2=O)Cc2c-1nc1cc(C)c(N)c3c1c2C=CS3 ; CC[C@@]1(O)C(=O)OCc2c1cc1n(c2=O)Cc2c-1nc1cc(C(F)(F)F)c(N)c3c1c2C=CS3 ; CC[C@@]1(O)C(=O)OCc2c1cc1n(c2=O)Cc2c-1nc1cc(Cl)c(N)c3c1c2C=CS3]

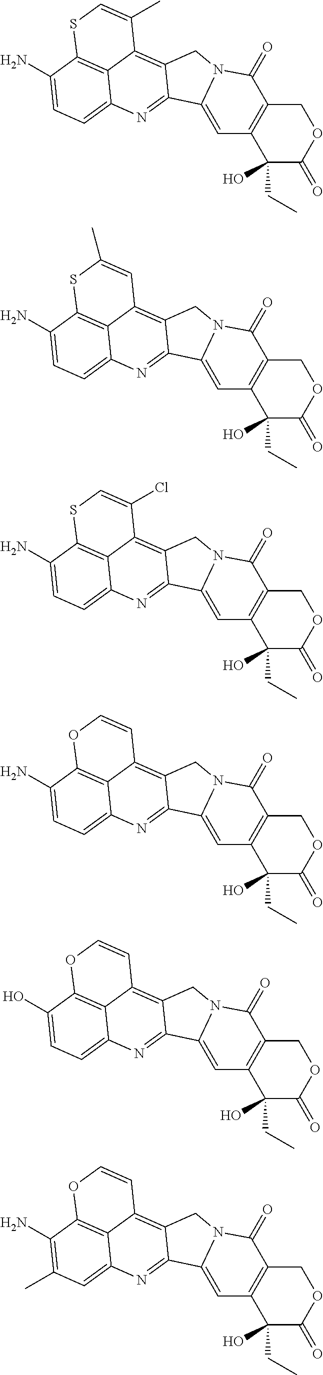 [SMILES: CC[C@@]1(O)C(=O)OCc2c1cc1n(c2=O)Cc2c-1nc1ccc(N)c3c1c2C(C)=CS3 ; CC[C@@]1(O)C(=O)OCc2c1cc1n(c2=O)Cc2c-1nc1ccc(N)c3c1c2C=C(C)S3 ; CC[C@@]1(O)C(=O)OCc2c1cc1n(c2=O)Cc2c-1nc1ccc(N)c3c1c2C(Cl)=CS3 ; CC[C@@]1(O)C(=O)OCc2c1cc1n(c2=O)Cc2c-1nc1ccc(N)c3c1c2C=CO3 ; CC[C@@]1(O)C(=O)OCc2c1cc1n(c2=O)Cc2c-1nc1ccc(O)c3c1c2C=CO3 ; CC[C@@]1(O)C(=O)OCc2c1cc1n(c2=O)Cc2c-1nc1cc(C)c(N)c3c1c2C=CO3]

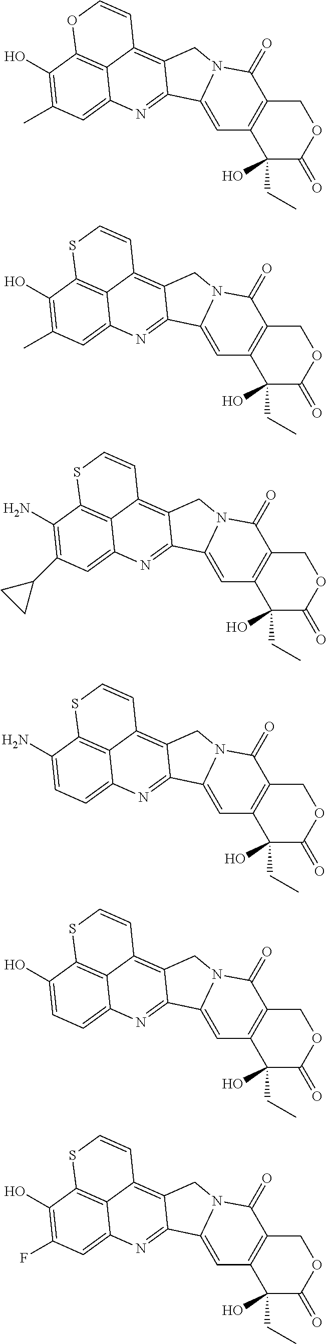 [SMILES: CC[C@@]1(O)C(=O)OCc2c1cc1n(c2=O)Cc2c-1nc1cc(C)c(O)c3c1c2C=CO3 ; CC[C@@]1(O)C(=O)OCc2c1cc1n(c2=O)Cc2c-1nc1cc(C)c(O)c3c1c2C=CS3 ; CC[C@@]1(O)C(=O)OCc2c1cc1n(c2=O)Cc2c-1nc1cc(C3CC3)c(N)c3c1c2C=CS3 ; CC[C@@]1(O)C(=O)OCc2c1cc1n(c2=O)Cc2c-1nc1ccc(N)c3c1c2C=CS3 ; CC[C@@]1(O)C(=O)OCc2c1cc1n(c2=O)Cc2c-1nc1ccc(O)c3c1c2C=CS3 ; CC[C@@]1(O)C(=O)OCc2c1cc1n(c2=O)Cc2c-1nc1cc(F)c(O)c3c1c2C=CS3]

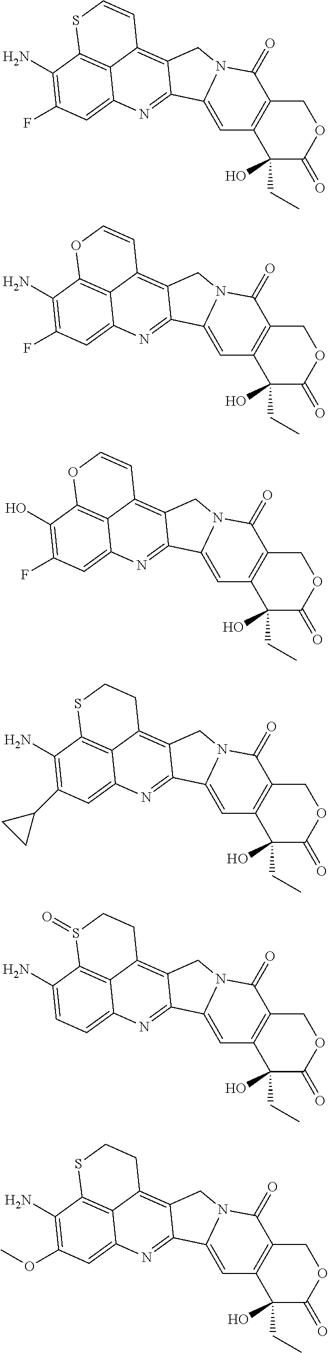 [SMILES: CC[C@@]1(O)C(=O)OCc2c1cc1n(c2=O)Cc2c-1nc1cc(F)c(N)c3c1c2C=CS3 ; CC[C@@]1(O)C(=O)OCc2c1cc1n(c2=O)Cc2c-1nc1cc(F)c(N)c3c1c2C=CO3 ; CC[C@@]1(O)C(=O)OCc2c1cc1n(c2=O)Cc2c-1nc1cc(F)c(O)c3c1c2C=CO3 ; CC[C@@]1(O)C(=O)OCc2c1cc1n(c2=O)Cc2c-1nc1cc(C3CC3)c(N)c3c1c2CCS3 ; CC[C@@]1(O)C(=O)OCc2c1cc1n(c2=O)Cc2c-1nc1ccc(N)c3c1c2CCS3=O ; CC[C@@]1(O)C(=O)OCc2c1cc1n(c2=O)Cc2c-1nc1cc(OC)c(N)c3c1c2CCS3]

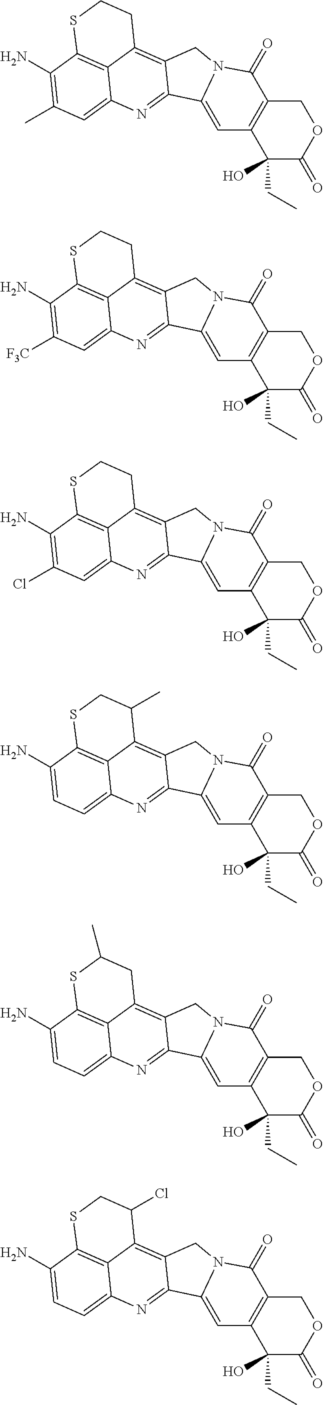 [SMILES: CC[C@@]1(O)C(=O)OCc2c1cc1n(c2=O)Cc2c-1nc1cc(C)c(N)c3c1c2CCS3 ; CC[C@@]1(O)C(=O)OCc2c1cc1n(c2=O)Cc2c-1nc1cc(C(F)(F)F)c(N)c3c1c2CCS3 ; CC[C@@]1(O)C(=O)OCc2c1cc1n(c2=O)Cc2c-1nc1cc(Cl)c(N)c3c1c2CCS3 ; CC[C@@]1(O)C(=O)OCc2c1cc1n(c2=O)Cc2c-1nc1ccc(N)c3c1c2C(C)CS3 ; CC[C@@]1(O)C(=O)OCc2c1cc1n(c2=O)Cc2c-1nc1ccc(N)c3c1c2CC(C)S3 ; CC[C@@]1(O)C(=O)OCc2c1cc1n(c2=O)Cc2c-1nc1ccc(N)c3c1c2C(Cl)CS3]

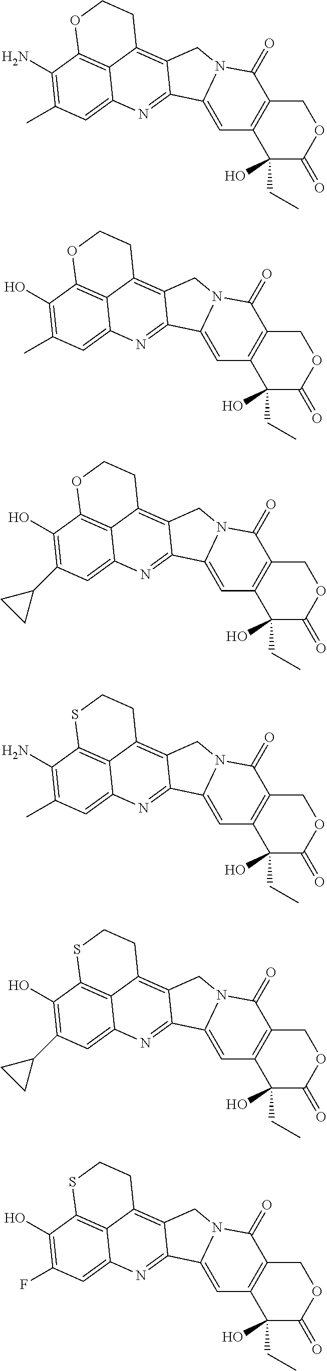 [SMILES: CC[C@@]1(O)C(=O)OCc2c1cc1n(c2=O)Cc2c-1nc1cc(C)c(N)c3c1c2CCO3 ; CC[C@@]1(O)C(=O)OCc2c1cc1n(c2=O)Cc2c-1nc1cc(C)c(O)c3c1c2CCO3 ; CC[C@@]1(O)C(=O)OCc2c1cc1n(c2=O)Cc2c-1nc1cc(C3CC3)c(O)c3c1c2CCO3 ; CC[C@@]1(O)C(=O)OCc2c1cc1n(c2=O)Cc2c-1nc1cc(C)c(N)c3c1c2CCS3 ; CC[C@@]1(O)C(=O)OCc2c1cc1n(c2=O)Cc2c-1nc1cc(C3CC3)c(O)c3c1c2CCS3 ; CC[C@@]1(O)C(=O)OCc2c1cc1n(c2=O)Cc2c-1nc1cc(F)c(O)c3c1c2CCS3]

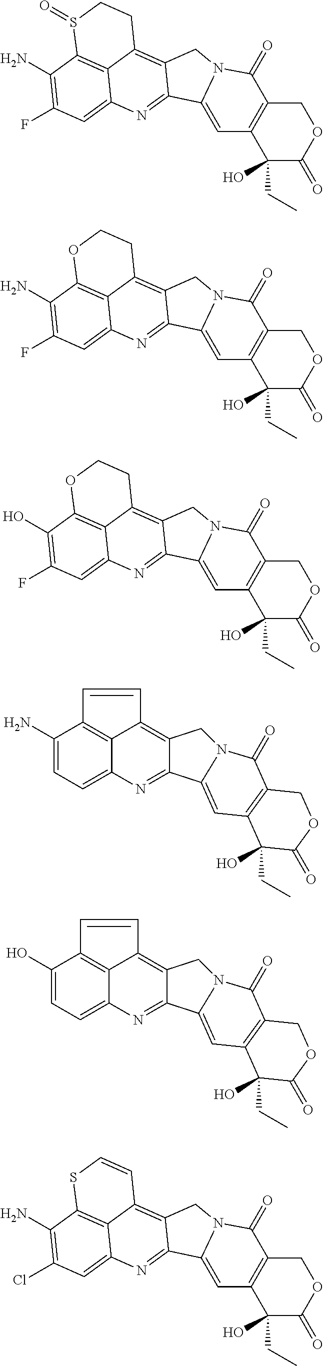 [SMILES: CC[C@@]1(O)C(=O)OCc2c1cc1n(c2=O)Cc2c-1nc1cc(F)c(N)c3c1c2CCS3=O ; CC[C@@]1(O)C(=O)OCc2c1cc1n(c2=O)Cc2c-1nc1cc(F)c(N)c3c1c2CCO3 ; CC[C@@]1(O)C(=O)OCc2c1cc1n(c2=O)Cc2c-1nc1cc(F)c(O)c3c1c2CCO3 ; CC[C@@]1(O)C(=O)OCc2c1cc1n(c2=O)Cc2c-1nc1ccc(N)c3c1c2C=C3 ; CC[C@@]1(O)C(=O)OCc2c1cc1n(c2=O)Cc2c-1nc1ccc(O)c3c1c2C=C3 ; CC[C@@]1(O)C(=O)OCc2c1cc1n(c2=O)Cc2c-1nc1cc(Cl)c(N)c3c1c2C=CS3]

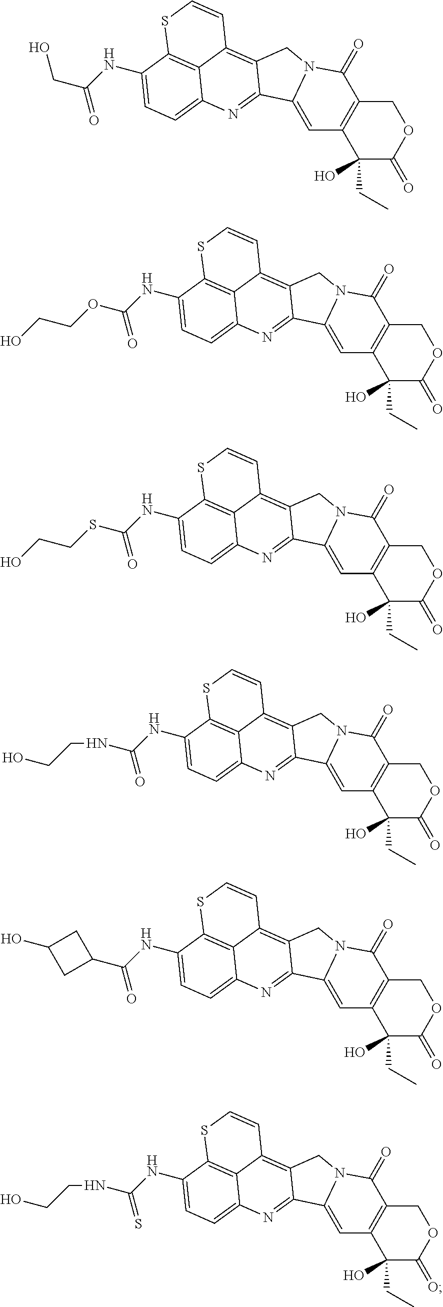 [SMILES: CC[C@@]1(O)C(=O)OCc2c1cc1n(c2=O)Cc2c-1nc1ccc(NC(=O)CO)c3c1c2C=CS3 ; CC[C@@]1(O)C(=O)OCc2c1cc1n(c2=O)Cc2c-1nc1ccc(NC(=O)OCCO)c3c1c2C=CS3 ; CC[C@@]1(O)C(=O)OCc2c1cc1n(c2=O)Cc2c-1nc1ccc(NC(=O)SCCO)c3c1c2C=CS3 ; CC[C@@]1(O)C(=O)OCc2c1cc1n(c2=O)Cc2c-1nc1ccc(NC(=O)NCCO)c3c1c2C=CS3 ; CC[C@@]1(O)C(=O)OCc2c1cc1n(c2=O)Cc2c-1nc1ccc(NC(=O)C3CC(O)C3)c3c1c2C=CS3 ; CC[C@@]1(O)C(=O)OCc2c1cc1n(c2=O)Cc2c-1nc1ccc(NC(=S)NCCO)c3c1c2C=CS3]

or the pharmaceutically acceptable salt, solvate, tautomer, mesomer, racemate, enantiomer, diastereomer, or combinations thereof.

In another embodiment, the cell-surface binding molecule T may be of any kind presently known, or which become known cell binding ligands, such as peptides and non-peptides. Generally, the cell-binding molecule T is an antibody; a single chain antibody; an antibody fragment that binds to the target cell; a monoclonal antibody; a single chain monoclonal antibody; or a monoclonal antibody fragment that binds the target cell; a chimeric antibody; a chimeric antibody fragment that binds to the target cell; a domain antibody; a domain antibody fragment that binds to the target cell; adnectins that mimic antibodies, DARPins; a lymphokine; a hormone; a vitamin; a growth factor; a colony stimulating factor; or a nutrient-transport molecule (a transferrin), a binding peptide, or protein, or antibody, or small affinity molecule attached on albumin, polymers, dendrimers, liposomes, nanoparticles, vesicles, (viral)capsids. In certain embodiments, the binding molecule T is a monoclonal antibody.

Also provided herein is a compound of the formula (I), (II), (III), (IV), (V), (VI), and (VII) and any embodiments thereof, including as defined in the Aspect 1, Aspect 2, and Aspect 3, or a pharmaceutically acceptable salt or solvate thereof, wherein the compound is a tautomer, mesomer, racemate, enantiomer, diastereomer or mixture thereof.

Another aspect of the present disclosure provides a ligand-drug conjugate, wherein the compound or a pharmaceutically acceptable salt, solvate, tautomer, mesomer, racemate, enantiomer, diastereomer, or combinations thereof as defined in the Aspect 3 used as a toxin.

Another aspect of the present disclosure provides a method for preparing the ligand-drug conjugate of formula (I), (II), and (III), and any embodiments thereof, or the pharmaceutically acceptable salt or solvate thereof, and optionally a tautomer, mesomer, racemate, enantiomer, diastereomer or mixture thereof.

Another aspect of the present disclosure further relates to a pharmaceutical composition comprising a therapeutically effective amount of 1) the ligand-drug conjugate or compound or the pharmaceutically acceptable salt or solvate thereof according to the present disclosure and optionally a tautomer, mesomer, racemate, enantiomer, diastereomer or mixture thereof, and 2) one or more pharmaceutically acceptable carrier(s), diluent(s) or excipient(s).

Another aspect of the present disclosure further relates to a use of 1) the ligand-drug conjugate or compound of the present disclosure, or the pharmaceutically acceptable salt or solvate thereof, and optionally a tautomer, mesomer, racemate, enantiomer, diastereomer or mixture thereof or 2) the pharmaceutical composition comprising the same according to the present disclosure in the preparation of a medicament for treating or preventing a tumor, and optionally the tumor is a cancer related to the expression of HER2, HER3 or EGFR.

Another aspect of the present disclosure further relates to a use of 1) the ligand-drug conjugate or compound, or the pharmaceutically acceptable salt or solvate thereof, and optionally a tautomer, mesomer, racemate, enantiomer, diastereomer or mixture thereof or 2) the pharmaceutical composition comprising the same according to the present disclosure in the preparation of a medicament for treating or preventing a cancer. In certain embodiments, the cancer is selected from the group consisting of breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblasfoma, sarcoma, lung cancer (for example, small cell lung cancer and non-small cell lung cancer), colon cancer, rectal cancer, colorectal cancer, leukemia (for example, acute lymphocytic leukemia, acute myeloid leukemia, acute promyelocytic leukemia, chronic myeloid leukemia, chronic lymphocytic leukemia), bone cancer, skin cancer, thyroid cancer, pancreatic cancer and lymphoma (for example, Hodgkin's lymphoma, non-Hodgkin's lymphoma, or recurrent anaplastic large cell lymphoma).

The active compound can be formulated into a form suitable for administration by ay appropriate route, and the active compound is preferably in the form of a unit dose, or in a form in which the patient can self-administer in a single dose. The form of the unit dose of the compound or composition of the present disclosure can be tablet, capsule, cachet, bottled portion, powder, granule, lozenge, suppository, regenerating powder or liquid preparation.

The dosage of the compound or composition in the treatment method of the present disclosure will generally vary according to the severity of the disease, the weight of the patient, and the relative efficacy of the compound. However, as a general guide, a suitable unit dose can be 0.1 to 1000 mg.

In addition to the active compound, the pharmaceutical composition of the present disclosure can also comprise one or more auxiliaries including filter (diluent), binder, wetting agent, disintegrant, excipient and the like. Depending on the administration mode, the composition can comprise 0.1 to 99% by weight of the active compound.

The pharmaceutical composition containing the active ingredient can be in a form suitable for oral administration, for example, a tablet, troche, lozenge, aqueous or oily suspension, dispersible powder or granule, emulsion, hard or soft capsule, syrup or elixir. An oral composition can be prepared according to any known method in the art for the preparation of pharmaceutical composition. Such composition can comprise binders, fillers, lubricants, disintegrants or pharmaceutically acceptable wetting agents and the like. Such composition can also comprise one or more components selected from the group consisting of sweeteners, flavoring agents, colorants and preservatives, in order to provide a pleasing and palatable pharmaceutical formulation.

An aqueous suspension comprises an active ingredient in admixture with excipients suitable fbr the manufacture of an aqueous suspension. The aqueous suspension can also comprise one or more preservative(s), one or more colorant(s), one or more flavoring agent(s), and one or more sweetener(s).

An oil suspension can be formulated by suspending the active ingredient in a vegetable oil. The oil suspension can comprise a thickener. The aforementioned sweeteners and flavoring agents can be added to provide a palatable formulation.

The pharmaceutical composition of the present disclosure can also be in the form of an oil-in-water emulsion.

The pharmaceutical composition can be in the form of a sterile injectable aqueous solution. Acceptable vehicles or solvents that can be used are water, Ringer's solution or isotonic sodium chloride solution. The sterile injectable formulation can be a sterile injectable oil-in-water micro-emulsion in which the active ingredient is dissolved in an oil phase. For example, the active ingredient is dissolved in a mixture of soybean oil and lecithin. The oil solution is then added to a mixture of water and glycerin, and processed to form a micro-emulsion. The injectable solution or micro-emulsion can be introduced into a patient's bloodstream by local bolus injection. Alternatively, the solution and micro-emulsion are preferably administrated in a manner that maintains a constant circulating concentration of the compound of the present disclosure. In order to maintain this constant concentration, a continuous intravenous delivery device can be used. An example of such a device is Deltec CADD-PLUS™ 5400 intravenous injection pump.

The pharmaceutical composition can be in the form of a sterile injectable aqueous or oily suspension fbr intramuscular and subcutaneous administration. Such a suspension can be formulated with suitable dispersants or wetting agents and suspending agents as described above according to known techniques. The sterile injectable formulation can also be a sterile injectable solution or suspension prepared in a nontoxic parenterally acceptable diluent or solvent. Moreover, sterile fixed oils can easily be used as a solvent or suspending medium.

The compound of the present disclosure can be administrated in the form of a suppository for rectal administration. These pharmaceutical compositions can be prepared by mixing the drug with a suitable non-irritating excipient that is solid at ordinary temperatures, but liquid in the rectum, thereby melting in the rectum to release the drug. Such materials include cocoa butter, glycerin gelatin, hydrogenated vegetable oil, a mixture of polyethylene glycols of various molecular weights and fatty acid esters thereof.

It is well known to those skilled in the art that the dosage of a drug depends on a variety of factors including, but not limited to the following factors: activity of a specific compound, age of the patient, weight of the patient, general health of the patient, behavior of the patient, diet of the patient, administration time, administration route, excretion rate, drug combination and the like. In addition, the optimal treatment, such as treatment mode, daily dose of the compound of formula (I) or the type of pharmaceutically accept-able salt thereof can be verified according to traditional therapeutic regimens.

### DETAILED DESCRIPTION

Unless otherwise stated, all technical and scientific terms used herein are consistent with the common understanding of those of ordinary skill in the art to which the present disclosure belongs. Although any methods and materials similar or equivalent to those described herein can be used in the practice or testing of the present disclosure, preferred methods and materials are described herein. When describing and protecting the present disclosure, the following terms are used in accordance with the following definitions.

Unless otherwise stated, the terms used in the specification and claims have the meanings described below.

“A” and “an,” as used in herein, mean one or more, unless the context clearly dictates otherwise.

“Ligand” refers to a compound capable of recognizing and binding to an antigen or receptor associated with a target cell. The role of the ligand is to deliver the drug to the target cell population that binds to the ligand. Such ligands include, but are not limited to, protein hormones, lectins, growth factors, antibodies, peptides or other molecules that can bind to cells. In an embodiment of the present disclosure, the ligand is represented by T for trastuzumab. The ligand can form a bond with the Linker via a heteroatom on the ligand. In certain embodiments, the ligand is an antibody or an antigen binding fragment thereof. The antibody is selected from the group consisting of chimeric antibody, humanized antibody, fully humanized antibody or murine antibody. In certain embodiments, the ligand is a monoclonal antibody.

The term “drug” refers to a cytotoxic drug as provided herein, being a chemical molecule that can strongly disrupt the normal growth of tumor cells. In principle, all cytotoxic drugs can kill tumor cells at a sufficiently high concentration. However, it they may cause the apoptosis of normal cell resulting in serious side effects, even while killing tumor cells due to the lack of specificity.

The term “linker,” “linker unit,” “linking fragment,” or “linking unit” refers to a chemical structural fragment or bond, which is linked to a ligand at one end and linked to a drug at another end. The preferred embodiments of the present disclosure are represented by L and L¹ to L⁴, wherein the L¹ end is linked to the ligand, and the L⁴ end is linked to the drug.

The linker, including extension unit, spacer unit, and amino acid unit, can be synthesized by methods known in the art, such as those described in US 2005-0238649A1. The linker can be a “cleavable linker” or “releasable linker” that facilitates the release of the drug in cell. For example, an acid labile linker (for example, hydrazone), a protease-sensitive (for example, peptidase-sensitive) linker, a light-labile linker, a dimethyl linker or a disulfide-containing linker can be used (Chari et al., Cancer Research 52: 127-131 (1992); U.S. Pat. No. 5,208,020).

The term “ligand-drug conjugate” means that a biologically active drug is linked to a ligand as provided herein through a stable linking unit. In the present disclosure, the “ligand-drug conjugate” is optionally an antibody-drug conjugate (ADC), which means that a toxic drug is linked to a monoclonal antibody or antibody fragment with biological activity through a stable linking unit.

The three-letter codes and one-letter codes for amino acids used in the present disclosure are as described in J. Biol. Chem, 243, p 3558 (1968).

The term “antibody” refers to immunoglobulin, a four-peptide chain structure connected together by interchain disulfide bond between two identical heavy chains and two identical light chains. Different immunoglobulin heavy chain constant regions exhibit different amino acid compositions and sequences, hence present different antigenicity. Accordingly, immunoglobulins can be divided into five types, or called immunoglobulin isotypes, namely IgM, IgD, IgG, IgA and IgE, with corresponding heavy chain i, y, a and e, respectively. According to the amino acid composition of hinge region and the number and location of heavy chain disulfide bonds, the same type of Ig can further be divided into different sub-types, for example, IgG can be divided into IgG1, IgG2, IgG3 and IgG4. Light chain can be divided into K or X chain based on different constant region. Each five types of Ig can have a K or X chain. The antibodies described in the present disclosure are optionally specific antibodies against the cell surface antigens on the target cells, non-limiting examples are one or more of the following antibodies: anti-HER2 (ErbB2) antibody, anti-EGFR antibody, anti-B7-H3 antibody, anti-c-Met antibody, anti-HER3 (ErbB3) antibody, anti-HER4 (ErbB4) antibody, anti-CD20 antibody, anti-CD22 antibody, anti-CD30 antibody, anti-CD33 antibody, anti-CD44 antibody, anti-CD56 antibody, anti-CD70 antibody, anti-CD73 antibody, anti-CD105 antibody, anti-CEA antibody, anti-A33 antibody, anti-Cripto antibody, anti-EphA2 antibody, anti-G250 antibody, anti-MUC1 antibody, anti-Lewis Y antibody, anti-VEGFR antibody, anti-GPNMB antibody, anti-Integrin antibody, anti-PSMA antibody, anti-Tenascin-C antibody, anti-SLC44A4 antibody or anti-Mesothelin antibody, and optionally Trastuzumab (trade name Herceptin), Pertuzumab (also known as 2C₄, trade name Peijeta), Nimotuzumab (trade name Taixinsheng), Enoblituzumab, Emibetuzumab, Inotuzumab, Pinatuzumab, Brentuximab, Gemtuzumab, Bivatuzumab, Lorvotuzumab, cBR96 and Glembatumumab.

About 110 amino acid sequence adjacent to the N-terminus of the antibody heavy chains or light chains is highly variable, known as variable region (Fv region); the rest of amino acid sequence adjacent to the C-terminus is relatively stable, known as constant region. The variable region includes three hypervariable regions (HVR) and four relatively conservative framework regions (FR). The three hypervariable regions, which determine the specificity of the antibody, are also known as the complementarity determining regions (CDR). Each light chain variable region (LCVR) or each heavy chain variable region (HCVR) consists of three CDR regions and four FR regions, with sequential order from the amino terminus to carboxyl terminus in the following order: FR1, CDR1, FR2, CDR2, FR3, CDR3, FR4. The three CDR regions of the light chain refer to LCDR1, LCDR2, and LCDR3; and the three CDR regions of the heavy chain refer to HCDR1, HCDR2, and HCDR3.

Antibodies of the present disclosure include murine antibodies, chimeric antibodies, humanized antibodies and fully humanized antibodies, and preferably humanized antibodies and fully humanized antibodies.

The term “murine antibody” in the present disclosure refers to the antibody prepared from murine according to the knowledge and skills of the field. During the preparation, the test subject is injected with specific antigen, and then a hybridoma expressing the antibody which possesses the desired sequence or functional characteristics is isolated.

The term “chimeric antibody,” is an antibody obtained by fusing a variable region of a murine antibody with a constant region of a human antibody, and the chimeric antibody can alleviate the murine antibody-induced immune response. To establish a chimeric antibody, a hybridoma secreting murine specific monoclonal antibody is established, and a variable region gene is cloned from the murine hybridoma cell; then a constant region gene of human antibody is cloned according to requirement; and the constant region gene of human is connected with the variable region gene of murine to form a chimeric gene, which is subsequently inserted into an expression vector; finally, the chimeric antibody molecule is expressed in an eukaryotic or prokaryotic system.

The term “humanized antibody” which is also known as CDR-grafted antibody, refers to an antibody generated by grafting murine CDR sequences into human antibody variable region framework, i.e., an antibody produced in different types of human germline antibody framework sequences. Humanized antibody can overcome heterologous responses induced by large number of murine protein components carried by chimeric antibody. Such framework sequences can be obtained from public DNA database covering germline antibody gene sequences or published references. For example, gemline DNA sequences of human heavy and light chain variable region genes can be found in “VBase” human germline sequence database (available on the world wide web at: www.mrccpe.com.ac.uk/vbase), as well as in Kabat, E A, et al. 1991 Sequences of Proteins of Immunological Interest, 5th Ed. To avoid a decrease in activity caused by the decreased immunogenicity, the framework sequences in the variable region of human antibody can be subjected to minimal reverse mutations or back mutations to maintain the activity. The humanized antibody of the present disclosure also comprises humanized antibody on which CDR affinity maturation is performed by phage display. Documents that further describe methods of using murine antibodies involved in humanization include, for example, Queen et al., Proc., Natl. Acad. Sci. USA, 88, 2869,1991 and Winter and colleagues' method [Jones et al., Nature, 321, 522(1986), Riechmann et al., Nature, 332, 323-327(1988), Verhoeyen et al., Science, 239, 1534(1988)].

The term “fully humanized antibody,” is also known as “fully humanized monoclonal antibody,” wherein the variable region and constant region of the antibody are both of human origin, eliminating immunogenicity and side effects. The development of monoclonal antibody has gone through four stages, namely: murine monoclonal antibody, chimeric monoclonal antibody, humanized monoclonal antibody and fully humanized monoclonal antibody. The antibody of the present disclosure is a fully humanized monoclonal antibody. The related technologies of fully humanized antibody preparation mainly include human hybridoma technology, EBV transformed B lymphocyte technology, phage display technology, transgenic mouse antibody preparation technology, single B cell antibody preparation technology and the like.

The term “antigen binding fragment” refers to one or more fragments of an antibody retaining the specific binding ability to the antigen. It has been shown that fragments of full-length antibody can be used to achieve the function of binding with an antigen. The examples of binding fragments in the term “antigen binding fragment” include (i) Fab fragment, a monovalent fragment composed of VL, VH, CL and CH1 domain; (ii) F(ab′)2 fragment, a bivalent fragment comprising two Fab fragments connected by a disulphide bond in the hinge region; (iii) Fd fragment, consisting of VH and CH: domains; (iv) Fv fragment, consisting of VH and VL domains of one-arm antibody; (v) single domain or dAb fragment (Ward et al. (1989) Nature 341:544-546) composed of VH domain; and (vi) an isolated complementary determining region (CDR) or (vii) a combination of two or more isolated CDRs optionally connected by a synthetic linker. In addition, although the VL domain and VH domain of the Fv fragment are encoded by two separate genes, they can be connected by a synthetic linker by using recombinant methods, thereby generating a single protein chain of a monovalent molecular formed by pairing the VL and VH domain (referred to as single chain Fv (scFv); see, e.g., Bird et al. (1988) Science: 242:423-426, and Huston et al. (1988) Proc. Natl. Acad. Sci USA 85:5879-5883). This single chain antibody is also intended to be included in the term “antigen binding fragment” of the antibody. Such antibody fragments are obtained using conventional techniques known by those skilled in the art, and screened for functional fragments by using the same method as that for an intact antibody. Antigen binding sites can be produced by recombinant DNA technology or by enzymatic or chemical disruption of an intact immunoglobulin. Antibodies can be antibodies of different isotypes, e.g., IgG (e.g., IgG1, IgG2, IgG3 or IgG4 subtype), IgA1, IgA2, IgD, IgE or IgM antibody.

Fab is an antibody fragment obtained by treating an IgG antibody molecule with a papain (which cleaves the amino acid residue at position 224 of the H chain). The Fab fragment has a molecular weight of about 50,000 and has antigen binding activity, in which about a half of the N-terminal side of H chain and the entire L chain are bound together through a disulfide bond.

The term “CDR” refers to one of the six hypervariable regions within the variable domain of an antibody that primarily contributes to antigen binding. One of the most commonly used definitions for the six CDRs is provided by Kabat E. A. et al. (1991) Sequences of proteins of immunological interest. NIHPublication 91-3242. As used herein, the Kabat definition of CDR only applies to CDR1, CDR2 and CDR3 of the light chain variable domain (CDR LI, CDR L2, CDR L3 or L1, L2, L3), as well as CDR2 and CDR3 of heavy chain variable domain (CDR H2, CDR H3 or H2, H3).

The term “antibody framework” refers to a portion of the variable domain VL or VH, which serves as a scaffold for the antigen binding loop (CDR) of the variable domain. Essentially, it is a variable domain without CDR.

The terms “specific binding,” “selective binding,” “selectively bind” and “specifically bind,” refer to the binding of an antibody to an epitope on a predetermined antigen. Typically, the antibody binds with an affinity (KD) of less than about 10⁻⁷M, such as approximately less than about 10⁻⁸ M, 10⁻⁹M or 10⁻¹⁰ M or less.

Methods for producing and purifying antibodies and antigen binding fragments are well known in the art, such as Cold Spring Harbor Antibody Technical Guide, Chapters 5-8 and 15. The antigen binding fragment can also be prepared by conventional methods. The antibodies or antigen binding fragments of the disclosure are genetically engineered to add one or more human FR regions in nonhuman CDR regions. The human FR germline sequence(s) can be obtained by aligning IMGT human antibody variable germlines gene databases and MOE software from the ImMunoGeneTics (IMGT) website at http://imgt.cines.fr or from the Journal of Immunoglobulins 20011SBN012441351.

The term “peptide” refers to a compound fragment between amino acid and protein, consisting of two or more amino acid molecules connected to each other through peptide bonds. Peptides are structural and functional fragments of proteins. Hormones, enzymes and the like are essentially peptides.

The term “toxin” refers to any substance that can have a harmful effect on the growth or proliferation of cells. Toxins can be small molecule toxins and their derivatives from bacteria, fungi, plants or animals, including Camptothecin derivatives such as exatecan, maytansinoid and its derivatives (CN101573384) such as DM1, DM3, DM4, auristatin F (AF) and its derivatives such as MMAF, MMAE, 3024 (WO 2016/127790 A1, compound 7), diphtheria toxin, exotoxin, ricin A chain, abrin A chain, modeccin, a-sarcin, Aleurites fordii toxic protein, dianthin toxic protein, Phytolaca americana toxic protein (PAPI, PAPII and PAP-S), Momordica charantia inhibitor, curcin, crotin, Sapaonaria officinalis inhibitor, gelonin, mitogellin, restrictocin, phenomycin, enomycin and trichothecenes.

The term “chemotherapeutic drug” refers to a chemical compound that can be used to treat tumors. This definition also includes antihormonal agents that act to modulate, reduce, block, or inhibit the effects of hormones that promote cancer growth, which are often in the form of systemic or holistic therapy. They can be hormones. Examples of chemotherapeutic drugs include alkylating agents, such as thiotepa; cyclosphamide; alkyl sulfonate such as busulfan, improsulfan and piposulfan; aziridine such as benaodopa and uredepa; aziridine and methylamelamine including altretamine, triethylenemelamine, and trimethylolomelamine; nitrogen mustards such as chlorambucil, chlornaphazine; melphalan, novembichin; nitrosoureas such as carmustine, chlorozotocin; antibiotic such as aclacinomycin, actinomycin, authramycin, azaserine, bleomycin, cactinomycin C, calicheamicin, carabicin, chromomycin, carzinophilin, actinomycin D, daunorubicin, detorubicin, doxorubicin, epirubicin, esorubicin, idarubicin, mycophenolic acid, nogalamycin, olivomycin, peplomycin, potfiromycin, puromycin, quelamycin, rodorubicin, streptonigrin; streptozocin, tuberculocidin, ubenimex, zinostatin, zorubicin; antimetabolites such as methotrexate, 5-fluorouracil (5-FU); folic acid analogs such as denopterin, methotrexate, pteropterin, trimetrexate; pterin analogs such as fludarabine, 6-mercaptopterin, thiomethopterin, thioguanopterin; pyrimidine analogs such as ancitabine, datrexate, 6-azuridine, carmofiir, doxitluridine, enocitabine, floxuridine; androgens such as calusterone, dromostanolong propionate, epitiostanol, testolactone; anti-adrenalines such as aminoglutethimide, mitotane, trilostane; folic acid supplements such as frolinic acid; aceglatone; aldophosphamideglycoside; aminolevulinic acid; amsacrine; bestrabucil; biasntrene; defbfamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pintostatin; phenamet; pirarubicin; podophyllinic acid; 2-ethylhydrazide; procarbazine; razoxane; sizofiran; spirogemanium; tenuazonic acid; triaziquone; trichlorrotriethylamine; urethan; vindesine; dacarbazine; mannomustine; mitobronitol; dibromodulcitol; pipobroman; gacytosine; arabinoside; cyclophosphamide; thiotepa; taxanes such as paclitaxel and docetaxel; chlorambucil; gemcitabine; 6-thioguanine; mercaptopurine; methotrexate; platinum analogs such as cisplatin and carboplatin; vinblastine; platinum; etoposide; ifbsfamide; mitomycin C; mitoxantrone; vincristine; vinorelbine; navelbine; novantrone; teniposide; daunorubicin; aminopterin; xeloda; ibandronate; topoisomerase inhibitor RFS2000; difluoromethylomithine; retinoic acid esperamicins; capecitabine; and pharmaceutically acceptable salt, acid or derivative of any of the above substances. This definition also includes antihormonal agents.

The carbon atom content of various hydrocarbon-containing moieties is indicated by a prefix designating the minimum and maximum number of carbon atoms in the moiety, i.e., the prefix Cᵢ-Cⱼ indicates a moiety of the integer “i” to the integer “j” carbon atoms, inclusive. Thus, for example, C₁-C₆ alkyl refers to alkyl of one to six carbon atoms, inclusive.

The term “alkyl” as used herein refers to a linear or branched-chain saturated hydrocarbyl substituent (i.e., a substituent obtained from a hydrocarbon by removal of a hydrogen); in one embodiment containing from one to eight carbon atoms, in another one to six carbon atoms and in yet another one to three carbon atoms. Non-limiting examples of such substituents include methyl, ethyl, propyl (including n-propyl and isopropyl), butyl (including n-butyl, isobutyl, sec-butyl and tert-butyl, pentyl, isoamyl, hexyl, heptyl, octyl and the like. In another embodiment containing one to three carbons and consisting of methyl, ethyl, n-propyl and isopropyl. The phrase “each ‘C₁-C₈ alkyl’ are optionally substituted with one to three R⁵” means that each “C₁-C₈ alkyl” in a recited list of groups can be substituted with one to three R⁵. For example, in the following list, “C₁-C₈ alkyl, (C₁-C₈ alkyl)NHC(O)O—, (C₁-C₈ alkyl)NH—, (C₁-C₈ alkyl)C(O)O—” each of the C₁-C₈ alkyl can be substituted with one to three R⁵. In addition to any group specifically recited in any of the embodiments or claims, in some embodiments, the alkyl is optionally substituted with 1 to 3 substituents independently selected from halo, —C₁-C₁₂alkyl (unsubstituted or substituted, in one embodiment with 1, 2, or 3 halo), aryl, —OH, —O C₁-C₁₂alkyl, —S(O)˜C₁-C₄alkyl (wherein n is 0, 1, or 2), —C₁-C₄alkylNH₂, —NHC₁-C₄alkyl, —C(═O)H, C(═O)OR^{a}, —OC(═O)R^{b}, OC(═O)NR^{a}R^{c}, OC(═O)heteroaryl, and OC(═O)(heterocyclic ring) wherein R^{a}, R^{b}, and R^{d} are independently hydrogen or —C₁-C₄alkyl and R^{b} is alkyl.

The term “alkylene” as used herein refers to a divalent alkyl group, as defined herein.

The term “-alkylene-cycloalkylene-” as used herein refers to an alkylene group, as defined herein, bonded to a cycloalkylene group as defined herein.

The term “alkoxy” refers to an —OR group, wherein R is alkyl, as defined herein, (i.e., a substituent obtained from a hydrocarbon alcohol by removal of the hydrogen from the OH); in one embodiment containing from one to six carbon atoms. Non-limiting examples of such substituents include methoxy, ethoxy, propoxy (including n-propoxy and isopropoxy), butoxy (including n-butoxy, isobutoxy, sec-butoxy and tert-butoxy), pentoxy, hexoxy and the like. In another embodiment having one to three carbons and consisting of methoxy, ethoxy, n-propoxy and isopropoxy. An alkoxy group which is attached to an alkyl group is referred to as an alkoxyalkyl. An example of an alkoxyalkyl group is methoxymethyl.

The term “alkoxyalkyl” as used herein refers to an alkyl group substituted with an alkoxy group, as defined herein.

The term “cycloalkyl” refers to a carbocyclic substituent obtained by removing a hydrogen from a saturated or a partially unsaturated (but does not comprise an aromatic ring) carbocyclic molecule, for example one having three to seven carbon atoms. The term “cycloalkyl” includes monocyclic saturated carbocycles. The term “C₃-C₇, cycloalkyl” means a radical of a three- to seven-membered ring system which includes the groups cyclopropyl, cyclobutyl, cyclopentyl, cyclohexyl, and cycloheptyl. The term “Cₛ-Cₛ cycloalkyl” means a radical of a three- to six-membered ring system which includes the groups cyclopropyl, cyclobutyl, cyclopentenyl, cyclopentyl, cyclohexenyl, and cyclohexyl. The cycloalkyl groups can also be bicyclic, polycyclic, endocyclic, or spirocyclic carbocycles. For example, the term “C₃-C₁₂ cycloalkyl” includes monocyclic carbocycles and bicyclic and spirocyclic cycloalkyl moieties such as bicydapentyl, bicydohexyl, bicycloheptyl, bicyclooctyl, bicyclononyl, spiropentyl, spirohexyl, spiroheptyl, spirooctyl and spironanyl. In addition to any group specifically recited in any of the embodiments or claims, in some embodiments, the cycloalkyl is optionally substituted with 1 to 3 substituents independently selected from halo, —C₁-C₁₂alkyl (unsubstituted or substituted, in one embodiment with 1, 2, or 3 halo), aryl, —OH, —O C₁-C₁₂alkyl, —S(O)˜C₁-C₄alkyl (wherein n is 0, 1, or 2), —C₁-C₄alkylNH₂, —NHC₁-C₄alkyl, —C(═O)H, C(═O)OR^{a}, —OC(═O)R^{b}, OC(═O)NR^{a}R^{c}, OC(═O)heteroaryl, and OC(═O)(heterocyclic ring) wherein R^{a}, R^{c}, and R^{d} are independently hydrogen or —C₁-C₄alkyl and R^{b} is alkyl.

The term “cycloalkylene” refers to a divalent cycloalkyl group, as defined herein.

The term “C₃-C₆ cycloalkoxy” refers to a three- to six-membered cycloalkyl group attached to an oxygen radical. Examples include cyclopropoxy, cyclobutoxy, cyclopentoxy and cyclohexoxy.

In some instances, the number of atoms in a cyclic substituent containing one or more heteroatoms (i.e., heteroaryl or heterocycloalkyl is indicated by the prefix “x- to y-membered,” wherein x is the minimum and y is the maximum number of atoms forming the cyclic moiety of the substituent. Thus, for example, “4 to 6-membered heterocycloalkyl” refers to a heterocycloalkyl containing from 4 to 6 atoms, including one to three heteroatoms, in the cyclic moiety of the heterocycloalkyl. Likewise, the phrase “5- to 6-membered heteroaryl” refers to a heteroaryl containing from 5 to 6 atoms, and “5- to 10-membered heteroaryl” refers to a heteroaryl containing from 5 to 10 atoms, each including one or more heteroatoms, in the cyclic moiety of the 30 heteroaryl. Furthermore, the phrases “5-membered heteroaryl” and “6-membered heteroaryl” refer to a five-membered heteroaromatic ring system and a six-membered heteroaromatic ring system, respectively. The heteroatoms present in these ring systems are selected from N, O and S.

The term “hydroxy” or “hydroxyl” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol. The terms cyano and nitrile refer to a —CN group. The term “oxo” means an oxygen which is attached to a carbon by a double bond (i.e., when R⁴ is oxo then R⁴ together with the carbon to which it is attached are a C═O moiety).

The term “hydroxyalkyl” refers to an alkyl group, as defined herein, substituted with 1, 2, or 3 hydroxy groups.

The term “halo” or “halogen” refers to fluorine (which may be depicted as —F), chlorine (which may be depicted as —C¹), bromine (which may be depicted as —Br), or iodine (which may be depicted as —I).

The term “haloalkyl” refers to an alkyl group, as defined herein, substituted with 1, 2, 3, 4, 5, or 6 halo groups. In some embodiments, haloalkyl includes chloroalkyl.

The term “heterocycloalkyl” refers to a substituent obtained by removing a hydrogen from a saturated or partially saturated ring structure containing a total of the specified number of atoms, such as 4 to 6 ring atoms or 4 to 12 atoms, wherein at least one of the ring atoms is a heteroatom (i.e., oxygen, nitrogen, or sulfur), with the remaining ring atoms being independently selected from the group consisting of carbon, oxygen, nitrogen, and sulfur. The sulfur may be oxidized [i.e., S(O) or S(O)₂] or not. In a group that has a heterocycloalkyl substituent, the ring atom of the heterocycloalkyl substituent that is bound to the group may be a nitrogen heteroatom, or it may be a ring carbon atom. Similarly, if the heterocycloalkyl substituent is in turn substituted with a group or substituent, the group or substituent may be bound to a nitrogen heteroatom, or it may be bound to a ring carbon atom. It is to be understood that a heterocyclic group may be monocyclic, bicyclic, polycyclic, endocyclic or spirocyclic. In addition to any group specifically recited in any of the embodiments or claims, in some embodiments, the heterocycloalkyl is optionally substituted with 1 to 3 substituents independently selected from halo, —C₁-C₁₂alkyl (unsubstituted or substituted, in one embodiment with 1, 2, or 3 halo), aryl, —OH, —O C₁-C₁₂alkyl, —S(O)˜C₁-C₄alkyl (wherein n is 0, 1, or 2), —C₁-C₄alkylNH₂, —NHC₁-C₄alkyl, —C(═O)H, C(═O)OR^{a}, —OC(═O)R^{b}, OC(═O)NR^{a}R^{c}, OC(═O)heteroaryl, and OC(═O)(heterocyclic ring) wherein R^{a}, R^{c}, and R^{d} are independently hydrogen or —C₁-C₄alkyl and R^{b} is alkyl.

The term “aryl” refers to a carbocyclic monocyclic or bicyclic ring system, wherein the monocyclic ring is aromatic and the bicyclic ring comprises at least one aromatic ring. The term “C₆-C₁₀ aryl” refers to carbocyclic systems with 6 to 10 atoms and includes phenyl, tetrahydronaphthyl, and naphthyl. In addition to any group specifically recited in any of the embodiments or claims, in some embodiments, the aryl is optionally substituted with 1 to 3 substituents independently selected from halo, —C₁-C₁₂alkyl (unsubstituted or substituted, in one embodiment with 1, 2, or 3 halo), aryl, —OH, —O C₁-C₁₂alkyl, —S(O)ₙC₁-C₄alkyl (wherein n is 0, 1, or 2), —C₁-C₄alkylNH₂, —NHC₁-C₄alkyl, —C(═O)H, C(═O)OR^{a}, —OC(═O)R^{b}, OC(═O)NR^{a}R^{c}, OC(═O)heteroaryl, and OC(═O)(heterocyclic ring) wherein R^{a}, R^{c}, and R^{d} are independently hydrogen or —C₁-C₄alkyl and R^{b} is alkyl.

The term “arylene” as used herein refers to a divalent aryl group, as defined herein.

The term “heteroalkyl” refers to an alkyl group, as defined herein, wherein one or more —CH₂— is replaced by a group independently selected from —O—, —S—, —S(O)—, —S(O)₂, and —NR— where R is hydrogen or alkyl, as defined herein, and/or wherein one or more —CH₃ group is replaced by a group independently selected from —OH, —SH, and —NH₂ where each R is independently hydrogen or alkyl. Heteroalkyl includes 2-thioethyl, 2-amino-prop-1-yl, 2-hydroxy-eth-1-yl, N-methyl-amino-ethyl, and the like. Hydroxyalkyl is a subset of heteroalkyl.

The term “heteroalkylene” refers to a divalent heteroalkyl, as defined herein.

The term “heteroaryl” refers to an aromatic ring structure containing the specified number of ring atoms in which at least one of the ring atoms is a heteroatom (i.e., oxygen, nitrogen, and/or sulfur), with the remaining ring atoms being carbon. Examples of heteroaryl substituents include 6-membered heteroaryl rings such as pyridyl, pyrazyl, pyrimidinyl, and pyridazinyl; and 5-membered heteroaryl rings such as triazolyl, imidazolyl, furanyl, thiophenyl, pyrazolyl, pyrrolyl, oxazolyl, isoxazolyl, thiazolyl, 1,2,3-oxadiazolyl, 1,2,4-oxadiazolyl, 1,2,5-oxadiazolyl, 1,3,4-oxadiazolyl and isothiazolyl. The heteroaryl group can also be a bicyclic heteroaromatic group such as indolyl, benzofuranyl, benzothienyl, benzimidazoly, benzothiazolyl, benzoxazolyl, benzoisoxazolyl, oxazolopyridinyl, imidazopyridinyl, imidazopyrimidinyl and the like. In a group that has a heteroaryl ring, the ring atom of the heteroaryl ring that is bound to the group may be a nitrogen atom, or it may be a ring carbon atom. Similarly, if the heteroaryl ring is in turn substituted with a group or substituent, the group or substituent may be bound to a nitrogen atom, or it may be bound to a ring carbon atom. The term “heteroaryl” also includes pyridyl N-oxides and groups containing a pyridine N-oxide ring. In addition, the heteroaryl group may contain an oxo group such as the one present in a pyridone group. Further examples include furyl, thienyl, oxazolyl, thiazolyl, imidazolyl, pyrazolyl, triazolyl, tetrazolyl, isoxazolyl, isothiazolyl, oxadiazolyl, thiadiazolyl, pyidinyl, pyridazinyl, pyrimidinyl, pyraziny, pyridin-2(1H)-onyl, pyridazin-2(1H)-onyl, pyrimidin-2(1H-onyl, pyrazin-2(1H)-onyl, imidazo[1,2-a]pyridinyl, and pyrazolo[1,5-alpyridinyl. The heteroaryl can be further substituted as defined herein.

Examples of single-ring heteroaryls and heterocycloalkyls include furanyl, dihydrofuranyl, tetrahydrofuranyl, thiophenyl, dihydrothiophenyl, tetrahydrothiophenyl, pyrrolyl, isopyralyl, pyrrolinyl, pyrolidinyl, imidazolyl, isoimidazolyl, imidazolinyl, imidazolidinyl, pyrazolyl, pyrazolinyl, pyrazalidinyl, tiazolyl, tetrazolyl, dithiolyl, oxathiolyl, oxazalyl, isoxazolyl, thiazolyl, isothiazolyl, thiazolinyl, isothiazolinyl, thiazolicdinyl, isothiazolidinyl, thiaoxadiazolyl, oxathiazolyl, Dxadiazolyl {including oxadiazolyl, 1,2,4-oxadiazolyl,1,2,5-oxadiazolyl, or 1,3,4-oxadiazoly), pyranyl (including 1,2-pyranyl or 1,4-pyranyl), dihydropyranyl, pyridinyl, piperidinyl, diazinyl (including pyridazinyl, pyrimidinyl, piperazinyl, triazinyl (including s-triazinyl, as-triazinyl and v-triazinyl), oxazinyl (including 2H-1,2-oxazinyl,6H-1,3-oxazinyl, or 2H-1,4-oxazinyl), isoxazinyl (including O-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 2H1,2,4-oxadiazinyl or 2H1,2,5-oxadiazinyl), and morpholinyl.

The term “heteroaryl” can also include, when specified as such, ring systems having two rings wherein such rings may be fused and wherein one ring is aromatic and the other ring is not fully part of the conjugated aromatic system (i.e., the heteroaromatic ring can be fused to a cycloalkyl an heterocycloalkyl ring). Non-limiting examples of such ring systems include 5,6,7,8-tetrahydroisoquinalinyl, 5,6,7,8-tetrahydroquinolinyl, 6,7-dihydro-5H-cyclopenta[b]pyridinyl, 6,7-dihydro-5H-cyclopenta[c]pyridinyl,1,4,5,6-tetrahydrocyclopenta[clpyrazolyl,2,4,5,6-tetrahydrocyclopenta[c]pyrazolyl,5,6-dihydro-4Hpyrolo[1,2-b]pyrazolyl,6,7-dihydro-5H-pyrrolo[1,2-b[1,2,4]triazolyl, 5,6,7,8-tetrahydro-[1,2.4]triazolo[1,5-a]pyridinyl, 4.5,6,7-tetrahydropyrazolo[1.5-a]pyridinyl,4,5,6,7-tetrahydro-1H-indazolyl and 4,5.6,7-tetrahydro-2H-indazolyl.

It is to be understood that if a carbocyclic or heterocyclic moiety may be bonded or otherwise attached to a designated group through differing ring atoms without denoting a specific point of attachment, then all possible points are intended, whether through a carbon atom or, for example, a trivalent nitrogen atom. For example, the term “pyridyl” means 2-, 3- or 4-pvridyl, the term “thienyl” means 2- or 3-thienyl, and so forth.

The term “heteroarylene” as used herein refers to a divalent heteroaryl group, as defined herein.

The term “amino protecting group” refers to a group which prevents an amino group from reaction when other parts of the molecular are subject to a reaction and can be easily removed.

Non-limiting examples include 9-fluorenylmethyloxycarbonyl, tert-butoxycarbonyl, acetyl, benzyl, allyl, p-methoxybenzyl and the like. These groups can be optionally substituted by one to three substituent(s) selected from the group consisting of halogen, alkoxy and nitro. The amino protecting group is preferably 9-fluorenylmethyloxycarbonyl.

The term “deuterated alkyl” refers to an alkyl group substituted by one or more deuterium atom(s), wherein the alkyl is as defined above.

The term “unsaturated” in the context of the term cycloalkyl, cycloalkylene, and heterocycle refers to a partially unsaturated, but not aromatic ring.

The term “fused” means bicyclic, tricyclic, or polycyclic structures comprised of at least two carbocyclic or heterocyclic structures sharing at least one chemical bond.

If substituents are described as “independently” having more than one variable, each instance of a substituent is selected independent of the other(s) from the list of variables available. Each substituent therefore may be identical to or different from the other substituent(s).

If substituents are described as being “independently selected” from a group, each instance of a substituent is selected independent of the other(s). Each substituent therefore may be identical to or different from the other substituent(s).

“Optional” or “optionally” means that the subsequently described event or circumstance may, but need not, occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or di-substituted with an alkyl group and situations where the aryl group is not substituted with the alkyl group.

“Substituted” refers to one or more hydrogen atoms in a group, preferably up to 5, and more preferably 1 to 3 hydrogen atoms, independently substituted by a corresponding number of substituents. It goes without saying that the substituents only exist in their possible chemical position. The person skilled in the art is able to determine whether the substitution is possible or impossible by experiments or theory without excessive effort. For example, the combination of amino or hydroxy having free hydrogen and carbon atoms having unsaturated bonds (such as olefinic) may be unstable.

As used herein, the term “a compound of Formula (I)” (or other formula number) is defined to include all forms of the compound of Formula 1, including hydrates, solvates, isomers, crystalline and non-crystalline forms, isomorphs, polymorphs, and metabolites thereof. For example, the compounds disclosed herein, or pharmaceutically acceptable salts thereof, may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm. Compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds provided herein may possess one or more asymmetric centers; such compounds can therefore be produced as individual (R)- or (S)-stereoisomers or as mixtures thereof. Unless indicated otherwise, the description or naming of a particular compound in the specification and Claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

A hydrogen (H) or carbon (C) substitution for compounds of the formula I to VIII include a substitution with any isotope of the respective atom. Thus, a hydrogen (H) substitution includes a ¹H, ²H (deuterium), or ³H (tritium) isotope substitution, as may be desired, for example, for a specific therapeutic or diagnostic therapy, or metabolic study application, or metabolic or chemical stability enhancement. Optionally, a compound of this disclosure may incorporate a known in the art radioactive isotope or radioisotope, such as ³H, ¹⁵O, ¹²C, or ¹³N isotope, to afford a respective radiolabeled compound of formula I.

A “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and Claims includes both one and more than one such carrier.

A “pharmaceutically acceptable salt” of a compound means a salt that is pharmaceutically acceptable and that possesses the desired pharmacological activity of the parent compound. Such salts include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2 hydroxyethanesulfonic acid, benzenesulfonic acid, 4-chlorobenzenesulfonic acid, 2-naphthalenesulfonic acid, 4-toluenesulfonic acid, camphorsulfonic acid, 4 methylbicyclo[2.2.2]oct-2-ene-1-carboxylic acid, glucoheptonic acid, 4,4′ methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Treating”, “treatment”, or “therapy” of a disease includes:
(1) preventing the disease, i.e., causing the clinical symptoms of the disease not to develop in a mammal that may be exposed to or predisposed to the disease but does not yet experience or display symptoms of the disease,
(2) inhibiting the disease, i.e., arresting or reducing the development of the disease or its clinical symptoms, or
(3) relieving the disease, i.e., causing regression of the disease or its clinical symptoms.

The term “pharmaceutical composition”, refers to a mixture of one or more of the compounds described herein or physiologically/pharmaceutically acceptable salts or pro drugs thereof with other chemical components, and other components such as physiologically/pharmaceutically acceptable carriers and excipients. The purpose of the pharmaceutical composition is to facilitate administration of a compound to an organism, which is conducive to the absorption of the active ingredient so as to show biological activity.

The term “solvate” refers to a pharmaceutically acceptable solvate formed by a ligand-drug conjugate of the present disclosure with one or more solvent molecule(s). Non-limiting examples of solvent molecules include water, ethanol, acetonitrile, isopropanol, DMSO, ethyl acetate.

The term “carrier” used in the composition of the present disclosure refers to a system that can change the way a drug enters the human body and distribution, control the drug release rate, and deliver the drug to the targeted organ. Drug carrier release and targeting systems can reduce drug degradation and loss, reduce side effects and improve bioavailability.

The term “excipient” is an adjunct in a pharmaceutical formulation other than a main drug, which can also be referred to as an adjuvant, such as adhesives, fillers, disintegrants, lubricants in tablets; matrix parts in the semisolid preparations ointment and cream; preservatives, anti-oxidants, flavoring agents, fragrances, co-solvents, emulsifiers, solubilizers, osmotic pressure regulators, colorants in liquid preparations and the like.

The term “diluent”, also known as filler, is primarily intended to increase the weight and volume of the tablet. The addition of diluent ensures a certain volume, reduces the dose deviation of the main components, and improves the compression profile of the drug. When the tablet contains an oily component, an absorbent is added to absorb the oily substance, thereby keeping the “dry” state to facilitate tablet formation. For example, diluent includes starch, lactose, inorganic salts of calcium, microcrystalline cellulose and the like.

The pharmaceutical composition can be in the form of a sterile injectable aqueous solution. Acceptable vehicles or solvents that can be used are water, Ringer's solution or isotonic sodium chloride solution. The sterile injectable formulation can be a sterile injectable oil-in-water micro-emulsion in which the active ingredient is dissolved in the oil phase. For example, the active ingredient is dissolved in a mixture of soybean oil and lecithin. The oil solution is then added to a mixture of water and glycerin, and processed to form a micro-emulsion. The injectable solution or micro-emulsion can be introduced into a patient's bloodstream by local bolus injection. Alternatively, the solution and micro-emulsion are preferably administrated in a manner that maintains a constant circulating concentration of the compound of the present disclosure. In order to maintain this constant concentration, a continuous intravenous delivery device can be used. An example of such a device is Deltec CADD-PLUS™ 5400 intravenous injection pump.

The pharmaceutical composition can be in the form of a sterile injectable aqueous or oily suspension for intramuscular and subcutaneous administration. Such a suspension can be formulated with suitable dispersants or wetting agents and suspending agents as described above according to known techniques. The sterile injectable formulation can also be a sterile injectable solution or suspension prepared in a nontoxic parenterally acceptable diluent or solvent, for example, a solution prepared in 1,3-butanediol. Moreover, sterile fixed oils can easily be used as a solvent or suspending medium. For this purpose, any blending fixed oils including synthetic mono- or di-glyceride can be employed. Moreover, fatty acids, such as oleic acid, can. also be employed in the preparation of an injection.

The term “drug loading”, refers to the average number of cytotoxic drugs loaded on each ligand in the compound of formula (I), and can also be expressed as the ratio of the number of drug to the number of antibody. The drug loading can range from 0 to 12, preferably from 1 to 10 cytotoxic drugs per ligand. In an embodiment of the present disclosure, the drug loading is expressed as n, and exemplary values can be an average of 1, 2, 3, 4, 5, 6, 7, 8, 9, 10. The average number of drugs per ADC molecule after coupling reaction can be determined by conventional methods such as UV/visible spectroscopy, mass spectrometry, ELISA test and HPLC characterization.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “mammal” refers to all mammals including humans, livestock, and companion animals.

The compounds described herein are generally named according to the IUPAC or CAS nomenclature system. Abbreviations which are well known to one of ordinary skill in the art may be used (e.g. “Ph” for phenyl, “Me” for methyl, “Et” for ethyl, “h” for hour or hours and “r.t.” or “rt” for room temperature).

### Synthesis Method of the Present Disclosure

### Scheme I:

A method for preparing the compound of formula (D4) according to formula (V) or the pharmaceutically acceptable salt or solvate thereof of the present disclosure, comprises the following step of:

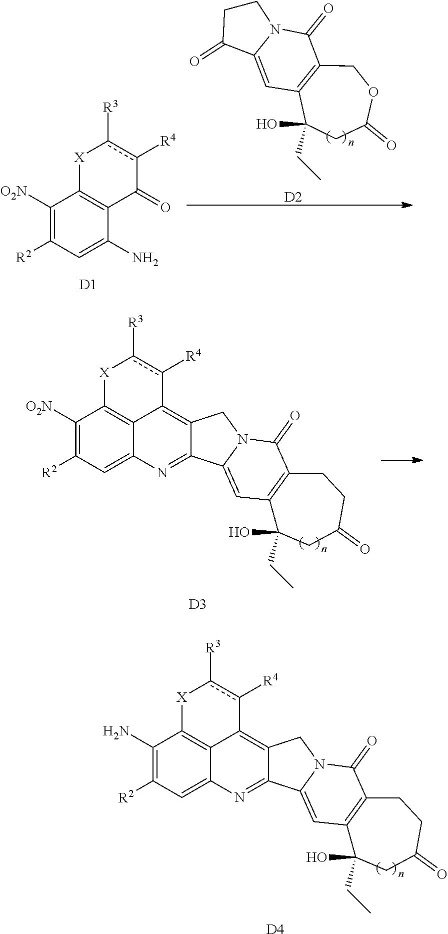

Step 1: reacting the compound of formula (D1) and D2 under acidic condition to obtain the compound of formula (D3);

Step 2: the compound of formula (D3) is reducted to obtain the compound of formula (D4);

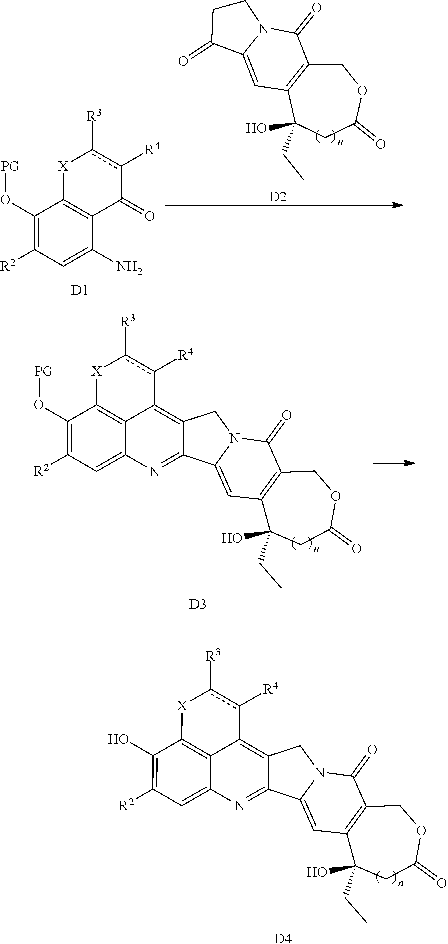

Step 1: reacting the compound of formula (D1) and D2 under acidic condition to obtain the compound of formula (D3);

Step 2: the compound of formula (D3) is deprotected to obtain the compound of formula (D4);
wherein: X, R², R³, R⁴, n, are as defined in formula (V) and any embodiments thereof, PG is an amino protecting group, and preferably benzyloxycarbonyl (Cbz).

The reagent that provides an alkaline condition includes organic bases and inorganic bases. The organic bases include, but are not limited to, triethylamine, diethylamine, N-methylmorpholine, pyridine, hexahydropyridine, N,N-diisopropylethylamine, n-butyl lithium, lithium diisopropylamide, potassium acetate, sodium tert-butoxide and potassium tert-butoxide. The inorganic bases include, but are not limited to, sodium hydride, potassium phosphate, sodium carbonate, potassium carbonate, cesium carbonate, sodium hydroxide and lithium hydroxide.

### Scheme II:

A method for preparing the compound of formula (D3 or D5 or D6) according to formula (IV) or the pharmaceutically acceptable salt or solvate thereof of the present disclosure, comprises the following steps of:

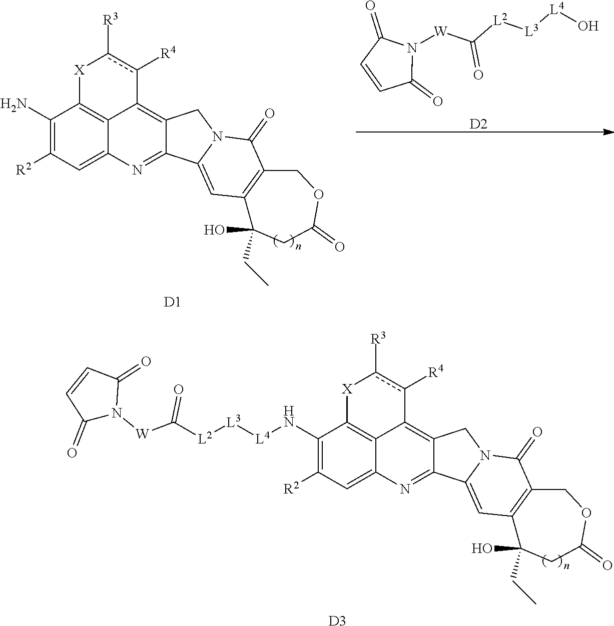

Step 1: the compound of formula (D1) and the compound of formula (D2) are reacted in the presence of a condensing agent to obtain the compound of formula (D3);

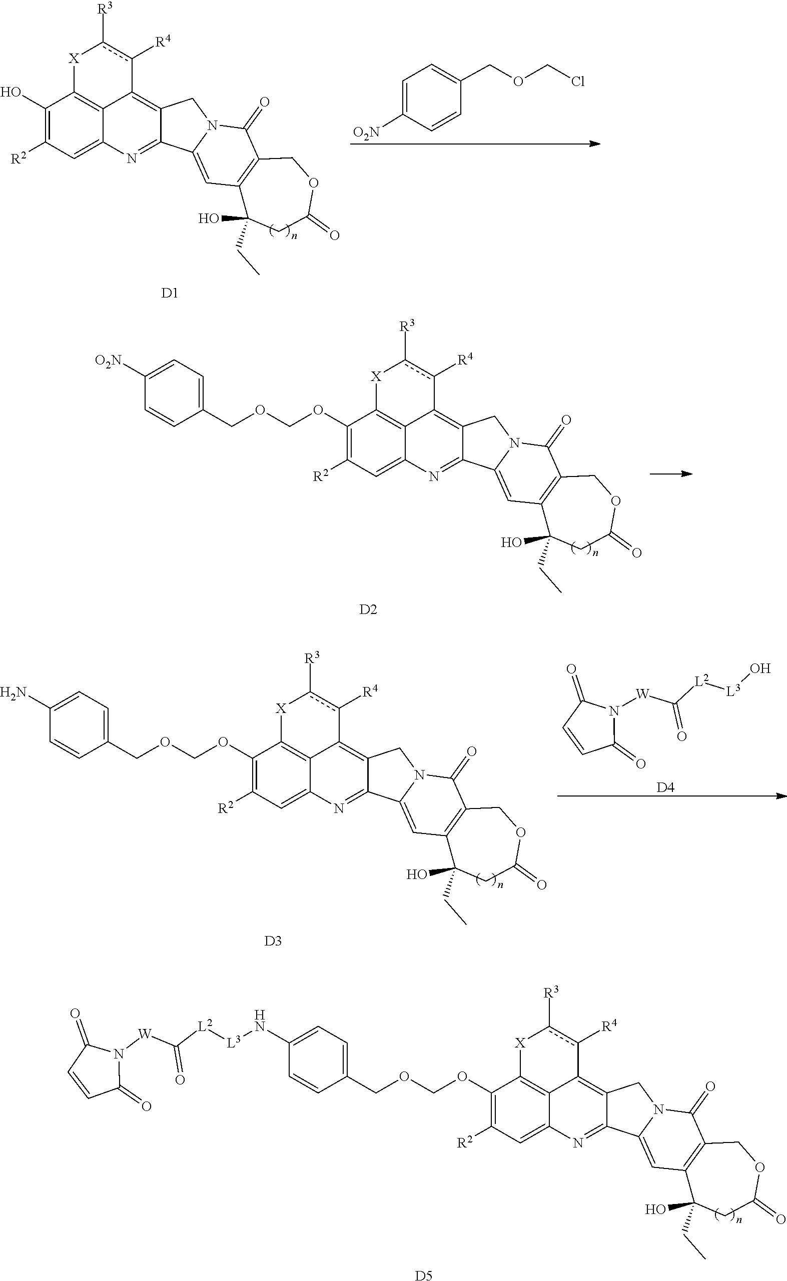

Step 1: reacting the compound of formula (D1) and 1-((chloromethoxy)methyl)-4-nitrobenzene to obtain the compound of formula (D2);

Step 2: the compound of formula (D2) is reducted to obtain the compound of formula (D3);

Step 3: the compound of formula (D3) and the compound of formula (D4) are reacted in the presence of a condensing agent to obtain the compound of formula (D5);

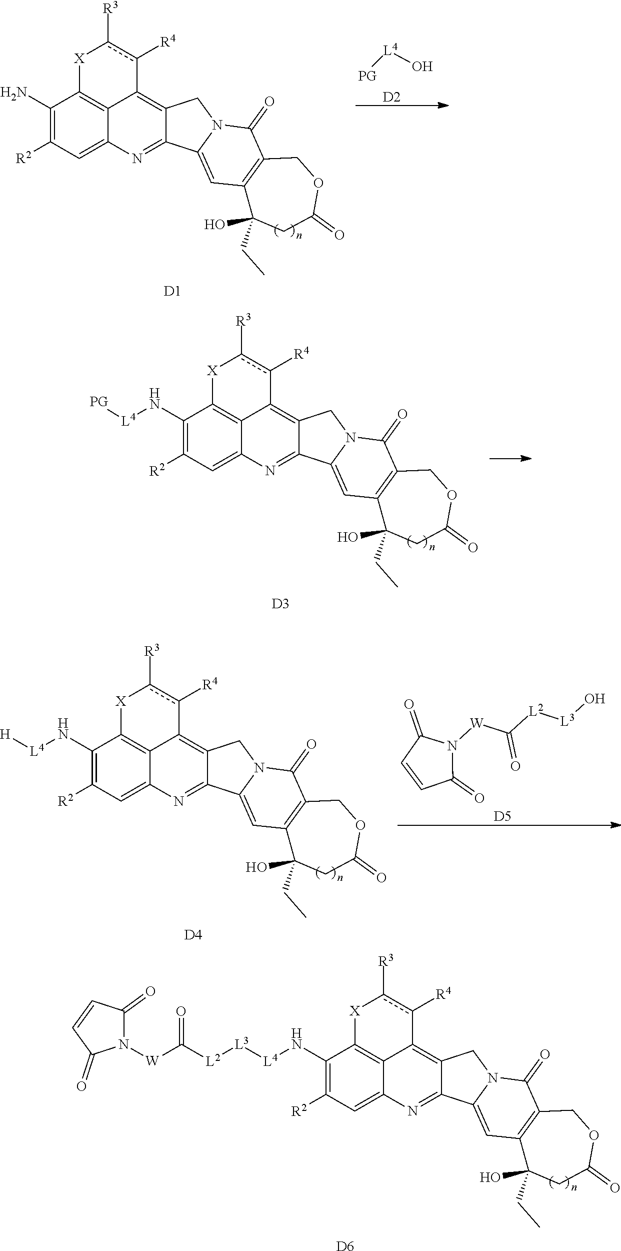

Wherein: X, R², R³, R⁴, n, w, L², L³, L⁴, are as defined in formula (IV) and any embodiments thereof, PG is an amino protecting group, and preferably benzyloxycarbonyl (Cbz).

The reagent that provides an alkaline condition includes organic bases and inorganic bases. The organic bases include, but are not limited to, triethylamine, diethylamine, N-methylmorpholine, pyridine, hexahydropyridine, N,N-diisopropylethylamine, n-butyl lithium, lithium diisopropylamide, potassium acetate, sodium tert-butoxide and potassium tert-butoxide. The inorganic bases include, but are not limited to, sodium hydride, potassium phosphate, sodium carbonate, potassium carbonate, cesium carbonate, sodium hydroxide and lithium hydroxide.

The condensing agent is selected from the group consisting of 4-(4.6-dimethoxy-1.3.5-triazin-2-yl)-4-meth-ylmorpholinium chloride, 1-hydroxybenzotriazole, 1-(3-di-methylaminopropyl)-3-ethylcarbodimide hydrochloride, N,N′-dicyclohexylcarbodimide, N,N′-disopropylcarbodimide, O-benzotriazole-N,N,N′,N′-tetramethylurea tetraffuoroborate, 1-hydroxybenzotriazole, 1-hydroxy-7-azobenzotriazole, O-benzotriazole-N,N,N′,N′-tetramethylurea hexafluorophosphate, 2-(7-azobenzotriazole)-N,N,N′,N′-tetramethylurea hexafluorophosphate, benzotriazol-1-yloxytris(dimethyl-amino)phosphonium hexafluorophosphate and benzotriazol-1-yl-oxytripyrrolidinyl phosphorus hexafluorophosphate, and preferably 4-(4.6-dimethoxy-1.3.5-triazin-2-y)-4-meth-ylmorpholinium chloride, 1-hydroxybenzotriazole and 1-(3-dimethylaminopropyl)-3-ethylcarbodimide hydrochloride.

### Scheme III:

A method for preparing the compound of formula (D9 or D11) according to formula (III) and any embodiments thereof, or the pharmaceutically acceptable salt or solvate thereof, of the present disclosure, comprises the following step of:

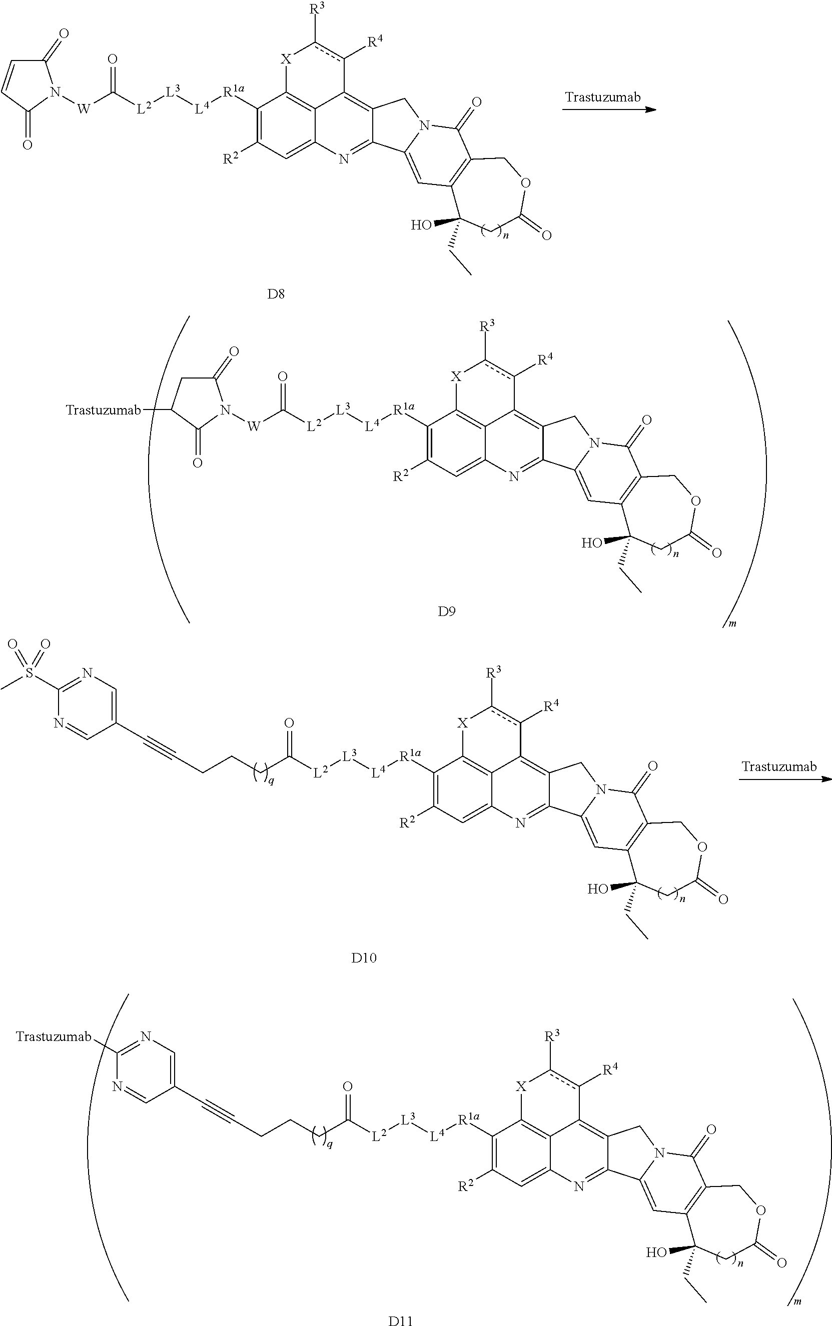

After reduction, T is coupled with the compound of formula D8 or D10 to give the ligand drug conjugates of formula D9 or D11; the reducing agent is preferably TCEP;
wherein:
T is a ligand;
X, R¹, R², R³, R⁴, n, w, L², L³, L⁴, q, are as defined in formula (III) or any embodiments thereof.

The present disclosure will be further described with reference to the following examples, but the examples should not be considered as limiting the scope of the present disclosure.

The experimental methods in the examples of the present disclosure for which the specific conditions are not indicated were carried out according to conventional conditions or the conditions recommended by the material or product manufacturers. The reagents for which the specific sources are not indicated are conventional reagents purchased from market.

### EXAMPLES

### Example 1

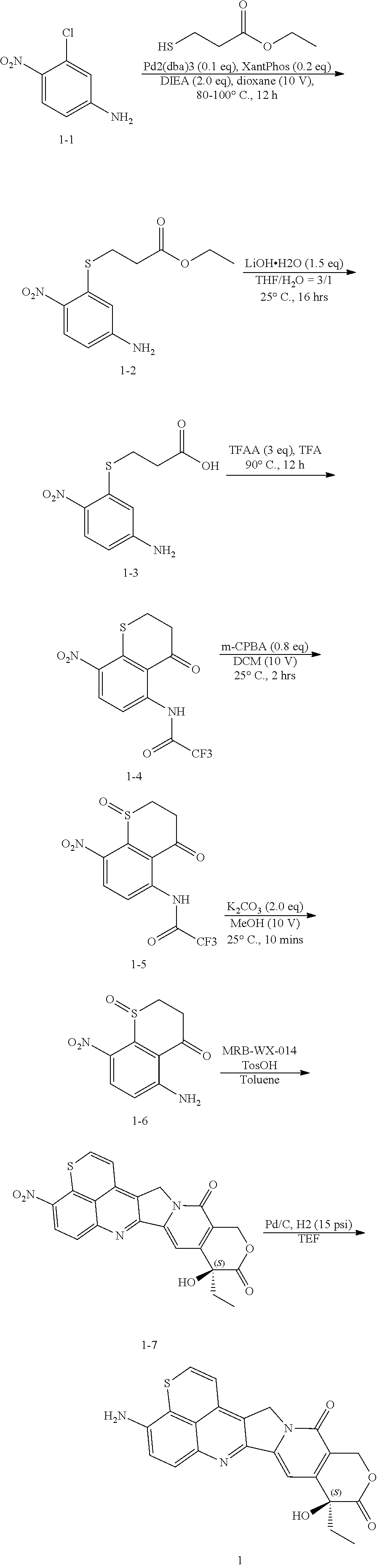

To a solution of 1-6 (70 mg, 0.291 mmol) in Toluene (4.00 mL) was added MRB-WX-014 (

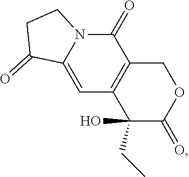 [SMILES: CC[C@@]1(O)C(=O)OCc2c1cc1n(c2=O)CCC1=O]

76.71 mg, 0.29 mmol) and 4-methylbenzenesulfonic acid (50.18 mg, 0.29 mmol) under N₂. The mixture was stirred at 110° C. for 12 h. LCMS showed the reaction was completed. The reaction mixture was cooled to RT and concentrated under reduced pressure. The residue was purified by prep-HPLC (Phenomenex C18 100*40 mm*3 um; mobile phase: [A: water (TFA)-B:ACN]; B %: 20%-50%, 8 min). 1-7 (15.00 mg, 0.03 mmol, 11.47% yield) was obtained as a yellow solid. LC-MS: 450.1 [M+H]⁺.

To a solution of 1-7 (10.00 mg, 0.02 mmol) in 2,2,2-trifluoroethan-1-ol (1.00 mL) was added Pd/C (10%, 2.28 mg, 0.002 mmol) under N₂. The suspension was degassed under vacuum and purged with H₂ for several times. The mixture was stirred at 25° C. for 20 mins under H₂ (15 Psi). LCMS showed the reaction was completed. The reaction mixture was filtered and the filtrate was purified by prep-HPLC (column: Phenomenex Luna C_{18 75*30} mm*3 um; mobile phase: [A:water(TFA)-B:ACN]; B %: 5%-35%, 8 min). The compound of Example 1 (3.96 mg, 0.009 mmol, 21.21% yield) was obtained as a yellow solid.

LC-MS: 420.2 [M+H]⁺.

¹H NMR (400 MHz, METHANOL-d₄) δ 7.66 (d, J=9.0 Hz, 1H), 7.56 (s, 1H), 7.38 (br dd, J=8.5, 3.8 Hz, 2H), 6.79 (br d, J=9.6 Hz, 1H), 5.66-5.55 (m, 1H), 5.40 (d, J=16.3 Hz, 1H), 5.06 (br s, 2H), 1.97 (br dd, J=7.3, 4.7 Hz, 2H), 1.02 (t, J=7.4 Hz, 3H).

### Example 2

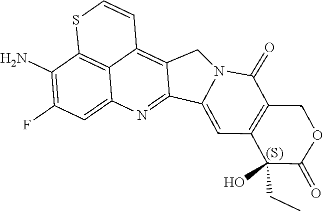

The compound of Example 2 was synthesized following procedures described for Example 1.

LC-MS: 438.1 [M+H]⁺.

¹H NMR (400 MHz, DMSO-d₆) δ 7.49 (d, J=12.4 Hz, 1H), 7.36 (d, J=10.0 Hz, 1H), 7.11 (s, 1H), 6.79 (d, J=10.0 Hz, 1H), 6.46 (s, 1H), 5.56 (s, 2H), 5.39 (s, 2H), 5.02 (s, 2H), 1.84 (td, J=14.0, 6.9 Hz, 2H), 0.86 (t, J=7.3 Hz, 3H).

### Example 4

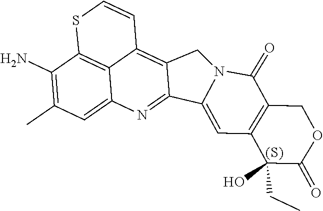

The compound of Example 4 was synthesized following procedures described for Example 1.

LC-MS: 434.0 [M+H]⁺.

¹H NMR (400 MHz, DMSO-d₆) δ 7.51 (s, 1H), 7.29 (d, J=10.0 Hz, 1H), 7.10 (s, 1H), 6.73 (d, J=10.0 Hz, 1H), 6.45 (s, 1H), 5.46-5.36 (m, 2H), 5.19-5.11 (m, 2H), 5.00 (s, 2H), 2.35 (s, 3H), 1.85 (td, J=13.4, 6.9 Hz, 2H), 0.87 (br t, J=7.3 Hz, 3H).

### Example 5

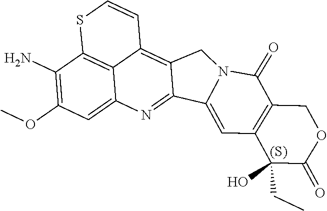

The compound of Example 5 was synthesized following procedures described for Example 1.

LC-MS: 450.2 [M+H]⁺.

¹H NMR (400 MHz, DMSO-d₆) δ 7.31 (d, J=10.0 Hz, 1H), 7.20 (s, 1H), 7.13 (s, 1H), 6.73 (d, J=10.0 Hz, 1H), 5.40 (s, 2H), 5.00 (s, 2H), 3.97 (s, 3H), 1.91-1.78 (m, 2H), 0.86 (t, J=7.3 Hz, 3H).

### Example 9

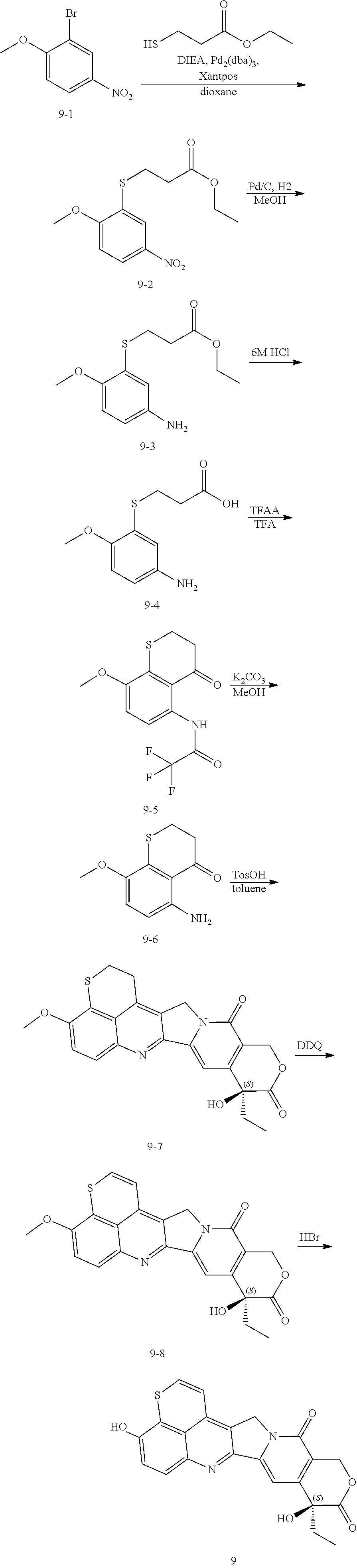

To a solution of 9-1 (20 g, 86.196 mmol) in dioxane (200 mL) was added ethyl 3-sulfanylpropanoate (13.88 g, 103.435 mmol), DIEA (30 mL, 22.28 g, 172.392 mmol), Xantphos (9.98 g, 17.239 mmol) and Pd₂ (dba)₃ (7.89 g, 8.620 mmol) at 25° C. The mixture was degassed and purged with N₂ for 3 times before being stirred at 90° C. for 4 h under N₂. LCMS, TLC (Petroleum ether/Ethyl acetate=3/1) showed 9-1 was consumed completely and desired MS was detected. The reaction mixture was cooled to RT, quenched with H₂O (100 mL) and extracted with Ethyl acetate (100 mL×2). The combined organic layers were washed with brine (100 mL), dried over Na₂SO₄, filtered and the filtrate was concentrated under reduced pressure. The residue was purified by SGC (Petroleum ether/Ethyl acetate=10/1 to 1/1). 9-2 (20 g, 70.099 mmol, 81.33%) was obtained as a yellow solid. LC-MS: 286.2 [M+H]⁺.

To a solution of 9-2 (25 g, 92.152 mmol) in MeOH (300 mL) was added Pd/C (10%, 1.96 g, 18.430 mmol) under N₂. The mixture was degassed and purged with H₂ for 3 times before being stirred under H₂ (15 psi) at 25° C. for 12 h. LCMS showed 9-2 was consumed completely. The reaction mixture was filtered and the filtrate was concentrated under reduced pressure to obtain crude 9-3 (25 g, 88.061 mmol, 95.56%) as a yellow solid, which was used directly for the next step without purification. LC-MS: 256.2 [M+H]⁺.

A solution of 9-3 (25 g, 97.913 mmol) in HCl solution (6M, 489.563 mL) was stirred for 12 h at 60° C. LCMS showed SM was consumed completely. The reaction mixture was cooled to RT and concentrated under reduced pressure. The residue was triturated with EtOAc/MTBE=1/1 (200 mL), filtered and the solid was collected. 9-4 (22 g, 96.797 mmol, 98.86%) was obtained as a yellow solid. LC-MS: 228.2 [M+H]⁺.

To a solution of 9-4 (11 g, 48.398 mmol) in TFA (110 mL) was added TFAA (101.65 g, 483.985 mmol) at 25° C. The mixture was stirred for 12 h at 25° C. LCMS and TLC (Petroleum ether/Ethyl acetate=3/1) showed 9-4 was consumed completely and desired MS was detected. The reaction mixture was concentrated under reduced pressure. The residue was purified by column chromatography (SiO₂, Petroleum ether/Ethyl acetate=10/1 to 1/1). 9-5 (12 g, 39.309 mmol, 81.22%) was obtained as a yellow solid. LC-MS: 304.1 [M−1]⁻.

To a solution of 9-5 (6 g, 19.655 mmol) in MeOH (60 mL) was added K₂CO₃ (5.43 g, 39.309 mmol) at 25° C. The mixture was stirred at 25° C. for 12 h. TLC (Petroleum ether/Ethyl acetate=3/1) showed 9-5 was consumed completely. The mixture was adjusted to pH=−4 by slowly addition of HCl solution (2M). The residue was diluted with H₂O (50 mL) and extracted with DCM (100 mL×2). The combined organic layers were dried over Na₂SO₄, filtered and the filtrate was concentrated under reduced pressure to give crude 9-6 (5 g, 19.115 mmol, 97.25% as yellow oil), which was used directly for the next step without purification.

To a solution of 9-6 (400 mg, 1.529 mmol) in toluene (10 mL) was added (4S)-4-ethyl-4-hydroxy-3,4,6,7,8,10-hexahydro-1H-pyrano[3,4-f]indolizine-3,6,10-trione (402.56 mg, 1.529 mmol) and 4-methylbenzenesulfonic acid (263.33 mg, 1.529 mmol) at 25° C. The mixture was stirred at 105° C. for 12 h. LCMS showed the SM was consumed completely and desired MS was detected. The reaction mixture was cooled to RT and concentrated under reduced pressure. The residue was triturated with Ethyl acetate (10.0 mL), filtered and the solid was collected. 9-7 (900 mg, 1.443 mmol, 94.39%) was obtained as a yellow solid. LC-MS: 437.2 [M+H]⁺.

To a solution of 9-7 (1 g, 2.291 mmol) in dioxane (5 mL) was added DDQ (0.78 g, 3.437 mmol) at 25° C. under N₂. The reaction mixture was stirred at 105° C. for 2 h. LCMS and HPLC showed most of 9-7 was consumed and desired MS was detected. The reaction mixture was concentrated under reduced pressure. The residue was triturated with Ethyl acetate (5.0 mL), filtered and the solid was collected. The residue was purified by prep-HPLC for 2 times (FA condition). 9-8 (50 mg, 0.115 mmol, 5.02%) was obtained as a brown solid. LC-MS: 435.1 [M+H]⁺.

A solution of 9-8 (25 mg, 0.058 mmol) in HBr (2 mL) was stirred at 100° C. for 1 h. LCMS (MCP230760-015-P1A) showed most of 9-8 was consumed and desired MS was detected. The mixture was purified by prep-HPLC (FA condition) without work up. The compound of Example 9 (6.64 mg, 0.015 mmol, 26.79%) was obtained as a red solid.

LC-MS: 421.2 [M+H]⁺.

¹H NMR (400 MHz, DMSO-d₆) δ 8.49-8.35 (m, 1H), 7.60 (br d, J=9.0 Hz, 1H), 7.32 (br d, J=9.0 Hz, 1H), 7.26 (br d, J=10.0 Hz, 1H), 7.14-7.08 (m, 1H), 6.70 (br d, J=10.0 Hz, 1H), 6.54-6.38 (m, 1H), 5.46-5.33 (m, 2H), 5.06-4.93 (m, 2H), 1.85 (tt, J=13.8, 7.1 Hz, 2H), 0.87 (br t, J=7.3 Hz, 3H).

The compounds below were synthesized following procedures described for Example 1.

| Ex | Structure | Name | [M + H]⁺ |
|---|---|---|---|
| 1. | 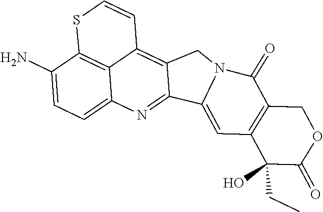 [SMILES: CC[C@@]1(O)C(=O)OCc2c1cc1n(c2=O)Cc2c-1nc1ccc(N)c3c1c2C=CS3] | (S)-4-amino-9-ethyl-9-hydroxy-12,15-dihydro-13H- pyrano[3′,4′:6,7]indolizino[1,2-b]thiopyrano[4,3,2- de]quinoline-10,13(9H)-dione | 420.2 |
| 2. | 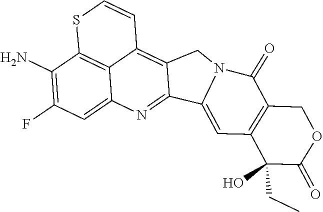 [SMILES: CC[C@@]1(O)C(=O)OCc2c1cc1n(c2=O)Cc2c-1nc1cc(F)c(N)c3c1c2C=CS3] | (S)-4-amino-9-ethyl-5-fluoro-9-hydroxy-12,15-dihydro- 13H-pyrano[3′,4′:6,7]indolizino[1,2-b]thiopyrano[4,3,2- de]quinoline-10,13(9H)-dione | 438.1 |
| 3. | 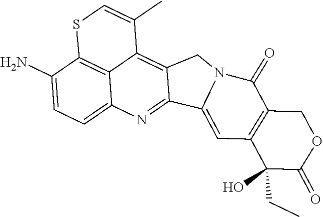 [SMILES: CC[C@@]1(O)C(=O)OCc2c1cc1n(c2=O)Cc2c-1nc1ccc(N)c3c1c2C(C)=CS3] | (S)-4-amino-9-ethyl-9-hydroxy-1-methyl-12,15-dihydro-13H- pyrano[3′,4′: 6,7]indolizino[1,2-b]thiopyrano[4,3,2-de]quinoline- 10,13(9H)-dione | 433.1 |
| 4. | 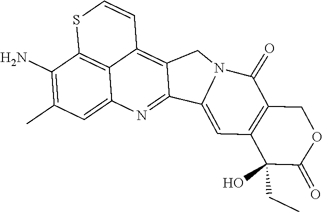 [SMILES: CC[C@@]1(O)C(=O)OCc2c1cc1n(c2=O)Cc2c-1nc1cc(C)c(N)c3c1c2C=CS3] | (S)-4-amino-9-ethyl-9-hydroxy-5-methyl-12,15-dihydro-13H- pyrano[3′,4′:6,7]indolizino[1,2-b]thiopyrano[4,3,2-de]quinoline- 10,13(9H)-dione | 434.0 |
| 5. | 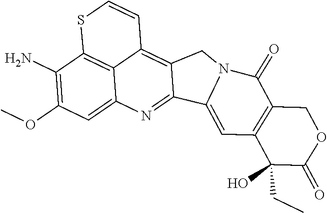 [SMILES: CC[C@@]1(O)C(=O)OCc2c1cc1n(c2=O)Cc2c-1nc1cc(OC)c(N)c3c1c2C=CS3] | (S)-4-amino-9-ethyl-9-hydroxy-5-methoxy-12,15-dihydro-13H- pyrano[3′,4′: 6,7]indolizino[1,2-b]thiopyrano[4,3,2-de]quinoline- 10,13(9H)-dione | 450.2 |
| 6. | 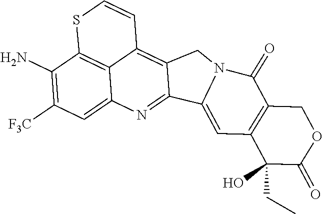 [SMILES: CC[C@@]1(O)C(=O)OCc2c1cc1n(c2=O)Cc2c-1nc1cc(C(F)(F)F)c(N)c3c1c2C=CS3] | (S)-4-amino-9-ethyl-9-hydroxy-5-(trifluoromethyl)-12,15- dihydro-13H-pyrano[3′,4′:6,7]indolizino[1,2-b] thiopyrano[4,3,2-de]quinoline- 10,13(9H)-dione | 487.1 |
| 7. | 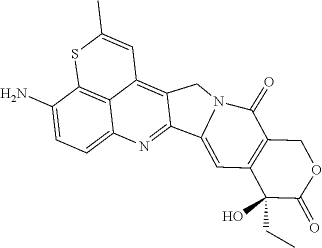 [SMILES: CC[C@@]1(O)C(=O)OCc2c1cc1n(c2=O)Cc2c-1nc1ccc(N)c3c1c2C=C(C)S3] | (S)-4-amino-9-ethyl-9-hydroxy-2-methyl-12,15-dihydro-13H- pyrano[3′,4′:6,7]indolizino[1,2-b]thiopyrano[4,3,2-de]quinoline- 10,13(9H)-dione | 433.1 |
| 8. | 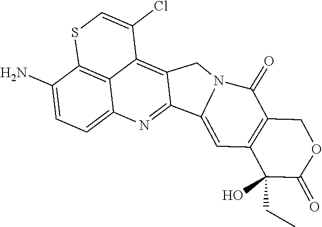 [SMILES: CC[C@@]1(O)C(=O)OCc2c1cc1n(c2=O)Cc2c-1nc1ccc(N)c3c1c2C(Cl)=CS3] | (S)-4-amino-1-chloro-9-ethyl-9-hydroxy-12,15-dihydro-13H- pyrano[3′,4′:6,7]indolizino[1,2-b]thiopyrano[4,3,2-de]quinoline- 10,13(9H)-dione | 453.1 |
| 9. | 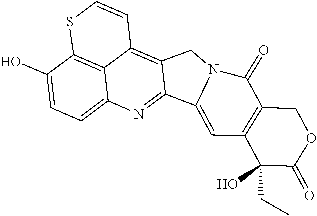 [SMILES: CC[C@@]1(O)C(=O)OCc2c1cc1n(c2=O)Cc2c-1nc1ccc(O)c3c1c2C=CS3] | (S)-9-ethyl-4,9-dihydroxy-12,15-dihydro-13H- pyrano[3′,4′:6,7]indolizino[1,2-b]thiopyrano[4,3,2- de]quinoline-10,13(9H)-dione | 421.2 |
| 10. | 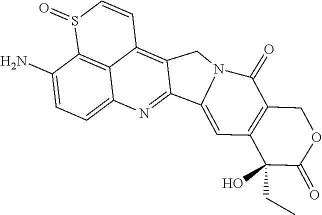 [SMILES: CC[C@@]1(O)C(=O)OCc2c1cc1n(c2=O)Cc2c-1nc1ccc(N)c3c1c2C=CS3=O] | (9S)-4-amino-9-ethyl-9-hydroxy-12,15-dihydro-13H- pyrano[3′,4′:6,7]indolizino[1,2-b]thiopyrano[4,3,2- de]quinoline-10,13(9H)-dione 3-oxide | 435.1 |
| 11. | 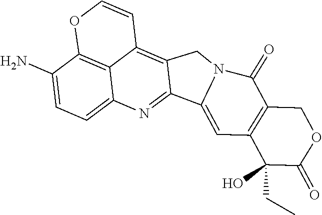 [SMILES: CC[C@@]1(O)C(=O)OCc2c1cc1n(c2=O)Cc2c-1nc1ccc(N)c3c1c2C=CO3] | (S)-4-amino-9-ethyl-9-hydroxy-12,15-dihydro-13H- pyrano[4,3,2-de]pyrano[3′,4′:6,7]indolizino[1,2- b]quinoline-10,13(9H)-dione | 403.2 |

### Example 12

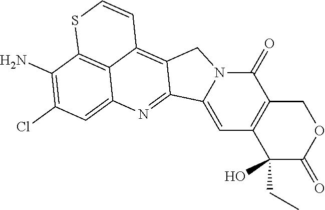 [SMILES: CC[C@@]1(O)C(=O)OCc2c1cc1n(c2=O)Cc2c-1nc1cc(Cl)c(N)c3c1c2C=CS3]

The compound of Example 12 was synthesized following procedures described for Example 1.

LC-MS: 454.1 [M+H]⁺.

¹H NMR (400 MHz, DMSO-d₆) δ 7.42-7.37 (m, 2H), 7.31-7.26 (m, 1H), 7.18 (br d, J=5.0 Hz, 1H), 6.87-6.79 (m, 1H), 6.74-6.58 (m, 1H), 6.54-6.41 (m, 1H), 5.52 (s, 1H), 5.43-5.28 (m, 2H), 5.03 (s, 1H), 2.00 (br d, J=7.5 Hz, 2H), 0.86 (br d, J=2.6 Hz, 3H).

### Example 13

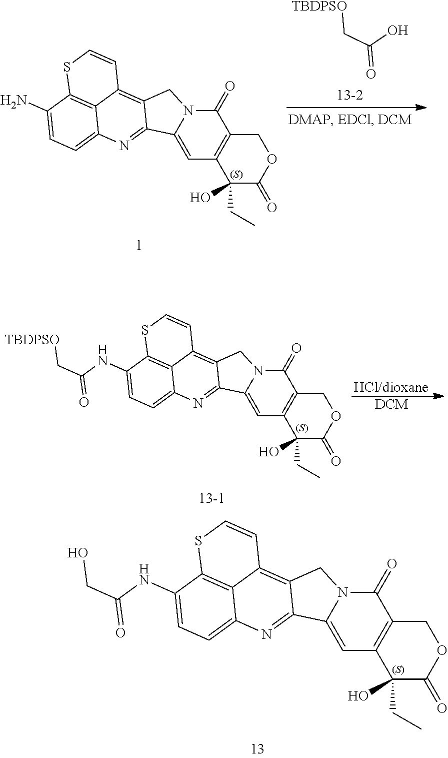

To a solution of the compound of Example 1 (150 mg, 0.358 mmol) in DCM (2.00 mL) was added compound 13-2 (167 mg, 0.536 mmol), DMAP (131 mg, 1.07 mmol) and EDCI (103 mg, 0.536 mmol) at 25° C. The mixture was stirred for 12 h at 25° C. LCMS showed the compound of Example 1 was consumed completely and desired MS was detected. The reaction mixture was concentrated under reduced pressure. The residue was purified by prep-HPLC (column: Phenomenex Luna C18 80*30 mm*3 um; mobile phase: [A: H₂O (0.1% TFA); B: ACN]; B %: 55.00%-90.00%, 8.00 min). 13-1 (80 mg, 0.112 mmol, 31.3% yield) was obtained as a brown solid. LC-MS: 716.2 [M+H]⁺.

To a solution of 13-1 (40.0 mg, 0.056 mmol) in DCM (4.00 mL) was added a solution of hydrogen chloride(g) in dioxane (0.140 mL, 0.559 mmol) at 25° C. and the mixture was stirred for 1 h at 25° C. LCMS showed 13-1 was consumed completely and desired MS was detected. The reaction mixture was concentrated under reduced pressure. The residue was purified by prep-HPLC (column: Phenomenex Luna C18 75*30 mm*3 um; mobile phase: [A: H₂O (0.1% TFA); B: ACN]; B %: 15.00%-45.00%, 8.00 min). 13 (17.0 mg, 0.036 mmol, 63.9% yield) was obtained as a brown solid.

LC-MS: 478.2 [M+H]⁺.

¹H NMR (400 MHz, DMSO-d₆) δ 7.57 (d, J=9.0 Hz, 1H), 7.27 (dd, J=9.5, 7.0 Hz, 2H), 6.83 (s, 1H), 6.72 (d, J=10.0 Hz, 1H), 5.61 (s, 1H), 5.48 (br d, J=2.6 Hz, 3H), 5.02 (s, 2H), 4.36-4.12 (m, 1H), 2.15-2.05 (m, 2H), 0.95-0.86 (m, 3H).

### Example 14

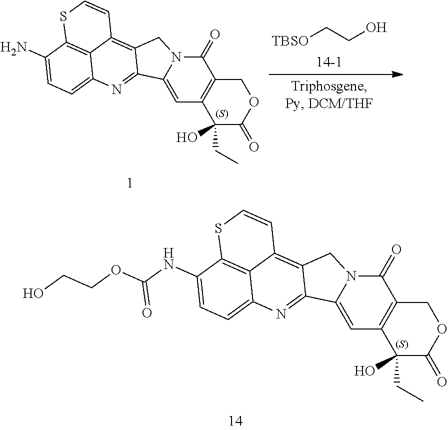

To a mixture of the compound of Example 1 (30.0 mg, 0.072 mmol) and Py (0.069 mL, 0.858 mmol) in DCM (5.00 mL) was added Triphosgene (25.5 mg, 0.086 mmol) at −78° C. The mixture was stirred at −78° C. for 3 h under N₂ before a solution of 14-1 (15.1 mg, 0.086 mmol) in THE (5.00 mL) was added at −78° C. The reaction mixture was warmed to 25° C. and stirred for 12 h. LCMS showed the SM was consumed completely and desired MS was detected. The reaction mixture was concentrated under reduce pressure. The residue was purified by prep-HPLC (column: 3_Phenomenex Luna C18 75*30 mm*3 um; mobile phase: [A: H₂O (0.2% FA); B: ACN]; B %: 10.00%-40.00%, 8.00 min). 14 (13.29 mg, 0.026 mmol, 36.37%) was obtained as a red solid.

LC-MS: 508.25 [M+H]⁺.

¹H NMR (400 MHz, MeOD-d₄) δ 7.80-7.74 (m, 1H), 7.72-7.66 (m, 1H), 7.59 (s, 1H), 7.35 (d, J=10.0 Hz, 1H), 6.90 (d, J=10.0 Hz, 1H), 5.65-5.56 (m, 1H), 5.41 (d, J=16.4 Hz, 1H), 5.13 (s, 2H), 4.29-4.23 (m, 2H), 3.87-3.75 (m, 2H), 2.09-1.88 (m, 2H), 1.03 (t, J=7.4 Hz, 3H).

### Example 15

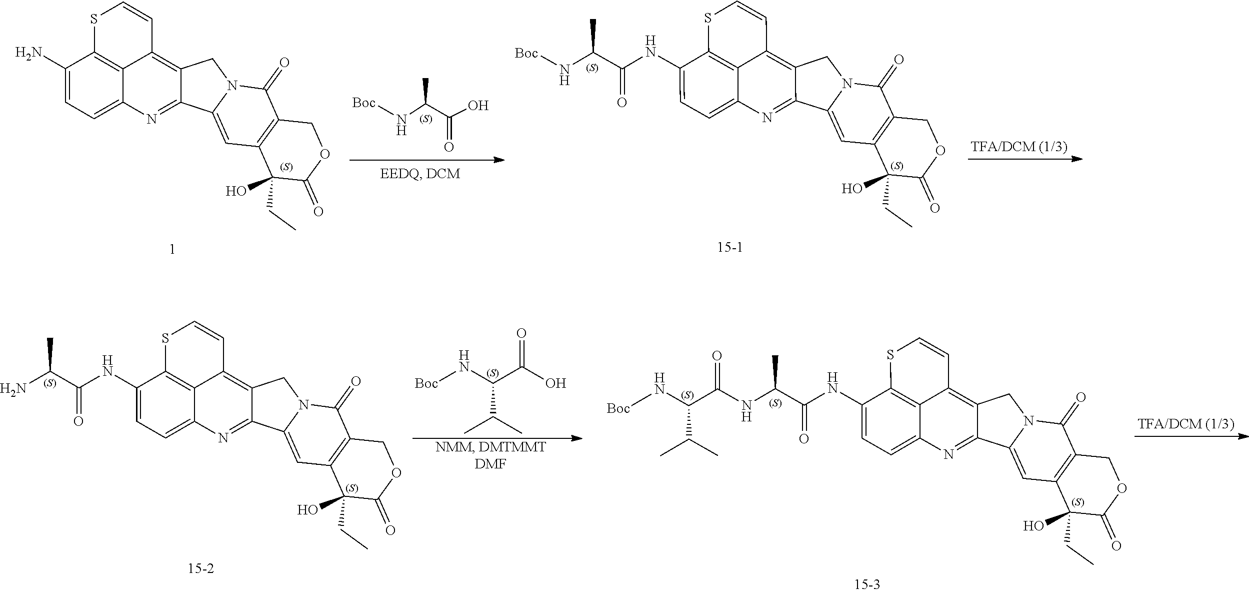

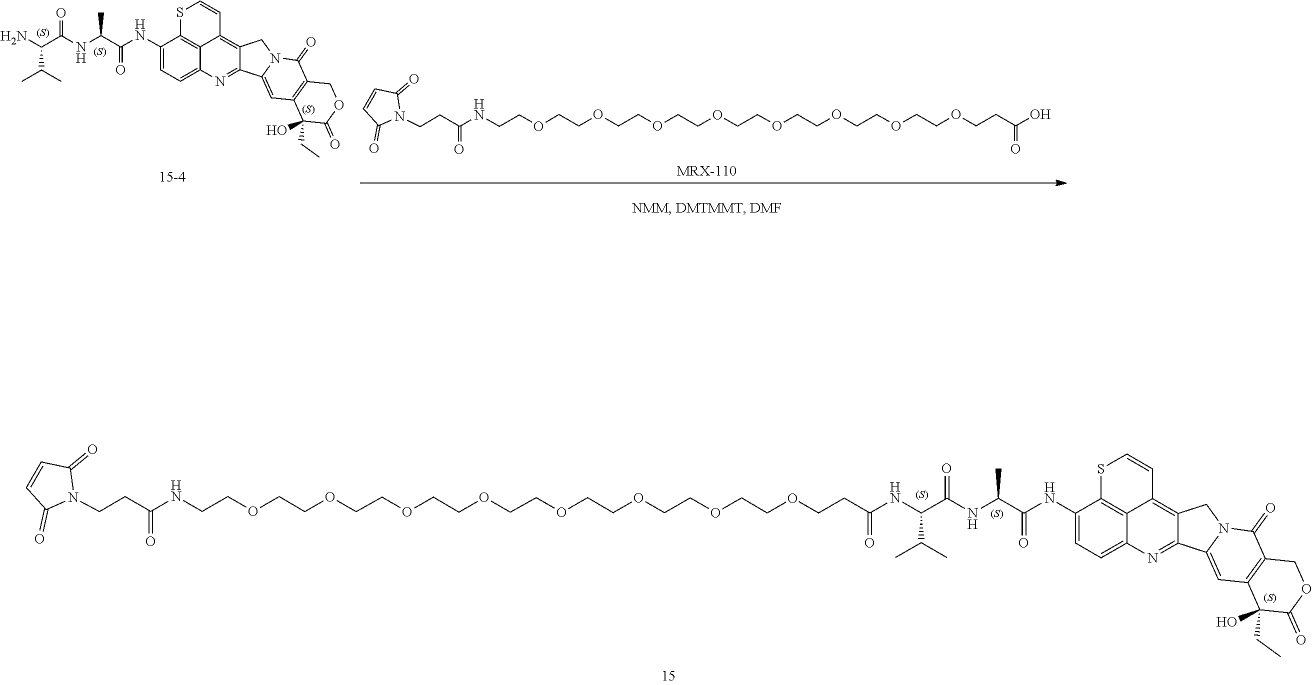

To a mixture of the compound of Example 1 (50.00 mg, 0.12 mmol) in DCM (10.00 mL) was added N-{[(2-methylprop-2-yl)oxy]carbonyl}-L-alanine (45.11 mg, 0.24 mmol) and EEDQ (58.96 mg, 0.24 mmol). The mixture was stirred at 25° C. for 72 h. LCMS showed the reaction was completed. The mixture was concentrated under reduced pressure. The residue was purified by prep-HPLC (column: Phenomenex luna C18 100*40 mm*5 um; mobile phase: [A: water (TFA)-B: ACN]; B %: 20%-45%, 8 min). 15-1 (10.00 mg, 0.02 mmol, 14.20% yield) was obtained as a yellow solid. LC-MS: 591.1 [M+H]⁺.

To a solution of 15-1 (10.00 mg, 0.02 mmol) in DCM (2.00 mL) was added TFA (0.70 mL, 9.14 mmol) and the mixture was stirred at 25° C. for 1 h. LCMS showed the reaction was completed. The reaction mixture was concentrated under reduced pressure to obtain the crude 15-2 (8.00 mg, 0.02 mmol, 96.33% yield) as yellow oil, which was used for the next step directly without purification. LC-MS: 491.1 [M+H]⁺.

To a solution of 15-2 (5.00 mg, 0.01 mmol) in DMF (2.00 mL) was added N-{[(2-methylprop-2-yl)oxy]carbonyl}-L-valine (2.21 mg, 0.01 mmol), NMM (0.004 mL, 0.04 mmol) and DMTMMT (3.20 mg, 0.01 mmol). The mixture was stirred at 25° C. for 12 h. LCMS showed the reaction was completed. The mixture was concentrated under reduced pressure. The residue was purified by prep-HPLC (column: Phenomenex Kinetex EVO C18 150*30 mm 5 um; mobile phase: [A: water (TFA)-B: ACN]; B %: 25%-55%, 20 min). 15-3 (3.00 mg, 0.004 mmol, 42.67% yield) was obtained as a yellow solid. LC-MS: 690.3 [M+H]⁺.

A solution of 15-3 (3.00 mg, 0.004 mmol) in TFA (0.30 mL) and DCM (0.90 mL) was stirred at 25° C. for 10 mins. LCMS showed the reaction was completed. The reaction mixture was concentrated under reduced pressure. 15-4 (2.00 mg, 0.003 mmol, crude) was obtained as yellow oil. LC-MS: 590.3 [M+H]⁺.

To a solution of 15-4 (2.00 mg, 0.003 mmol) in DMF (1.00) was added MRX-110 (2.01 mg, 0.003 mmol), NMM (0.001 mL, 0.014 mmol) and DMTMMT (1.07 mg, 0.003 mmol). The mixture was stirred at 25° C. for 12 h. LCMS showed the reaction was completed. The residue was purified by prep-HPLC (column: Phenomenex Gemini-NX 150*30 mm*5 um; mobile phase: [A: water (TFA)-B: ACN]; B %: 15%-40%, 20 min). 15 (1.22 mg, 0.001 mmol, 30.89% yield) was obtained as a yellow solid.

LC-MS: 583.5 [½M+H]⁺.

¹H NMR (400 MHz, METHANOL-d₄) δ 8.32 (br d, J=6.2 Hz, 1H), 8.08-7.99 (m, 1H), 7.73 (d, J=9.0 Hz, 1H), 7.57 (s, 1H), 7.55 (s, 1H), 7.33 (d, J=10.0 Hz, 1H), 6.88 (s, 1H), 6.81 (s, 2H), 5.60 (s, 1H), 5.41 (s, 1H), 3.76 (t, J=6.8 Hz, 4H), 3.65-3.59 (m, 30H), 3.52-3.45 (m, 4H), 2.57 (br t, J=5.6 Hz, 2H), 2.45 (t, J=6.9 Hz, 2H), 2.22-2.08 (m, 1H), 2.00-1.87 (m, 2H), 1.55 (br d, J=7.1 Hz, 3H), 1.04-0.98 (m, 9H)

The compounds below were synthesized following procedures described for Example 15.

| Ex. | Structure | Name | [M + H]⁺ |
|---|---|---|---|
| 15 | 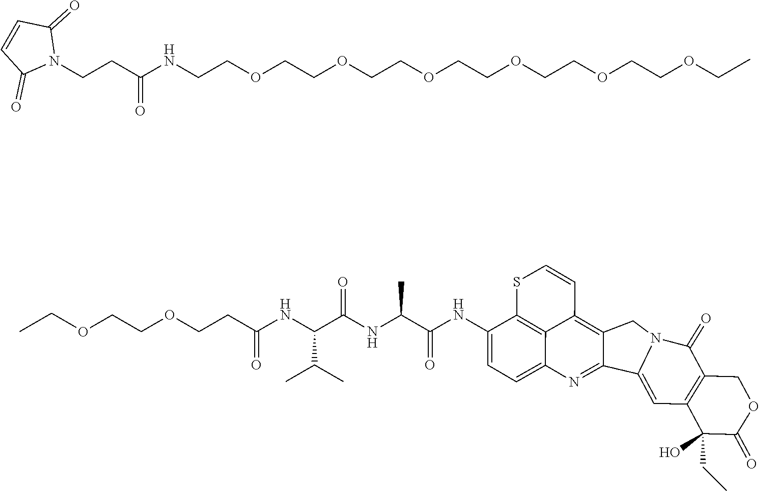 | 1-(3-(2,5-dioxo-2,5-dihydro- 1H-pyrrol-1-yl)propanamido)- N-((S)-1-(((S)-1-(((S)-9-ethyl- 9-hydroxy-10,13-dioxo- 9,10,13,15-tetrahydro-12H- pyrano[3′,4′:6,7]indolizino[1,2- b]thiopyrano[4,3,2- de]quinolin-4-yl)amino)-1- oxopropan-2-yl)amino)-3- methyl-1-oxobutan-2-yl)- 3,6,9,12,15,18,21,24- octaoxaheptacosan-27-amide | / |
| 16 | 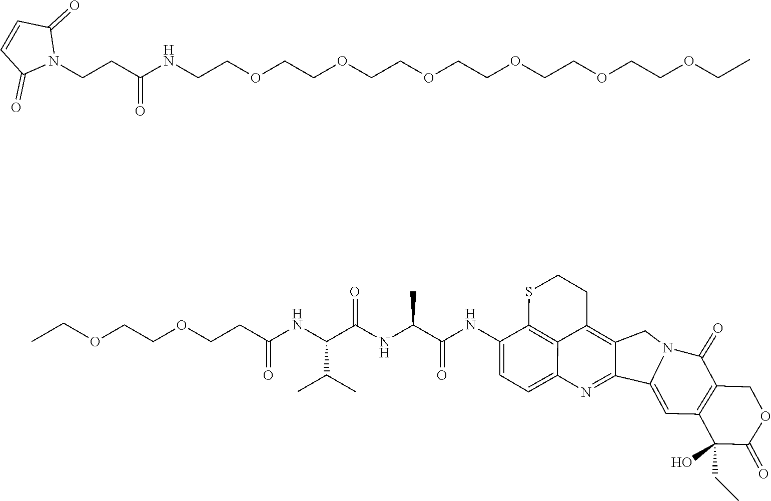 | 1-(3-(2,5-dioxo-2,5-dihydro- 1H-pyrrol-1-yl)propanamido)- N-((S)-1-(((S)-1-(((S)-9-ethyl- 9-hydroxy-10,13-dioxo- 1,2,9,10,13,15-hexahydro- 12H-pyrano[3′,4′:6,7]indolizino [1,2-b]thiopyrano[4,3,2- de]quinolin-4-yl)amino)-1- oxopropan-2-yl)amino)-3- methyl-1-oxobutan-2-yl)- 3,6,9,12,15,18,21,24- octaoxaheptacosan-27-amide | 1165.5 |
| 17 | 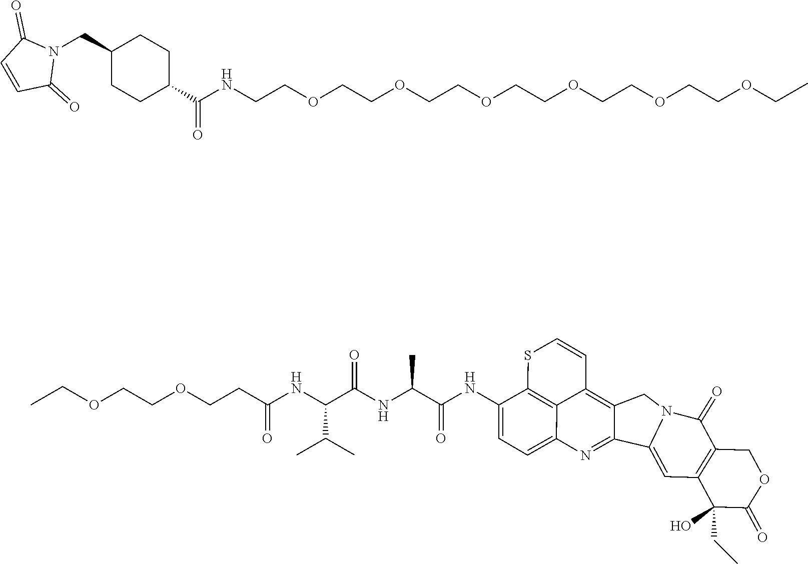 | (1r,4r)-4-((2,5-dioxo-2,5- dihydro-1H-pyrrol-1- yl)methyl)-N-((29S,32S)-33- (((S)-9-ethyl-9-hydroxy-10,13- dioxo-9,10,13,15-tetrahydro- 12H-pyrano[3′,4′:6,7]indolizino[1,2- b]thiopyrano[4,3,2- de]quinolin-4-yl)amino)-29- isopropyl-32-methyl-27,30,33- trioxo-3,6,9,12,15,18,21,24- octaoxa-28,31- diazatritriacontyl)cyclohexane- 1-carboxamide | 1231.5 |
| 18 | 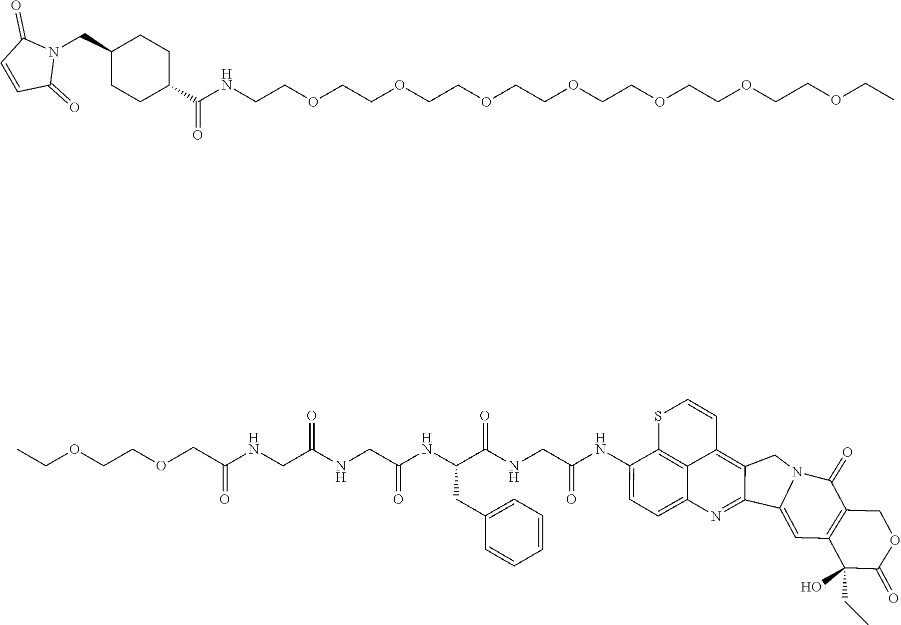 | (1r,4r)-N-((5S)-5-benzyl-1-((9- ethyl-9-hydroxy-10,13-dioxo- 9,10,13,15-tetrahydro-12H- pyrano[3′,4′:6,7]indolizino[1,2- b]thiopyrano[4,3,2- de]quinolin-4-yl)amino)- 1,4,7,10,13-pentaoxo- 15,18,21,24,27,30,33,36,39- nonaoxa-3,6,9,12- tetraazahentetracontan-41-yl)- 4-((2,5-dioxo-2,5-dihydro-1H- pyrrol-1-yl)methyl)cyclohexane-1- carboxamide | 1409.6 |
| 19 | 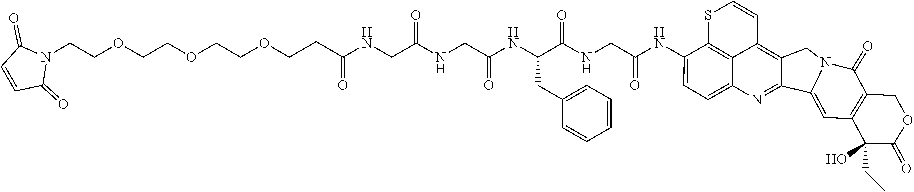 [SMILES: CC[C@@]1(O)C(=O)OCc2c1cc1n(c2=O)Cc2c-1nc1ccc(NC(=O)CNC(=O)[C@H](Cc3ccccc3)NC(=O)CNC(=O)CNC(=O)CCOCCOCCOCCN3C(=O)C=CC3=O)c3c1c2C=CS3] | (2S)-2-(1-(2,5-dioxo-2,5- dihydro-1H-pyrrol-1-yl)- 12,15-dioxo-3,6,9-trioxa- 13,16-diazaoctadecan-18- amido)-N-(2-((9-ethyl-9- hydroxy-10,13-dioxo- 9,10,13,15-tetrahydro-12H- pyrano[3′,4′:6,7]indolizino[1,2- b]thiopyrano[4,3,2-de] quinolin-4-yl)amino)-2-oxoethyl)-3- phenylpropanamide | 1020.3 |
| 20 | 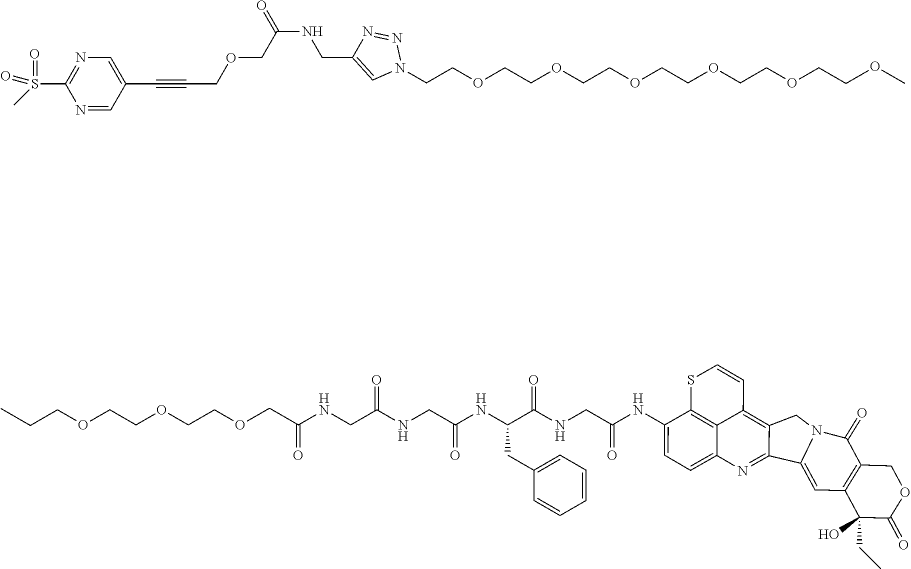 | N-(2-((2-(((2S)-1-((2-((9-ethyl- 9-hydroxy-10,13-dioxo- 9,10,13,15-tetrahydro-12H- pyrano[3′,4′:6,7]indolizino[1,2- b]thiopyrano[4,3,2- de]quinolin-4-yl)amino)-2- oxoethyl)amino)-1-oxo-3- phenylpropan-2-yl)amino)-2- oxoethyl)amino)-2-oxoethyl)- 29-(4-((2-((3-(2- (methylsulfonyl)pyrimidin-5- yl)prop-2-yn-1- | 1523.5 |
| | | yl)ocy)acetamido)methyl)-1H- | |
| | | 1,2,3-triazol-1-yl)- | |
| | | 3,6,9,12,15,18,21,24,27- | |
| | | nonaoxanonacosanamide | |

### Example 21

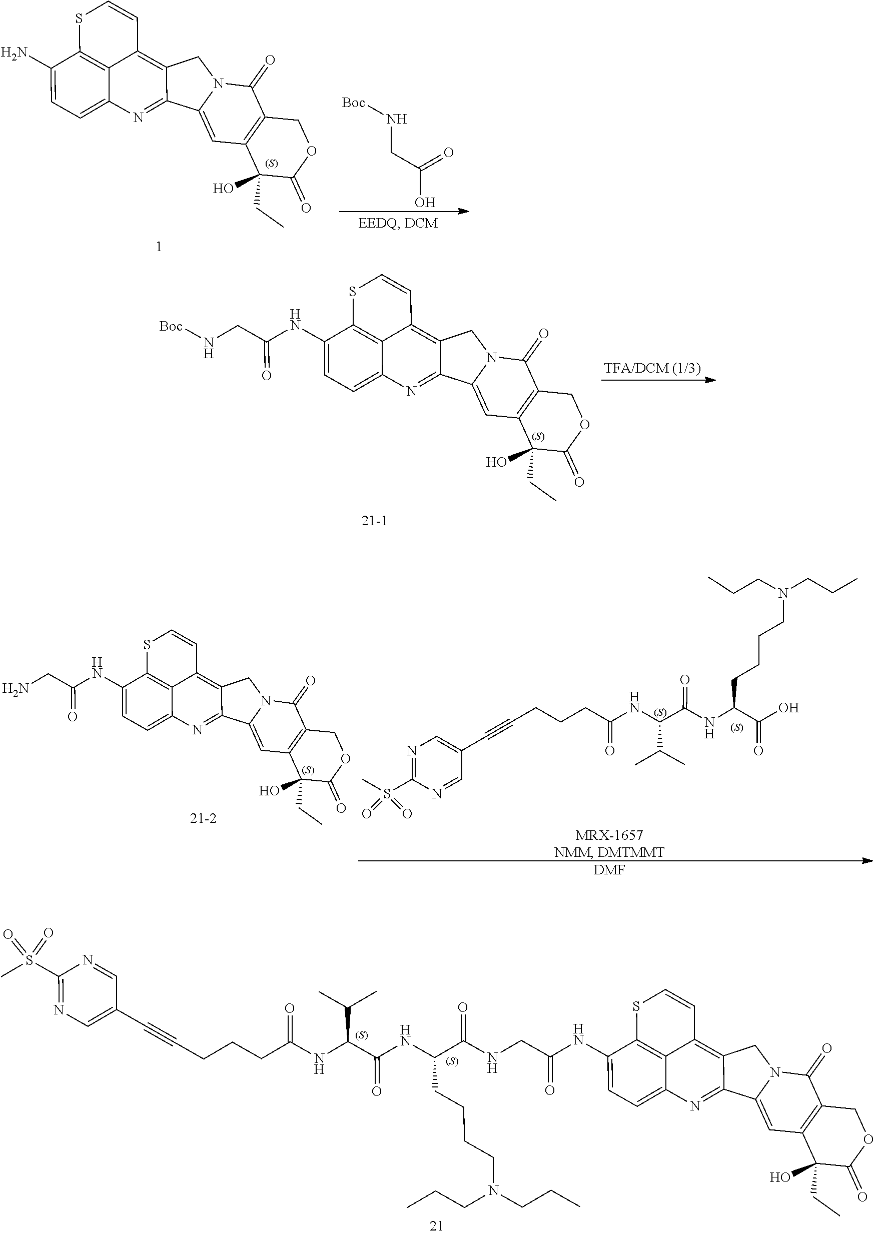

To a mixture of the compound of Example 1 (0.20 g, 0.48 mmol) in DCM (10.00 mL) was added N-{[(2-methylprop-2-yl)oxy]carbonyl}glycine (167.06 mg, 0.95 mmol) and EEDQ (235.82 mg, 0.95 mmol). The mixture was stirred at 25° C. for 12 h. LCMS showed the reaction was completed. The mixture was concentrated under reduced pressure. The residue was purified by prep-HPLC (column: Phenomenex Gemini-NX 150*30 mm*5 um; mobile phase: [A: water (TFA)-B: ACN]; B %: 15%-45%, 20 min) to obtain 21-1 (50.00 mg, 0.09 mmol, 18.19% yield) as red solid. LC-MS: 577.2 [M+H]⁺.

To a solution of 21-1 (50 mg, 0.087 mmol) in DCM (2.00 mL) was added TFA (0.70 mL, 9.14 mmol) and the mixture was stirred at 25° C. for 1 h. LCMS showed the reaction was completed. The reaction mixture was concentrated under reduced pressure to obtain crude 21-2 (40 mg, 0.084 mmol, 96.81% yield) as yellow oil, which was used directly for the next step without purification. LC-MS: 477.2 [M+H]⁺.

To a mixture of 21-2 (15.0 mg, 0.025 mmol) and MRX-1657 (14.7 mg, 0.025 mmol) in DMF (2.00 mL) was added NMM (0.014 mL, 0.127 mmol) and DMTMMT (8.33 mg, 0.025 mmol) at 25° C. The mixture was stirred at 25° C. for 12 h. LCMS showed the SM was consumed completely and desired MS was detected.

The reaction mixture was purified by prep-HPLC (column: Agilent Poroshell EC C18 3*30 mm, 2.7 um; mobile phase: [A: H₂O (0.1% TFA); B: MeOH]; B %: 15.00%-55.00%, 15.00 min). 21 (3.80 mg, 0.004 mmol, 14.4%) was obtained as a red solid.

LC-MS: 1038.3 [M+H]⁺.

¹H NMR (400 MHz, DMSO-d₆) δ 8.90 (s, 2H), 7.76 (d, J=9.0 Hz, 1H), 7.62-7.58 (m, 2H), 7.37 (d, J=10.0 Hz, 1H), 6.92 (d, J=10.1 Hz, 1H), 5.61 (d, J=16.6 Hz, 1H), 5.41 (d, J=16.6 Hz, 1H), 5.14 (s, 2H), 4.44-4.34 (m, 1H), 4.17 (s, 2H), 4.14-4.09 (m, 1H), 3.19-3.13 (m, 3H), 3.11-3.05 (m, 3H), 2.63-2.54 (m, 2H), 2.52-2.45 (m, 2H), 2.13-2.05 (m, 1H), 1.97 (td, J=13.3, 6.7 Hz, 5H), 1.86-1.67 (m, 8H), 1.54 (br dd, J=6.1, 3.7 Hz, 2H), 1.34 (br d, J=2.9 Hz, 2H), 1.05-0.98 (m, 16H).

### Example 22

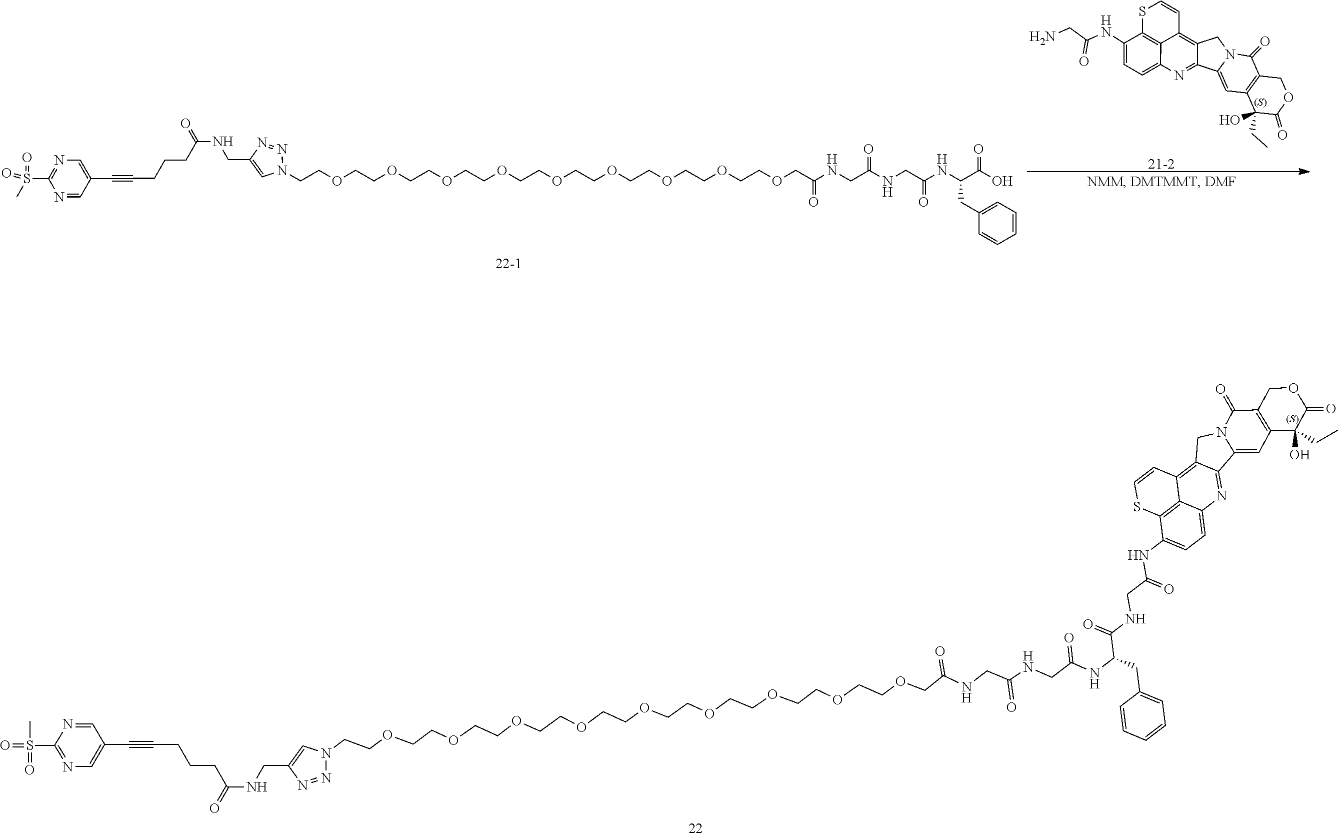

To a solution of 22-1 (65.0 mg, 0.04 mmol) in DMF (2.00 mL) was added 21-2 (20.4 mg, 0.04 mmol), NMM (0.02 mL, 0.17 mmol) and DMTMMT (13.43 mg, 0.04 mmol). The mixture was stirred at 25° C. for 12 hrs. LCMS showed the reaction was completed. The mixture was purified by prep-HPLC (column: Phenomenex Gemini-NX 150*30 mm*5 um; mobile phase: [A: water (TFA)-B: ACN]; B %: 15%-45%, 20 min. 22 (11.81 mg, 0.01 mmol, 18.14% yield) was obtained as a yellow solid.

LC-MS: 762.2 [½M+H]⁺.

¹H NMR (400 MHz, METHANOL-d₄) δ 8.98-8.90 (m, 2H), 8.40 (br s, 1H), 8.30 (br d, J=3.4 Hz, 1H), 8.19 (br d, J=7.4 Hz, 1H), 7.95-7.89 (m, 1H), 7.66-7.59 (m, 1H), 7.56-7.50 (m, 1H), 7.45 (s, 1H), 7.30-7.26 (m, 5H), 7.20 (br dd, J=8.3, 4.3 Hz, 1H), 6.77 (br d, J=9.9 Hz, 1H), 5.58 (s, 1H), 5.38 (br s, 1H), 4.57-4.51 (m, 3H), 4.47-4.42 (m, 3H), 4.21-4.13 (m, 2H), 4.07 (d, J=2.8 Hz, 1H), 4.03 (s, 2H), 3.96-3.92 (m, 3H), 3.88-3.84 (m, 3H), 3.69-3.66 (m, 3H), 3.61-3.56 (m, 30H), 3.37 (s, 1H), 3.36-3.35 (m, 3H), 2.61-2.54 (m, 2H), 2.41 (q, J=7.3 Hz, 2H), 2.00-1.89 (m, 4H), 1.27 (br t, J=7.1 Hz, 2H), 1.00 (t, J 7.3 Hz, 3H).

### Example 23: Preparation of Antibody-Drug Conjugates (e.g., ADC-1)

### Antibodies for Exemplary ADCs

Antibodies for the examples' ADC compounds were prepared according to conventional methods, for example, vector construction, eukaryotic cell transfection such as HEK2943 cell (Life Technologies Cat. No. 11625019) transfection, purification, and expression. Antibodies prepared included trastuzumab light chain (SEQ. ID NO. 1), trastuzumab heavy chain (SEQ. ID NO. 2), pertuzumab light chain (SEQ. ID NO. 3), pertuzumab heavy chain (SEQ. ID NO. 4), B7H3 antibody light chain (SEQ. ID NO. 5), and B7H3 antibody heavy chain (SEQ. ID NO. 6).

### General Process of Conjugation

A formulated aqueous solution of tris(2-carboxy-ethyl) phosphine (10 mM, 0.082 mL, 0.82 μmol) was added to a PBS-buffered aqueous solution of antibody (0.05 M PBS-buffered aqueous solution with pH=6.5; 2.5 ml, 9.96 mg/ml, 0.168 umol) at 37° C. The reaction solution was placed in a water bath shaker and shaken at 37° C. for 3 hours before stopping the reaction. The reaction solution was cooled to 25° C. in a water bath and diluted to 5.0 mg/ml. 2.0 ml of the solution was taken for the next reaction.

The linker-camptothecin compound (2.1 mg, 2.02 umol) was dissolved in 0.10 mL of DMSO, and then added to 2.0 ml of the above solution. The reaction solution was placed in a water bath shaker, and shaked at 25° C. for 3 hours before stopping the reaction. The reaction solution was desalted and purified with a Sephadex G25 gel column (elution phase:0.05 M PBS-buffered aqueous solution with pH=6.5, containing 0.001 M EDTA) to obtain the PBS-buffered solution of the exemplary product ADC, which was stored at 4° C.

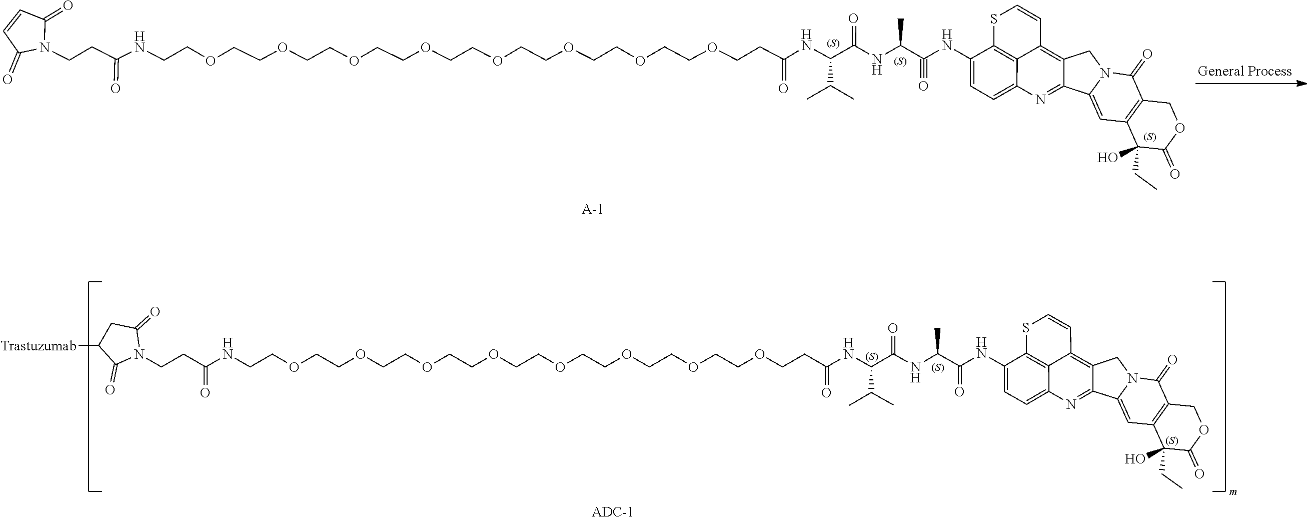

The analysis of drug loading of the ADC (UV method) was carried out according to the method of U.S. Patent Application Publication No. US 2021/0353764 (i.e., paragraphs [0702]ff).

The analysis of drug loading of the ADC (LC-MS method) was carried out according to the method of U.S. Pat. No. 11,572,414 (i.e., col. 2, line 51, to col. 3, line 15).

The ADC aggregation levels was determined by Size Exclusion Chromatography (SEC). All samples were filtered through 0.22 m filter prior to HPLC-SEC analysis.

The HPLC method was conducted as follows:
Instrument: Thermo Ultimate 3000
Column: Waters, XBridge BEH 200 Å SEC 3.5 m (7.8×300 mm)
Mobile Phase: PBS with 15% isopropanol, pH 7.4
Flow Rate: 0.5 ml/min, 30 min.

The ADCs below were synthesized following the general procedures described in Example 23.

| | | | | SEC | | |
|---|---|---|---|---|---|---|
| | | Payload | | Aggre- | LC- | |
| ADC | | Example | Yield | gation | MS- | UV- |
| Name | Structure | # | (%) | (%) | DAR | DAR |
| ADC-2 | 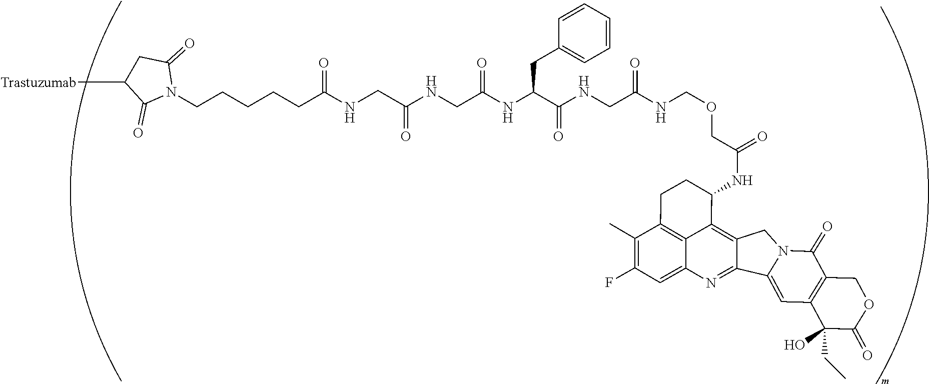 | Dxd | 80.0% | 3.04% | 8.0 | / |
| ADC-3 | 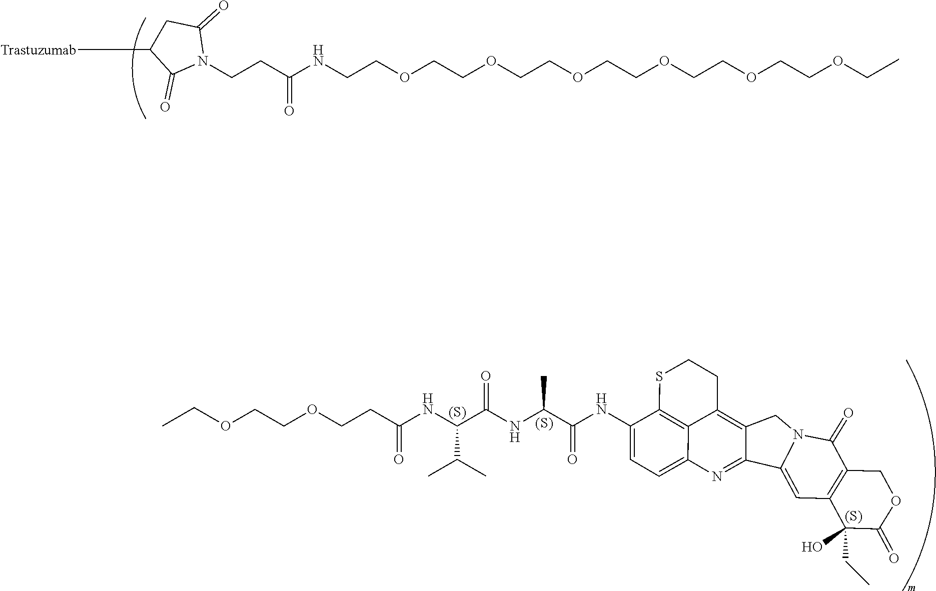 | / | / | / | 7.13 | / |
| ADC-4 | 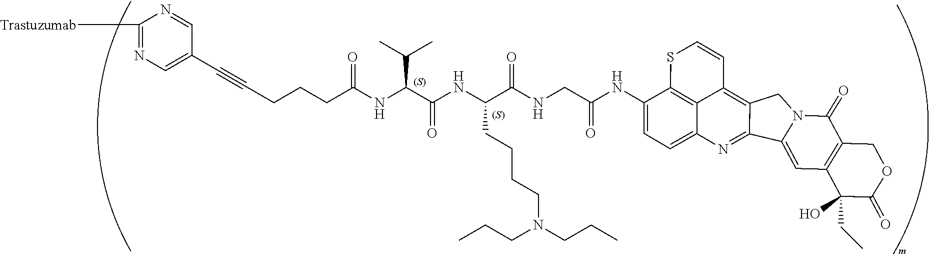 | 1 | / | / | 7.66 | / |
| ADC-5 | 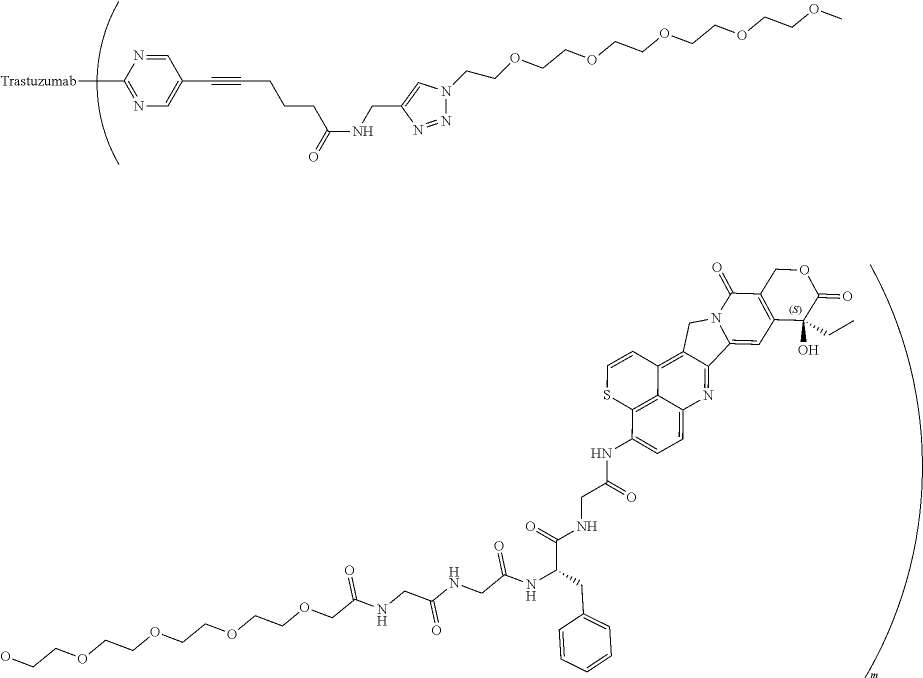 | 1 | / | / | 7.26 | / |

### Example 24

### In Vitro Cytotoxicity Test of Camptothecin Payloads

Human lung adenocarcinoma cell line A549, NCI-N87, and SK-BR-3 were used to evaluate the cytotoxicity of small molecule fragment of the present invention. These cells were seeded to a 96-well plate at 2000 cells per well. After overnight incubation under 500 CO₂ and 37° C., each diluted Compound was added. Cell viability was evaluated after 3 days using a CellTiter-Glo luminescent cell viability assay kit from Promega Corp. and according to the manufacturer's instructions. The results are shown in Table 1 below.

TABLE 1
Cytotoxicity on A549 by Compounds of Formula (II)

| | A549 | SK-BR-3 | NCI-N87 |
|---|---|---|---|
| | Cytotoxicity | Cytotoxicity | Cytotoxicity |
| Example # | IC₅₀ (nM) | IC₅₀ (nM) | IC₅₀ (nM) |
| 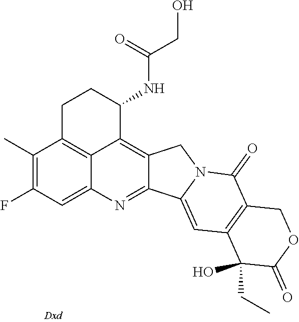 [SMILES: CC[C@@]1(O)C(=O)OCc2c1cc1n(c2=O)Cc2c-1nc1cc(F)c(C)c3c1c2[C@@H](NC(=O)CO)CC3] | 162.7 | 802 | 26 |
| 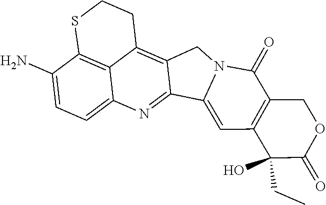 [SMILES: CC[C@@]1(O)C(=O)OCc2c1cc1n(c2=O)Cc2c-1nc1ccc(N)c3c1c2CCS3] | 22.3 | / | / |
| 1 | 10.7 | 38.6 | 5.2 |
| 2 | 0.8 | / | / |
| 13 | 26.4 | 52.3 | 21.7 |

As shown in Table 1, the potency of compounds of Formula (11) higher than the comparator compound (Dxd).

### Example 25

### In Vitro Cytotoxicity Test of ADCs

Cancer cell lines with different level of Her2 expression, including NCI-N87, Calu-3, SK-BR-3, CAPAN-1 and CFPAC-1 cells, were used to test the cytotoxicity of ADC of the present invention. These cell lines were seeded to a 96-well plate at 1000-4000 cells per well. After overnight incubation under 500 CO₂ and 37° C., each diluted Compound was added. Cell viability was evaluated after 6 days using a CellTiter-Glo luminescent cell viability assay kit from Promega Corp. and according to the manufacturer's instructions. The results are shown in Table 2 below.

TABLE 2
In vitro cytotoxicity test of ADCs

| | | Cytotoxicity IC₅₀, nM | | | | |
|---|---|---|---|---|---|---|
| | LC-MS- | SK-BR- | Calu- | NCI- | CAPAN- | CFPAC- |
| Example | DAR | 3 | 3 | N87 | 1 | 1 |
| ADC-1 | 4.4 | 1.7 | / | 4.0 | / | / |
| ADC-2 | 8.0 | 1.2 | 2.9 | 3.2 | 40.7 | 122.3 |

Calu-3, NCI-N87 and SK-BR-3 are all Her2 high-expression cancer cell lines, CAPAN-1 and CFPAC-1 are reported as Her2 low-expression cell lines. The ADCs of the present invention, display comparable or higher cytotoxicity in the Her2 high-expression cancer cell lines, but surprisingly with significantly higher potency in the Her2 low-expression cell lines.

### Example 26

### Rat PK Study

Male SD rat, 180-200 g (BK lab. Animal Ltd.), 3 Rats/group. Blood samples were collected by orbital vein after IV administration 1 mg/kg dosage at 0.083, 0.5, 1, 2, 4, and 8 h into heparin-containing tubes, centrifugated at 10000 rpm for 3 min to get plasma. All samples were stored at −20° C. until they were sent to Suzhou Chengyao Biotech Co., Ltd. for analysis. The results are shown in Table 3 below.

TABLE 3
Rat PK results

| Compound | 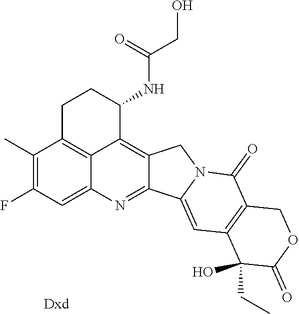 | 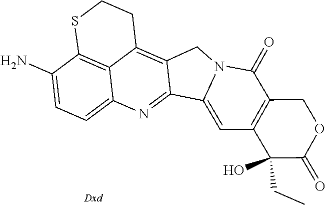 | Example 1 |
|---|---|---|---|
| T_{1/2} (h) | 0.5 | 2.1 | 0.3 |
| Vss (L/kg) | 20.6 | 7.2 | 0.9 |
| MRT0-∞ (h) | 0.5 | 1.9 | 0.2 |

As shown in Table 3, the compound of the present invention is less stable in plasma. The Vss of the compound of the present invention is significantly reduced, indicating that the drug is mainly concentrated in the plasma; And with a shorter half-life, it can be cleared faster, reducing the risk of damage to external tumor tissues. The reduced Vss and increased instability of free toxin is favored for ADC, because it has a potential to reduce systemic exposure of toxin once it is released from the ADC, and therefore attenuate toxicity caused by the toxin.

### Example 27

### In Vitro Bystander Killing of ADC

Target cell SK-BR-3 and GFP-labelled tool cell Flip in 293 mGFP, plated either individually or mixed and cultured for 1 day, were treated with 4-fold serial dilution of ADCs of the present invention solution for 5 days. (The mixed cells were prepared by mixing these two cells and seeding 40 μL/well with a final density of 750 cells/well for SK-BR-3 and 250 cells/well for Flip in 293 mGFP, respectively.) At the end of treatment, the number of live Flip in 293 mGFP cells was determined by High-Content Screening (HCS) Assays in DPC and FITC channel. The ADCs of the present invention have a bystander killing effect.

### Example 28

### In Vitro Plasma Stability Study of ADC

A solution of ADC of the present invention was prepared at 1.6 mg/mL and added to human plasma, and the mixture was cultured at 37° C. A 40 uL sample was taken at each time point of 0, 6, 18, 24, 72, and 96 hours. After normal processing, the plasma samples were tested for the free payload by a LCMSMS method. The ADCs of the present invention have a good stability in human plasma.

The LCMSMS method had the following features:
Instrument: Thermo Orbitrap Exploris240
Column: ACQUITY UPLC® BEH C18 1.7 μm (2.1×150 mm)
Mobile Phase: 0.1% FA in ACN
Flow Rate: 0.3 ml/min

### Example 29

### In Vivo Efficacy Study of ADCs

The NCI-N87 cell line was used to create the CDX (Cell Line Derived Xenograft) NCI-N87 xenograft mouse model. Each 6-8 weeks old nu/nu nude mouse was subcutaneously injected into the right flank with 10⁷ cells in 200 μL of a Matrigel-NCI-N87 cell suspension. The injection sites were palpated up to three times weekly until tumors are established to an average size of 300 mm³ as measured via digital calipers. Animals were randomized into treatment groups. ADCs of the present invention were administrated by i.v. injection. Tumor size was measured and recorded weekly. After treatment with the ADCs of the present invention, tumor growth is greatly inhibited.

The representative data taken in their entirety reveal a surprisingly superior therapeutic potential for the compounds described herein, with the beneficial unexpected advantages in areas of potency, efficacy, and bystander killing. The dramatic and surprising improvement in in vivo efficacy for ADCs provided herein offers marked benefits for human or mammal therapy, including but not limited to better clinical cure rate, a reduced effective drug dose, and reduced possible adverse effects.

SEQUENCE LISTING

Trastuzumab Light chain
SEQ ID NO. 1
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
Trastuzumab Heavy chain
SEQ ID NO. 2
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
The following is the sequence of Pertuzumab:
Pertuzumab Light chain
SEQ ID NO. 3
DIQMTQSPSSLSASVGDRVTITCKASQDVSIGVAWYQQKPGKAPKLLIY
SASYRYTGVPSRFSGSGSGTDFTLTISSLQPEDFATYYCQQYYIYPYTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
Pertuzumab Heavy chain
SEQ ID NO. 4
EVQLVESGGGLVQPGGSLRLSCAASGFTFTDYTMDWVRQAPGKGLEWVA
DVNPNSGGSIYNQRFKGRFTLSVDRSKNTLYLQMNSLRAEDTAVYYCAR
NLGPSFYFDYWGQGTLVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQK
SLSLSPGK
The following is the sequence of B7H3 antibody:
B7H3 Light chain
SEQ ID NO. 5
DTWTQEPSFSVSPGGTVTLTCGLSSGSVSTSHYPSWYQQTPGQAPRMLI
YNTNTRSSGVPDRFSGSILGNKAALTITGAQADDESDYYCAIHVDRDIW
VFGGGTKLTVLGQPKANPTVTLFPPSSEELQANKATLVCLISDFYPGAV
TVAWKADGSPVKAGVETTKPSKQSNNKYAASSYLSLTPEQWKSHRSYSC
QVTHEGSTVEKTVAPTEC
B7H3 Heavy chain
SEQ ID NO. 6
QVQLVQSGGGWQPGTSLRLSCAASGFIFSSSAMHWVRQAPGKGLEWVAV
ISYDGSNKYYVDSVKGRFTISRDNSKNTLYLQMNSLRAEDTAVYYCARS
ARLYASFDYWGQGALVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

What is claimed is:

1. A ligand-drug conjugate or a pharmaceutically acceptable salt or solvate thereof, wherein the ligand-drug conjugate comprises a drug unit D¹ of Formula I: 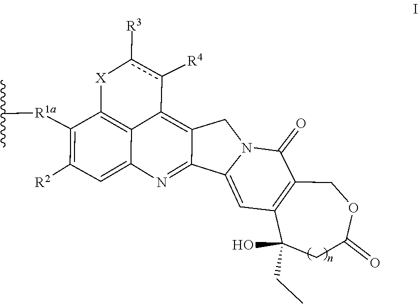
Wherein:
X is either absent, or selected from —O—, —S—, —S(O₂)—, or —S(O)—;
R^{1a} is —NH—R^{1b}—O—, —NH—, or —O—; optionally, R^{1a} is —NH— or —O—;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R² is selected from H, halo, hydroxy, cyano, nitro, C₃-C₈ cycloalkyl, C₁-C₈ alkyl, C₁-C₈ alkoxy, R⁵—(C₁-C₈ alkylene)-, amino, (C₁-C₈ alkyl)NHC(O)O—, (C₃-C₈ cycloalkyl)NHC(O)O—, (C₁-C₈ alkyl)NH—, azetidin-1-yl, pyrrolidin-1-yl, piperidin-1-yl, azepane-1-yl, (C₁-C₈ alkyl)C(O)O—, or (C₁-C₈ alkyl)C(O)NH—; wherein azetidin-1-yl, pyrrolidin-1-yl, piperidin-1-yl, azepane-1-yl, C₁-C₈ alkoxy, each “C₃-C₈ cycloalkyl”, and each “C₁-C₈ alkyl” are independently optionally substituted with one to four R⁶;
R³ and R⁴ are independently selected from H, halo, hydroxy, cyano, C₁-C₈ alkyl, C₁-C₈ alkoxy, C₃-C₈ cycloalkyl, aryl, heteroaryl, or amino; wherein C₁-C₈ alkyl, C₁-C₈ alkoxy, C₃-C₈ cycloalkyl, aryl, and heteroaryl are independently optionally substituted with one to four R⁶;
R⁵ is selected from C₁-C₈ alkoxy, C₃-C₈ cycloalkyl, aryl, or heteroaryl; wherein C₁-C₈ alkoxy, C₃-C₈ cycloalkyl, aryl, and heteroaryl are independently optionally substituted with one to four R⁶;
R⁶ at each occurrence is independently selected from halo, hydroxy, cyano, amino, C₁-C₈ alkyl, C₃-C₈ cycloalkyl, aryl, or heteroaryl;
and wherein a line with a dotted line represents either a single bond or a double bond;
n is an integer from 0 or 1.

2. The ligand-drug conjugate or a pharmaceutically acceptable salt or solvate thereof according to claim 1, wherein D¹ is represented by Formula II: 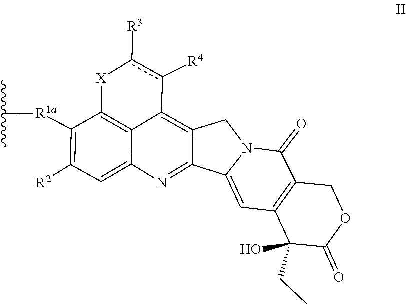
X is —O— or —S—;
R^{1a} is —NH—R^{1b}—O—, —NH—, or —O—; optionally, R^{1a} is —NH— or —O—;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R² is selected from H, halo, hydroxy, cyano, nitro, C₃-C₆ cycloalkyl, C₁-C₆ alkyl, C₁-C₆ alkoxy, R⁵—(C₁-C₃ alkylene)-, amino; wherein C₁-C₆ alkoxy, C₃-C₆ cycloalkyl, and C₁-C₆ alkyl are independently optionally substituted with one to four R⁶;
R³ and R⁴ are independently selected from H, halo, hydroxy, cyano, C₁-C₆ alkyl, C₁-C₆ alkoxy, or C₃-C₆ cycloalkyl; wherein C₁-C₆ alkyl, C₁-C₆ alkoxy, and C₃-C₆ cycloalkyl are independently optionally substituted with one to four R⁶;
R⁵ is C₁-C₆ alkoxy or C₃-C₆ cycloalkyl; wherein C₁-C₆ alkoxy and C₃-C₆ cycloalkyl are independently optionally substituted with one to four R⁶;
R⁶ at each occurrence is independently selected from halo, hydroxy, cyano, amino, C₁-C₃ alkyl, or C₃-C₆ cycloalkyl;
and wherein a line with a dotted line represents either a single bond or a double bond.

3. The ligand-drug conjugate or a pharmaceutically acceptable salt or solvate thereof according to claim 1, wherein R², R³ and R⁴ are independently selected from H, halo, C₁-C₃ alkyl, or C₁-C₃ alkoxy and wherein C₁-C₃ alkoxy and C₁-C₃ alkyl are independently optionally substituted with one to four halo.

4. The ligand-drug conjugate or a pharmaceutically acceptable salt or solvate thereof according to claim 1, wherein a line with a dotted line represents a double bond.

5. The ligand-drug conjugate or a pharmaceutically acceptable salt or solvate thereof according to claim 1, wherein D¹ is selected from structures below:

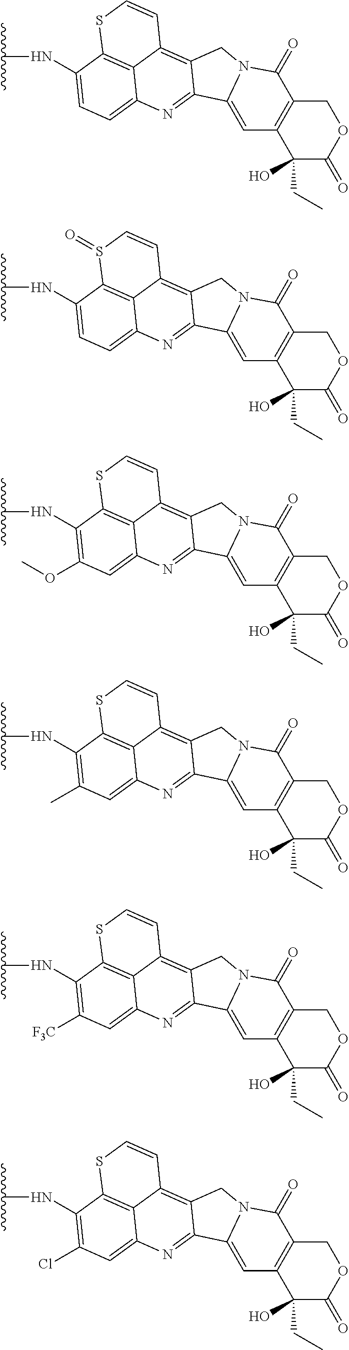 [SMILES: *Nc1ccc2nc3c(c4c2c1SC=C4)Cn1c-3cc2c(c1=O)COC(=O)[C@]2(O)CC ; *Nc1ccc2nc3c(c4c2c1S(=O)C=C4)Cn1c-3cc2c(c1=O)COC(=O)[C@]2(O)CC ; *Nc1c(OC)cc2nc3c(c4c2c1SC=C4)Cn1c-3cc2c(c1=O)COC(=O)[C@]2(O)CC ; *Nc1c(C)cc2nc3c(c4c2c1SC=C4)Cn1c-3cc2c(c1=O)COC(=O)[C@]2(O)CC ; *Nc1c(C(F)(F)F)cc2nc3c(c4c2c1SC=C4)Cn1c-3cc2c(c1=O)COC(=O)[C@]2(O)CC ; *Nc1c(Cl)cc2nc3c(c4c2c1SC=C4)Cn1c-3cc2c(c1=O)COC(=O)[C@]2(O)CC]

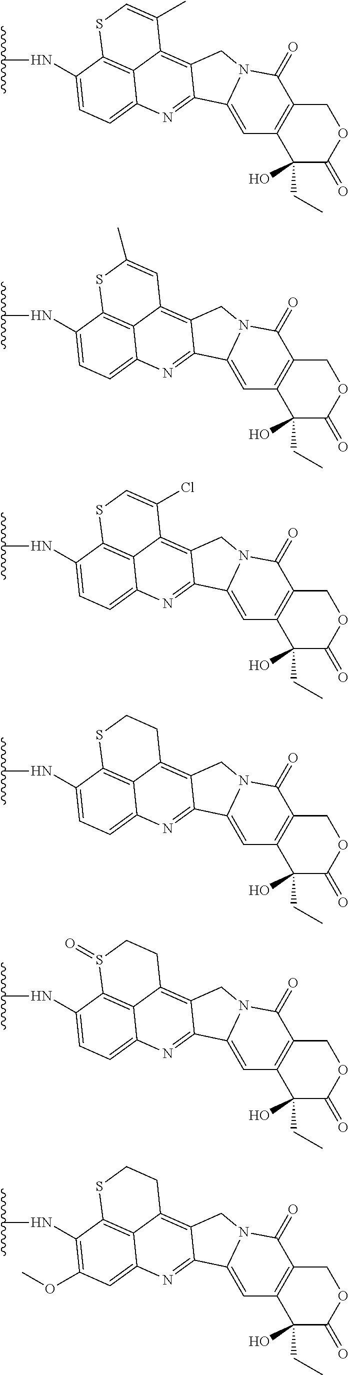 [SMILES: *Nc1ccc2nc3c(c4c2c1SC=C4C)Cn1c-3cc2c(c1=O)COC(=O)[C@]2(O)CC ; *Nc1ccc2nc3c(c4c2c1SC(C)=C4)Cn1c-3cc2c(c1=O)COC(=O)[C@]2(O)CC ; *Nc1ccc2nc3c(c4c2c1SC=C4Cl)Cn1c-3cc2c(c1=O)COC(=O)[C@]2(O)CC ; *Nc1ccc2nc3c(c4c2c1SCC4)Cn1c-3cc2c(c1=O)COC(=O)[C@]2(O)CC ; *Nc1ccc2nc3c(c4c2c1S(=O)CC4)Cn1c-3cc2c(c1=O)COC(=O)[C@]2(O)CC ; *Nc1c(OC)cc2nc3c(c4c2c1SCC4)Cn1c-3cc2c(c1=O)COC(=O)[C@]2(O)CC]

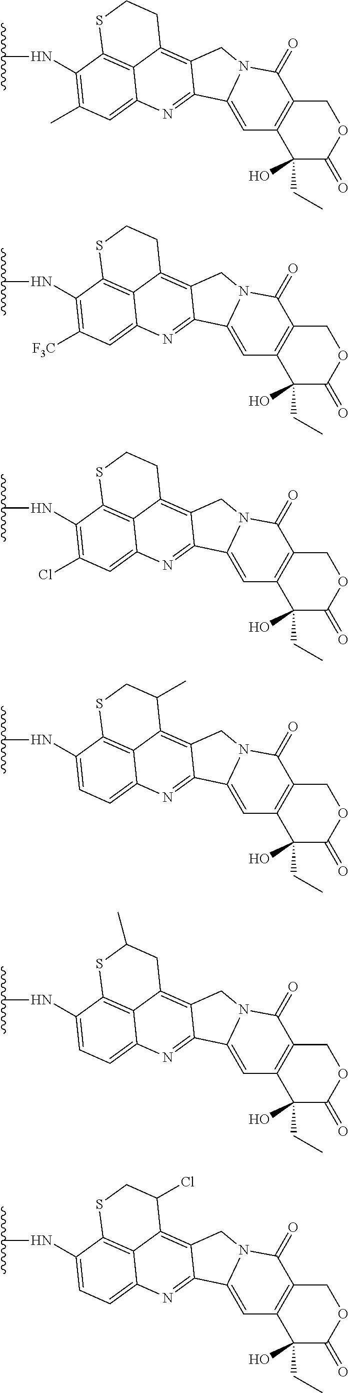 [SMILES: *Nc1c(C)cc2nc3c(c4c2c1SCC4)Cn1c-3cc2c(c1=O)COC(=O)[C@]2(O)CC ; *Nc1c(C(F)(F)F)cc2nc3c(c4c2c1SCC4)Cn1c-3cc2c(c1=O)COC(=O)[C@]2(O)CC ; *Nc1c(Cl)cc2nc3c(c4c2c1SCC4)Cn1c-3cc2c(c1=O)COC(=O)[C@]2(O)CC ; *Nc1ccc2nc3c(c4c2c1SCC4C)Cn1c-3cc2c(c1=O)COC(=O)[C@]2(O)CC ; *Nc1ccc2nc3c(c4c2c1SC(C)C4)Cn1c-3cc2c(c1=O)COC(=O)[C@]2(O)CC ; *Nc1ccc2nc3c(c4c2c1SCC4Cl)Cn1c-3cc2c(c1=O)COC(=O)[C@]2(O)CC]

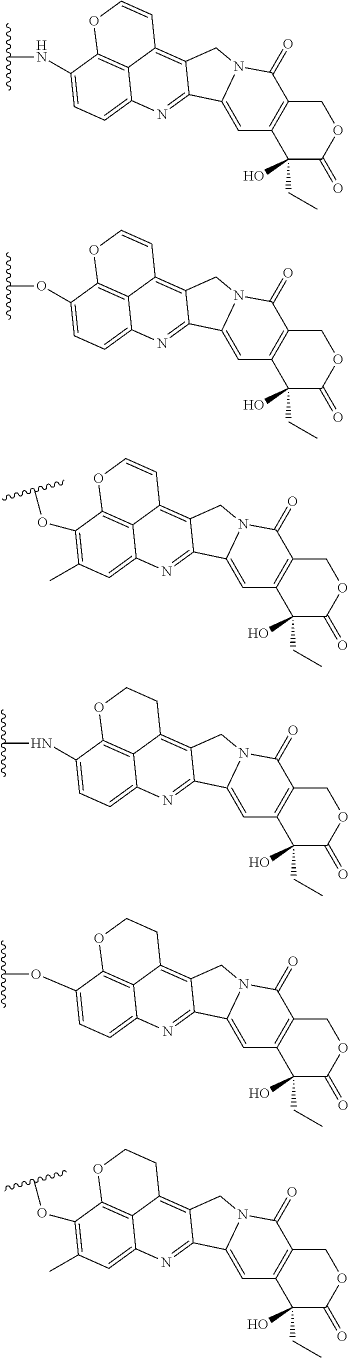 [SMILES: *Nc1ccc2nc3c(c4c2c1OC=C4)Cn1c-3cc2c(c1=O)COC(=O)[C@]2(O)CC ; *Oc1ccc2nc3c(c4c2c1OC=C4)Cn1c-3cc2c(c1=O)COC(=O)[C@]2(O)CC ; *Oc1c(C)cc2nc3c(c4c2c1OC=C4)Cn1c-3cc2c(c1=O)COC(=O)[C@]2(O)CC ; *Nc1ccc2nc3c(c4c2c1OCC4)Cn1c-3cc2c(c1=O)COC(=O)[C@]2(O)CC ; *Oc1ccc2nc3c(c4c2c1OCC4)Cn1c-3cc2c(c1=O)COC(=O)[C@]2(O)CC ; *Oc1c(C)cc2nc3c(c4c2c1OCC4)Cn1c-3cc2c(c1=O)COC(=O)[C@]2(O)CC]

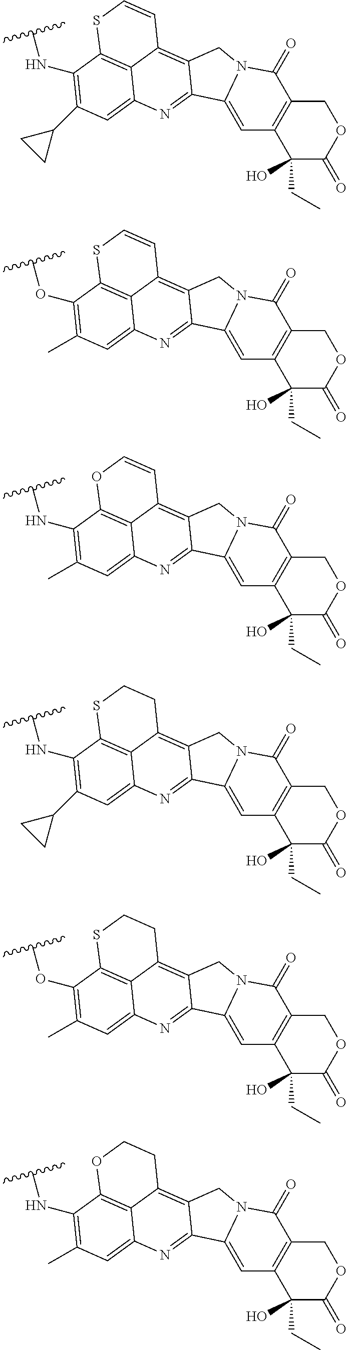 [SMILES: *Nc1c(C2CC2)cc2nc3c(c4c2c1SC=C4)Cn1c-3cc2c(c1=O)COC(=O)[C@]2(O)CC ; *Oc1c(C)cc2nc3c(c4c2c1SC=C4)Cn1c-3cc2c(c1=O)COC(=O)[C@]2(O)CC ; *Nc1c(C)cc2nc3c(c4c2c1OC=C4)Cn1c-3cc2c(c1=O)COC(=O)[C@]2(O)CC ; *Nc1c(C2CC2)cc2nc3c(c4c2c1SCC4)Cn1c-3cc2c(c1=O)COC(=O)[C@]2(O)CC ; *Oc1c(C)cc2nc3c(c4c2c1SCC4)Cn1c-3cc2c(c1=O)COC(=O)[C@]2(O)CC ; *Nc1c(C)cc2nc3c(c4c2c1OCC4)Cn1c-3cc2c(c1=O)COC(=O)[C@]2(O)CC]

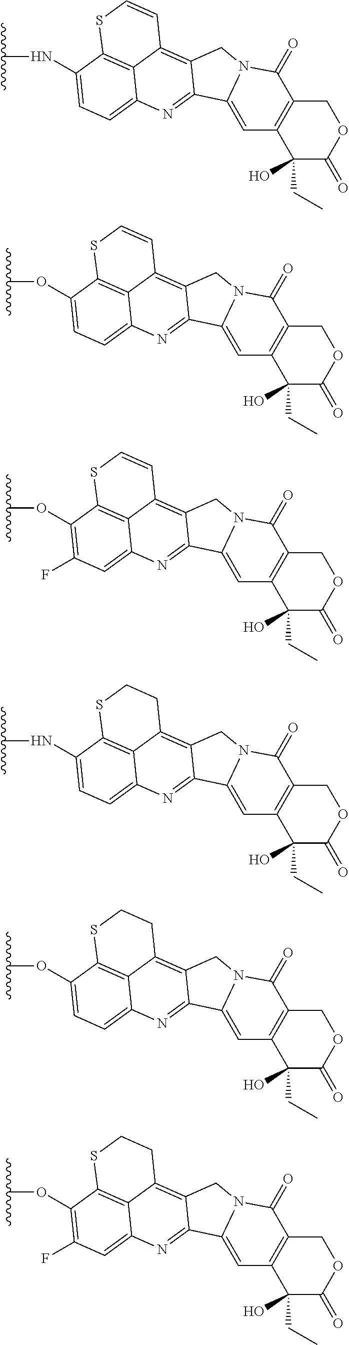 [SMILES: *Nc1ccc2nc3c(c4c2c1SC=C4)Cn1c-3cc2c(c1=O)COC(=O)[C@]2(O)CC ; *Oc1ccc2nc3c(c4c2c1SC=C4)Cn1c-3cc2c(c1=O)COC(=O)[C@]2(O)CC ; *Oc1c(F)cc2nc3c(c4c2c1SC=C4)Cn1c-3cc2c(c1=O)COC(=O)[C@]2(O)CC ; *Nc1ccc2nc3c(c4c2c1SCC4)Cn1c-3cc2c(c1=O)COC(=O)[C@]2(O)CC ; *Oc1ccc2nc3c(c4c2c1SCC4)Cn1c-3cc2c(c1=O)COC(=O)[C@]2(O)CC ; *Oc1c(F)cc2nc3c(c4c2c1SCC4)Cn1c-3cc2c(c1=O)COC(=O)[C@]2(O)CC]

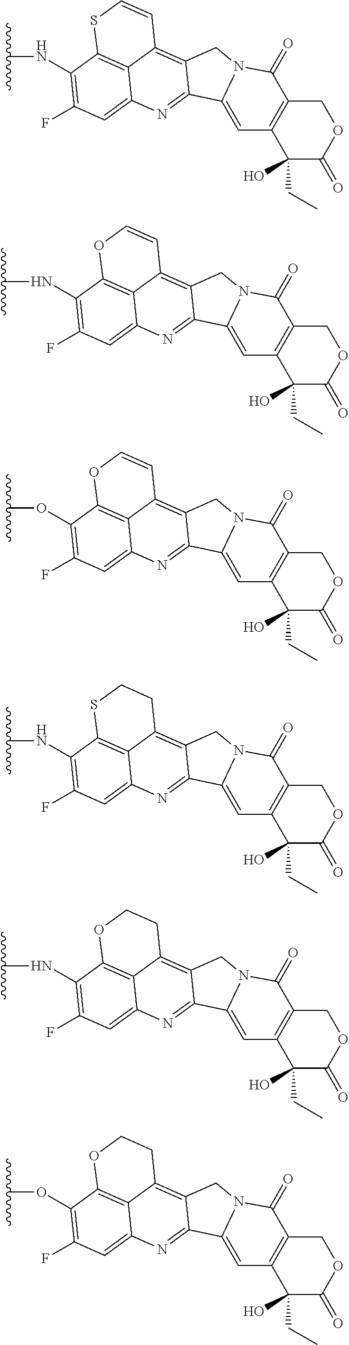 [SMILES: *Nc1c(F)cc2nc3c(c4c2c1SC=C4)Cn1c-3cc2c(c1=O)COC(=O)[C@]2(O)CC ; *Nc1c(F)cc2nc3c(c4c2c1OC=C4)Cn1c-3cc2c(c1=O)COC(=O)[C@]2(O)CC ; *Oc1c(F)cc2nc3c(c4c2c1OC=C4)Cn1c-3cc2c(c1=O)COC(=O)[C@]2(O)CC ; *Nc1c(F)cc2nc3c(c4c2c1SCC4)Cn1c-3cc2c(c1=O)COC(=O)[C@]2(O)CC ; *Nc1c(F)cc2nc3c(c4c2c1OCC4)Cn1c-3cc2c(c1=O)COC(=O)[C@]2(O)CC ; *Oc1c(F)cc2nc3c(c4c2c1OCC4)Cn1c-3cc2c(c1=O)COC(=O)[C@]2(O)CC]

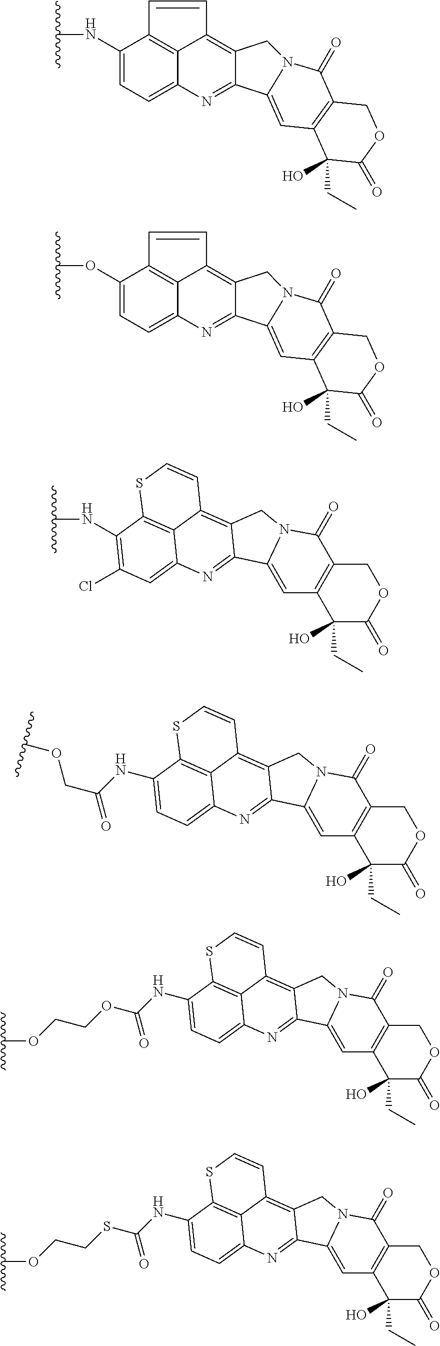 [SMILES: *Nc1ccc2nc3c(c4c2c1C=C4)Cn1c-3cc2c(c1=O)COC(=O)[C@]2(O)CC ; *Oc1ccc2nc3c(c4c2c1C=C4)Cn1c-3cc2c(c1=O)COC(=O)[C@]2(O)CC ; *Nc1c(Cl)cc2nc3c(c4c2c1SC=C4)Cn1c-3cc2c(c1=O)COC(=O)[C@]2(O)CC ; *OCC(=O)Nc1ccc2nc3c(c4c2c1SC=C4)Cn1c-3cc2c(c1=O)COC(=O)[C@]2(O)CC ; *OCCOC(=O)Nc1ccc2nc3c(c4c2c1SC=C4)Cn1c-3cc2c(c1=O)COC(=O)[C@]2(O)CC ; *OCCSC(=O)Nc1ccc2nc3c(c4c2c1SC=C4)Cn1c-3cc2c(c1=O)COC(=O)[C@]2(O)CC]

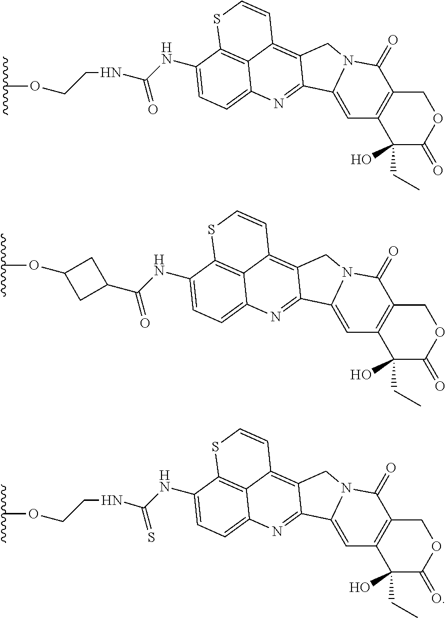 [SMILES: *OCCNC(=O)Nc1ccc2nc3c(c4c2c1SC=C4)Cn1c-3cc2c(c1=O)COC(=O)[C@]2(O)CC ; *OC1CC(C(=O)Nc2ccc3nc4c(c5c3c2SC=C5)Cn2c-4cc3c(c2=O)COC(=O)[C@]3(O)CC)C1 ; *OCCNC(=S)Nc1ccc2nc3c(c4c2c1SC=C4)Cn1c-3cc2c(c1=O)COC(=O)[C@]2(O)CC]

6. The ligand-drug conjugate or a pharmaceutically acceptable salt or solvate thereof according to claim 1, being a ligand-drug conjugate of formula III or a pharmaceutically acceptable salt or solvate thereof: 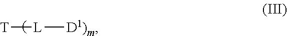
wherein:
T is a targeting or binding ligand;
L is a releasable linker;
D¹ is as defined in any one of claims 1-7; and
m is an integer or a decimal from 1 to 10.

7. The ligand-drug conjugate or a pharmaceutically acceptable salt or solvate thereof according to claim 6, wherein L is -L¹-L²-L³-L⁴-,
L¹ is selected from the group consisting of 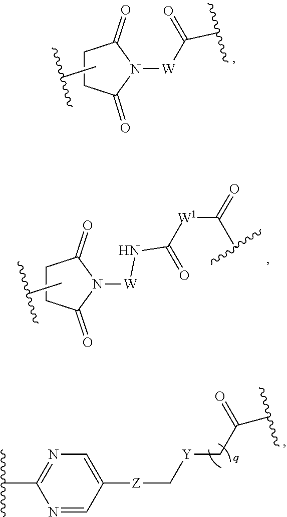
—CH₂—C(O)—NR⁷-W-C(O)—, —CH(COOH)—C(O)—NR⁷-W-C(O)—, and —C(O)-W-C(O)—; wherein W and W¹ are independently selected from the group consisting of C₁-C₈ alkylene, —(C₁-C₈ alkylene)-cycloalkylene-, arylene, -arylene-(C₁-C₈ alkylene)-, heteroarylene, and linear heteroalkylene, wherein the linear heteroalkylene comprise 1 to 8 carbon atom(s), and 1 to 6 heteroatom(s) selected from the group consisting of N, O and S, SO, SO₂, and wherein the —(C₁-C₈ alkylene)-cycloalkylene-, linear heteroalkylene, arylene, and heteroarylene are each independently optionally further substituted by one or more substituent(s) selected from the group consisting of halogen, hydroxy, cyano, amino, alkyl, chloroalkyl, deuterated alkyl, alkoxy and cycloalkyl; wherein q is an integer selected from 1 to 6; wherein Z is selected from the group consisting of C₁-C₆ alkylene, C₁-C₆ alkenylene, C₁-C₆ alkynylene, arylene, and heteroarylene; wherein Y is selected from the group consisting of —O—, —S—, —C(R⁷) (R⁸)—, and heteroarylene; wherein the left side of each of the L1 groups provided above is attached to T;
L² is selected from the group consisting of —NR⁸(CH₂CH₂O)ₚ¹CH₂CH₂C(O)—, —NR⁸(CH₂CH₂O)ₚ¹CH₂CH₂—, —NR⁸(CH₂CH₂O)ₚ¹CH₂C(O)—, —S(CH₂)ₚ¹C(O)—, —N R⁸—(CH₂)ₚ²- (1H-1,2,3-triazole-1,4-diyl)-(CH₂CH₂O)ₚ¹ CH₂C(O), —N R⁸—(CH₂)ₚ²-(1H-1,2,3-triazole-1,4-diyl)-(CH₂CH₂O)ₚ¹CH₂CH₂NHC(O)CH₂OCH₂C(O), —N R⁸ (CH₂CH₂O)ₚ¹ CH₂CH₂NHC(O)CH₂OCH₂C(O), and a chemical bond, wherein p¹ is an integer selected from 1 to 20; wherein p² is an integer selected from 1 to 3; wherein the left side of each of the L² groups provided above is attached to L¹;
L³ is a peptide residue composed of 2 to 7 amino acids, wherein the amino acids are optionally further substituted by one or more substituent(s) selected from the group consisting of halogen, hydroxy, cyano, amino, alkyl, chloroalkyl, deuterated alkyl, alkoxy and cycloalkyl; wherein the left side of each of the L³ groups provided above is attached to L²;
L⁴ is selected from the group consisting of —NR⁹(CR¹⁰R¹¹)ₜ—, 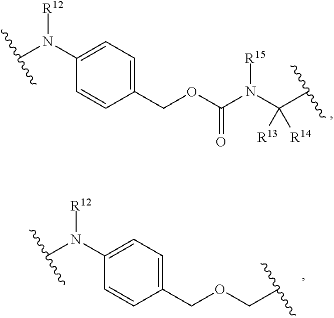
and a chemical bond, wherein t is an integer selected from 1 to 6; wherein in the presence of L⁴ groups, the left side of each of the L⁴ groups provided above is attached to the right side of L³ and the right side of each of the L⁴ groups provided above is attached to D¹;
R⁷, R⁸, R⁹, and R¹² are each independently selected from the group consisting of H, alkyl, haloalkyl, deuterated alkyl and hydroxyalkyl;
R¹⁰, R¹¹, R¹³, and R¹⁴ are each independently selected from the group consisting of H, halogen, alkyl, haloalkyl, deuterated alkyl, and hydroxyalkyl; and
R¹⁵ is selected from —CH₂CH₂SO₂CH₃, and —CH₂CH₂N(CH₃)₂.

8. The ligand-drug conjugate or a pharmaceutically acceptable salt or solvate thereof according to claim 6, wherein:
L¹ is selected from the group consisting of 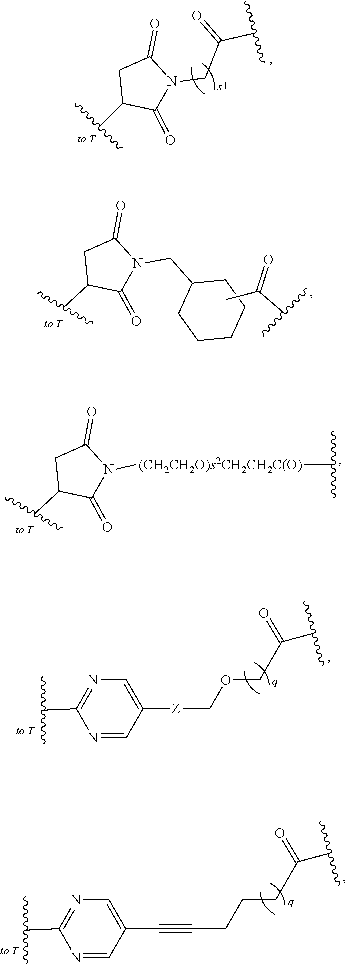
and —CH(COOH)—C(O)—NH—C₆H₄—(CH₂)s³—C(O)— (wherein the left hand side of this group is attached to T), wherein s¹ is an integer selected from 2 to 8; s² is an integer selected from 1 to 3; s³ is an integer selected from 1 to 8; q is an integer selected from 1 to 3; and Z is selected from the group consisting of C₁-C₆ alkylene, C₁-C₆ alkenylene, and C₁-C₆ alkynylene;
L² is selected from the group consisting of —NR⁸(CH₂CH₂O)ₚ¹CH₂CH₂C(O)—, —NR⁸(CH₂CH₂O)ₚ¹CH₂C(O)—, —N R⁸—(CH₂)ₚ²—(1H-1,2,3-triazole-1,4-diyl)-(CH₂CH₂O)ₚ¹ CH₂C(O), and a chemical bond; wherein R⁸ is selected from the group consisting of H, alkyl, haloalkyl, deuterated alkyl and hydroxyalkyl; p¹ is an integer selected from 6 to 12; p² is an integer selected from 1 to 3;
L³ is a peptide residue composed of 2 to 7 amino acids, wherein the amino acids are selected from Phenylalanine (F), Glycine (G), Valine (V), Lysine (K), Citrulline, Serine (S), Alanine (A), Glutamic acid (E), and Aspartic acid (D); optionally a peptide residue composed of 1, 2 or more Phenylalanine and Glycine; optionally is a peptide residue composed of 4 amino acids; optionally is a peptide residue GGFG or VA;
L⁴ is —NR⁹(CR¹⁰R¹¹)ₜ—, 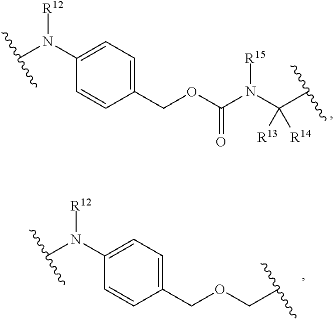
or a chemical bond; wherein R⁹, R¹⁰, R¹¹, R¹², R¹³, and R¹⁴ are each independently selected from H and alkyl; R¹⁵ is selected from —CH₂CH₂SO₂CH₃, and —CH₂CH₂N(CH₃)₂; t is 1 or 2; optionally L⁴ is a chemical bond.

9. The ligand-drug conjugate or a pharmaceutically acceptable salt or solvate thereof according to claim 6, wherein L is selected from structures below:

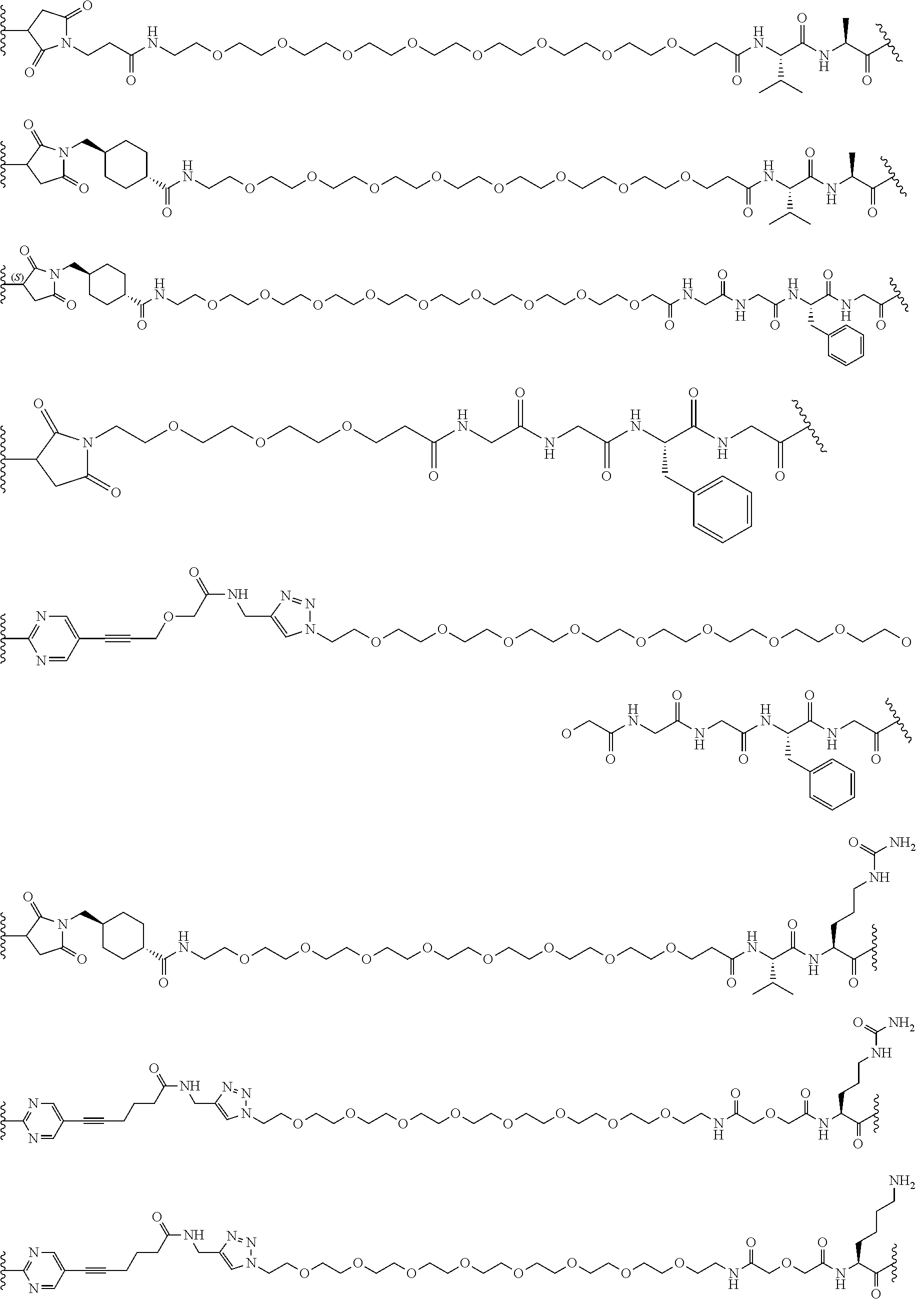

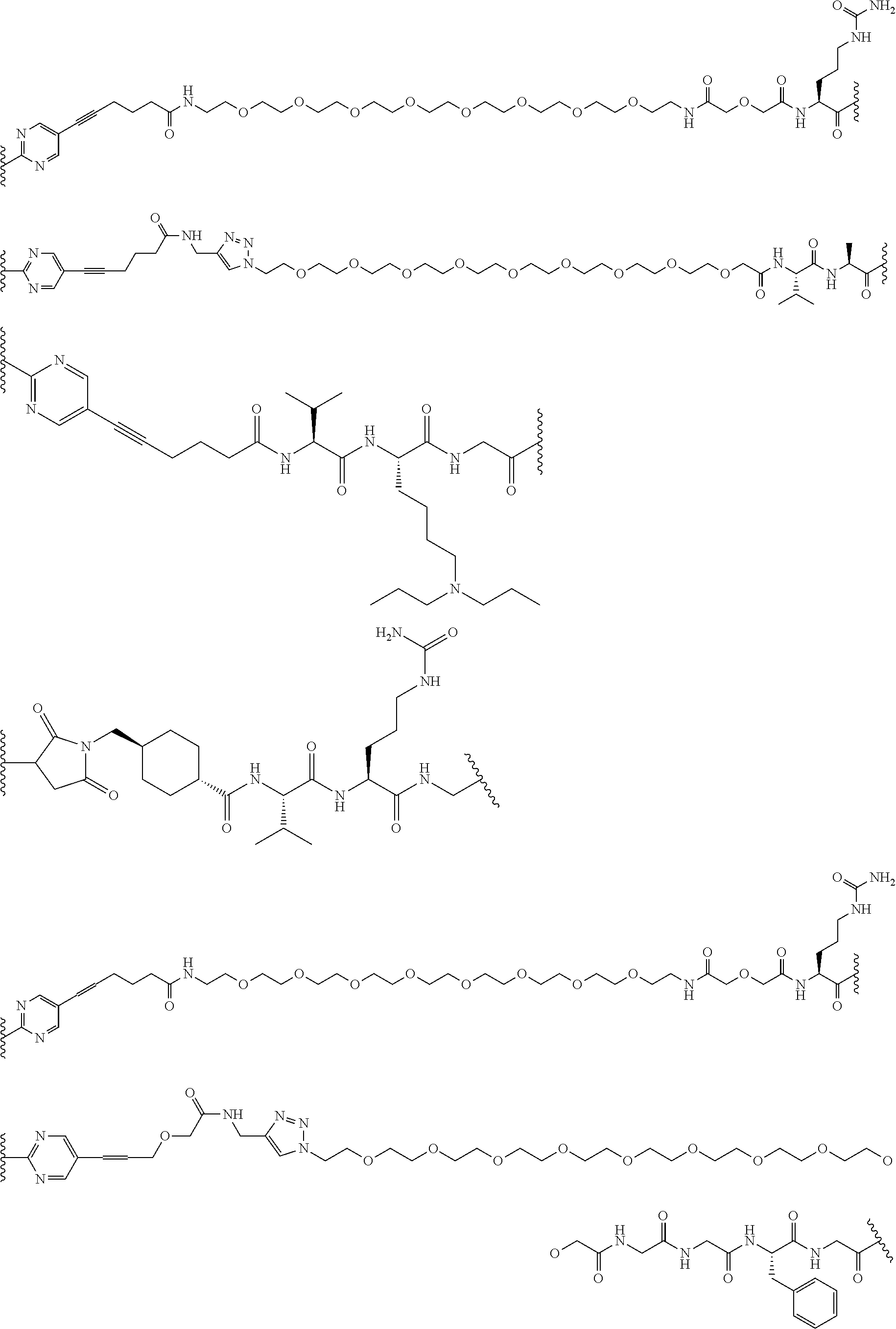

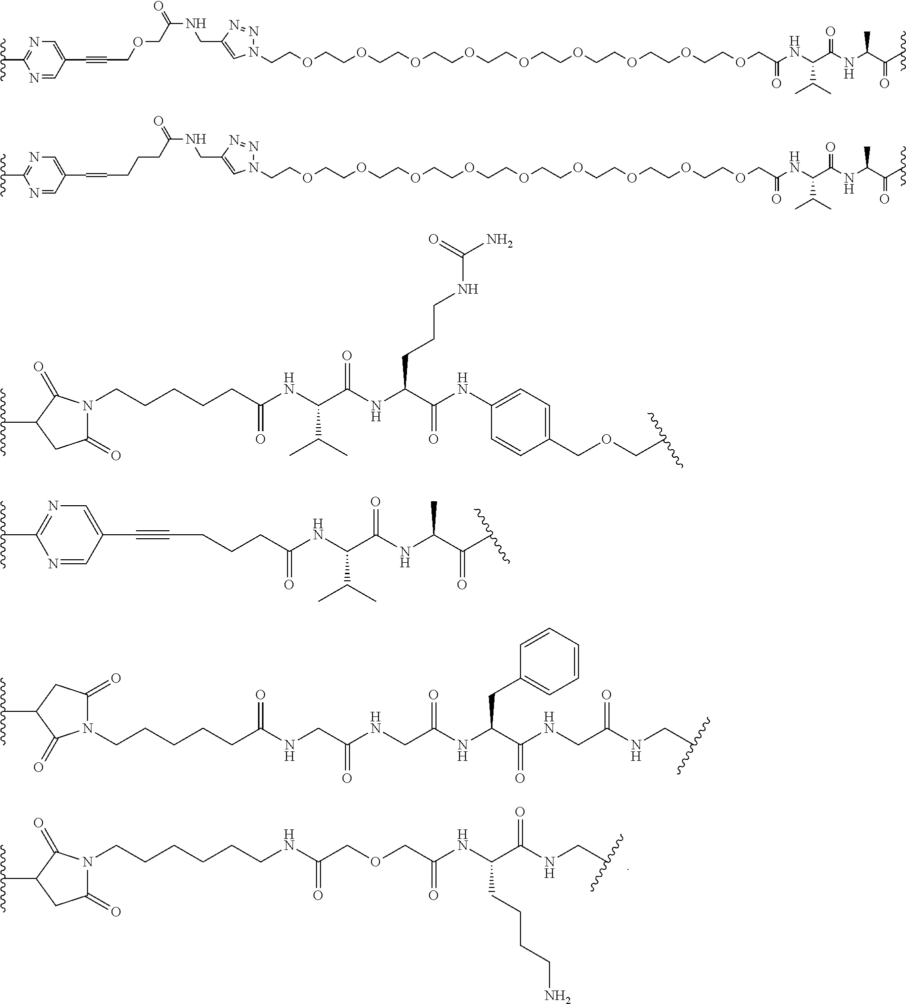

10. The ligand-drug conjugate or a pharmaceutically acceptable salt or solvate thereof according to claim 6, selected from:

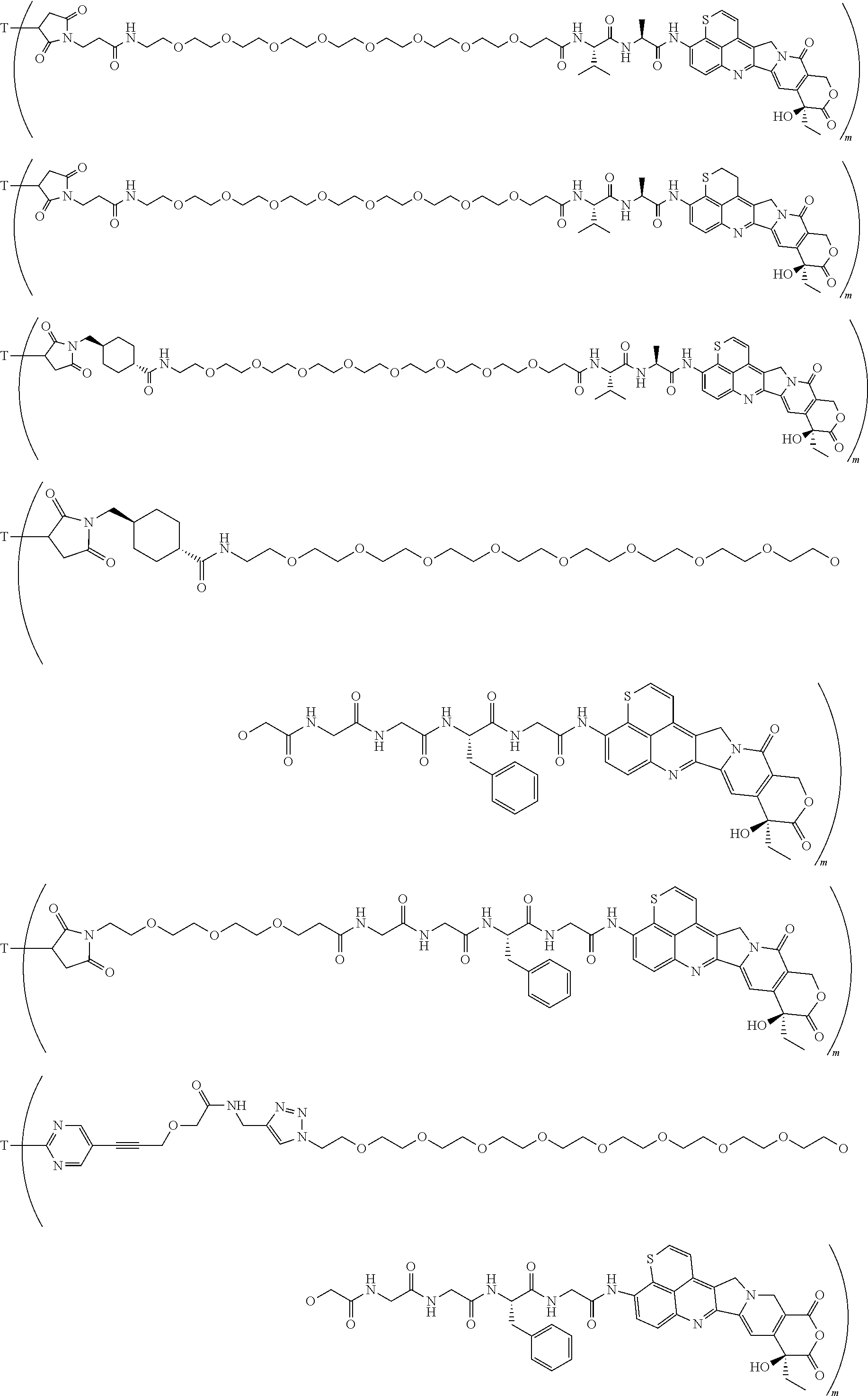

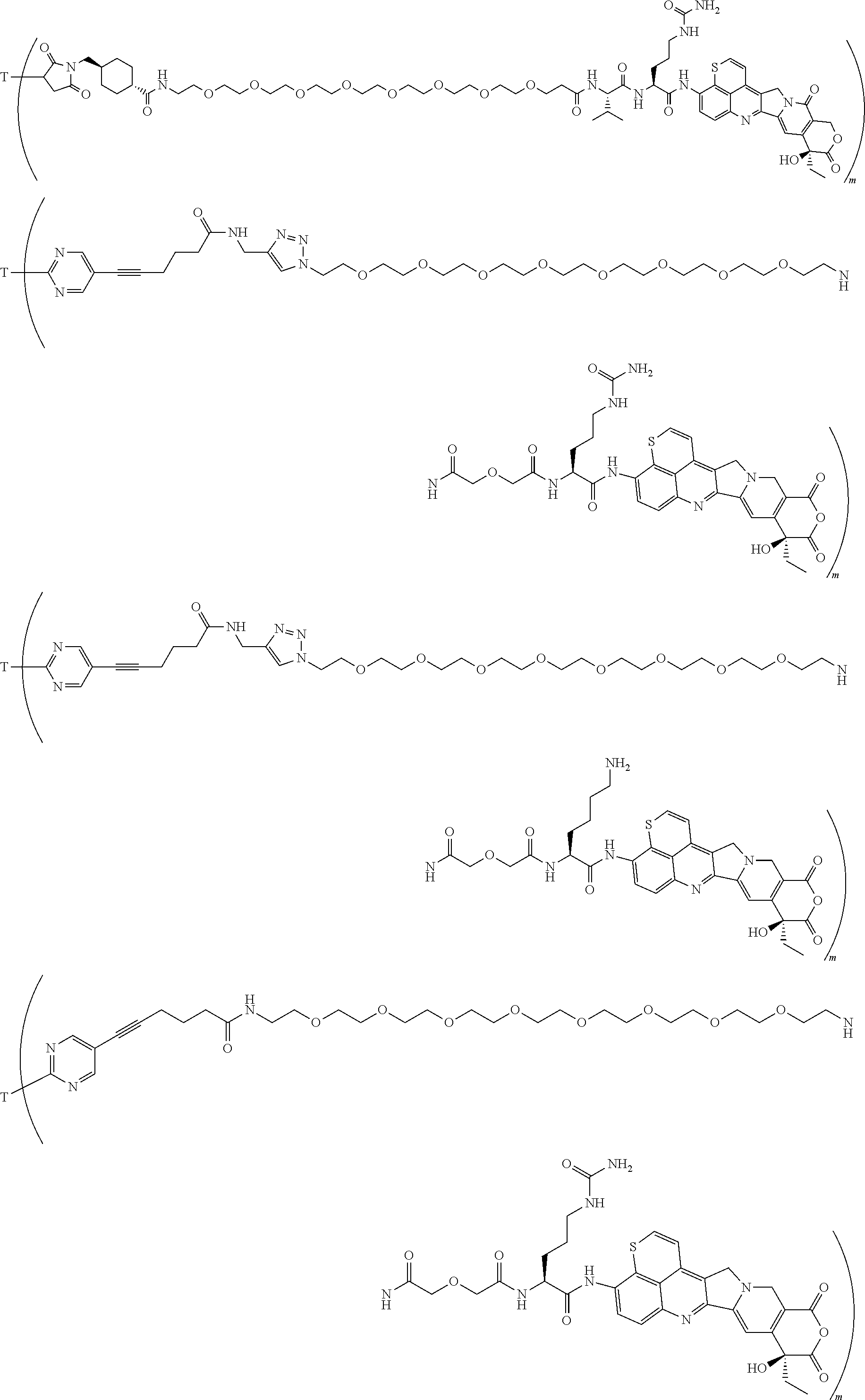

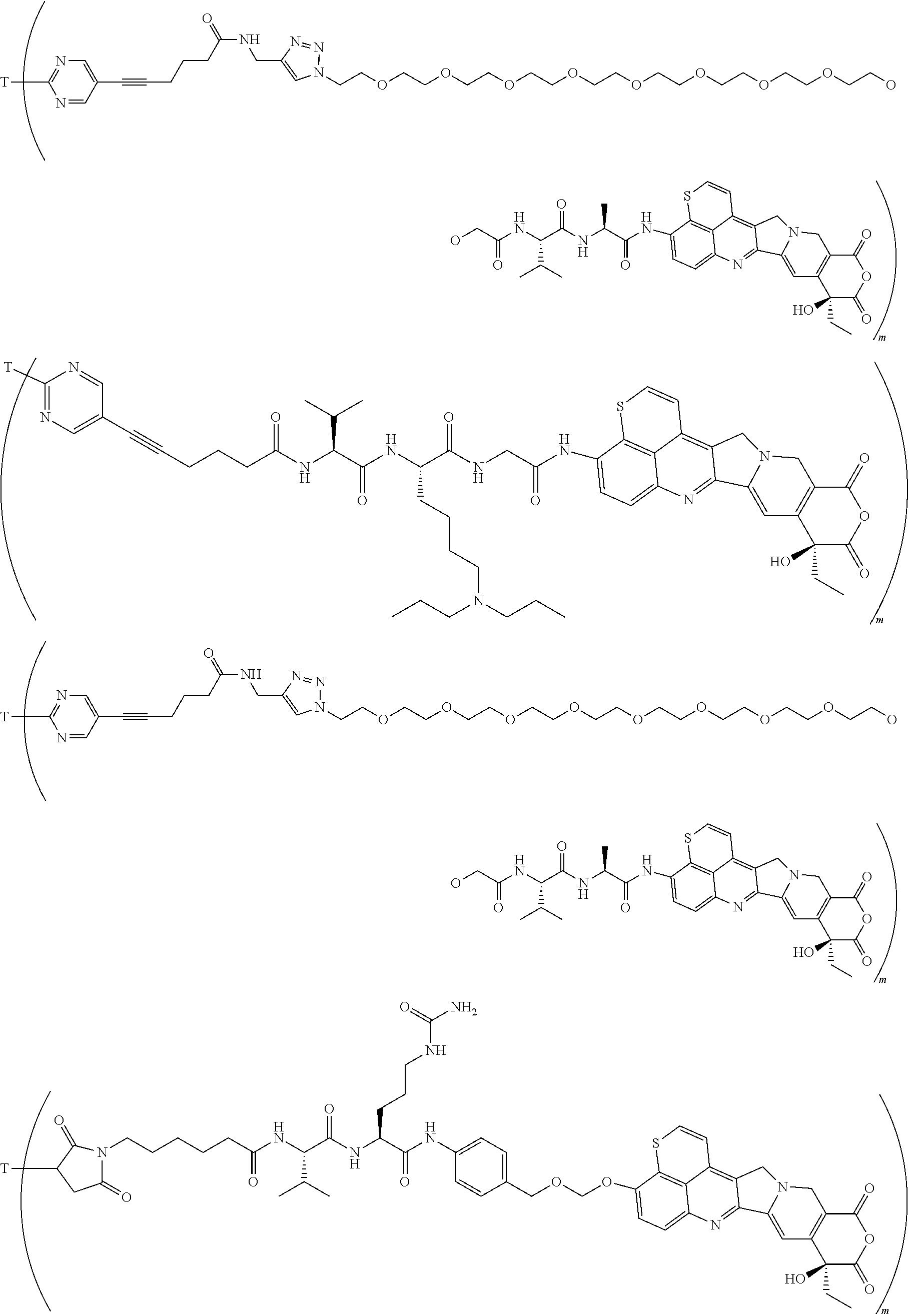

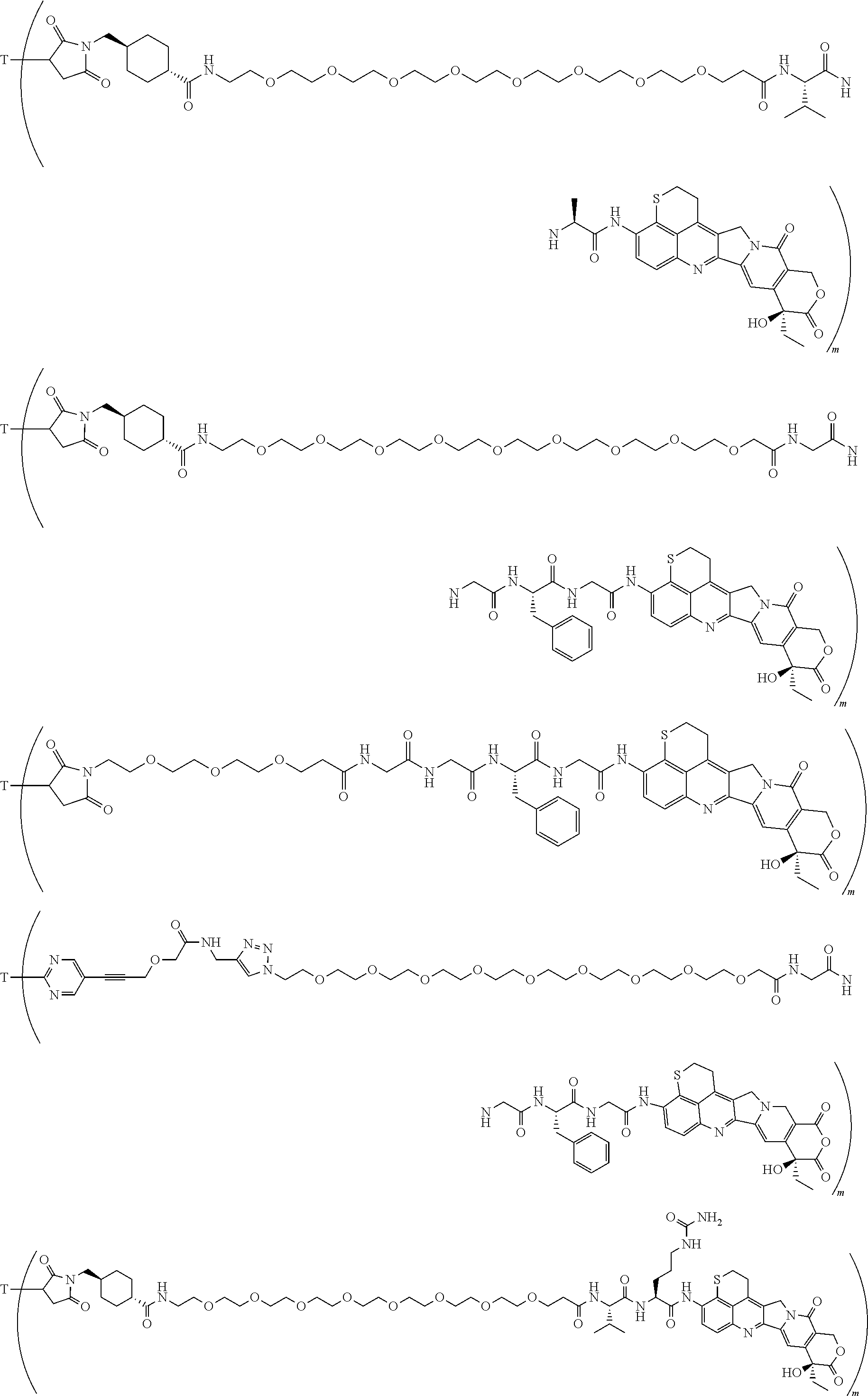

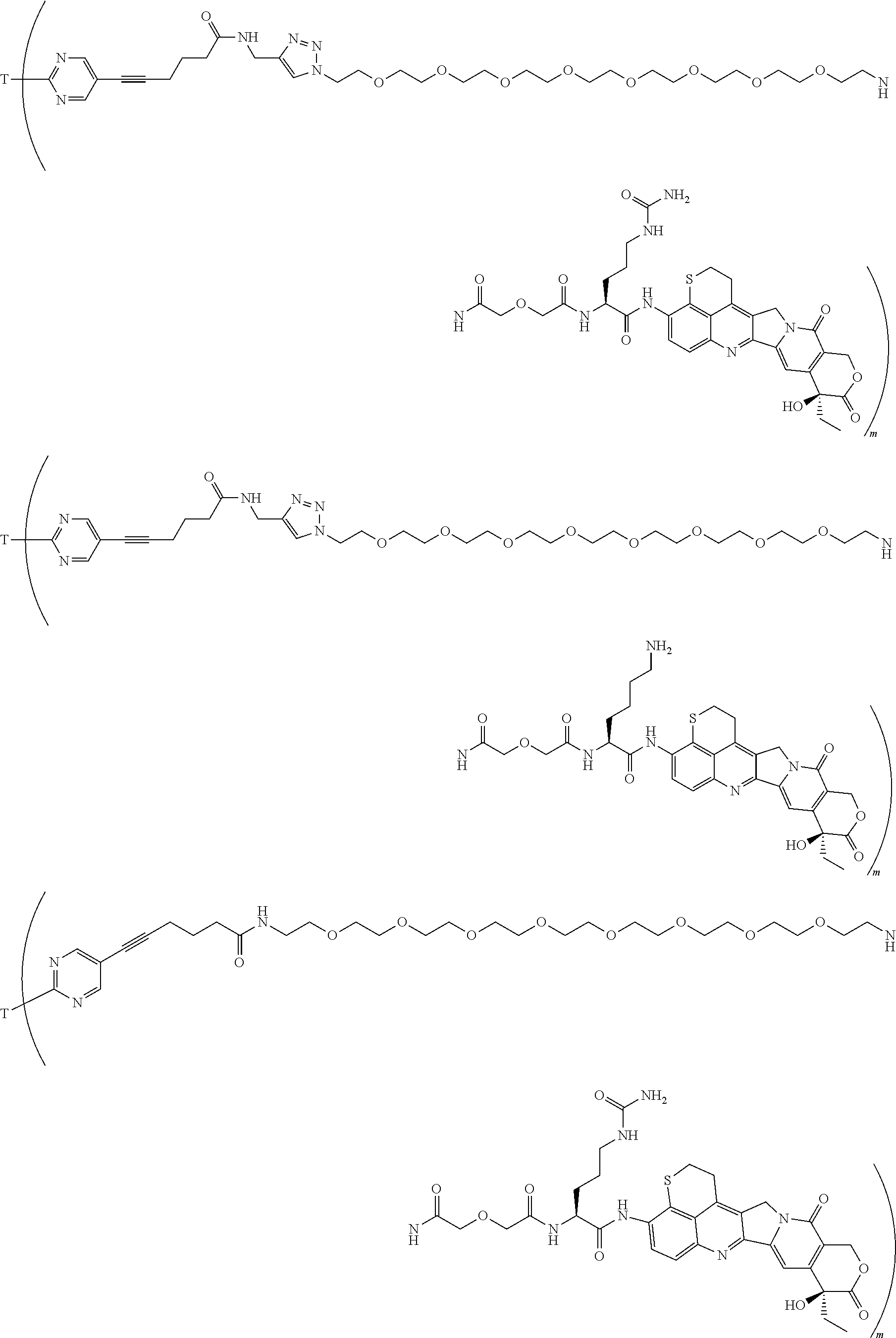

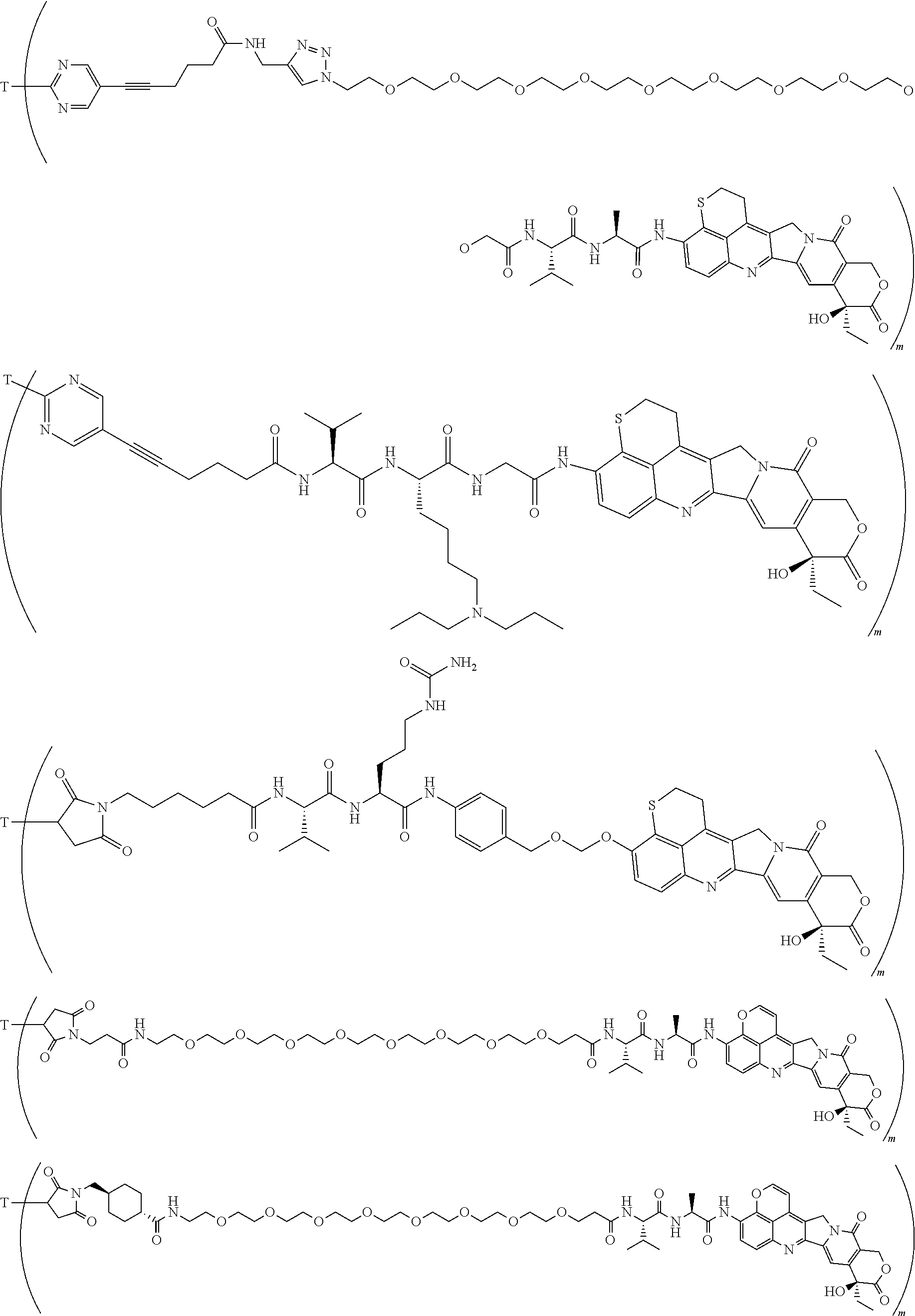

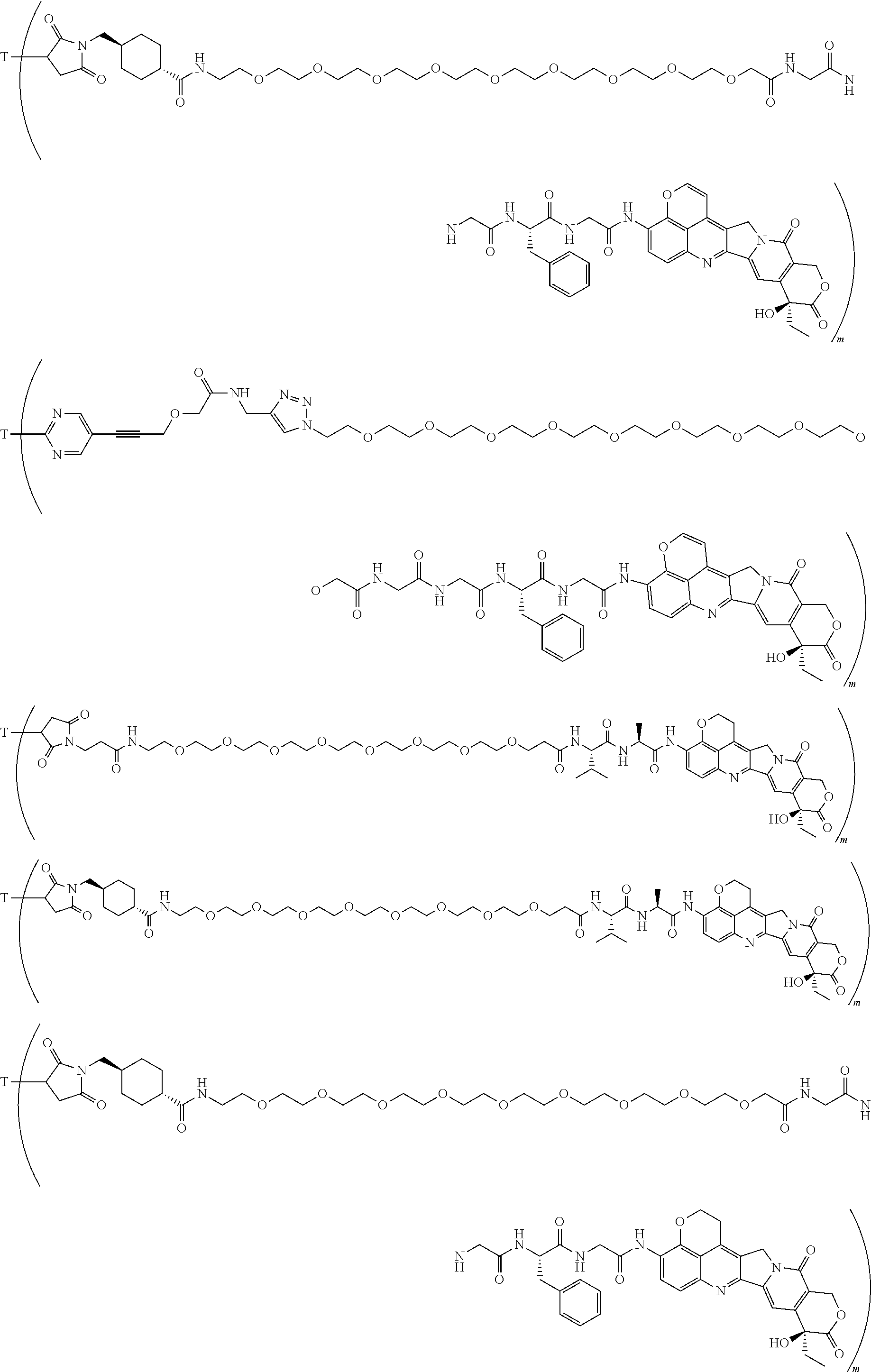

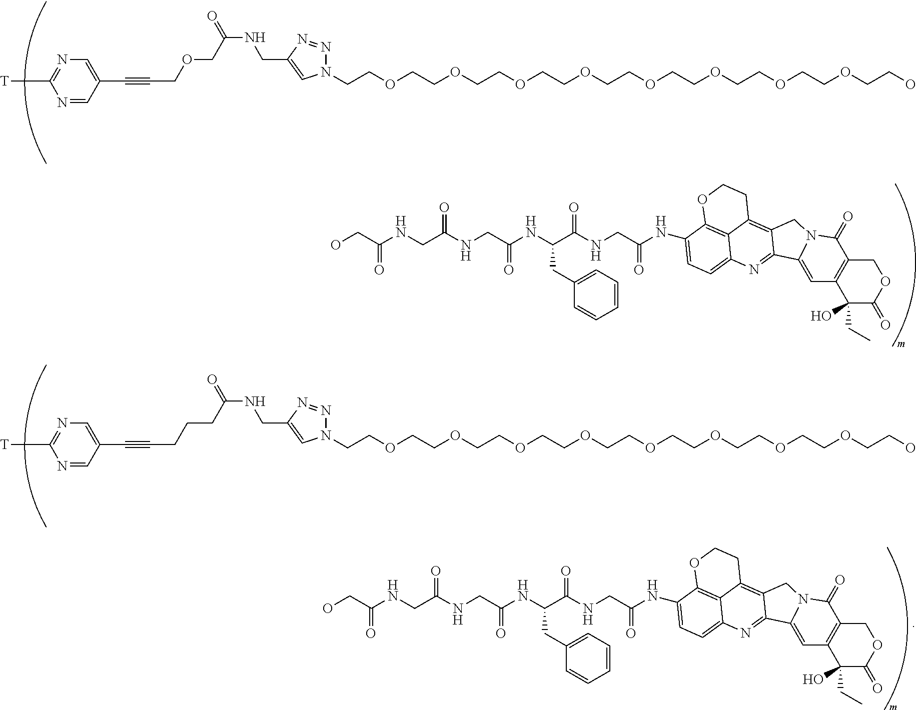

11. The ligand-drug conjugate or a pharmaceutically acceptable salt or solvate thereof according to claim 6, wherein T is a targeting antibody or ligand binding to antigen; wherein the antibody is selected from chimeric antibody, humanized antibody and human antibody; optionally wherein T is a monoclonal antibody.

12. The ligand-drug conjugate or a pharmaceutically acceptable salt or solvate thereof according to claim 6, wherein T is a targeting antibody or ligand binding to antigen; wherein T is selected from anti-Her2 (ErbB2) antibody, anti-EGFR antibody, anti-B₇H₃ antibody, anti-c-MET antibody, anti-Her3 (ErbB3) antibody, anti-Her4 (ErbB4) antibody, anti-CD20 antibody, anti-CD22 antibody, anti-CD30 antibody, anti-CD33 antibody, anti-CD44 antibody, anti-CD56 antibody, anti-CD70 antibody, anti-CD73 antibody, anti-CD105 antibody, anti-CEA antibody, anti-A33 antibody, anti-Cripto antibody, anti-EphA2 antibody, anti-G250 antibody, anti-MICI antibody, anti-Lewis Y antibody, anti-VEGFR antibody, anti-GPNMB antibody, anti-Integrin antibody, anti-PSMA antibody, anti-Tenascin-C antibody, anti-SLC44A4 antibody or anti-Mesothelin antibody, and anti-ROR1 antibody or the fragment binding to the antigen.

13. The ligand-drug conjugate or a pharmaceutically acceptable salt or solvate thereof according to claim 6, wherein T is selected from Trastuzumab, Pertuzumab, Nimotuzumab, Enoblituzumab, Emibetuzumab, Inotuzumab, Pinatuzumab, Brentuximab, Gemtuzumab, Bivatuzumab, Lorvotuzumab, cBR96 and Glembatumumab or the fragment binding to the antigen.

14. The ligand-drug conjugate or a pharmaceutically acceptable salt or solvate thereof according to claim 6, wherein T is Trastuzumab, m is an integer or a decimal from 1 to 10; optionally m is an integer or a decimal from 3 to 8.

15. A compound of formula (IV) or a pharmaceutically acceptable salt, solvate, tautomer, mesomer, racemate, enantiomer, diastereomer, or combinations thereof
L-D¹ (IV),
Wherein D¹ is represented by Formula I: 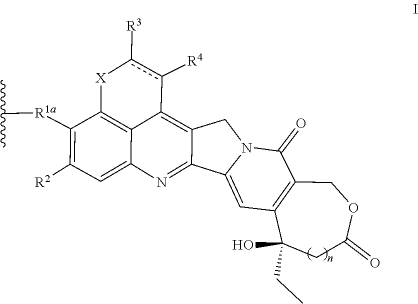
Wherein:
X is either absent, or selected from —O—, —S—, —S(O₂)—, or —S(O)—;
R^{1a} is —NH—R^{1b}—O—, —NH—, or —O—; optionally, R^{1a} is —NH— or —O—;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R² is selected from H, halo, hydroxy, cyano, nitro, C₃-C₈ cycloalkyl, C₁-C₈ alkyl, C₁-C₈ alkoxy, R⁵—(C₁-C₈ alkylene)-, amino, (C₁-C₈ alkyl)NHC(O)O—, (C₃-C₈ cycloalkyl)NHC(O)O—, (C₁-C₈ alkyl)NH—, azetidin-1-yl, pyrrolidin-1-yl, piperidin-1-yl, azepane-1-yl, (C₁-C₈ alkyl)C(O)O—, or (C₁-C₈ alkyl)C(O)NH—; wherein azetidin-1-yl, pyrrolidin-1-yl, piperidin-1-yl, azepane-1-yl, C₁-C₈ alkoxy, each “C₃-C₈ cycloalkyl”, and each “C₁-C₈ alkyl” are independently optionally substituted with one to four R⁶;
R³ and R⁴ are independently selected from H, halo, hydroxy, cyano, C₁-C₈ alkyl, C₁-C₈ alkoxy, C₃-C₈ cycloalkyl, aryl, heteroaryl, or amino; wherein C₁-C₈ alkyl, C₁-C₈ alkoxy, C₃-C₈ cycloalkyl, aryl, and heteroaryl are independently optionally substituted with one to four R⁶;
R⁵ is selected from C₁-C₈ alkoxy, C₃-C₈ cycloalkyl, aryl, or heteroaryl; wherein C₁-C₈ alkoxy, C₃-C₈ cycloalkyl, aryl, and heteroaryl are independently optionally substituted with one to four R⁶;
R⁶ at each occurrence is independently selected from halo, hydroxy, cyano, amino, C₁-C₈ alkyl, C₃-C₈ cycloalkyl, aryl, or heteroaryl;
and wherein a line with a dotted line represents either a single bond or a double bond;
n is an integer from 0 or 1;
and wherein L is L^{1a}-L²-L³-L⁴-,
L^{1a} is selected from the group consisting of 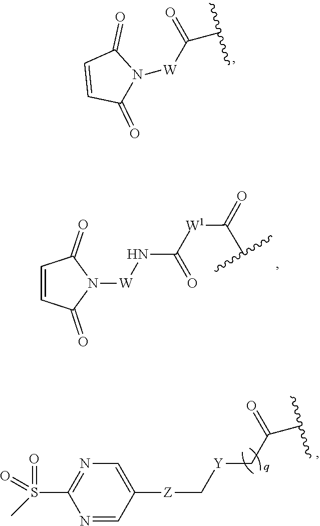
and COOH-W-C(O)—; wherein W and W¹ are independently selected from the group consisting of C₁-C₈ alkylene, —(C₁-C₈ alkylene)-cycloalkylene-, arylene, -arylene-(C₁-C₈ alkylene)-, heteroarylene, and linear heteroalkylene, wherein the linear heteroalkylene comprise 1 to 8 carbon atom(s), and 1 to 6 heteroatom(s) selected from the group consisting of N, O and S, SO, SO₂, and wherein the —(C₁-C₈ alkylene)-cycloalkylene-, linear heteroalkylene, arylene, and heteroarylene are each independently optionally further substituted by one or more substituent(s) selected from the group consisting of halogen, hydroxy, cyano, amino, alkyl, chloroalkyl, deuterated alkyl, alkoxy and cycloalkyl; wherein q is an integer selected from 1 to 6; wherein Z is selected from the group consisting of C₁-C₆ alkylene, C₁-C₆ alkenylene, C₁-C₆ alkynylene, arylene, and heteroarylene; wherein Y is selected from the group consisting of —O—, —S—, —C(R⁷) (R⁸)-, and heteroarylene;
L² is selected from the group consisting of —NR⁸(CH₂CH₂O)ₚ¹CH₂CH₂C(O)—, —NR⁸(CH₂CH₂O)ₚ¹CH₂CH₂—, —NR⁸(CH₂CH₂O)ₚ¹CH₂C(O)—, —S(CH₂)ₚ¹C(O)—, —N R⁸—(CH₂)ₚ²—(1H-1,2,3-triazole-1,4-diyl)-(CH₂CH₂O)ₚ¹ CH₂C(O), —N R⁸—(CH₂)ₚ²—(1H-1,2,3-triazole-1,4-diyl)-(CH₂CH₂O)ₚ¹CH₂CH₂NHC(O)CH₂OCH₂C(O), —N R⁸ (CH₂CH₂O)ₚ¹ CH₂CH₂NHC(O)CH₂OCH₂C(O), and a chemical bond, wherein p¹ is an integer selected from 1 to 20; wherein p² is an integer selected from 1 to 3; wherein the left side of each of the L² groups provided above is attached to L¹;
L³ is a peptide residue composed of 2 to 7 amino acids, wherein the amino acids are optionally further substituted by one or more substituent(s) selected from the group consisting of halogen, hydroxy, cyano, amino, alkyl, chloroalkyl, deuterated alkyl, alkoxy and cycloalkyl; wherein the left side of each of the L³ groups provided above is attached to L²;
L⁴ is selected from the group consisting of —NR⁹(CR¹⁰R¹¹)ₜ—, 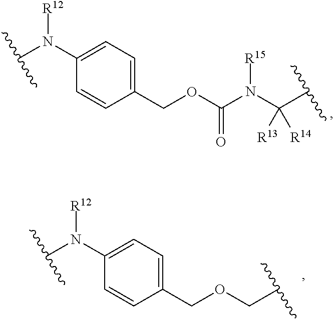
and a chemical bond, wherein t is an integer selected from 1 to 6; wherein the left side of each of the L⁴ groups provided above is attached to the right side of L³ and the right side of each of the L⁴ groups is attached to D¹;
R⁷, R⁸, R⁹, and R¹² are each independently selected from the group consisting of H, alkyl, haloalkyl, deuterated alkyl and hydroxyalkyl;
R¹⁰, R¹¹, R¹³, and R¹⁴ are each independently selected from the group consisting of H, halogen, alkyl, haloalkyl, deuterated alkyl, and hydroxyalkyl; and
R¹⁵ is selected from —CH₂CH₂SO₂CH₃, and —CH₂CH₂N(CH₃)₂.

16. The compound or a pharmaceutically acceptable salt, solvate, tautomer, mesomer, racemate, enantiomer, diastereomer, or combinations thereof according to claim 15, wherein D¹ is represented by Formula II: 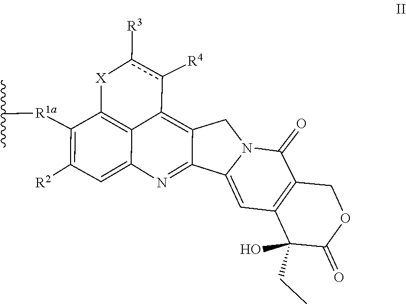
X is —O— or —S—;
R^{1a} is —NH—R^{1b}—O—, —NH—, or —O—; optionally, R^{1a} is —NH— or —O—;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R² is selected from H, halo, hydroxy, cyano, nitro, C₃-C₆ cycloalkyl, C₁-C₆ alkyl, C₁-C₆ alkoxy, R⁵—(C₁-C₃ alkylene)-, amino; wherein C₁-C₆ alkoxy, C₃-C₆ cycloalkyl, and C₁-C₆ alkyl are independently optionally substituted with one to four R⁶;
R³ and R⁴ are independently selected from H, halo, hydroxy, cyano, C₁-C₆ alkyl, C₁-C₆ alkoxy, or C₃-C₆ cycloalkyl; wherein C₁-C₆ alkyl, C₁-C₆ alkoxy, and C₃-C₆ cycloalkyl are independently optionally substituted with one to four R⁶;
R⁵ is C₁-C₆ alkoxy or C₃-C₆ cycloalkyl; wherein C₁-C₆ alkoxy and C₃-C₆ cycloalkyl are independently optionally substituted with one to four R⁶;
R⁶ at each occurrence is independently selected from halo, hydroxy, cyano, amino, C₁-C₃ alkyl, or C₃-C₆ cycloalkyl;
and wherein a line with a dotted line represents either a single bond or a double bond.

17. The compound or a pharmaceutically acceptable salt, solvate, tautomer, mesomer, racemate, enantiomer, diastereomer, or combinations thereof according to claim 15, wherein R², R³ and R⁴ are independently selected from H, halo, C₁-C₃ alkyl, or C₁-C₃ alkoxy and wherein C₁-C₃ alkoxy and C₁-C₃ alkyl are independently optionally substituted with one to four halo.

18. The compound or a pharmaceutically acceptable salt, solvate, tautomer, mesomer, racemate, enantiomer, diastereomer, or combinations thereof according to claim 15, wherein a line with a dotted line represents a double bond.

19. The compound or a pharmaceutically acceptable salt, solvate, tautomer, mesomer, racemate, enantiomer, diastereomer, or combinations thereof according to claim 15, wherein D¹ is selected from structures below:

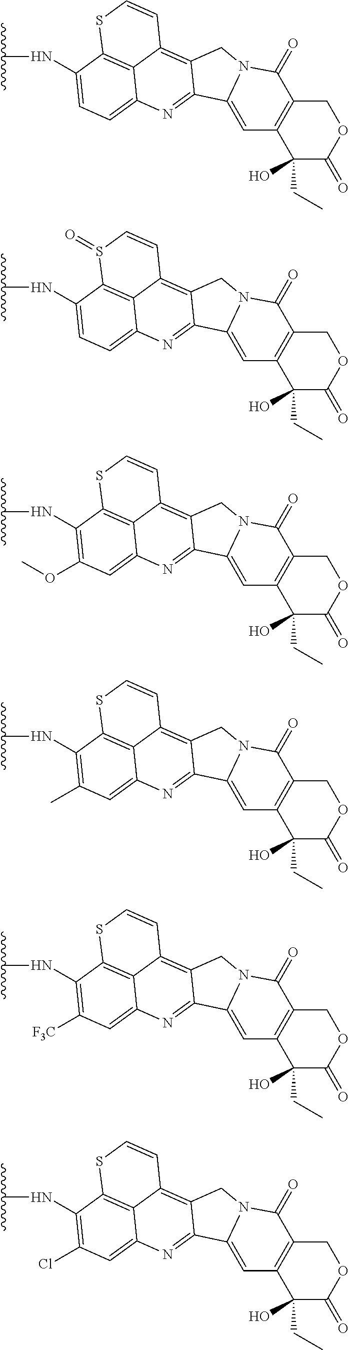 [SMILES: *Nc1ccc2nc3c(c4c2c1SC=C4)Cn1c-3cc2c(c1=O)COC(=O)[C@]2(O)CC ; *Nc1ccc2nc3c(c4c2c1S(=O)C=C4)Cn1c-3cc2c(c1=O)COC(=O)[C@]2(O)CC ; *Nc1c(OC)cc2nc3c(c4c2c1SC=C4)Cn1c-3cc2c(c1=O)COC(=O)[C@]2(O)CC ; *Nc1c(C)cc2nc3c(c4c2c1SC=C4)Cn1c-3cc2c(c1=O)COC(=O)[C@]2(O)CC ; *Nc1c(C(F)(F)F)cc2nc3c(c4c2c1SC=C4)Cn1c-3cc2c(c1=O)COC(=O)[C@]2(O)CC ; *Nc1c(Cl)cc2nc3c(c4c2c1SC=C4)Cn1c-3cc2c(c1=O)COC(=O)[C@]2(O)CC]

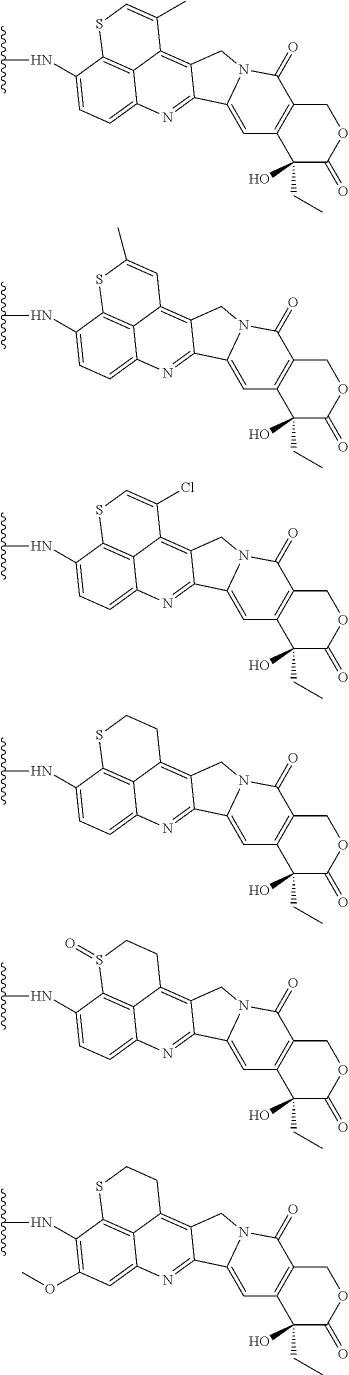 [SMILES: *Nc1ccc2nc3c(c4c2c1SC=C4C)Cn1c-3cc2c(c1=O)COC(=O)[C@]2(O)CC ; *Nc1ccc2nc3c(c4c2c1SC(C)=C4)Cn1c-3cc2c(c1=O)COC(=O)[C@]2(O)CC ; *Nc1ccc2nc3c(c4c2c1SC=C4Cl)Cn1c-3cc2c(c1=O)COC(=O)[C@]2(O)CC ; *Nc1ccc2nc3c(c4c2c1SCC4)Cn1c-3cc2c(c1=O)COC(=O)[C@]2(O)CC ; *Nc1ccc2nc3c(c4c2c1S(=O)CC4)Cn1c-3cc2c(c1=O)COC(=O)[C@]2(O)CC ; *Nc1c(OC)cc2nc3c(c4c2c1SCC4)Cn1c-3cc2c(c1=O)COC(=O)[C@]2(O)CC]

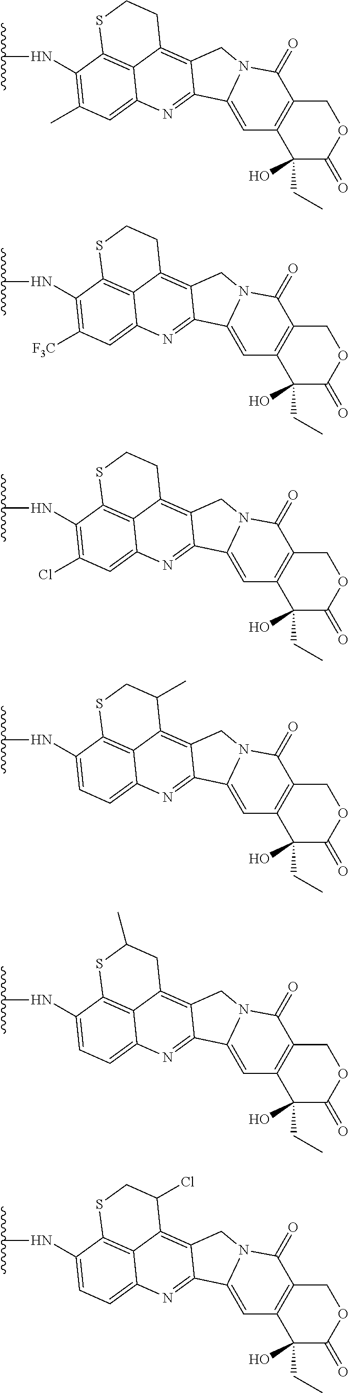 [SMILES: *Nc1c(C)cc2nc3c(c4c2c1SCC4)Cn1c-3cc2c(c1=O)COC(=O)[C@]2(O)CC ; *Nc1c(C(F)(F)F)cc2nc3c(c4c2c1SCC4)Cn1c-3cc2c(c1=O)COC(=O)[C@]2(O)CC ; *Nc1c(Cl)cc2nc3c(c4c2c1SCC4)Cn1c-3cc2c(c1=O)COC(=O)[C@]2(O)CC ; *Nc1ccc2nc3c(c4c2c1SCC4C)Cn1c-3cc2c(c1=O)COC(=O)[C@]2(O)CC ; *Nc1ccc2nc3c(c4c2c1SC(C)C4)Cn1c-3cc2c(c1=O)COC(=O)[C@]2(O)CC ; *Nc1ccc2nc3c(c4c2c1SCC4Cl)Cn1c-3cc2c(c1=O)COC(=O)[C@]2(O)CC]

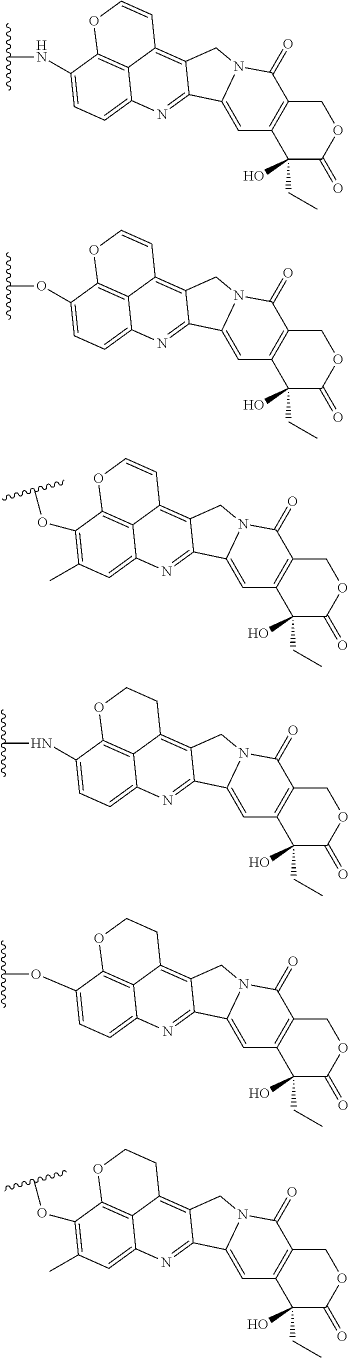 [SMILES: *Nc1ccc2nc3c(c4c2c1OC=C4)Cn1c-3cc2c(c1=O)COC(=O)[C@]2(O)CC ; *Oc1ccc2nc3c(c4c2c1OC=C4)Cn1c-3cc2c(c1=O)COC(=O)[C@]2(O)CC ; *Oc1c(C)cc2nc3c(c4c2c1OC=C4)Cn1c-3cc2c(c1=O)COC(=O)[C@]2(O)CC ; *Nc1ccc2nc3c(c4c2c1OCC4)Cn1c-3cc2c(c1=O)COC(=O)[C@]2(O)CC ; *Oc1ccc2nc3c(c4c2c1OCC4)Cn1c-3cc2c(c1=O)COC(=O)[C@]2(O)CC ; *Oc1c(C)cc2nc3c(c4c2c1OCC4)Cn1c-3cc2c(c1=O)COC(=O)[C@]2(O)CC]

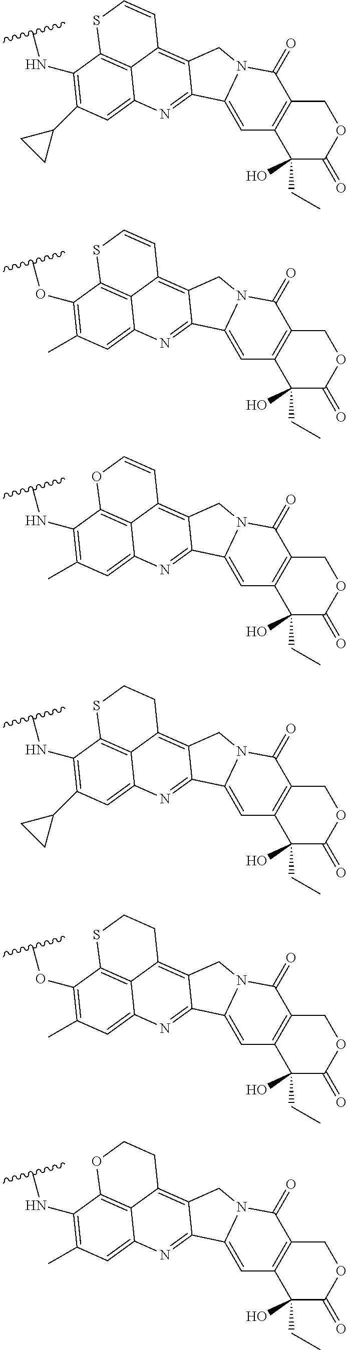 [SMILES: *Nc1c(C2CC2)cc2nc3c(c4c2c1SC=C4)Cn1c-3cc2c(c1=O)COC(=O)[C@]2(O)CC ; *Oc1c(C)cc2nc3c(c4c2c1SC=C4)Cn1c-3cc2c(c1=O)COC(=O)[C@]2(O)CC ; *Nc1c(C)cc2nc3c(c4c2c1OC=C4)Cn1c-3cc2c(c1=O)COC(=O)[C@]2(O)CC ; *Nc1c(C2CC2)cc2nc3c(c4c2c1SCC4)Cn1c-3cc2c(c1=O)COC(=O)[C@]2(O)CC ; *Oc1c(C)cc2nc3c(c4c2c1SCC4)Cn1c-3cc2c(c1=O)COC(=O)[C@]2(O)CC ; *Nc1c(C)cc2nc3c(c4c2c1OCC4)Cn1c-3cc2c(c1=O)COC(=O)[C@]2(O)CC]

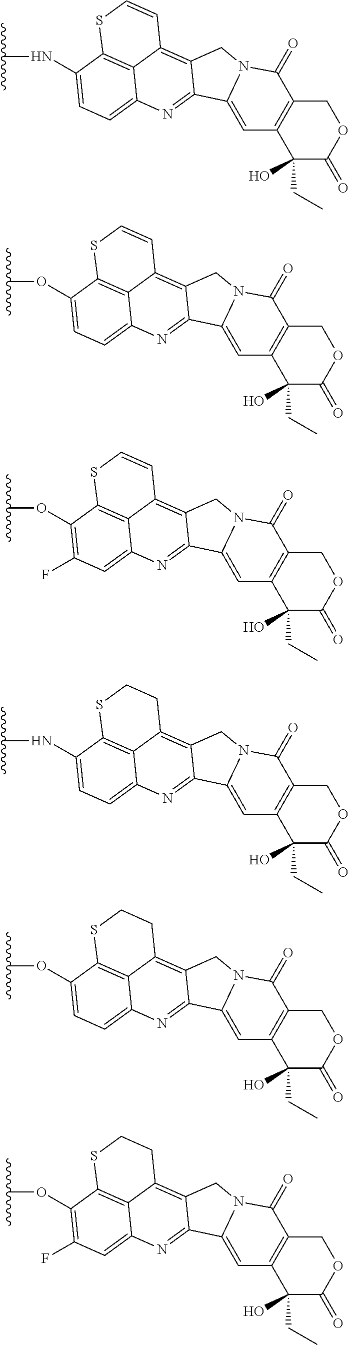 [SMILES: *Nc1ccc2nc3c(c4c2c1SC=C4)Cn1c-3cc2c(c1=O)COC(=O)[C@]2(O)CC ; *Oc1ccc2nc3c(c4c2c1SC=C4)Cn1c-3cc2c(c1=O)COC(=O)[C@]2(O)CC ; *Oc1c(F)cc2nc3c(c4c2c1SC=C4)Cn1c-3cc2c(c1=O)COC(=O)[C@]2(O)CC ; *Nc1ccc2nc3c(c4c2c1SCC4)Cn1c-3cc2c(c1=O)COC(=O)[C@]2(O)CC ; *Oc1ccc2nc3c(c4c2c1SCC4)Cn1c-3cc2c(c1=O)COC(=O)[C@]2(O)CC ; *Oc1c(F)cc2nc3c(c4c2c1SCC4)Cn1c-3cc2c(c1=O)COC(=O)[C@]2(O)CC]

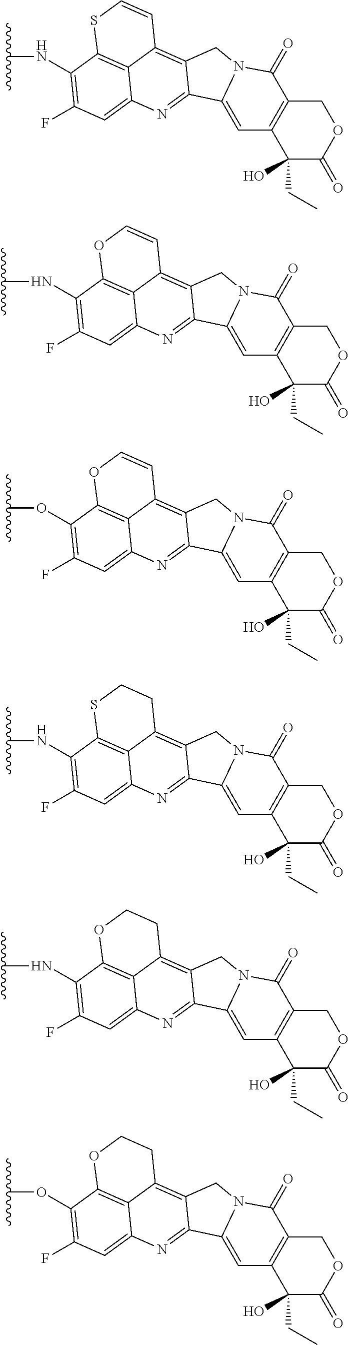 [SMILES: *Nc1c(F)cc2nc3c(c4c2c1SC=C4)Cn1c-3cc2c(c1=O)COC(=O)[C@]2(O)CC ; *Nc1c(F)cc2nc3c(c4c2c1OC=C4)Cn1c-3cc2c(c1=O)COC(=O)[C@]2(O)CC ; *Oc1c(F)cc2nc3c(c4c2c1OC=C4)Cn1c-3cc2c(c1=O)COC(=O)[C@]2(O)CC ; *Nc1c(F)cc2nc3c(c4c2c1SCC4)Cn1c-3cc2c(c1=O)COC(=O)[C@]2(O)CC ; *Nc1c(F)cc2nc3c(c4c2c1OCC4)Cn1c-3cc2c(c1=O)COC(=O)[C@]2(O)CC ; *Oc1c(F)cc2nc3c(c4c2c1OCC4)Cn1c-3cc2c(c1=O)COC(=O)[C@]2(O)CC]

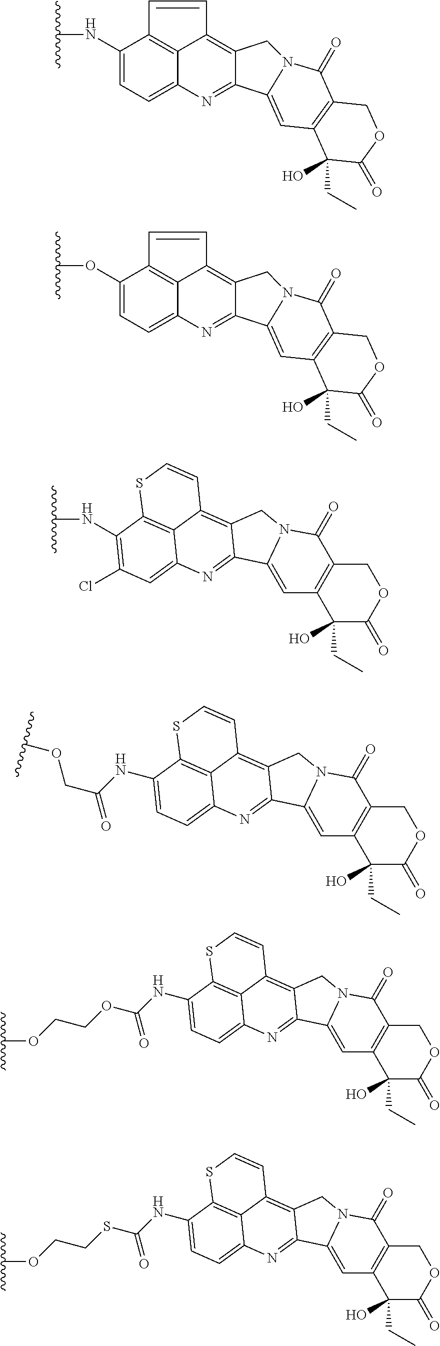 [SMILES: *Nc1ccc2nc3c(c4c2c1C=C4)Cn1c-3cc2c(c1=O)COC(=O)[C@]2(O)CC ; *Oc1ccc2nc3c(c4c2c1C=C4)Cn1c-3cc2c(c1=O)COC(=O)[C@]2(O)CC ; *Nc1c(Cl)cc2nc3c(c4c2c1SC=C4)Cn1c-3cc2c(c1=O)COC(=O)[C@]2(O)CC ; *OCC(=O)Nc1ccc2nc3c(c4c2c1SC=C4)Cn1c-3cc2c(c1=O)COC(=O)[C@]2(O)CC ; *OCCOC(=O)Nc1ccc2nc3c(c4c2c1SC=C4)Cn1c-3cc2c(c1=O)COC(=O)[C@]2(O)CC ; *OCCSC(=O)Nc1ccc2nc3c(c4c2c1SC=C4)Cn1c-3cc2c(c1=O)COC(=O)[C@]2(O)CC]

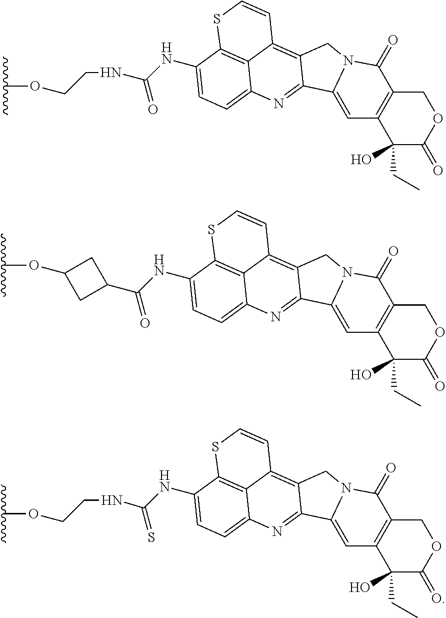 [SMILES: *OCCNC(=O)Nc1ccc2nc3c(c4c2c1SC=C4)Cn1c-3cc2c(c1=O)COC(=O)[C@]2(O)CC ; *OC1CC(C(=O)Nc2ccc3nc4c(c5c3c2SC=C5)Cn2c-4cc3c(c2=O)COC(=O)[C@]3(O)CC)C1 ; *OCCNC(=S)Nc1ccc2nc3c(c4c2c1SC=C4)Cn1c-3cc2c(c1=O)COC(=O)[C@]2(O)CC]

20. The compound or a pharmaceutically acceptable salt, solvate, tautomer, mesomer, racemate, enantiomer, diastereomer, or combinations thereof according to claim 15, wherein L is L^{1a}-L²-L³-,
L^{1a} is 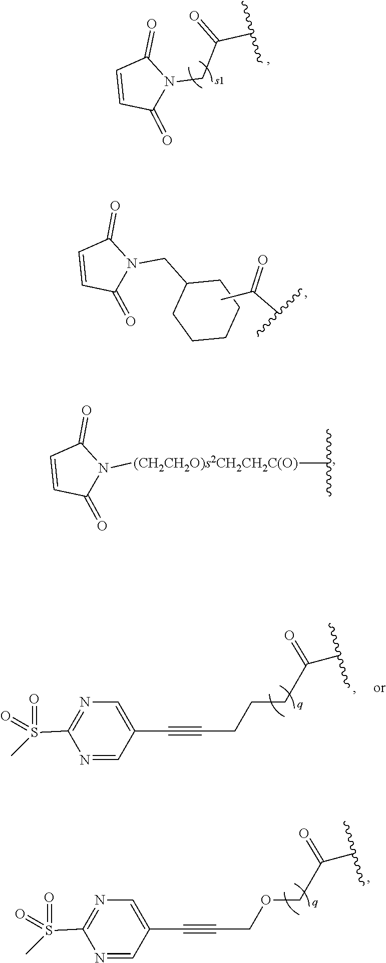
wherein s¹ is an integer selected from 2 to 8; s² is an integer selected from 1 to 3; and q is an integer selected from 1 to 3;
L² is selected from the group consisting of —NR⁸(CH₂CH₂O)ₚ¹CH₂CH₂C(O)—, —NR⁸(CH₂CH₂O)ₚ¹CH₂C(O)—, —N R⁸—(CH₂)ₚ²—(1H-1,2,3-triazole-1,4-diyl)-(CH₂CH₂O)ₚ¹ CH₂C(O), and a chemical bond, wherein R⁸ is selected from the group consisting of H, alkyl, haloalkyl, deuterated alkyl and hydroxyalkyl; p¹ is an integer selected from 6 to 12; p² is an integer selected from 1 to 3; wherein the left side of each of the L² groups provided above is attached to L¹;
L³ is a peptide residue GGFG or VA; wherein the left side of each of the L³ groups provided above is attached to L²;
and wherein the right side each of the L³ groups provided above is connected to D¹.

21. The compound or a pharmaceutically acceptable salt, solvate, tautomer, mesomer, racemate, enantiomer, diastereomer, or combinations thereof according to claim 15, the compound selected from structures below:

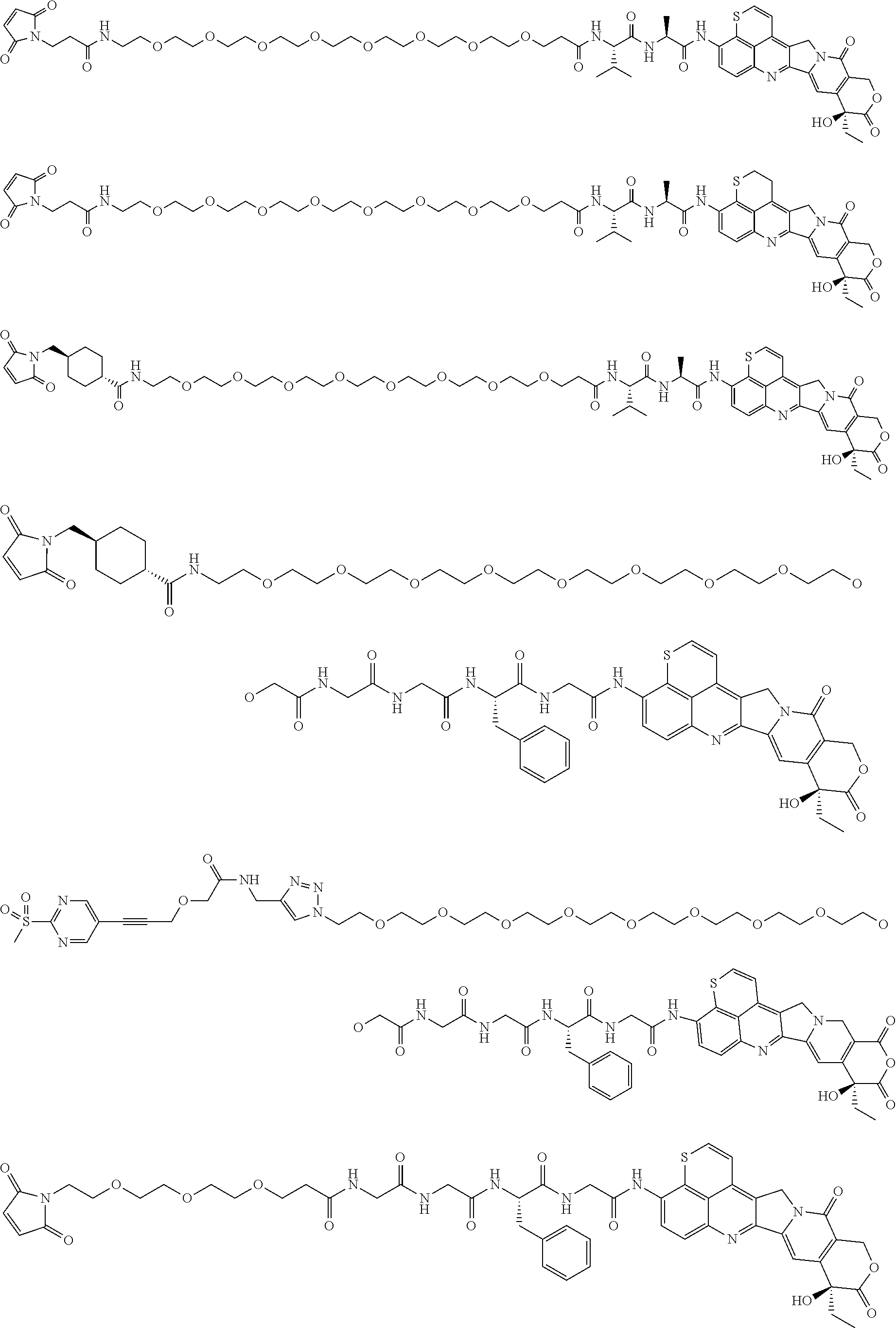

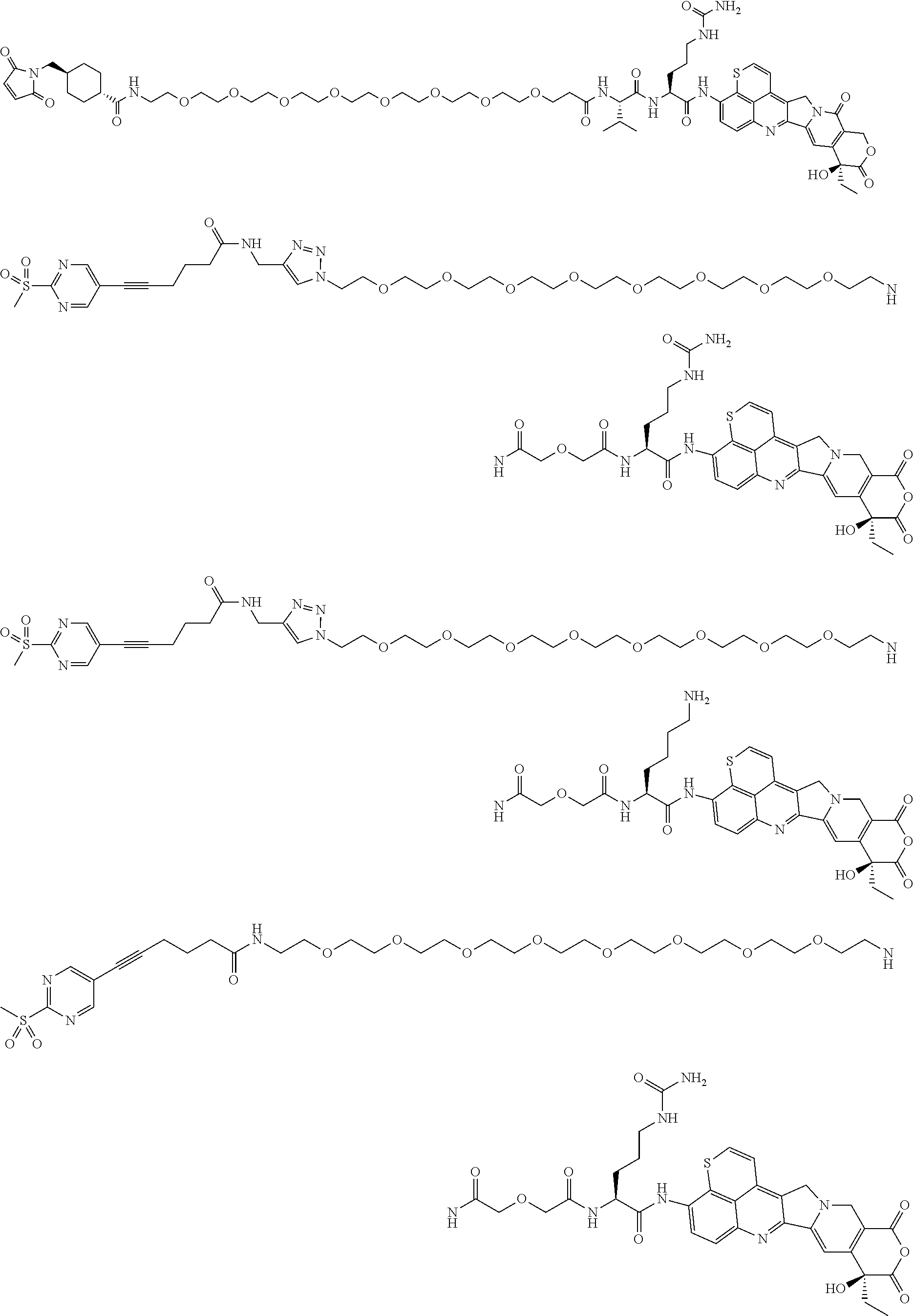

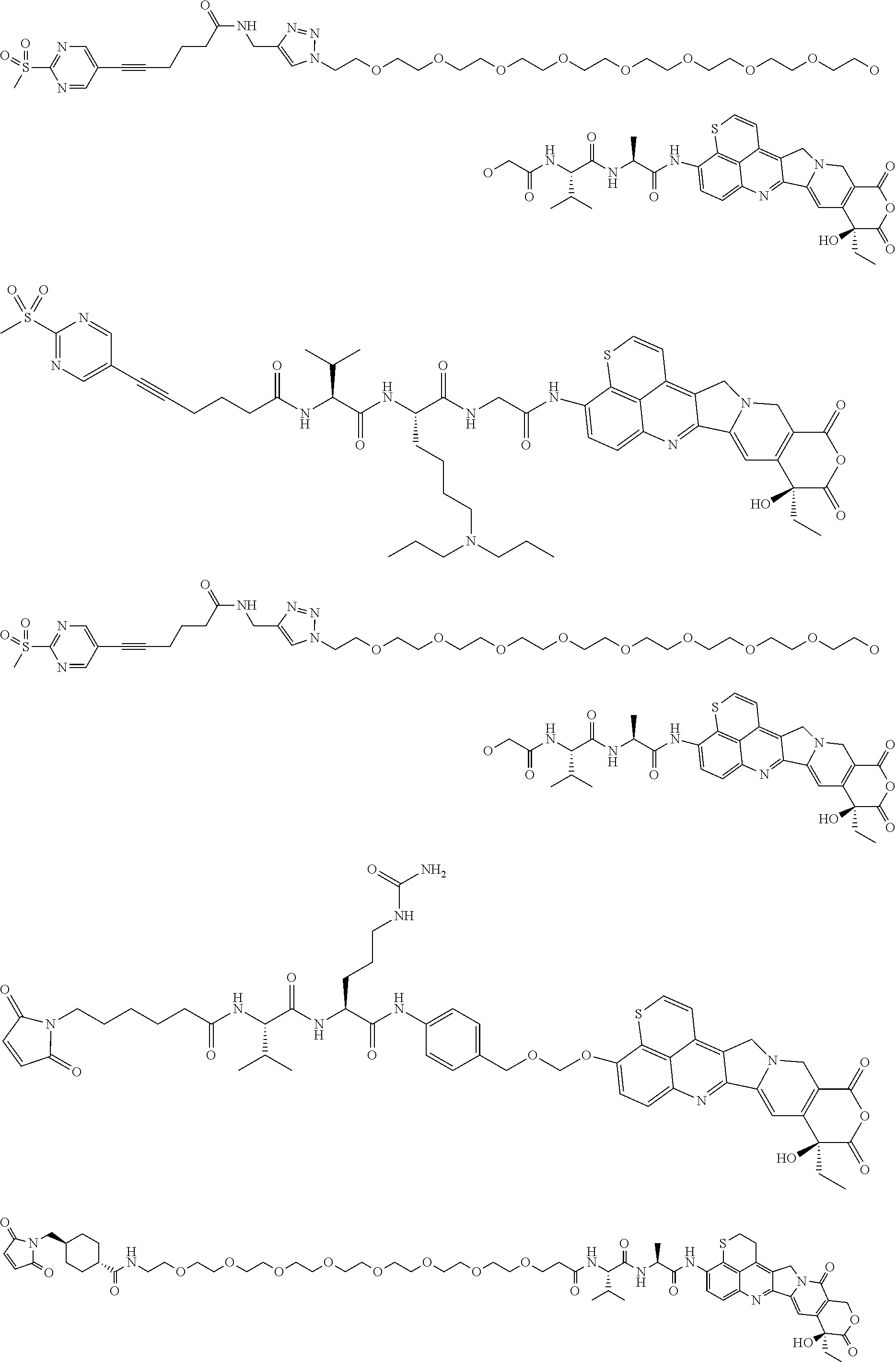

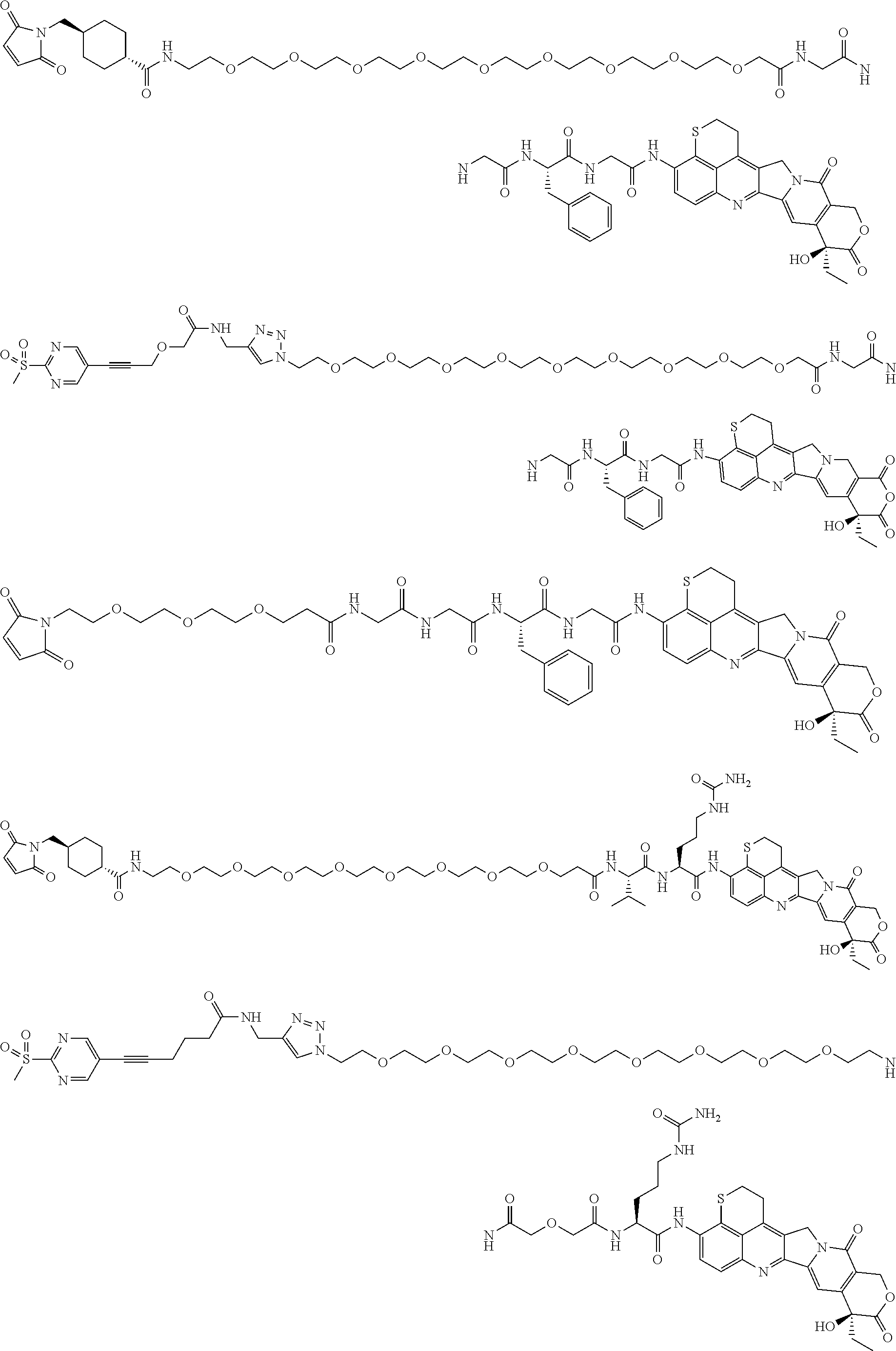

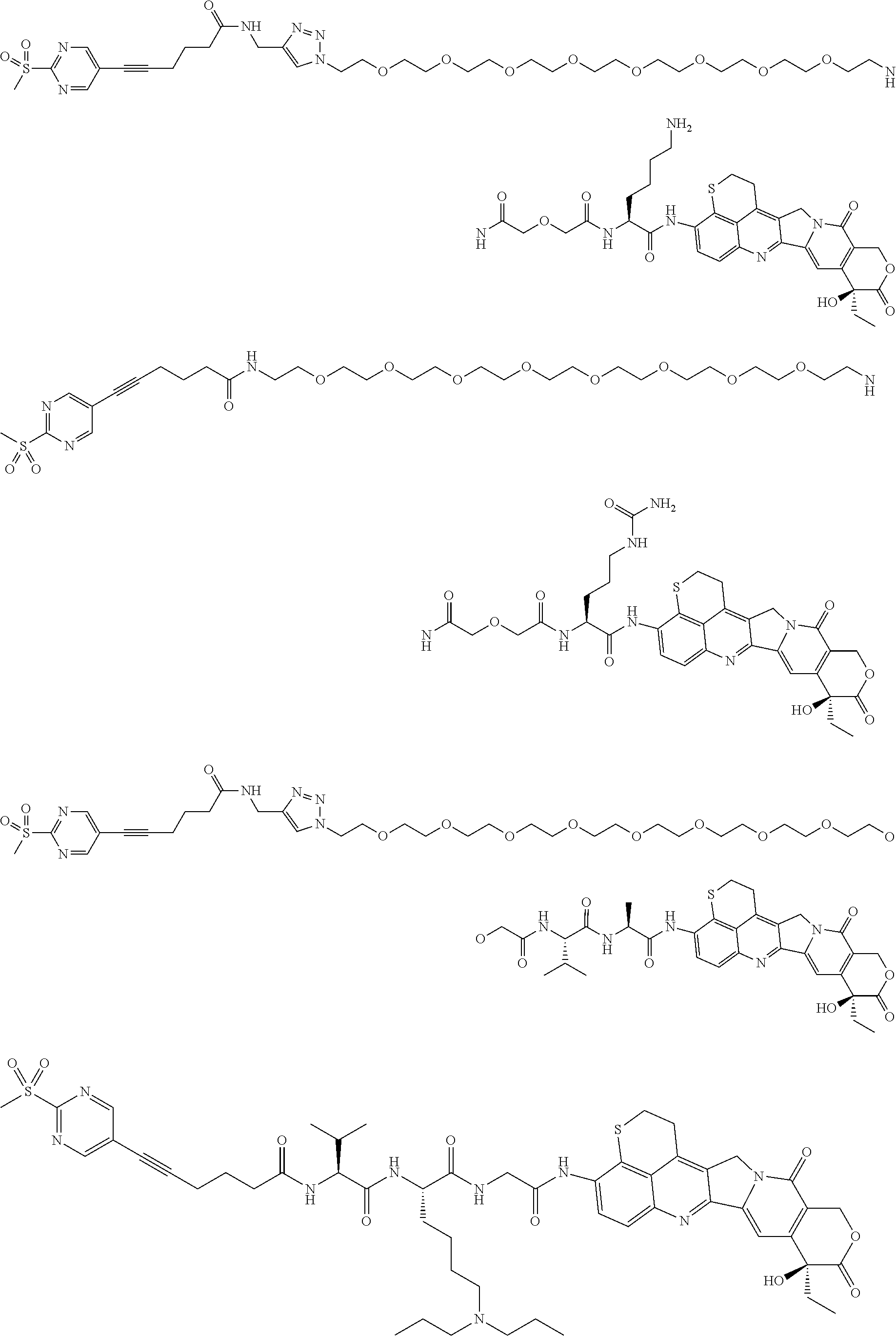

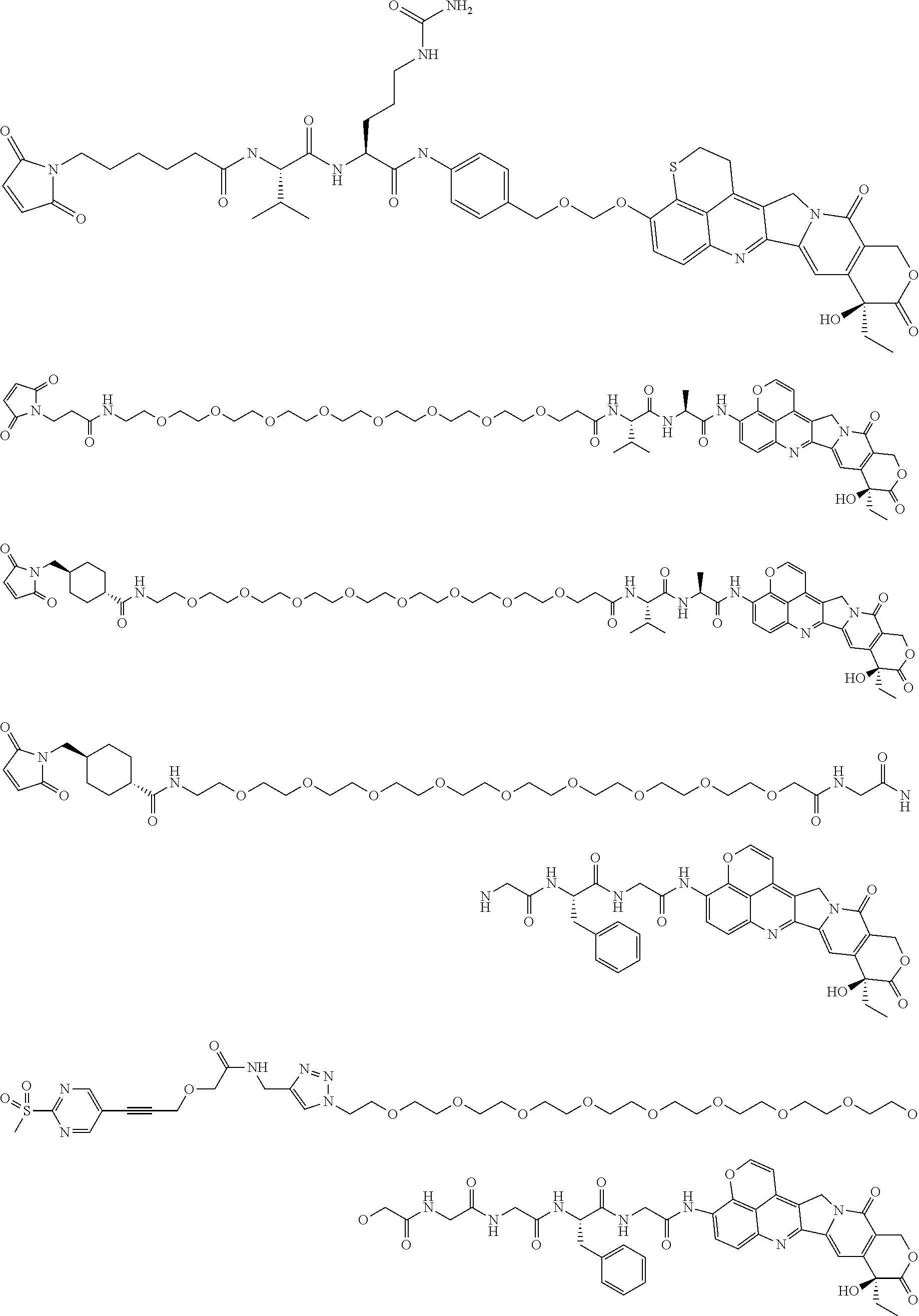

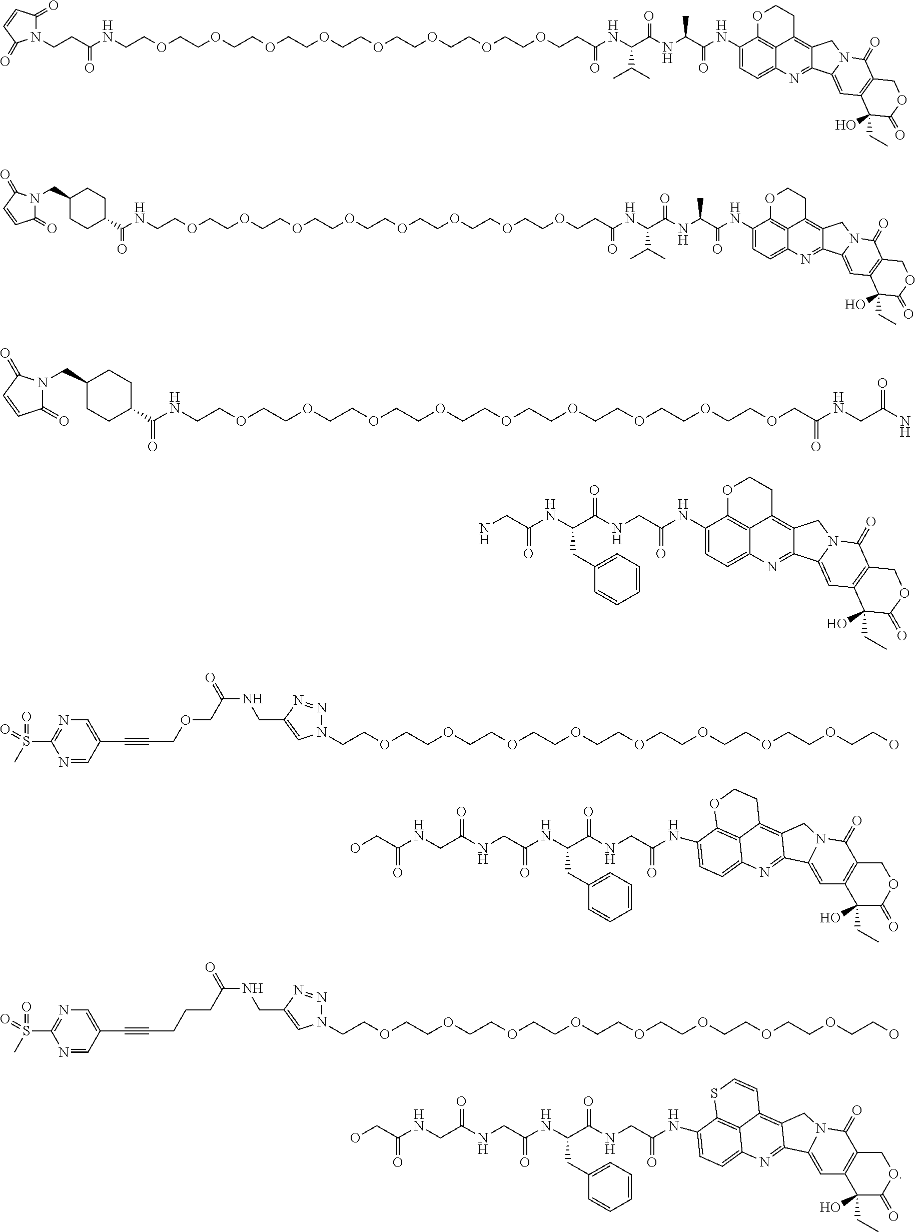

22. A compound of formula (V) or a pharmaceutically acceptable salt, solvate, tautomer, mesomer, racemate, enantiomer, diastereomer, or combinations thereof 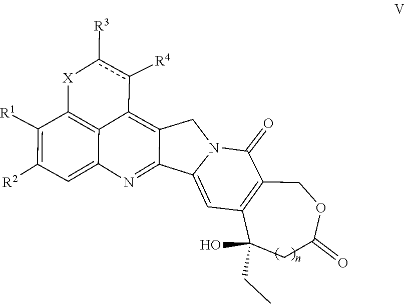
Wherein:
X is either absent, or selected from —O—, —S—, —S(O₂)—, or —S(O)—;
R¹ is —NH—R^{1b}—OH, hydroxy or amino; optionally, R¹ is hydroxy or amino;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R² is selected from H, halo, hydroxy, cyano, nitro, C₃-C₈ cycloalkyl, C₁-C₈ alkyl, C₁-C₈ alkoxy, R⁵—(C₁-C₈ alkylene)-, amino, (C₁-C₈ alkyl)NHC(O)O—, (C₃-C₈ cycloalkyl)NHC(O)O—, (C₁-C₈ alkyl)NH—, azetidin-1-yl, pyrrolidin-1-yl, piperidin-1-yl, azepane-1-yl, (C₁-C₈ alkyl)C(O)O—, or (C₁-C₈ alkyl)C(O)NH—; wherein azetidin-1-yl, pyrrolidin-1-yl, piperidin-1-yl, azepane-1-yl, C₁-C₈ alkoxy, each “C₃-C₈ cycloalkyl”, and each “C₁-C₈ alkyl” are independently optionally substituted with one to four R⁶;
R³ and R⁴ are independently selected from H, halo, hydroxy, cyano, C₁-C₈ alkyl, C₁-C₈ alkoxy, C₃-C₈ cycloalkyl, aryl, heteroaryl, or amino; wherein C₁-C₈ alkyl, C₁-C₈ alkoxy, C₃-C₈ cycloalkyl, aryl, and heteroaryl are independently optionally substituted with one to four R⁶;
R⁵ is selected from C₁-C₈ alkoxy, C₃-C₈ cycloalkyl, aryl, or heteroaryl; wherein C₁-C₈ alkoxy, C₃-C₈ cycloalkyl, aryl, and heteroaryl are independently optionally substituted with one to four R⁶;
R⁶ at each occurrence is independently selected from halo, hydroxy, cyano, amino, C₁-C₈ alkyl, C₃-C₈ cycloalkyl, aryl, or heteroaryl;
and wherein a line with a dotted line represents either a single bond or a double bond;
n is an integer from 0 or 1;
provided that when X is —O— or —S—, and a line with a dotted line represents a single bond, then R², R³, and R⁴ are not all H and are not amino.

23. The compound or a pharmaceutically acceptable salt, solvate, tautomer, mesomer, racemate, enantiomer, diastereomer, or combinations thereof according to claim 22, wherein the compound represented by Formula VII: 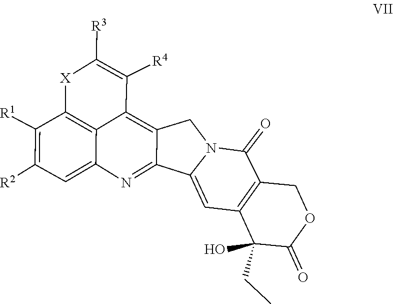
Wherein:
X is selected from —O—, —S—, —S(O₂)—, or —S(O)—;
R¹ is —NH—Rib-OH, hydroxy or amino; optionally, R¹ is hydroxy or amino;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R² is selected from H, halo, hydroxy, cyano, nitro, C₃-C₆ cycloalkyl, C₁-C₆ alkyl, C₁-C₆ alkoxy, R⁵—(C₁-C₃ alkylene)-, amino; wherein C₁-C₆ alkoxy, C₃-C₆ cycloalkyl, and C₁-C₆ alkyl are independently optionally substituted with one to four R⁶;
R³ and R⁴ are independently selected from H, halo, hydroxy, cyano, C₁-C₆ alkyl, C₁-C₆ alkoxy, or C₃-C₆ cycloalkyl; wherein C₁-C₆ alkyl, C₁-C₆ alkoxy, and C₃-C₆ cycloalkyl are independently optionally substituted with one to four R⁶;
R⁵ is C₁-C₆ alkoxy or C₃-C₆ cycloalkyl; wherein C₁-C₆ alkoxy and C₃-C₆ cycloalkyl are independently optionally substituted with one to four R⁶;
R⁶ at each occurrence is independently selected from halo, hydroxy, cyano, amino, C₁-C₃ alkyl, or C₃-C₆ cycloalkyl.

24. The compound or a pharmaceutically acceptable salt, solvate, tautomer, mesomer, racemate, enantiomer, diastereomer, or combinations thereof according to claim 22, wherein R², R³ and R⁴ are independently selected from H, halo, C₁-C₃ alkyl, or C₁-C₃ alkoxy and wherein C₁-C₃ alkoxy and C₁-C₃ alkyl are independently optionally substituted with one to four halo.

25. The compound or a pharmaceutically acceptable salt, solvate, tautomer, mesomer, racemate, enantiomer, diastereomer, or combinations thereof according to claim 22, wherein:
X is —S—;
R¹ is —NH—R^{1b}—OH or amino; optionally, R¹ is amino;
R^{1b} is selected from —C(O)—(C₁-C₆ alkylene)-, —C(O)—O—(C₁-C₆ alkylene)-, —C(O)—S—(C₁-C₆ alkylene)-, —C(O)—NH—(C₁-C₆ alkylene)-, —C(O)—(C₃-C₈ cycloalkylene)-, or —C(S)—NH—(C₁-C₆ alkylene)-; wherein C₁-C₆ alkylene and C₃-C₈ cycloalkylene are independently optionally substituted with one to four R⁶;
R², R³ and R⁴ are independently selected from H, halo, C₁-C₃ alkyl, or C₁-C₃ alkoxy and wherein C₁-C₃ alkoxy and C₁-C₃ alkyl are independently optionally substituted with one to four halo.

26. The compound or a pharmaceutically acceptable salt, solvate, tautomer, mesomer, racemate, enantiomer, diastereomer, or combinations thereof according to claim 22, the compound selected from structures below:

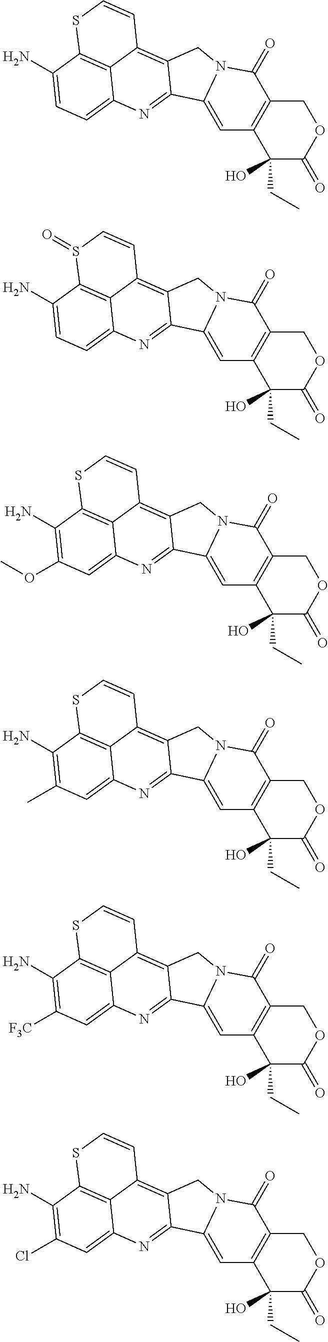 [SMILES: CC[C@@]1(O)C(=O)OCc2c1cc1n(c2=O)Cc2c-1nc1ccc(N)c3c1c2C=CS3 ; CC[C@@]1(O)C(=O)OCc2c1cc1n(c2=O)Cc2c-1nc1ccc(N)c3c1c2C=CS3=O ; CC[C@@]1(O)C(=O)OCc2c1cc1n(c2=O)Cc2c-1nc1cc(OC)c(N)c3c1c2C=CS3 ; CC[C@@]1(O)C(=O)OCc2c1cc1n(c2=O)Cc2c-1nc1cc(C)c(N)c3c1c2C=CS3 ; CC[C@@]1(O)C(=O)OCc2c1cc1n(c2=O)Cc2c-1nc1cc(C(F)(F)F)c(N)c3c1c2C=CS3 ; CC[C@@]1(O)C(=O)OCc2c1cc1n(c2=O)Cc2c-1nc1cc(Cl)c(N)c3c1c2C=CS3]

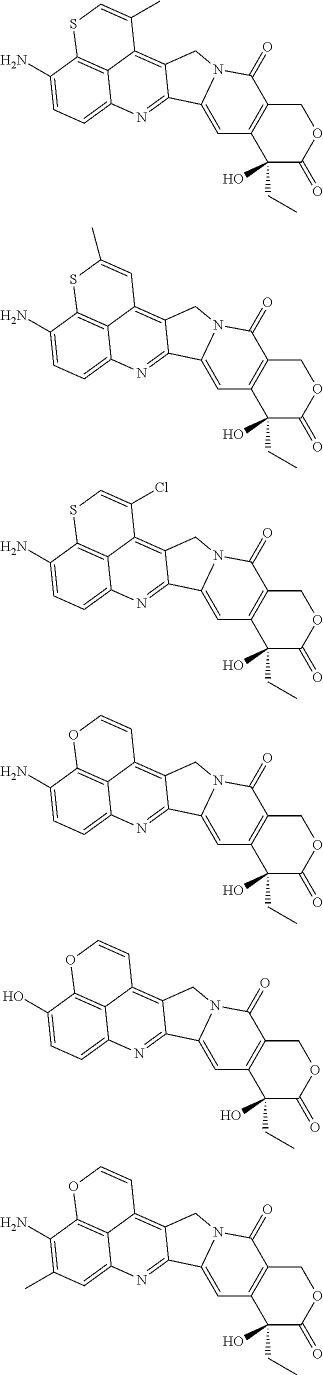 [SMILES: CC[C@@]1(O)C(=O)OCc2c1cc1n(c2=O)Cc2c-1nc1ccc(N)c3c1c2C(C)=CS3 ; CC[C@@]1(O)C(=O)OCc2c1cc1n(c2=O)Cc2c-1nc1ccc(N)c3c1c2C=C(C)S3 ; CC[C@@]1(O)C(=O)OCc2c1cc1n(c2=O)Cc2c-1nc1ccc(N)c3c1c2C(Cl)=CS3 ; CC[C@@]1(O)C(=O)OCc2c1cc1n(c2=O)Cc2c-1nc1ccc(N)c3c1c2C=CO3 ; CC[C@@]1(O)C(=O)OCc2c1cc1n(c2=O)Cc2c-1nc1ccc(O)c3c1c2C=CO3 ; CC[C@@]1(O)C(=O)OCc2c1cc1n(c2=O)Cc2c-1nc1cc(C)c(N)c3c1c2C=CO3]

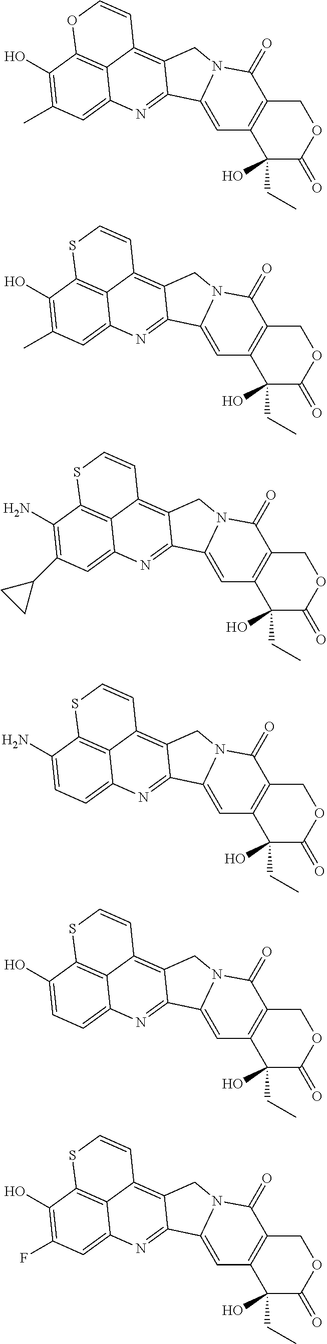 [SMILES: CC[C@@]1(O)C(=O)OCc2c1cc1n(c2=O)Cc2c-1nc1cc(C)c(O)c3c1c2C=CO3 ; CC[C@@]1(O)C(=O)OCc2c1cc1n(c2=O)Cc2c-1nc1cc(C)c(O)c3c1c2C=CS3 ; CC[C@@]1(O)C(=O)OCc2c1cc1n(c2=O)Cc2c-1nc1cc(C3CC3)c(N)c3c1c2C=CS3 ; CC[C@@]1(O)C(=O)OCc2c1cc1n(c2=O)Cc2c-1nc1ccc(N)c3c1c2C=CS3 ; CC[C@@]1(O)C(=O)OCc2c1cc1n(c2=O)Cc2c-1nc1ccc(O)c3c1c2C=CS3 ; CC[C@@]1(O)C(=O)OCc2c1cc1n(c2=O)Cc2c-1nc1cc(F)c(O)c3c1c2C=CS3]

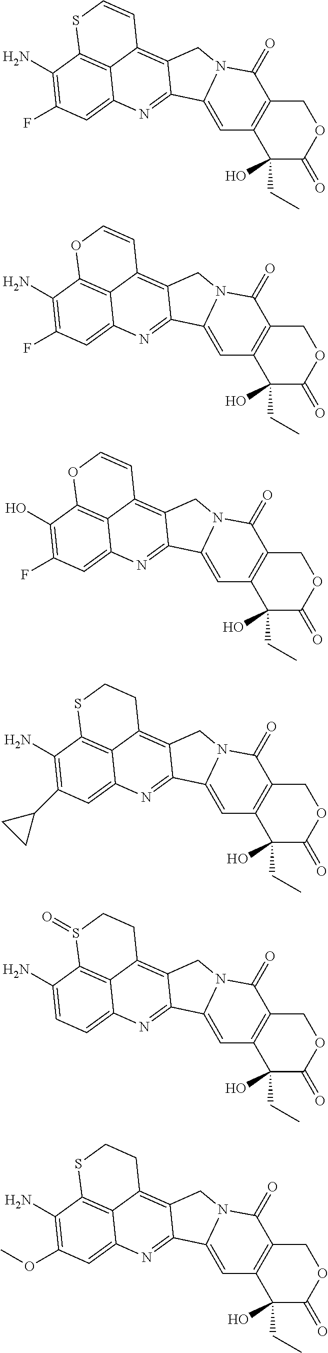 [SMILES: CC[C@@]1(O)C(=O)OCc2c1cc1n(c2=O)Cc2c-1nc1cc(F)c(N)c3c1c2C=CS3 ; CC[C@@]1(O)C(=O)OCc2c1cc1n(c2=O)Cc2c-1nc1cc(F)c(N)c3c1c2C=CO3 ; CC[C@@]1(O)C(=O)OCc2c1cc1n(c2=O)Cc2c-1nc1cc(F)c(O)c3c1c2C=CO3 ; CC[C@@]1(O)C(=O)OCc2c1cc1n(c2=O)Cc2c-1nc1cc(C3CC3)c(N)c3c1c2CCS3 ; CC[C@@]1(O)C(=O)OCc2c1cc1n(c2=O)Cc2c-1nc1ccc(N)c3c1c2CCS3=O ; CC[C@@]1(O)C(=O)OCc2c1cc1n(c2=O)Cc2c-1nc1cc(OC)c(N)c3c1c2CCS3]

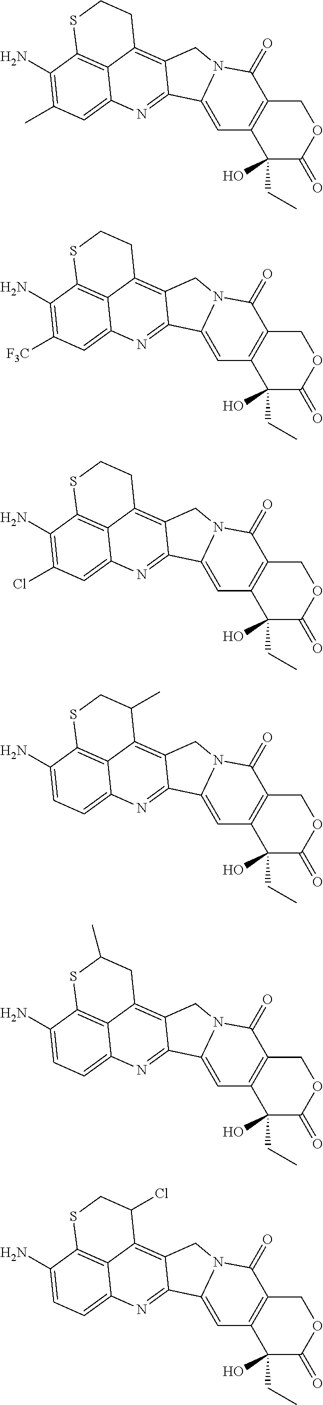 [SMILES: CC[C@@]1(O)C(=O)OCc2c1cc1n(c2=O)Cc2c-1nc1cc(C)c(N)c3c1c2CCS3 ; CC[C@@]1(O)C(=O)OCc2c1cc1n(c2=O)Cc2c-1nc1cc(C(F)(F)F)c(N)c3c1c2CCS3 ; CC[C@@]1(O)C(=O)OCc2c1cc1n(c2=O)Cc2c-1nc1cc(Cl)c(N)c3c1c2CCS3 ; CC[C@@]1(O)C(=O)OCc2c1cc1n(c2=O)Cc2c-1nc1ccc(N)c3c1c2C(C)CS3 ; CC[C@@]1(O)C(=O)OCc2c1cc1n(c2=O)Cc2c-1nc1ccc(N)c3c1c2CC(C)S3 ; CC[C@@]1(O)C(=O)OCc2c1cc1n(c2=O)Cc2c-1nc1ccc(N)c3c1c2C(Cl)CS3]

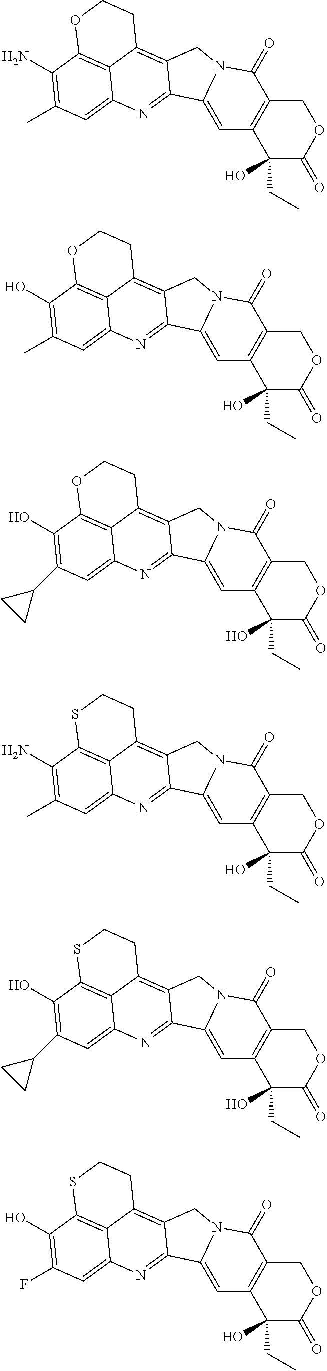 [SMILES: CC[C@@]1(O)C(=O)OCc2c1cc1n(c2=O)Cc2c-1nc1cc(C)c(N)c3c1c2CCO3 ; CC[C@@]1(O)C(=O)OCc2c1cc1n(c2=O)Cc2c-1nc1cc(C)c(O)c3c1c2CCO3 ; CC[C@@]1(O)C(=O)OCc2c1cc1n(c2=O)Cc2c-1nc1cc(C3CC3)c(O)c3c1c2CCO3 ; CC[C@@]1(O)C(=O)OCc2c1cc1n(c2=O)Cc2c-1nc1cc(C)c(N)c3c1c2CCS3 ; CC[C@@]1(O)C(=O)OCc2c1cc1n(c2=O)Cc2c-1nc1cc(C3CC3)c(O)c3c1c2CCS3 ; CC[C@@]1(O)C(=O)OCc2c1cc1n(c2=O)Cc2c-1nc1cc(F)c(O)c3c1c2CCS3]

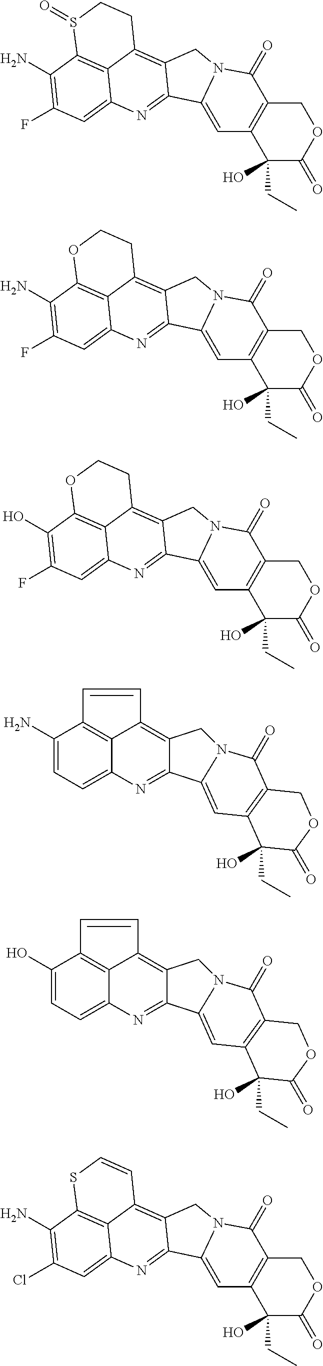 [SMILES: CC[C@@]1(O)C(=O)OCc2c1cc1n(c2=O)Cc2c-1nc1cc(F)c(N)c3c1c2CCS3=O ; CC[C@@]1(O)C(=O)OCc2c1cc1n(c2=O)Cc2c-1nc1cc(F)c(N)c3c1c2CCO3 ; CC[C@@]1(O)C(=O)OCc2c1cc1n(c2=O)Cc2c-1nc1cc(F)c(O)c3c1c2CCO3 ; CC[C@@]1(O)C(=O)OCc2c1cc1n(c2=O)Cc2c-1nc1ccc(N)c3c1c2C=C3 ; CC[C@@]1(O)C(=O)OCc2c1cc1n(c2=O)Cc2c-1nc1ccc(O)c3c1c2C=C3 ; CC[C@@]1(O)C(=O)OCc2c1cc1n(c2=O)Cc2c-1nc1cc(Cl)c(N)c3c1c2C=CS3]

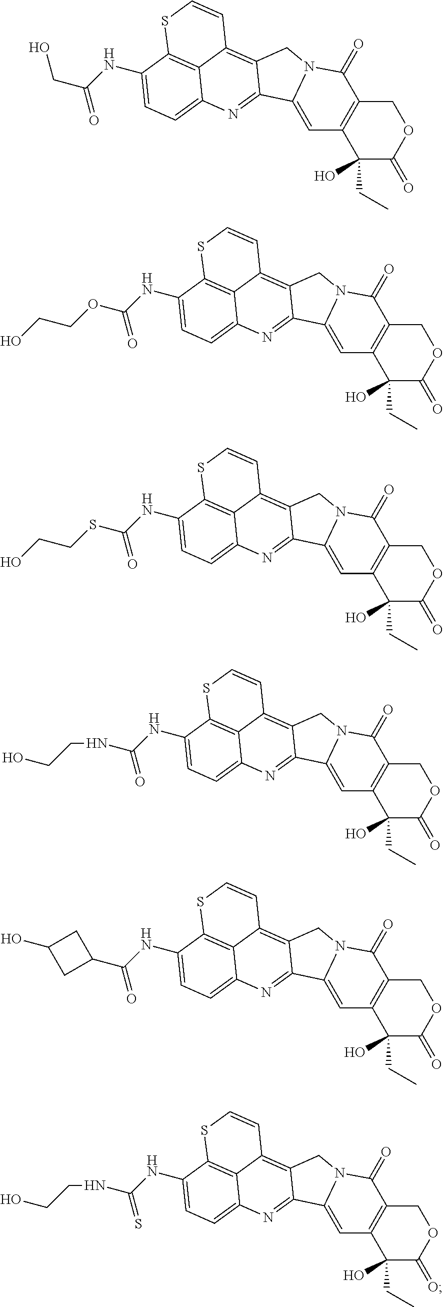 [SMILES: CC[C@@]1(O)C(=O)OCc2c1cc1n(c2=O)Cc2c-1nc1ccc(NC(=O)CO)c3c1c2C=CS3 ; CC[C@@]1(O)C(=O)OCc2c1cc1n(c2=O)Cc2c-1nc1ccc(NC(=O)OCCO)c3c1c2C=CS3 ; CC[C@@]1(O)C(=O)OCc2c1cc1n(c2=O)Cc2c-1nc1ccc(NC(=O)SCCO)c3c1c2C=CS3 ; CC[C@@]1(O)C(=O)OCc2c1cc1n(c2=O)Cc2c-1nc1ccc(NC(=O)NCCO)c3c1c2C=CS3 ; CC[C@@]1(O)C(=O)OCc2c1cc1n(c2=O)Cc2c-1nc1ccc(NC(=O)C3CC(O)C3)c3c1c2C=CS3 ; CC[C@@]1(O)C(=O)OCc2c1cc1n(c2=O)Cc2c-1nc1ccc(NC(=S)NCCO)c3c1c2C=CS3]

27. A pharmaceutical composition, comprising a therapeutically effective amount of the ligand-drug conjugate or the pharmaceutically acceptable salt or solvate thereof according to claim 1, and pharmaceutically acceptable carrier(s), diluent(s), or excipient(s).

28. A pharmaceutical composition, comprising a therapeutically effective amount of the compound or the pharmaceutically acceptable salt, solvate, tautomer, mesomer, racemate, enantiomer, diastereomer, or combinations thereof according to claim 22, and pharmaceutically acceptable carrier(s), diluent(s), or excipient(s).

29. A method of treating cancer, the method comprising administering to a subject in need thereof the ligand-drug conjugate or the pharmaceutically acceptable salt or solvate thereof according to claim 1.

30. A method of treating cancer, the method comprising administering to a subject in need thereof the compound or the pharmaceutically acceptable salt, solvate, tautomer, mesomer, racemate, enantiomer, diastereomer, or combinations thereof according to claim 22.

31. The method of claim 29, wherein the cancer is selected from the group consisting of breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer (for example, small cell lung cancer and non-small cell lung cancer), colon cancer, rectal cancer, colorectal cancer, leukemia (for example, acute lymphocytic leukemia, acute myeloid leukemia, acute promyelocytic leukemia, chronic myeloid leukemia, chronic lymphocytic leukemia), bone cancer, skin cancer, thyroid cancer, pancreatic cancer, and lymphoma (for example, Hodgkin's lymphoma, non-Hodgkin's lymphoma, or recurrent anaplastic large cell lymphoma).

32. A method of treating cancer, the method comprising administering to a subject in need thereof the ligand-drug conjugate or the pharmaceutically acceptable salt or solvate thereof according to claim 27.

33. A method of treating cancer, the method comprising administering to a subject in need thereof the compound or the pharmaceutically acceptable salt, solvate, tautomer, mesomer, racemate, enantiomer, diastereomer, or combinations thereof according to claim 28.
